SEQUENCE LISTING (60 sequences)

SEQ ID NO: 1 - protein, 1543 aa, Artificial Sequence
  Description of Artificial Sequence; note = synthetic construct
MTLSRGPYSELDKMSLFQDLKLKRRKIDSRCSSDGESIADTSTSSPDLLAPMSPKLCDSGSAGASLGASLPLPLALPLPMALPLPMSLPLPLTAASSAVTVSLAAVVAAVAETGGAGAGGAGTAVTASGAGPCVSTSSTTAAAATSSTSSLSSSSSSSSSTSSSTSSASPTAGASSTATCPASSSSSSGNGSGGKSGSIKQEHTEIHSSSSAISAAAASTVMSPPPAEATRSSPATPEGGGPAGDGSGATGGGNTSGGSTAGVAINEHQNNGNGSGGSSRASPDSLEEKPSTTTTTGRPTLTPTNGVLSSASAGTGISTGSSAKLSEAGMSVIRSVKEERLLNVSSKMLVFHQQREQETKAVAAAAAAAAAGHVTVLVTPSRIKSEPPPPASPSSTSSTQRERERERDRERDRERERERDRDREREREQSISSSQQHLSRVSASPPTQLSHGSLGPNIVQTHHLHQQLTQPLTLRKSSPPTEHLLSQSMQHLTQQQAIHLHHLLGQQQQQQQASHPQQQQQQQHSPHSLVRVKKEPNVGQRHLSPHHQQQSPLLQHHQQQQQQQQQQQQHLHQQQQQQQHHQQQPQALALMHPASLALRNSNRDAAILFRVKSEVHQQVAAGLPHLMQSAGGAAAAAAAAVAAQRMVCFSNARINGVKPEVIGGPLGNLRPVGVGGGNGSGSVQCPSPHPSSSSSSSQLSPQTPSQTPPRGTPTVIMGESCGVRTMVWGYEPPPPSAGQSHGQHPQQQQQSPHHQPQQQQQQQQQQSQQQQQQQQQQSLGQQQHCLSSPSAGSLTPSSSSGGGSVSGGGVGGPLTPSSVAPQNNEEAAQLLLSLGQTRIQDMRSRPHPFRTPHALNMERLWAGDYSQLPPGQLQALNLSAQQQQWGSSNSTGLGGVGGGMGGRNLEAPHEPTDEDEQPLVCMICEDKATGLHYGIITCEGCKGFFKRTVQNRRVYTCVADGTCEITKAQRNRCQYCRFKKCIEQGMVLQAVREDRMPGGRNSGAVYNLYKVKYKKHKKTNQKQQQQAAQQQQQQAAAQQQHQQQQQHQQHQQHQQQQLHSPLHHHHHQGHQSHHAQQQHHPQLSPHHLLSPQQQQLAAAVAAAAQHQQQQQQQQQQQQQAKLMGGVVDMKPMFLGPALKPELLQAPPMHSPAQQQQQQQQQQQQQQASPHLSLSSPHQQQQQQQGQHQNHHQQQGGGGGGAGGGAQLPPHLVNGTILKTALTNPSEIVHLRHRLDSAVSSSKDRQISYEHALGMIQTLIDCDAMEDIATLPHFSEFLEDKSEISEKLCNIGDSIVHKLVSWTKKLPFYLEIPVEIHTKLLTDKWHEILILTTAAYQALHGKRRGEGGGSRHGSPASTPLSTPTGTPLSTPIPSPAQPLHKDDPEFVSEVNSHLSTLQTCLTTLMGQPIAMEQLKLDVGHMVDKMTQITIMFRRIKLKMEEYVCLKVYILLNKGTWFDLQNPFIQCSCYLLVRFVNPAEVELESIQERYVQVLRSYLQNSSPQNPQARLSELLSHIPEIQAAASLLLESKMFYVPFVLNSASIR

SEQ ID NO: 2 - DNA, 4632 bp, Artificial Sequence
  Description of Artificial Sequence; note = synthetic construct
atgacactgagccgtggcccgtacagcgagctcgataaaatgagcctttttcaagacctcaaactcaaacggcgcaaaatcgattcgcgatgcagcagtgacggcgagtccatagcggacacgtccacctcgtcgccggacctgctggcgcccatgtcgccgaagctctgcgacagcggctcggcgggggcgtcgctgggggcatcgctgcccctgccgctggccctgcccctgccaatggccctgccactgcccatgtcgctgcccctgcccctcacggcggcatcttcggcggtcaccgtttcgctggcagcggtcgtggccgcggtggccgagacgggtggcgcgggcgcgggaggagctgggacagcagtaacagcgtcgggagcaggaccatgcgtctccacgtcgtctacgacggcagcggcagccacatcctcgacctcctcgctctcgtcctcctcctcttcgtcatcctccacgtcctccagcacttcctccgcctcgccgacagctggagcctcctccacggccacctgccccgccagcagcagcagcagcagtggaaacggaagtgggggcaaaagtggtagcatcaagcaggagcacacggagatacactcgtcgagcagtgcgatttcggcggccgccgcctcaacggtgatgtcaccgccgcccgctgaggcgacgagatccagtccagccacgcccgagggaggcggaccagctggcgacggaagtggagcaacgggaggcggaaacacgagcggcggatcaacggctggagtggccattaatgaacaccaaaacaatggcaatggcagcggcgggagcagtcgagcctctcccgattcgctggaagagaagccctctaccacaacgaccacaggtcgtccaacgctcacgcccacgaatggggtgctgtcctccgcctcggcgggcacggggatttccacaggaagcagcgccaagctgagcgaggctggtatgagtgtgatacggtccgtgaaggaggagcgcttgctcaacgtatccagcaagatgctggtgttccatcagcagcgggagcaagagaccaaagcagtggcggctgcagcagcagcagcagcggcgggccatgtgacggttctagtgacgccatcgcgcatcaaatcggagccaccgccgccggcttcaccctcctctacatccagcacacaaagggaaagggaacgggaacgcgatcgagagagggatcgcgaaagggaacgcgagcgggaccgggaccgggaacgggaacgggaacagtccatcagctcctcgcagcagcacctaagtcgggtctccgccagtccacccactcagctgtcccacggcagcctgggacccaacattgtgcagacgcaccatcttcaccagcaactcacacagccgctgacgctgcgcaagagcagcccgcccacagagcacctgctcagtcagtccatgcaacatctcacacagcagcaggcgatccacctgcatcacctacttggccagcagcagcagcagcagcaggcgtcgcatccccagcagcaacagcagcagcaacactcgccccactccctggtgcgggtgaaaaaggaaccgaatgttggtcagcggcacttatcgccgcatcaccaacaacagtcgccactcctgcagcaccaccaacagcagcagcagcagcaacaacaacagcaacagcatctgcatcagcaacagcaacagcagcagcatcaccagcagcagccccaggcactggccctgatgcatccggcttccctggcgctaaggaacagcaatcgggatgcggccattctgtttcgggtgaagagcgaagtgcaccagcaggtggccgccgggctgccgcatctgatgcagtccgctggtggggcagcggccgccgccgcagcagctgtggccgctcagcgaatggtatgcttcagcaatgccaggatcaatggcgttaagccggaggtgattggaggaccgctgggcaacctgcggcccgtgggcgtcggtggcggaaacggaagtggctccgtgcagtgcccctcgccgcatccatcctcctcgtcgtcatcctcgcagctgtcgccgcagacgccctcccagacgccgccccgaggcacgcccaccgtcataatgggcgagagctgcggggtgcgcaccatggtctggggctacgagcctccgccaccctcggcgggccagtcccacggccagcacccgcaacagcaacagcagtcgccccaccaccagccgcaacaacaacagcagcagcaacaacagcagtcgcagcagcaacagcaacagcagcagcaacagtcgctgggccagcagcagcactgcctctcctcgccgtcggcgggatcgctgacgccctcctcttcgtccggcggtggttcggtatctggcggcggagtgggcggaccactcacaccctcctcggtggcgccgcagaataacgaggaggccgcccaactcctgctctccctgggacagacacgcatccaggacatgagatcacggccacaccccttccgcacaccgcacgcccttaatatggagcggctgtgggcgggagactactcgcaattgccgcccggccagctgcaggctctgaatctcagtgcccaacagcagcagtggggcagcagcaactccacgggtcttggtggcgtaggcggcggcatgggcggacgcaacctggaggcgccgcacgagccgaccgacgaggacgaacagccgctcgtttgcatgatctgcgaggacaaggccaccggcctgcactacggcatcatcacctgcgaggggtgcaagggcttcttcaagcggacggtgcagaaccgacgagtctacacctgcgtggcggacggcacctgcgagataaccaaagcacagcgcaaccgttgtcagtattgtcgatttaagaagtgcatcgagcagggcatggtgctgcaagccgttcgcgaggatcgcatgccgggcggtcgcaacagtggcgccgtctacaatttgtacaaggtgaagtacaagaagcacaagaagaccaatcagaagcagcagcagcaggccgcccagcagcagcagcagcaggcggcggcgcagcagcagcaccagcaacagcagcagcatcaacagcaccagcaacatcagcaacagcagttgcactcgccgctccaccatcaccaccaccagggccaccagtcgcaccacgcgcagcagcagcaccacccacagctgtcgccgcaccacctgctgtcgccgcagcagcagcaacttgccgccgcggtggcagcagctgcgcagcaccaacagcaacagcaacaacagcagcaacagcagcagcaggccaagctgatgggcggcgtggtggacatgaagcccatgttcctcggccccgctttgaagccggagttgctgcaagcaccccccatgcacagtccggcccagcaacaacaacagcagcagcagcagcagcagcaacagcaggcctcgccgcatctctcgcttagctcaccgcaccagcagcagcagcagcagcagggacagcaccaaaaccaccaccagcaacaaggtgggggtggcggaggagctggtggaggagctcaactgccgccgcacctggtgaacggaacgatactgaagacggccctaaccaatcccagcgagattgtacatctgcgccaccgcctcgactcggcggtcagttcgtccaaggaccgacagatctcgtacgagcacgccttaggcatgatccagacactgatcgactgcgacgcgatggaggacatagccacactgccgcacttcagcgagttccttgaggacaagtcggagattagcgagaaactgtgcaacatcggcgattccatagtccacaagctggtgtcgtggacaaaaaagttgcccttctacctggagatcccggtggagatacataccaaactactgacggacaagtggcacgagatccttatcctgaccacggccgcctaccaggcgttgcatggcaagcggcgtggcgagggaggaggcagcaggcatggttcgccggcgtcaacgccactgagcacgcccactggtacgccgttgagcacaccgataccctcgcccgcccagccactgcacaaggacgacccggagtttgtcagcgaggtgaactcgcacctgagcacactgcaaacctgcttgaccacgctaatgggccagccgatagcgatggagcagctgaagctggacgtcgggcacatggtggacaagatgacccagatcaccatcatgttccggcgaatcaagctcaagatggaggagtacgtctgcctgaaggtttacatactgctaaacaaaggtacgtggttcgatttgcaaaacccattcatacagtgctcatgttaccttctcgttcgttttgtaaatccagcagaagtggaactggagagcatccaggagcggtacgtccaggtgctgcgctcctacctgcaaaactcctcgccgcagaatccgcaggcgaggctcagtgaactgctctcccacataccagagatccaggctgcggctagcctgctgctcgagagcaagatgttctatgtgcccttcgtgctcaactcggcgagcataaggtag

SEQ ID NO: 3 - protein, 803 aa, Artificial Sequence
  Description of Artificial Sequence; note = synthetic construct
MLLEMDQQQATVQFISSLNISPFSMQLEQQQQPSSPALAAGGNSSNNAASGSNNNSASGNNTSSSSNNNNNNNNDNDAHVLTKFEHEYNAYTLQLAGGGGSGSGNQQHHSNHSNHGNHHQQQQQQQQQQQQHQQQQQEHYQQQQQQNIANNANQFNSSSYSYIYNFDSQYIFPTGYQDTTSSHSQQSGGGGGGGGGNLLNGSSGGSSAGGGYMLLPQAASSSGNNGNPNAGHMSSGSVGNGSGGAGNGGAGGNSGPGNPMGGTSATPGHGGEVIDFKHLFEELCPVCGDKVSGYHYGLLTCESCKGFFKRTVQNKKVYTCVAERSCHIDKTQRKRCPYCRFQKCLEVGMKLEAVRADRMRGGRNKFGPMYKRDRARKLQVMRQRQLALQALRNSMGPDIKPTPISPGYQQAYPNMNIKQEIQIPQVSSLTQSPDSSPSPIAIALGQVNASTGGVIATPMNAGTGGSGGGGLNGPSSVGNGNSSNGSSNGNNNSSTGNGTSGGGGGNNAGGGGGGTNSNDGLHRNGGNGNSSCHEAGIGSLQNTADSKLCFDSGTHPSSTADALIEPLRVSPMIREFVQSIDDREWQTQLFALLQKQTYNQVEVDLFELMCKVLDQNLFSQVDWARNTVFFKDLKVDDQMKLLQHSWSDMLVLDHLHHRIHNGLPDETQLNNGQVFNLMSLGLLGVPQLGDYFNELQNKLQDLKFDMGDYVCMKFLILLNPSVRGIVNRKTVSEGHDNVQAALLDYTLTCYPSVNDKFRGLVNILPEIHAMAVRGEDHLYTKHCAGSAPTQTLLMEMLHAKRKG

SEQ ID NO: 4 - DNA, 3269 bp, Artificial Sequence
  Description of Artificial Sequence; note = synthetic construct
ctacgcaaaataaaacgtacatgaaatgttattagaaatggatcagcaacaggcgaccgtacagtttatatcgtcgctgaatatatcgccgttcagcatgcagctggagcagcagcagcagccctccagtcccgctctggccgccggtggcaacagcagcaacaacgcggccagcggtagcaacaacaacagcgccagcggcaacaacaccagcagcagcagcaacaacaacaacaacaataacaacgacaatgatgcacacgttctaacgaaattcgagcacgaatacaatgcctacacgttgcagttggccggaggcggtgggagtggcagcggcaatcagcagcaccacagcaaccacagcaaccacggcaaccaccaccagcagcagcagcaacaacagcaacagcagcagcaacatcagcagcagcagcaagaacactaccagcagcaacagcaacagaatatcgccaacaatgccaatcaattcaactcctcgtcctactcgtatatatacaatttcgattcacagtatatattcccgacaggctaccaggacaccacctcctcacactcgcaacagagcggaggaggcggtggcggcggcggtggcaacctgctaaacggcagctccggcggcagctccgccggcggtggctacatgctgctcccccaggcggccagctccagtggcaataatggcaatccgaatgccggccacatgtcctccggttccgtgggcaatggcagcggaggcgctggcaatggcggagcgggcggcaactccggtcccggcaatcccatgggcggtacgagcgccacgccgggacacggcggcgaggtgatcgacttcaagcacctgttcgaggagctttgccccgtgtgtggcgacaaggtgagcggctaccactacggcctgctcacctgcgagtcctgcaagggattcttcaagcgcaccgtgcagaacaagaaggtctacacctgcgtggcggagcggtcgtgccacatcgacaagacgcagcgcaagcggtgtccctactgccgattccagaagtgcctcgaggtgggcatgaagctagaggctgttcgagcggatagaatgcgtggtggacgcaacaaattcggacccatgtacaaacgggatcgcgcgcggaagttgcaagtgatgcggcagcggcagttggcgctgcaagcgctgcgcaactcgatgggtccggacatcaagccaacgccgatctcgccgggctaccagcaagcatatccaaatatgaacattaagcaggaaattcaaatacctcaggtatcctcactcacccaatctccggactcgtcgcccagccccatagcaattgcgttgggacaggtgaacgcgagcacgggcggtgttatagccacgcccatgaacgccggcactggcggcagtgggggcggtggtctgaacggaccaagttccgtgggcaacggcaatagcagcaacggcagcagcaacggcaacaacaacagcagcacgggcaacggaacgtccggaggaggaggtggcaataatgcgggcggcggaggaggaggaaccaattccaacgatggcctgcatcgcaacggcggcaatggcaacagcagttgccacgaggctggaataggatctctgcagaacacggccgactcgaaattgtgcttcgattctggcacacatccatcgagcacagccgacgcgctaatcgagccattaagagtctcaccgatgattcgtgaatttgtgcaatctattgacgatcgggaatggcagacgcaactgtttgccctgctgcagaagcaaacctacaaccaggtggaagtggatctcttcgagctgatgtgcaaagtgctcgaccagaatttgttctcgcaagtagactgggcacggaacaccgtcttcttcaaggatctgaaggtcgacgaccaaatgaagctgctgcagcattcctggtcggacatgcttgttctggatcacctgcatcatcgaatccataacggcctgcccgacgagacgcaactgaacaatggtcaggtgttcaatctgatgagtctgggtttgttgggagtgccacagctgggcgattacttcaacgagctgcagaacaagctgcaggacctgaaattcgatatgggcgactatgtctgcatgaaattcctaatcctgttgaatccaagtgtacggggtattgtcaaccggaagaccgtctccgagggacatgataatgtgcaagccgctttgctggactacaccctcacctgctatccgtcagtgaatgacaaattcagagggctagttaacatcttaccggaaatccatgccatggccgttcgcggcgaggatcacctgtacaccaagcactgtgccggcagtgcgcccacccaaacgctgctcatggagatgctgcacgccaagcgcaagggatagaggccgggagaacgtgacacggaatacttaatcatttatgaaatgtaaataacaaggcgggaaggccctcggggcaaccgggtcatggaaggcgaacgaaggatacagcagaattccgtattatgaatatgggaatgcatcatcactactaccaccaactatcacacctatacacacacatgcacacatttgttgattcaatgttaattattattacgtttacggttaggtctagtttacgtttaactaattaattaatttgtcttaaattaattcgtgttttatttgtagtccctgataaagcaattttaaaacacttgaacctaaacgagaatatgtagtagatgtatggatttaaatttaaatacggcaaggagaaacacacttttttaggcattacaaaacaaaagaagcatgagaaattttatttttatatacctatatgaatacgatacttatggatacaaatctatatatatttttatgtaaattggcgtacttttagcgtcctacatattttttaattagaatttggttatactatagttttgaaattagtatcgttcccacttgaagatcgattcttgtatttttttgcgccaagtgtcttgcatagtatttgcgtctaatctaatggcaacaaaaaaaatattggaaaatccatacaaagaaaatgaaaacaaagcaaatttaggtgttcatggtatgaatgtatgtgtatattataattgtaatttcatctaagtgtaagaaaacaatgcaaacaactacctacaacaagataatgaagagcaagaaattatataaattaataaaggtcgtgttaaaaact

SEQ ID NO: 5 - protein, 487 aa, Artificial Sequence
  Description of Artificial Sequence; note = synthetic construct
MYTQRMFDMWSSVTSKLEAHANNLGQSNVQSPAGQNNSSGSIKAQIEIIPCKVCGDKSSGVHYGVITCEGCKGFFRRSQSSVVNYQCPRNKQCVVDRVNRNRCQYCRLQKCLKLGMSRDAVKFGRMSKKQREKVEDEVRFHRAQMRAQSDAAPDSSVYDTQTPSSSDQLHHNNYNSYSGGYSNNEVGYGSPYGYSASVTPQQTMQYDISADYVDSTTYEPRSTIIDPEFISHADGDINDVLIKTLAEAHANTNTKLEAVHDMFRKQPDVSRILYYKNLGQEELWLDCAEKLTQMIQNIIEFAKLIPGFMRLSQDDQILLLKTGSFELAIVRMSRLLDLSQNAVLYGDVMLPQEAFYTSDSEEMRLVSRIFQTAKSIAELKLTETELALYQSLVLLWPERNGVRGNTEIQRLFNLSMNAIRQELETNHAPLKGDVTVLDTLLNNIPNFRDISILHMESLSKFKLQHPNVVFPALYKELFSIDSQQDLT

SEQ ID NO: 6 - DNA, 4262 bp, Artificial Sequence
  Description of Artificial Sequence; note = synthetic construct
gaattcattcaactgcaaagagcagccaaattgcgcatacgccgcgtatggccgtcggtgtgagtgcccgtgttcatcagcggttgcatcaactgataccaagtgtacataactacagctacaattgcaactatttcaccaatcaacggcagcggcaacaacatcagcaacagcaccggcaaacgtttgaaacgtcaccaaagcttcgcatttcccactaataattatgtatacgcaacgtatgtttgacatgtggagcagcgtcacttcgaaactggaagcacacgcaaacaatctcggtcaaagcaacgtccaatcgccggcgggacaaaacaactccagcggttccattaaagctcaaattgagataattccatgcaaagtctgcggcgacaagtcatccggcgtgcattacggagtgatcacctgcgagggctgcaagggattctttcgaagatcgcagagctccgtggtcaactaccagtgtccgcgcaacaagcaatgtgtggtggaccgtgttaatcgcaaccgatgtcaatattgtagactgcaaaagtgcctaaaactgggaatgagccgtgatgctgtaaagttcggcaggatgtccaagaagcagcgcgagaaggtcgaggacgaggtacgcttccatcgggcccagatgcgggcacaaagcgacgcggcaccggatagctccgtatacgacacacagacgccctcgagcagcgaccagctgcatcacaacaattacaacagctacagcggcggctactccaacaacgaggtgggctacggcagtccctacggatactcggcctccgtgacgccacagcagaccatgcagtacgacatctcggcggactacgtggacagcaccacctacgagccgcgcagtacaataatcgatcccgaatttattagtcacgcggatggcgatatcaacgatgtgctgatcaagacgctggcggaggcgcatgccaacacaaataccaaactggaagctgtgcacgacatgttccgaaagcagccggatgtgtcgcgcattctctactacaagaatctgggccaagaggaactctggctggactgcgccgagaagcttacacaaatgatacagaacataatcgaatttgctaagctcataccgggattcatgcgcctaagtcaggacgatcagatattactgctgaagacgggctcctttgagctggcgattgttcgcatgtccagactgcttgatctctcacagaacgcggttctctacggcgacgtgatgctgccccaggaggcgttctacacatccgactcggaagagatgcgtctggtgtcgcgcatcttccaaacggccaagtcgatagccgaactcaaactgactgaaaccgaactggcgctgtatcagagcttagtgctgctctggccagaacgcaatggagtgcgtggtaatacggaaatacagaggcttttcaatctgagcatgaatgcgatccggcaggagctggaaacgaatcatgcgccgctcaagggcgatgtcaccgtgctggacacactgctgaacaatatacccaatttccgcgatatttccatcttgcacatggaatcgctgagcaagttcaagctgcagcacccgaatgtcgtttttccggcgctgtacaaggagctgttctcgatagattcgcagcaggacctgacataacaagagcagcagccgttcctggagacgaccgcggacgatgttgccgaggatgcggctgccgccggatgtgtcctgccgccggtggcgccccctgccgggcagcaaccagcgctgctcgaggactgagggccgcaggatgtggcaacaataattatttgagtaaacactgcactgcgcatgcagcagatacaagaactttatcatgatttaagctagcatacaaccaaggatgtgatcctcgccaaggactcacttaaaaagaactctatctatatacatatatatattatatatgacagagcggatgacgcaaagggaagggaaaatatttcaaaaatattgttaactcagttaagacttttgcttcgtagagaaccgaaaccgaaaccgattgcatttcgagcaaggggcatcaaactgattttcgaggttatactatacatatatacacacaaacacacacacacacatatatatatatgtaacttccaaactttcatatcctggcccgagcagatcagatcgtctaagtacttaaaaccaagcgaaattctctacaccgcacaacccaggacccgtagaccccaataattcagttcggttagtgttaaccccagaaagcccgattccgatcccgcctaggttgtctttgccttacgttgtaactaaagtatgtgtattatatatacagcaaatgtatgtataactatgtcgtatcggttatatgcctaacaacattattttttgtaaacaacaaaatcgaatatctcggaaaatgtgttcttataattatattgattaatgcaattacaatatatttacaatttaccgttacgtttttacattatacataagacgcaagagaaggaaacggaagtttaaggattagaaagctgaataagaaaaggcttaaggacgagctgagtagcagttaaagtgagcgagaaatcgaatgaataccagaaaatttcaagcaagcacataaaagtatgcaatattttgtttaaaaacaactttttattagtttcttaaatataacataattacgtacatacacacacgtatatatagggctatatatatctatatatatatatatatacatgatagacaaatcccaatccggttccaaggtttagtaaaaataaagagaaataaaacgaaaaacaaaaacttttgatatgaaatcctacgcataattaacaacttttattgtttctaagacttaaacttaattaaaatggaaaccaaaacagactgacggaccgaccccgacagcatgccacgccctcccccgccccaccctccacagatcctggcagaaatttcaaaggagtttgatacacaaatcgagaaaagaaattttcaaaaaaataatataaagacaagcaaacggcgacttttttggttgatacatttgaaaagaatatacaattaaatatctgactgactatacaaagacgttacacacacgcatacacatacacacacatacacgcatacacacacagcttacgatacataaattagttaaacttagagtaaacaaacaacaacaaacacattggatagtaggtgataattggtgtgtcttaaataaaccttaacccctccccgacccccgcccacttgcttaatacccaacgccccaaaaagccccacatttctactaaatgaaaagcttaatcaaaacttttttgaaattattcaagtgaaaatttcagcaggcaggcataaatattaattaacattaattatagcaaggaaacttataaataaaatgtatacaacaaaactacaaaaattaaataaattacattttgcaaattccacaaaaaataaaacatgattttgcaaattcacttaaaatcctttccctgaatccaagcaaaaatatttacactagcttacatagaactgggacgaggacatgaatatttcaattgagaaaaaaatctatgttaatgtaatcgatcgatttggacatatttaagttcgacatttttggccttacaaaacaaaaaacaaaaagaagaaacctaaagtactttatatatatacaaaccatatatacaatatagagaatacaaaactagttttaatttatacaaagcaagggagcagctttcaaactcaaaacaaaaatatccccgaaaaaaacaacaactttgttaaaaactgcgcataataaagaaaataataaacaaagttaatctataatataaattgaagttaagttgatttgagcggtcgacaacaagaacataaatgtatctttaaatgatatatgtattgttaaatttgtatgctaagtttttagaaaggttacatttttaaagaataataacaaaagatcgcgaactcgacaaggtgtaaaatgagtacatttaaattaaaatttagcatatataatgcataaatattatgttacgatatttacatttatataaaacaaaacaaaaacactaaagaaaaccgaaaaaacagaagtcccatattaaaaatgaaataaaatgagcagaacctataaactgataagggaattctgaatattaaaaaaaaaaagaaaaca

SEQ ID NO: 7 - protein, 723 aa, Artificial Sequence
  Description of Artificial Sequence; note = synthetic construct
MSPPKNCAVCGDKALGYNFNAVTCESCKAFFRRNALAKKQFTCPFNQNCDITVVTRRFCQKCRLRKCLDIGMKSENIMSEEDKLIKRRKIETNRAKRRLMENGTDACDADGGEERDHKAPADSSSSNLDHYSGSQDSQSCGSADSGANGCSGRQASSPGTQVNPLQMTAEKIVDQIVSDPDRASQAINRLMRTQKEAISVMEKVISSQKDALRLVSHLIDYPGDALKIISKFMNSPFNALTVFTKFMSSPTDGVEIISKIVDSPADVVEFMQNLMHSPEDAIDIMNKFMNTPAEALRILNRILSGGGANAAQQTADRKPLLDKEPAVKPAAPAERADTVIQSMLGNSPPISPHDAAVDLQYHSPGVGEQPSTSSSHPLPYIANSPDFDLKTFMQTNYNDEPSLDSDFSINSIESVLSEVIRIEYQAFNSIQQAASRVKEEMSYGTQSTYGGCNSAANNSQPHLQQPICAPSTQQLDRELNEAEQMKLRELRLASEALYDPVDEDLSALMMGDDRIKPDDTRHNPKLLQLINLTAVAIKRLIKMAKKITAFRDMCQEDQVALLKGGCTEMMIMRSVMIYDDDRAAWKVPHTKENMGNIRTDLLKFAEGNIYEEHQKFITTFDEKWRMDENIILIMCAIVLFTSARSRVIHKDVIRLEQNSYYYLLRRYLESVYSGCEARNAFIKLIQKISDVERLNKFIINVYLNVNPSQVEPLLREIFDLKNH

SEQ ID NO: 8 - DNA, 2832 bp, Artificial Sequence
  Description of Artificial Sequence; note = synthetic construct
gttattgggattggcctggagcactcggacggacagtaattcattaaaatatgtggtgataacgcgagctgccgaatctgcgtgcaattcgtgcgtttgacgtgggtactaactgctatgctgtcgcgcggacagttgttctgatacgcagagttcctgcctcaccacacacgaccacctccattaaaaccagccaccccccccagcgcctcctccaccgacagcagctgctccaccgcaccaccaggagaggggcaattaaaaaatcaatcagagggccctaattgaaagctgccaccgtcgaaatgtcgccgccgaagaactgcgcggtgtgcggggacaaggctctgggctacaacttcaatgcggtcacctgcgagagctgcaaggcgttcttccgacggaacgcgctggccaagaagcagttcacctgccccttcaaccaaaactgcgacatcactgtggtcactcgacgcttctgccagaaatgccgcctgcgcaagtgcctggatatcgggatgaagagtgaaaacattatgtccgaggaggacaagctgatcaagcggcgcaagatcgagaccaaccgggccaagcgacgcctcatggagaacggcacggatgcgtgcgacgccgatggcggcgaggaaagggatcacaaagcgccggcggatagcagcagcagcaaccttgaccactactcggggtcacaggactcgcagagctgcggctcggcggacagcggggccaatgggtgctccggcagacaggccagttcgccgggcacacaggtcaatccgcttcagatgacggccgagaagatagtcgaccagatcgtatccgacccggatcgagcctcgcaggccatcaaccggttgatgcgcacgcagaaagaggctatatcggtgatggagaaggtaatcagctcacaaaaggacgccttaaggctggtgtcgcatttgatcgactatccaggcgacgcactcaagatcatttcaaagtttatgaactcgccctttaacgcgctgacagtattcaccaaattcatgagctcacccacggacggcgttgaaattatctcaaagatagttgattcgcccgcggacgtggtggagttcatgcagaacttgatgcactcgccagaggacgccatcgatataatgaacaagttcatgaataccccagcggaggcgctgcgcattcttaaccgaatcctaagcggcggaggagcgaacgcagcccagcagacagcagaccgcaagccattgctggacaaggagccggcggtgaagcctgcagcgccagcggagcgagctgatactgtcattcaaagcatgctgggcaacagtccgccaatttcgccacatgatgctgccgtggatctgcagtaccactcgcccggtgtcggggagcagcccagtacatcgagtagccaccccttgccttacatagccaactcgccggacttcgatctgaagaccttcatgcagaccaactacaacgacgagcccagtctggacagtgattttagcattaactcaatcgaatcggtgctatccgaggtgatccgcattgagtaccaggccttcaatagcatacaacaagcggcatcgcgcgtaaaggaggagatgtcctacggcactcagtctacgtacggtggatgcaattcggctgcaaacaatagccagccgcacctgcagcaacccatctgcgccccatccacccagcagttggatcgcgagctaaacgaggcggagcaaatgaagctgcgggagctgcgactggccagcgaggctctttatgatcccgtggacgaggacctcagcgccctgatgatgggcgatgatcgcattaagcccgacgacactcgccacaacccaaagctattgcagctgatcaatctgacggcggtggccatcaagcggcttatcaaaatggccaagaagattacagcattccgtgacatgtgccaggaggaccaggtggccctactcaaaggtggctgcacagaaatgatgataatgcgctccgtaatgatttacgacgacgatcgcgccgcctggaaggtaccccataccaaagagaacatgggcaacatacgcactgacctgctcaagtttgccgaaggcaatatctacgaggagcaccaaaagttcatcacaacgtttgacgagaagtggcgcatggacgagaacataatcctgatcatgtgtgccattgtcctttttacctcggctcgatcgcgagtgatacacaaagacgtgattagattggaacagaattcctactattatcttctgcgaagatatctggagagtgtttattctggctgtgaggcgagaaacgcgtttatcaagctaatccaaaagatttcagatgtggagcgtctgaacaagttcataattaatgtctatttgaatgttaacccatcccaggtggagcccttgctgcgtgaaatattcgatttgaaaaatcactagacaaccgatgcgtgtcgggcatttaatgcctatgttgatgcccaatgatgaatggtcaacaagctgtagttgttgttgttgttgatgtctgttttatcttgtcgcttgtaatgttagattttaatcgaatgtgattgttagatttgcatatactgcatagattttatatttctacatcaaagagagcatatttaggataccaagtgcaaagcaacacaatctatatgtaatgtacaccgtttacctagtttcaaataaactagacgataatgcaataactaacttggaagcgtgggttctgtgcaaaaaggaaaaaagacaaaaaaaataaactgactttgagaaccagtggtaa

SEQ ID NO: 9 - protein, 704 aa, Artificial Sequence
  Description of Artificial Sequence; note = synthetic construct
MMKHPQDLSVTDDQQLMKVNKVEKMEQELHDPESESHIMHADALASAYPAASQPHSPIGLALSPNGGGLGLSNSSNQSSENFALCNGNGNAGSAGGGSASSGSNNNNSMFSPNNNLSGSGSGTNSSQQQLQQQQQQQSPTVCAICGDRATGKHYGASSCDGCKGFFRRSVRKNHQYTCRFARNCVVDKDKRNQCRYCRLRKCFKAGMKKEAVQNERDRISCRRTSNDDPDPGNGLSVISLVKAENESRQSKAGAAMEPNINEDLSNKQFASINDVCESMKQQLLTLVEWAKQIPAFNELQLDDQVALLRAHAGEHLLLGLSRRSMHLKDVLLLSNNCVITRHCPDPLVSPNLDISRIGARIIDELVTVMKDVGIDDTEFACIKALVFFDPNAKGLNEPHRIKSLRHQILNNLEDYISDRQYESRGRFGEILLILPVLQSITWQMIEQIQFAKIFGVAHIDSLLQEMLLGGELADNPLPLSPPNQSNDYQSPTHTGNMEGGNQVNSSLDSLATSGGPGSHSLDLEVQHIQALIEANSADDSFRAYAASTAAAAAAAVSSSSSAPASVAPASISPPLNSPKSQHQHQQHATHQQQQESSYLDMPVKHYNGSRSGPLPTQHSPQRMHPYQRAVASPVEVSSGGGGLGLRNPADITLNEYNRSEGSSAEELLRRTPLKIRAPEMLTAPAGYGTEPCRMTLKQEPETGY

SEQ ID NO: 10 - DNA, 3248 bp, Artificial Sequence
  Description of Artificial Sequence; note = synthetic construct
agttgaattccagtgacgttggaagaaacaactgcaaaaggcaaaaacaaagacaatgtttataagctgtatattccgctttgattgatataaatgaatatatgcagtgcgccagttatacaactgccctgcaaaagtcactcattaaataaaaaacgcccgagatgaatttcacagcggcggcaacaagtgcaataatagtaaaaaatcaaaagccaaacaacgaaatctctcccaaaaaaacgaagaagcgtgtcgcggtgccaaaaagaaaacaaaaatagaaaaatacacaacaaaataatacggagaaacgttaattataacgagccacaaaatcgcataaagaaatcaacaagtgtgtgtctgcctttttttccatattcgctttcattcatgcggtcaactcaacaataacaactcaaaatagcaacaacaacaataacaatatcaacaagagcagcagcagtcgctgataaaagccctgcagctaaaacaacaacaaaacaacaaagatagttagaaagaacatcgtctggccattgagctttaattgccggtcattacttcattactatgtgattggatcttcccgacccacttgtaaataaaaagtaaaaatactggttatgaagcatgatgaagcatccgcaggatctgagtgtcacggatgaccagcagttaatgaaggtgaacaaggtggagaagatggagcaggagttgcacgaccccgaatcggagagccacataatgcacgcggatgccctggcctctgcctatccggctgcctcgcagccccacagtccgatcggcctcgccctcagccccaatggcggtgggctgggactgagcaacagtagcaaccagagcagcgagaactttgcgctctgcaacggaaacggaaatgcgggcagcgcaggaggcggaagtgccagcagtggcagcaacaacaacaacagcatgttctcacccaacaacaacttgagcggaagcggaagtgggactaacagcagtcagcagcaattgcagcagcaacaacaacagcaatcaccgacggtctgcgccatttgtggagatcgggcgacgggcaaacattatggagcctccagctgcgacggctgcaaaggattcttcaggaggagtgtcaggaaaaatcatcagtacacttgcagatttgcgcgaaactgcgttgtggacaaggacaaacggaatcagtgccgctactgccggctgaggaagtgcttcaaggcgggcatgaagaaggaggcggtgcaaaacgagcgggatcgcattagctgccgccgcacctccaatgacgacccggatccgggcaatgggctgtctgtgatttccttggttaaggcggagaatgagtcgcgtcagtcgaaggcaggcgctgccatggagccaaacattaacgaggacctctccaacaagcagttcgcgagcatcaacgatgtctgcgagtcgatgaagcagcagctgctgaccctggtggaatgggctaagcagattccggcctttaacgagctgcagctggatgaccaggtggcactgctacgcgcccatgctggcgagcatttgctcctcggcctgtctcgtcgttcgatgcacttgaaggatgttctcctgctgagcaacaattgtgtgatcacaaggcactgtccagatccccttgtgtcgccgaatttggacatctcccggatcggcgcccgtatcatcgatgaactggtgacggtcatgaaggatgtgggtatcgatgacactgaattcgcttgcatcaaggccctagtcttcttcgatcccaatgccaagggtcttaatgaaccgcatcgcatcaaatcgctacggcatcagatactcaataatctcgaggactacatatcagatcggcaatacgagtcgcgcggtcgctttggcgagattctgctcatcctgccggttctgcagtctattacctggcagatgatcgagcagatccagtttgccaagatctttggagtggcccacattgattcattactgcaggaaatgttgttgggaggagagttggccgacaatcctctgccgctatcgccgcccaatcagtcaaatgactaccagagtcccacccacacaggcaacatggagggcggtaatcaagttaactcctctctggactcgctggccacgtccggtggtcctggctcgcatagtctggacctggaggtgcagcacattcaggctcttatcgaggcgaacagtgcggatgattccttccgggcctacgcggccagcactgcagcggcagccgctgcagccgtctcgtcctcctcctctgcacccgcatccgttgctccagcctcgatctctcctccgctcaacagccccaagtcacaacatcaacatcagcaacatgcgacgcatcagcaacaacaggagagctcctacttggacatgcccgtcaagcactacaatggcagtcggtccggaccgctgccaacacagcacagtccccagaggatgcatccctaccaaagagcagtcgcctcgccggtcgaagtgtccagcgggggcggcggattgggtctgcgcaatcctgccgatattacgctcaacgagtacaaccggagcgagggtagcagtgccgaggagctgctgcgacgaactccactgaagatccgggctcccgagatgctaaccgcacccgctggttatggaacggaaccctgtcgcatgacacttaaacaggagccagagactggttactagaagaataacgaacggtgcaatatgcagtttgcaataggacaccccttaagcacacaacccatacacatacaggccctctcttgctgtactccccaccaagtgctatatagagatgaaattgaaatgaagaacttacttaattgttatgccttgaaccattttgatactttttattagtcctaagtaggtattttggaaattgttgcttaatttttaatgtttaacgcagttgcaatatatttttggagtcatattttgctcaagaagtttattatatacaattatactatatatatacaccatttagcatgtactgagtttgttggttatttggttatcttatacttgtgcgtggatcacaaaacattcatataaggccatgcaatatattgttttaggttagggtgttgtctagattatgctgaaagtgtaatatatatttaattttaaacaaagaactatttttatatgaatatgtataatatacaaactatttc

SEQ ID NO: 11 - protein, 556 aa, Artificial Sequence
  Description of Artificial Sequence; note = synthetic construct
MDEDCFPPLSGGWSASPPAPSQLQQLHTLQSQAQMSHPNSSNNSSNNAGNSHNNSGGYNYHGHFNAINASANLSPSSSASSLYEYNGVSAADNFYGQQQQQQQQSYQQHNYNSHNGERYSLPTFPTISELAAATAAVEAAAAATVSSPSVGGPPPVRRASLPVQRTVSPAGSTAQSPKLAKITLNQRHSHAHAHALQLNSAPNSAASSPASADLQAGRLLQAPSQLCAVCGDTAACQHYGVRTCEGCKGFFKRTVQKGSKYVCLADKNCPVDKRRRNRCQFCRFQKCLVVGMVKEVVRTDSLKGRRGRLPSKPKSPQESPPSPPISLITALVRSHVDTTPDPSCLDYSHYEEQSMSEADKVQQFYQLLTSSVDVIKQFAEKIPGYFDLLPEDQELLFQSASLELFVLRLAYRARIDDTKLIFCNGTVLHRTQCLRSFGEWLNDIMEFSRSLHNLEIDISAFACLCALTLITERHGLREPKKVEQLQMKIIGSLRDHVTYNAEAQKKQHYFSRLLGKLPELRSLSVQGLQRIFYLKLEDLVPAPALIENMFVTTLPF

SEQ ID NO: 12 - DNA, 5181 bp, Artificial Sequence
  Description of Artificial Sequence; note = synthetic construct
ctcgcccattggagggcccctgtcctgtggcagcagcttgcccagcttccaggagacctactccttgaagtacaacagcagcagcggtagcagcccccagcaggcgtcctcctcctccaccgccgcccccacgcccactgaccaggtgctgaccctcaagatggacgaggactgcttcccgcctctgtccggcggctggagtgccagtccgcccgccccctcccagctccagcagctgcacaccctgcagtctcaggcccagatgtcgcatcccaacagcagcaacaacagcagcaacaacgcgggcaacagccacaacaacagtgggggctacaactaccacggccacttcaatgccatcaatgccagcgccaatctgtcgcccagctcctcggccagttccctctacgaatataatggtgtttccgcagcggacaacttctacggacaacagcagcagcagcaacagcaaagctatcagcaacataactacaactcgcacaatggcgagcgttactcgctgcccacgtttcccacgatttcggagctggctgcggccactgctgctgtcgaagctgcggcggcggccacagtctcctccccttcggtgggcggtccgccgccagtacgccgagcatcgctgccggttcagcgaaccgtttcgccagccggctccacggcgcagagccccaagctggccaagatcacactgaaccagcggcactcccatgcccatgcccatgccctacagctcaactcggcacccaattcggcggcaagttcgccagcgagtgcggatctgcaggcgggccgtttgctccaggctccgtcgcagctgtgtgccgtttgtggcgacaccgccgcctgccagcattatggagtgcgaacctgcgagggatgcaagggattcttcaagcggaccgtgcagaagggctccaagtatgtctgcctagcggacaagaattgcccggtggacaagaggcgccgcaaccgttgccagttctgccggttccagaagtgcctggtcgtaggcatggtcaaggaagtggtgcgcacggactcgttgaagggtcgccgcgggagactgccctcaaaaccgaaatcgccccaggagtcgccaccatcaccacccatctcgttgatcacggccctggttcgcagccatgtcgacacgactccggatccctcgtgcctggactacagccactatgaggagcagtcgatgagcgaggcagataaggtgcaacagttttaccagctgctgaccagctccgtggacgtgatcaagcagttcgccgagaagattcccggctacttcgatctcctgccggaggatcaggagctgctcttccagagcgcatcgctggaactgttcgtcctgcggctggcctatcgcgccaggatcgatgacaccaagctgatcttctgcaacggcacggtgctccaccgcacccagtgcctgcgctccttcggcgagtggctcaacgacatcatggagttcagccgcagcctgcacaacctggagatcgacatctccgccttcgcctgcctctgtgccctaaccctgatcacagaacgccatggcctgcgggagccgaagaaggtggagcagctccagatgaagatcattggcagtctgcgcgaccacgtcacctacaatgccgaggcccagaagaagcagcactacttcagccgcctgctgggcaagctgccggagctgaggtccctgagtgtccagggactgcagaggatcttctacctgaagctggaggacctggtgcccgcgccagctctcatcgagaacatgttcgtcaccacattgcccttctagaggcgatcatcaagcgtatcatcacaacttgcttccttaaactagcccctaagttatgcctcctaggatatacagagaaaggaccccataggacggacgcaactagctttagtagaaccctgaaataaataaatctcacaacagcaaaaacaaaaccgaaccgaacagaaatgaagcgaatagcagacccaggccatatctttagtgtagagctaggtagttagccggacagccccggctccttcgataattacggacatgcatatttgagagggggtttccagtgcacagcctatggctcctgcgtgactcgtcagcaccgcgagctccaacttgttgacgttaattgttaaattgtttaatttcaactgtcaaaaccggaatcaacggccgggcacgcaatggcaacactttctatccccggacttcgaagcctgctcaacattcggcactacggacggacaaacaacggacagaaacagaactcactcttgctctcttgccttttgctaacttctagtcaattgatttaggcgaatcaaataaataaataaataaaataagggcgtgcagcagtagtgttatataatttctatgccagaccccagcggttctcttcaaggaaatcccccaatgagttgcacaaattgggataaagtacgatagcctattattcttatatttcttttaaaagctcgaagatagatgagaactgtgtggaaatccactatcatatcatatagttgctataagccgtgcttgccctaagctaagttagacccgcataaagttgatagcccaaccaagtatttcggttatttcctagactaaggtcctaatagttataggctaagactattctgttcgatttatcaatgcaccaaacagtgcacaatgagagtataagtaccttcttgtgatgattgtgtctgacacagagagagttgcacacaagcacacaaactagccgataagttactaaatacgatctaatatctaatatatataatataatataatatatataagtccaagtattcggaaatccaagaacccttgcataaccgcagttcgtacgttccaaacgagaaaagaactttatttaatcctagaccactccatctaagttctcaaagaatcgtatgtggatcgttggatctgtctctctatatatgtgtgtgtgttatctcgatagaaaacccctctatgtgattttgtgatagattggcattgaactctatatatttatatatatatgtctataatatatatacacgcataaatatatatttttatgtctaacttttgtatggtttattttatacgtaccacttttctttgataacaaaaagtaaaaaactcgttagatagcaaatatttcaaaggtatgttacgaggacttttcaaagtaccagtctttagcgactttccaattaacgttcgtattaacgaaagacagattttctatgtgttaaattgaagacttctataactataactaaatgcaagctaagagcaaaaacacaaatccacaaatccccaaagtgaataacatatctcttcaagctttcgagtgcacggaacacgtagaaccgaaacccaagtgttactaaatccatttaataatcggcaagccgggggcgtcggcgtggttaatacgttctcattacctatacaatttagatagatcattattaaattattgtacatgtagcacatgaaatgttcgacaactagattttgtaccatcttaaagaagaacctaggccaagctaaactaagtataaactatgatctgcatgcggctgagctgtagctatgagaaatatacctgcgtggatctaagtgaaatgggacactttgaatttagatatgaaacgttctaaacgcgacgtactaactctcccaactgcgaactctaccaattaagagaaattcccagaaaatgtgtcaggatttcaaagcgtcccatctcacttgaacccacccaatcaacaaatacaaatcctagggaagttgagaggttcagcaaccatagagcaatatttcataagaaaacgcaccttaaattaccgaaaaacatagattaacctgatcttgtaacgtttgggagcgataataagccaggattaaacaggaacagttaggtgaccaaatcagttcgaaacgagatgatagataggttcgggttcgaaaccctaaacgcgatgccattttagccgttacaacattggatatcaaccatgcacatgaatatgaatatgaatatgaatattatagagatatatctagctataggaacctactttgtacctacacgacatggaaacatcaaacctacatgcatatttacacacatatattttgaatagagcgacgacttttacaagttgcgtacaaagctatagctatagcttgatatggccatcccagagcgagcatatacatatattttgggttattgttcttttgtaattttataaatgcatacatatttattgtactacgtgaatgtcaagtgtggattcatatttttgagatacagctacaaaacgaaacaaaagaaaataaaacaaaacagaagagtaaacgtgaaatttttcgatgaaacaattttaaatgagaactttttaatattgctattaaaggatatacatatacacactaacatacatatatattttactatgtaacggatagaattaagctagatgcagcgcataaagctttatacaacaaattgaaaagcaacagaagaaattggcacaaattaaatttatatagcataattagacgtccttcgcaagataatgttattcgtaataagagcgtcaatcggtacatcgggcgctatttcccactacacccccaaccacacaatagataacctaagctatgtatgtacattagctatgtatatccagcccacttatgcgcctactactagaaatgcagaaagcagaaagagaggtgaaacctatagacgctatcacaaatgtctatctgatagacatcggtactaccaatgctatattgccagttgtgtaatttactcttatttgatcgtttcatttaccagttaagaacccaaatcatataagtgttatgatggaagaactataacttgcaattcaattaactctgcaatacgataacaagcaaagcgaatcatttcatttcgatttaatctttaattatatatacttaaacgatgtaagcccaaaacaaacgttttttctatatctgtcttttgagcaaattagttatacgcaaaaccaaaccgtatttacataaatgtatacaaaacaaatcgtatattttcattggtttgaaataaatacataaaacaa

SEQ ID NO: 13 - protein, 278 aa, Artificial Sequence
  Description of Artificial Sequence; note = synthetic construct
MSNFSACAVCGDQSSGKHYGVSCCDGCSCFFKRSVRRGSSYACIALVGNCVVDKARRNWCPSCRFQRCLAVGMNAAAVQEERGPRNQQVALYRTGRRQAPPSQAAPSPTPHSQALHFQILAQILVTCLRQAKANEQFALLDRCQQDAIFQVVWSEIFVLRASHWSLDISAMIDGCGDEQLKRLICEAHQLRADVLELNFMESLILCRKELAINAEYAVILGSHSKAALISLARYTLQQSNYLRFGQLLLGLRQLCLRRFDCALSCMFRSVVRDILKTL

SEQ ID NO: 14 - DNA, 837 bp, Artificial Sequence
  Description of Artificial Sequence; note = synthetic construct
atgtcgaacttcagtgcctgcgcagtgtgcggcgatcagagctccgggaagcactacggcgtgtcctgctgcgatgggtgctcctgctttttcaagcggagcgtgcggcgcgggagcagctacgcctgcatcgctctggtcgggaactgtgtggtggacaaggcgcggcggaactggtgtccctcctgccgcttccagcgatgcctggccgtgggaatgaacgctgctgcggttcaggaggagcgcggtccgcgcaaccagcaggtggctctctaccgcactggccggagacaagctccgccatctcaggcggcgccatccccgacgccccactcccaggcgctgcacttccagatcctcgcccagatccttgtcacgtgcctgcgccaggcgaaggccaacgagcagttcgctctgttggatcgctgccaacaagacgccatctttcaggtggtgtggagcgagatcttcgtcctgcgagcgtcccactggtctctggacatcagcgccatgatcgacggctgcggcgatgagcagctcaaacggctcatttgcgaggcccaccagctaagggccgacgtcctggaactcaactttatggagtccctaatcctgtgcagaaaagaattggccatcaatgcggagtatgccgttatcctgggaagccactctaaagccgccctgatctccttagcccgctacaccctgcagcaatccaactacctgcggttcggacaactgctccttggtctgaggcagctgtgcctgaggcgcttcgactgcgcgctttcttgtatgtttcgcagcgtggtcagggacatcttaaaaacactttag

SEQ ID NO: 15 - protein, 281 aa, Artificial Sequence
  Description of Artificial Sequence; note = synthetic construct
MGMRREAVQRGRVPPTQPGLAGMHGQYQIANGDPMGIAGFNGHSYLSSYISLLLRAEPYPTSRYGQCMQPNNIMGIDNICELAARLLFSAVEWAKNIPFFPELQVTDQVALLRLVWSELFVLNASQCSMPLHVAPLLAAAGLHASPMAADRVVAFMDHIRIFQEQVEKLKALHVDSAEYSCLKAIVLFTTDACGLSDVTHIESLQEKSQCALEEYCRTQYPNQPTRFGKLLLRLPSLRTVSSQVIEQLFFVRLVGKTPIETLIRDMLLSGNSFSWPYLPSM

SEQ ID NO: 16 - DNA, 2866 bp, Artificial Sequence
  Description of Artificial Sequence; note = synthetic construct
ctaaattgttgttttcaaaagaaatgaatttctttccactcctttcagaattcaagaataaatattgaagcaatatggcttcccttgttcaaaccgatcaatcgttgcaaatctttcttcaagcgctcggtgcgacgtaatctaacttactcttgccgcggcagcagaaactgtcccatagatcaacaccatcgcaatcaatgtcaatattgtcgattgaagaagtgcctcaaaatgggcatgagacgcgaagctgttcaacgtggacgcgtaccacccactcagcccggtctggccggcatgcatgggcagtaccagattgccaacggggatcccatgggcattgccggctttaacgggcactcgtacctcagttcctacatctcgctcctgctgcgggcggaaccgtatccgacttcgcgatatggccagtgcatgcaacccaacaacattatgggcatcgacaacatctgcgaactggccgcccgactgctcttctcggcggtcgagtgggccaagaacatacccttcttcccggagctgcaggtgaccgaccaggtggccctgctccggctcgtctggtcagagctcttcgtcctaaacgccagccagtgctccatgccgctccatgtggcgccactgctggccgccgccggacttcatgcctccccgatggccgccgatcgtgtggtggccttcatggaccacatccgcatcttccaggagcaggtggagaagctgaaggcgctgcatgtcgactccgcggagtactcctgcctcaaggcgatcgtgctcttcaccaccgatgcctgcggcctgtccgatgtgacgcacattgaatccctgcaagagaagtcgcagtgcgccctcgaggaatactgccggacccagtatcccaaccagcccacgagattcggcaagctgcttctcagactgccatcgctgcgaacggtctcctcacaagtcattgagcaattgttttttgtgcgtctagtcggaaaaacgccaattgaaacgctgatacgcgatatgctgctgagcggcaacagtttctcctggccctatctgccttcgatgtgacacacgatgtggcgccaattgacaacaacttgatcatcggccgcagctgtggcggctgcaacgctcaacatcaattccggcggaggcggcatcggcatcggcggcgggggcagtggcagtggcggtggcggtagtggaggcggtggcggagtcgttggatgtggcagccacaacgttgtcgctgccagtcatgaccagctcgccaatgttgctgtcatgcagcaaacatacggcagcggcggcagcagcagcagcagcatcagcggttgccacaacggtaacaacggcagcggcggcagcatttgcaatcagcagatcaacaactacggcaacaacagcaacaacaatgtcggcaatcatatgagtgcaggcagttttttcggtgggtccaacaacagcatccacagtagtggcaatagcaataccgattatatgaccacgccagccaccgcttatgcgacaccagcgacagcagccacatccacggtgaacaccacaacgatgctgtctaattactgcgatgccgccaccatgatgatggccgctgctgcagtcaatgcaaatcaatgcctgcagcaacatcaccagcgcatgttgctcgcgggcagcagcaacagcagcagcaacaacagcagcagcaacagcaacggcgcagcagcaatgccctcctcatcctcgtctggctcactgtcatctgcctcatcgaccccaacagcaacagcaactgcgactgcaattgcaacagcaacagcaactgcagcagcaacagccgcgcagcaacaacagcaacaatcgccgccaaatttaatcgatatcagcgaagttcctctcattgtggatgtcaagtagtgtaattatttatgcatctagaaatggggctataaaccaaccttgtagataccccgccccgcccccaccactaccacaaaaaccataaaaccccaaaaaaaaaacaattgaaaaatgtaaaaaaaaaaagttggaggatgagcgccgcgtagcttaattgactaattttccatttgtagcttttgttgtaactttgtacataactcctcgaaaaattcaagtttttctctaggccaccccagctgtgagcaaaaccaatctcagctgacatatccaagagaacttcaaaagtgaagcccccaaaaaaagtaagaaggcgccaaaaaaacgtctttacatatgaatgtgtataatatttaaatggcactgagttctacttaattttagaccacaaacacttgaaaaaatcaatgaaaaaataagaattgtggaaagagaaaaatcccccctaacactttcaaaagacaaaacataaagatagttaaaatatttatatatgtaatgtagcatatacacgtatatagtacatatatgaatatataaacgaaactctactcccagtggtttgcagaaatataccaaaaattttaagctatgtttacttgatgtgtggcaatttttatgtgtgctttagcaattttatttttactttaagtaaaatttaaaatttataaacattcgattctcgactggtttttctcggcggatgtatctcaaagatgcttctgtatgggaaggccgaattgttgaaatacgaatgcaaaatttagcgaattttttatttagtaaccattacgagtaaaaacacaaaatgttcagtgcaagtttcagttcttaaacgattttttcgtaagcttaagcattatcttatttatgtgtatagagtatgaaaagttttctatattttgtaataataaaaatttgcgtttataatgaa

SEQ ID NO: 17 - protein, 452 aa, Artificial Sequence
  Description of Artificial Sequence; note = synthetic construct
MQSSEGSPDMMDQKYNSVRLSPAASSRILYHVPCKVCRDHSSGKHYGIYACDGCAGFFKRSIRRSRQYVCKSQKQGLCVVDKTHRNQCRACRLRKCFEVGMNKDAVQHERGPRNSTLRRHMAMYKDAMMGAGEMPQIPAEILMNTAALTGFPGVPMPMPGLPQRAGHHPAHMAAFQPPPSAAAVLDLSVPRVPHHPVHQGHHGFFSPTAAYMNALATRALPPTPPLMAAEHIKETAAEHLFKNVNWIKSVRAFTELPMPDQLLLLEESWKEFFILAMAQYLMPMNFAQLLFVYESENANREIMGMVTREVHAFQEVLNQLCHLNIDSTEYECLRAISLFRKSPPSASSTEDLANSSILTGSGSPNSSASAESRGLLESGKVAAMHNDARSALHNYIQRTHPSQPMRFQTLLGVVQLMHKVSSFTIEELFFRKTIGDITIVRLISDMYSQRKI

SEQ ID NO: 18 - DNA, 1885 bp, Artificial Sequence
  Description of Artificial Sequence; note = synthetic construct
gagtccacatcggagtaaccaaggatatatcgaatatatcacacaatccgcaataccgccgtccacccaaaccgttaaaacaaaaatccaaaacgactcaaagatacaccagtgccaagtgaaattcaatttgtgcaagcgtttctacaaaaatcgccaaaattacgccccacatcggtatgcagtcgtcggagggttcaccagacatgatggatcagaaatacaacagcgtgcgtctttcgccagcggcatcgagtcgcattctataccatgtgccctgcaaagtctgcagagatcacagctccggcaagcattacggcatctacgcctgtgatggctgcgccggattcttcaagaggagcattcggagatcccggcagtatgtgtgcaagtcgcagaagcagggactctgtgtggtggacaagacgcacaggaaccaatgtagggcttgccgactgaggaagtgctttgaggtcggaatgaacaaggatgcagtgcagcacgagcggggaccgcggaactccactctgcgtcgccacatggccatgtacaaggatgccatgatgggcgccggcgagatgccacaaatacccgccgaaattctgatgaacacggctgccttgaccggctttcctggagtaccgatgcccatgcctggcctgccccagagggctggtcatcatcctgctcacatggctgccttccagccgccaccatcggctgccgctgtcttggacttatccgtgccacgagtgccccatcacccggtgcaccaaggacaccacggtttcttctcgcccaccgccgcctacatgaatgccctggccactcgggccctgccccccactcctccgctgatggcagctgagcacatcaaggaaaccgcggcggaacacctattcaagaacgtcaactggatcaagagcgtacgggccttcaccgaactgcccatgccggatcagctgctcctgctggaggagtcctggaaggagttcttcatcctggccatggcccagtacctaatgcccatgaatttcgcccagctgctgttcgtctacgagtccgagaatgccaaccgggagatcatgggcatggtgacccgcgaggtgcacgccttccaggaggtgctgaaccaactgtgccatctgaacattgacagcaccgagtacgagtgtctgagggctatttcgctcttccgtaagtcaccaccgtcggcaagttctaccgaggatttagccaacagctcaatcctgacaggaagcggcagcccgaactcctcggcctctgctgaatccaggggtcttctggagtcgggaaaagtggcggccatgcacaacgatgcccggagtgcgctgcacaactacatccagaggacccatccctcgcagcccatgcgattccagacgctcttgggcgtggtgcagctgatgcacaaggtctcaagcttcaccatcgaggagctgttcttccgaaagaccatcggcgacatcaccattgtgcgcctcatctccgacatgtacagtcagcgcaagatctgaaaagtatgtagagcctagactaatcgccgcactcgaagtgccttccaagtgctgggaactgtgataatctcggaagaagcgctttggacaatactcgatcagtgaaatcaacgatttctcatatccaggagtcgagccttaaaatacgtacacaacactcaccttaataccttacctaaacagaactcgaagtaatcttagctaaagtctctcagaccatccagatgtgtttcaaattgcattcgcaaaagtttcaactttgcctgttaaatacgtcaatcgtagttttaaacactttagttttaagcgcatattattagctttaggatttggaaaaataattattc

SEQ ID NO: 19 - protein, 691 aa, Artificial Sequence
  Description of Artificial Sequence; note = synthetic construct
MGTAGDRLLDIPCKVCGDRSSGKHYGIYSCDGCSGFFKRSIHRNRIYTCKATGDLKGRCPVDKTHRNQCRACRLAKCFQSAMNKDAVQHERGPRKPKLHPQLHHHHHHAAAAAAAAHHAAAAHHHHHHHHHAHAAAAHHAAVAAAAASGLHHHHHAMPVSLVTNVSASFNYTQHISTHPPAPAAPPSGFHLTASGAQQGPAPPAGHLHHGGAGHQHATAFHHPGHGHALPAPHGGVVSNPGGNSSAISGSGPGSTLPFPSHLLHHNLIAEAASKLPGITATAVAAVVSSTSTPYASAAQTSSPSSNNHNYSSPSPSNSIQSISSIGSRSGGGEEGLSLGSESPRVNVETETPSPSNSPPLSAGSISPAPTLTTSSGSPQHRQMSRHSLSEATTPPSHASLMICASNNNNNNNNNNNNGEHKQSSYTSGSPTPTTPTPPPPRSGVGSTCNTASSSSGFLELLLSPDKCQELIQYQVQHNTLLFPQQLLDSRLLSWEMLQETTARLLFMAVRWVKCLMPFQTLSKNDQHLLLQESWKELFLLNLAQWTIPLDLTPILESPLIRERVLQDEATQTEMKTIQEILCRFRQITPDGSEVGCMKAIALFAPETAGLCDVQPVEMLQDQAQCILSDHVRLRYPRQATRFGRLLLLLPSLRTIRAATIEALFFKETIGNVPIARLLRDMYTMEPAQVDK

SEQ ID NO: 20 - DNA, 3043 bp, Artificial Sequence
  Description of Artificial Sequence; note = synthetic construct
gtcagcccaggcgatccgcatttgcgtccgcagcaggtttccgatttcagaactctgattccagcggcagcgaatcgcgtcggcatctgaacatttgaaaataatctaaaattgcaagtgactttgtgcaccggttacactaaaattgttaacaaatcgccatatattctgaatttaaatttaaagtgcgcagtgcggaatataaatcagagcaaactggatacgttagggttcaaatacttccatcaacggaaaatgggcacagcgggcgatcgcctgttggacattccctgcaaggtgtgtggcgatcgcagctccggcaagcactatggaatctacagctgcgatggctgctccggttttttcaagcggagcattcatcgcaatcggatttacacctgtaaggccaccggcgatctcaagggtcgctgtccggtggacaagacccatcggaatcagtgtcgcgcctgtcgcctggccaagtgcttccagtcggccatgaacaaggatgctgtgcagcacgagcgcggtcctaggaaacccaagttgcacccgcaactgcatcatcatcatcatcatgctgctgccgccgccgctgcagcgcatcatgcagcagccgcccatcaccatcaccatcatcaccaccacgcccacgcagcggccgcccatcatgcggcagtggctgcagcggctgcctccgggctgcatcaccaccaccacgccatgcccgtctcgctggtgaccaatgtctcggcctcgttcaactatacgcagcacatctccacgcatccgcctgctccggcggcgccacccagtggctttcacctgacggccagtggcgcccagcagggaccagctccaccagctggccacctgcaccatggtggagccggacatcagcacgccacggccttccaccatccgggacatggacacgcgctgcctgccccacatggcggcgtcgtcagcaatcccggcggcaactcgagcgcaatctccggcagcggtcccggctccacgctgcccttcccctcgcacctgctgcaccacaatctgatagcggaggcggccagcaagctgccgggcatcactgccacagccgttgcggcggtggtgtcctccactagcacgccctacgcctcggcggcccagacgtcgtcgcctagtagcaacaaccacaactactcctcgccctcgcccagcaactccatccagtccatctcgagcattggatcgcgcagcggtggtggcgaggagggcctcagcctgggcagcgagagtccgcgcgtcaatgtggaaacggagacaccttcgccatcgaactcgccgccccttagtgctggtagcatttcgccagcgcccacgttgaccacctcgtcgggatcgccgcagcaccgccagatgtcgcggcacagcctcagtgaggcaaccacgccgcccagccacgcctctctcatgatttgcgccagcaacaataacaataacaacaataataataacaataatggagagcacaagcagtcgagctacacatccggatcaccgacacccacaacgcccacgccgccaccgccgcgttctggtgtaggttccacctgcaacacggccagcagctccagcggcttcctggagctgctgctcagtccggacaagtgccaggagctcatccagtaccaggtgcagcacaacacgctgctcttcccgcaacagctgttggactcgcggctgctctcctgggagatgctgcaggagacgacggcgcgactgctcttcatggcggtgcgctgggtcaagtgcctcatgcccttccagacgctctccaagaacgaccagcatttgctgctccaggaatcctggaaggagctcttcctgctcaacctcgcccaatggactataccgctggatctaacgcccatactggaatcaccgctcatccgcgaacgggtgctgcaggacgaggccacacaaacggagatgaagacgatccaggagatcctctgccgcttccgccagatcacacccgacggcagcgaggtgggctgcatgaaggccatcgccctgttcgcacccgaaaccgccggcctgtgcgacgtgcagccggtggagatgttgcaggatcaggcgcagtgcatcctctccgaccatgtgcgactgcgctaccctcgccaagcaacccgcttcggcaggctgctgctcctgctgccctcgctgcgcaccatccgggcggccaccatcgaggcgctgttcttcaaggagaccatcggcaatgtgcccattgctcgactgctgcgcgacatgtacaccatggaaccggcacaggtggacaagtgaaccggccacgcatgacagtcgaaatgaaatcaaaatcgattccctagcacctaagcgccacccatcggtcgtcgtcatatgcgaacttatttgtattccaatgcgacccgaatcctattcagattcactgcggcaggaggcggtccaaatgtggggcggaagctgcagatgctatggttcgcaggacgccatgtaatggaggcgtatgtactaaccgcgctcctccattggcgatgcagtccgcgatgatggcgcactcccacacccacacccgtacccacaccttgatttatcgccggcaatgcgtcggagtctccttactttcgcttcgttttctaacatttgtatccttattttatttcatctttttccacggatttttcgttttgactgcctgggcggcactctttatttatctttcattcgacgttttgtcgtcgcttttctaaaaattccccatgttatttcaacctggcaaggacctcgcagtcccattcccgcgcccttacttacaaatcacttcccatcccacatccagcaattccgtggtttgaattctttcgtgcattgactacgaaataccctttaatcagacaaataaagaatattagttgtaattcttttttctgcaatccagctctaaaacgggtttcttaatcgaaatcgataaatgtaaaaattatacatatcctttaccaacattgtttgccta

SEQ ID NO: 21 - protein, 532 aa, Artificial Sequence
  Description of Artificial Sequence; note = synthetic construct
MATGRSLLFRVPWYVCLCVCAESAEPGVYWRLRLRLGLPTLAGPHTNTLTLTARTSSCRSIKKERIKASQQANAPPELPLKVSVDVNIIIAAHSQRRRIGLVRFHQRESEDRPLAVASPRLQINMEPTAMNPKKLHSPQRHCYTPPPAPMHGQAPPPTSTGVAPPTQPPPPHPAAPNVPNGRLLSWNHSAAAAAAAAAAQAAANSMNHSSAAEGSSMTRIKGQNLGLICVVCGDTSSGKHYGILACNGCSGFFKRSVRRKLIYRCQAGTGRCVVDKAHRNQCQACRLKKCLQMGMNKDDDSIDVTNDNEEPHAVSRSDSSFIMPQFMSPNLYTHQHETVYETSARLLFMAVKWAKNLPSFARLSFRDQVILLEESWSELFLLNAIQWCIPLDPTGCALFSVAEHCNNLENNANGDTCITKEELAADVRTLHEIFCKYKAVLVDPAEFACLKAIVLFRPETRGLKDPAQIENLQDQAHHTKTQFTAQIARFGRLLLMLPLLRMISSHKIESIYFQRTIGNTPMEKVLCDMYKN

SEQ ID NO: 22 - DNA, 1599 bp, Artificial Sequence
  Description of Artificial Sequence; note = synthetic construct
atggcgaccgggcgttctctgctctttcgagtgccttggtatgtgtgcttgtgtgtgtgcgcagagagcgcagagccgggtgtttattggagattgcgattgcggcttggcttacccacactcgcagggccgcacaccaacacactaacactaacagcgaggacaagctcctgccgcagcatcaagaaggaacgaatcaaagcaagccaacaagcaaatgcgccaccagagttgccactaaaagtctccgttgacgttaacatcatcatcgcggcacactcgcagcgccgtcggatcggattggttcggtttcatcagcgggaatcagaggaccgtccacttgccgtcgcctctccacgattgcaaattaatatggagcctactgcgatgaacccgaaaaaactccacagtccgcagcggcattgctacactccgccgccggcgccgatgcacggacaggcgcctccacctacatcaacgggcgtggccccgcccacacagccaccgccccctcatcccgccgccccaaacgtgcccaatggtcgattgctgagctggaatcacagtgccgctgcagctgctgcggcggcggcagcccaagcggcagccaactccatgaaccactcgtcggcggcggagggttcatcgatgacccggattaagggtcagaacctgggcctcatctgcgtggtgtgcggcgacaccagctcgggaaagcactacggaatcctagcctgcaatggctgctccggattcttcaaacgcagcgtgcggcggaaactcatttatcgctgccaggcgggaacgggacgctgtgtggtggacaaagctcatcggaatcaatgccaggcctgcaggctcaagaagtgccttcaaatgggaatgaacaaggacgacgactccatagatgtaaccaacgacaacgaggagccgcatgcagtcagcagatcggattcgagtttcattatgccgcagttcatgtcgcccaatctgtacacccatcaacacgaaacagtttacgagacaagtgcccggctgctcttcatggccgtcaagtgggccaagaacctgcccagctttgcaagactttcctttcgggatcaggtaattttgctggaggagtcctggtcggagctgttcctgctgaacgcaatccaatggtgcattcccctggatcccaccggctgcgccctcttctcggtggcggagcactgcaataatctagagaacaatgccaatggcgacacttgcataacaaaggaggagctggcggcggatgtgcgaacgctccacgagatcttctgcaaatacaaggcggtgctggtggaccccgctgaattcgcgtgcctcaaggcgatagttctcttccggccggaaacgcgcggacttaaagatccggcgcagatagagaatcttcaggatcaggcgcaccacacaaagacgcagttcaccgcccagatagccagattcggacgactccttctcatgctgccgttgctgcgcatgatcagctcccacaagattgagtccatctattttcagcgcactattgggaacacgcccatggaaaaggtgctctgtgacatgtataagaactag

SEQ ID NO: 23 - protein, 484 aa, Artificial Sequence
  Description of Artificial Sequence; note = synthetic construct
MSDGVSILHIKQEVDTPSASCFSPSSKSTATQSGTNGLKSSPSVSPERQLCSSTTSLSCDLHNVSLSNDGDSLKGSGTSGGNGGGGGGGTSGGNATNASAGAGSGSVRDELRRLCLVCGDVASGFHYGVASCEACKAFFKRTIQGNIEYTCPANNECEINKRRRKACQACRFQKCLLMGMLKEGVRLDRVRGGRQKYRRNPVSNSYQTMQLLYQSNTTSLCDVKILEVLNSYEPDALSVQTPPPQVHTTSITNDEASSSSGSIKLESSVVTPNGTCIFQNNNNNDPNEILSVLSDIYDKELVSVIGWAKQIPGFIDLPLNDQMKLLQVSWAEILTLQLTFRSLPFNGKLCFATDVWMDEHLAKECGYTEFYYHCVQIAQRMERISPRREEYYLLKALLLANCDILLDDQSSLRAFRDTILNSLNDVVYLLRHSSAVSHQQQLLLLLPSLRQADDILRRFWRGIARDEVITMKKLFLEMLEPLAR

SEQ ID NO: 24 - DNA, 2529 bp, Artificial Sequence
  Description of Artificial Sequence; note = synthetic construct
ccctggtcaggtctggttcaccaaaaaagaaaataaaattacatttcaatctttccaatatgcaaatatctgcacgaaaaccagcgagaacagcatgctcacaataaagagcccccaaacaatgtgactcgtatccgcgcagagtgacgtttcgtgccttgcccgagtgccaaatccaaatcccaatccaggcgcacaaaatcgatgcagatgctgtctgcattctcatagaaagtgcaactgaataaccgatggtcgccaaaagccacgatgtccagtaataatgaccagtgaataaacaattatgactcgagcatcgaaaaatgctgaggaacgaatacataagcaataacaagaaggtgctcaactcggaccaaaacaagtactacatgctaacggtcgaggaggccgatatgtattgacgttgttacagtggagctgattacacaaaagatcctcagaacgattttatccaaggcacgaacatgtccgacggcgtcagcatcttgcacatcaaacaggaggtggacactccatcggcgtcctgctttagtcccagctccaagtcaacggccacgcagagtggcacaaacggcctgaaatcctcgccctcggtttcgccggaaaggcagctctgcagctcgacgacctctctatcctgcgatttgcacaatgtatccttaagcaatgatggcgatagtctgaaaggaagtggtacaagtggcggcaatggcggaggaggaggtggtggtacgagtggtggaaatgcgaccaatgcgagtgccggagctggatcgggatccgtcagggacgagctccgccgattgtgtttggtttgtggcgatgtggccagtggattccactatggtgtggcgagttgtgaggcttgcaaagcgttctttaaacgcaccatccaaggcaacatcgagtacacgtgtccggcgaacaacgagtgtgagattaacaagcggagacgcaaggcctgccaagcgtgtcgcttccagaaatgtctactaatgggcatgctcaaggagggtgtgcgcttggatcgagttcgtggaggacggcagaagtaccgaaggaatcctgtatcaaactcttaccagactatgcagctgctataccaatccaacaccacctcgctgtgcgatgtcaagatactggaggtgctcaattcatatgagccggatgccttgagcgtccaaacgccgccgccgcaagtccacacgactagcataactaatgatgaggcctcatcctcctcgggcagcataaaactggagtccagcgttgttacgcccaatgggacttgcattttccaaaacaacaacaacaatgatcccaatgagatactaagcgtccttagtgatatttacgacaaggaattggtcagcgtcattggctgggccaagcagatacctggctttatagatctgccacttaacgaccagatgaagcttctccaggtgtcgtgggcagagatcctgacgctccagctgaccttccggtccctaccgttcaatggcaagttatgcttcgccacggatgtctggatggatgaacatttggccaaggagtgcggttacacggagttctactaccactgcgtccagatcgcacagcgcatggaaagaatatcgccacgaagggaggagtactacttgctaaaggcgctcctgctggccaactgcgacattctgctggatgatcagagttccctgcgcgcatttcgtgatacgattcttaattctctaaacgatgtggtctacttgctgcgtcattcgtcggccgtgtcgcatcagcaacaattgctgcttttgctgccttcgctgcggcaggcggatgatatcctgcgaagattttggcgtggaattgcacgcgatgaagtcattaccatgaagaaactgttcctcgagatgctcgagccgctggccaggtgaaaaggattatgcgggcgcccaaactagttgatctagctgataagcaaaggtgcaaatatagtcttaggtatatatggatgtatactagagtagattaagcgtaggataagccatgtatataaatagtaaaatacttgtcgggtaagattagttcgcagaaaaaatctcttttaatggactaccaactacagcaactggaaaaccctacttatcttctagaatcggggtgtgcttacactggttaaaggcgcatataggtgttatgtgtctaaagttgtgagtcacagatcttcaataatttgttcaattctcactggttctgatatatgtatatgccgcaaccttctgatgtaacgtatgaatttgtgggcacttttaaaatacgatagtggttctacaatacaatggattatactgtttctaagtgtcatgtaacccagtgattctgtgtctatgtggtacacatgcggtcaaaagaatagcaatgtcgtccgtgaataataaaccgtttgtaactgttgtttccatactccctaagttctgtattctttggggattttcttttcctaaacaaattcaaattagtttt

SEQ ID NO: 25 - protein, 601 aa, Artificial Sequence
  Description of Artificial Sequence; note = synthetic construct
MDGVKVETFIKSEENRAMPLIGGGSASGGTPLPGGGVGMGAGASATLSVELCLVCGDRASGRHYGAISCEGCKGFFKRSIRKQLGYQCRGAMNCEVTKHHRNRCQFCRLQKCLASGMRSDSVQHERKPIVDRKEGIIAAAGSSSTSGGGNGSSTYLSGKSGYQQGRGKGHSVKAESAATPPVHSAPATAFNLNENIFPMGLNFAELTQTLMFATQQQQQQQQQHQQSGSYSPDIPKADPEDDEDDSMDNSSTLCLQLLANSASNNNSQHLNFNAGEVPTALPTTSTMGLIQSSLDMRVIHKGLQILQPIQNQLERNGNLSVKPECDSEAEDSGTEDAVDAELEHMELDFECGGNRSGGSDFAINEAVFEQDLLTDVQCAFHVQPPTLVHSYLNIHYVCETGSRIIFLTIHTLRKVPVFEQLEAHTQVKLLRGVWPALMAIALAQCQGQLSVPTIIGQFIQSTRQLADIDKIEPLKISKMANLTRTLHDFVQELQSLDVTDMEFGLLRLILLFNPTLLQQRKERSLRGYVRRVQLYALSSLRRQGGIGGGEERFNVLVARLLPLSSLDAEAMEELFFANLVGQMQMDALIPFILMTSNTSGL

SEQ ID NO: 26 - DNA, 2288 bp, Artificial Sequence
  Description of Artificial Sequence; note = synthetic construct
attggaacaaggagattttattgcgttagaaaaggttcaaaataggcacaaagtgcctgaaaatatcgtaactgaccggaagtaacataactttaaccaagtgcctcgaaaaatagatgtttttaaaagctcaagaatggtgataacagacgtccaataagaattttcaaagagccaaatgtttgggtttcagttatttatacagccgacgactattttttagccgcctgctgtggcgacaatggacggcgttaaggttgagacgttcatcaaaagcgaagaaaaccgagcgatgcccttgatcggaggaggcagtgcctcaggcggcactcctctgccaggaggcggcgtgggaatgggagccggagcatccgcaacgttgagcgtggagctgtgtttggtgtgcggggaccgcgcctccgggcggcactacggagccataagctgcgaaggctgcaagggattcttcaagcgctcgatccggaagcagctgggctaccagtgtcgcggggctatgaactgcgaggtcaccaagcaccacaggaatcggtgccagttctgtcgactacagaagtgcctggccagcggcatgcgaagtgattctgtgcagcacgagaggaaaccgattgtggacaggaaggaggggatcatcgctgctgccggtagctcatccacttctggcggcggtaatggctcgtccacctacctatccggcaagtccggctatcagcaggggcgtggcaaggggcacagtgtaaaggccgaatccgcggccacgcctccagtgcacagcgcgccagcaacggccttcaatttgaatgagaatatattcccgatgggtttgaatttcgcagaactaacgcagacattgatgttcgctacccaacagcagcagcaacaacagcaacagcatcaacagagtggtagctattcgccagatattccgaaggcagatcccgaggatgacgaggacgactcaatggacaacagcagcacgctgtgcttgcagttgctcgccaacagcgccagcaacaacaactcgcagcacctgaactttaatgctggggaagtacccaccgctctgcctaccacctcgacaatggggcttattcagagttcgctggacatgcgggtcatccacaagggactgcagatcctgcagcccatccaaaaccaactggagcgaaatggtaatctgagtgtgaagcccgagtgcgattcagaggcggaggacagtggcaccgaggatgccgtagacgcggagctggagcacatggaactagactttgagtgcggtgggaaccgaagcggtggaagcgattttgctatcaatgaggcggtctttgaacaggatcttctcaccgatgtgcagtgtgcctttcatgtgcaaccgccgactttggtccactcgtatttaaatattcattatgtgtgtgagacgggctcgcgaatcatttttctcaccatccatacccttcgaaaggttccagttttcgaacaattggaagcccatacacaggtgaaactcctgagaggagtgtggccagcattaatggctatagctttggcgcagtgtcagggtcagctttcggtgcccaccattatcgggcagtttattcaaagcactcgccagctagcggatatcgataagatcgaaccgttgaagatctcgaagatggcaaatctcaccaggaccctgcacgactttgtccaggagctccagtcactggatgttactgatatggagtttggcttgctgcgtctgatcttgctcttcaatccaacgctcttgcagcagcgcaaggagcggtcgttgcgaggctacgtccgcagagtccaactctacgctctgtcaagtttgagaaggcagggtggcatcggcggcggcgaggagcgctttaatgttctggtggctcgccttcttccgctcagcagcctggacgcagaggccatggaggagctgttcttcgccaacttggtggggcagatgcagatggatgctcttattccgttcatactgatgaccagcaacaccagtggactgtaggcggaattgagaagaacagggcgcaagcagattcgctagactgcccaaaagcaagactgaagatggaccaagtgcgggcaatacatgtagcaactaggcaaatcccattaattatatatttaatatatacaatatatagtttaggatacaatattctaacataaaaccatgggtttattgttgttcacagataaaatggaatcgatttcccaataaaagcgaatatgtttttaaacagaat

SEQ ID NO: 27 - protein, 508 aa, Artificial Sequence
  Description of Artificial Sequence; note = synthetic construct
MDNCDQDASFRLSHIKEEVKPDISQLNDSNNSSFSPKAESPVPFMQAMSMVHVLPGSNSASSNNNSAGDAQMAQAPNSAGGSAAAAVQQQYPPNHPLSGSKHLCSICGDRASGKHYGVYSCEGCKGFFKRTVRKDLTYACRENRNCIIDKRQRNRCQYCRYQKCLTCGMKREAVQEERQRGARNAAGRLSASGGGSSGPGSVGGSSSQGGGGGGGVSGGMGSGNGSDDFMTNSVSRDFSIERIIEAEQRAETQCGDRALTFLRVGPYSTVQPDYKGAVSALCQVVNKQLFQMVEYARMMPHFAQVPLDDQVILLKAAWIELLIANVAWCSIVSLDDGGAGGGGGGLGHDGSFERRSPGLQPQQLFLNQSFSYHRNSAIKAGVSAIFDRILSELSVKMKRLNLDRRELSCLKAIILYNPDIRGIKSRAEIEMCREKVYACLDEHCRLEHPGDDGRFAQLLLRLPALRSISLKCQDHLFLFRITSDRPLEELFLEQLEAPPPPGLAMKLE

SEQ ID NO: 28 - DNA, 2488 bp, Artificial Sequence
  Description of Artificial Sequence; note = synthetic construct
aaaaatgtcgacgcgaaaaaaggtatttattcattagtcagaaagtctggcattctttgtttgttggtaaaaagcgcaattgtttggaggcgagcgaataaagtgcgctgctccatcggctcaagattatgtaaatgcagcaacgaccccaccaacaacgaaactgcaacctgctccacttggcccaacggaccaatagcggacggacggacacggtggcgttggcaaagtgaaaccccaacagagaggcgaaagcgagccaagacacaccacatacacacgaagagaacgagcaagaagaaaccggtaggcggaggaggcgctgcccccagttcctccaatatacccagcaccacatcacaagcccaggatggacaactgcgaccaggacgccagctttcggctgagccacatcaaggaggaggtcaagccggacatctcgcagctgaacgacagcaacaacagcagcttttcgcccaaggccgagagtcccgtgcccttcatgcaggccatgtccatggtccacgtgctgcccggctccaactccgccagctccaacaacaacagcgctggagatgcccaaatggcgcaggcgcccaattcggctggaggctctgccgccgctgcagtccagcagcagtatccgcctaaccatccgctgagcggcagcaagcacctctgctctatttgcggggatcgggccagtggcaagcactacggcgtgtacagctgtgagggctgcaagggcttctttaaacgcacagtgcgcaaggatctcacatacgcttgcagggagaaccgcaactgcatcatagacaagcggcagaggaaccgctgccagtactgccgctaccagaagtgcctaacctgcggcatgaagcgcgaagcggtccaggaggagcgtcaacgcggcgcccgcaatgcggcgggtaggctcagcgccagcggaggcggcagtagcggtccaggttcggtaggcggatccagctctcaaggcggaggaggaggaggcggcgtttctggcggaatgggcagcggcaacggttctgatgacttcatgaccaatagcgtgtccagggatttctcgatcgagcgcatcatagaggccgagcagcgagcggagacccaatgcggcgatcgtgcactgacgttcctgcgcgttggtccctattccacagtccagccggactacaagggtgccgtgtcggccctgtgccaagtggtcaacaaacagctcttccagatggtcgaatacgcgcgcatgatgccgcactttgcccaggtgccgctggacgaccaggtgattctgctgaaagccgcttggatcgagctgctcattgcgaacgtggcctggtgcagcatcgtttcgctggatgacggcggtgccggcggcgggggcggtggactaggccacgatggctcctttgagcgacgatcaccgggccttcagccccagcagctgttcctcaaccagagcttctcgtaccatcgcaacagtgcgatcaaagccggtgtgtcagccatcttcgaccgcatattgtcggagctgagtgtaaagatgaagcggctgaatctcgaccgacgcgagctgtcctgcttgaaggccatcatactgtacaacccggacatacgcgggatcaagagccgggcggagatcgagatgtgccgcgagaaggtgtacgcttgcctggacgagcactgccgcctggaacatccgggcgacgatggacgctttgcgcaactgctgctgcgtctgcccgctttgcgatcgatcagcctgaagtgccaggatcacctgttcctcttccgcattaccagcgaccggccgctggaggagctctttctcgagcagctggaggcgccgccgccacccggcctggcgatgaaactggagtagggtcccgactctaaagtctcccccgttctccatccgaaaaatgtttcattgtgattgcgtttgtttgcatttctcctctctatcccttataccctacaaaagccccctaatattacgcaaaatgtgtatgtaattgtttattttttttttattacctaatattattattattattgatatagaaaatgttttccttaagatgaagattagcctcctcgacgtttatgtcccagtaaacgaaaaacaaacaaaatccaaaacttgaaaagaacacaaaacacgaacgagaaaatgcacacaagcaaagtaaaagtaaaagttaaactaaagctaaacgagtaaagatattaaaataacggttaaaattaatgcatagttatgatctacagacgtatgtaaacatacaaattcagcataaatatatatgtcagcaggcgcatatctgcggtgctggccccgttctaaatcaattgtaattactttttaacataaatttacccaaaacgttatcaattagatgcgagatacaaaaatcaccgacgaaaaccaacaaaatatatctatgtataaaaaatataaactgcataacaa

SEQ ID NO: 29 - protein, 906 aa, Artificial Sequence
  Description of Artificial Sequence; note = synthetic construct
MGEELPILKGILKGNVNYHNAPVRFGRVPKREKARILAAMQQSTQNRGQQRALATELDDQPRLLAAVLRAHLETCEFTKEKVSAMRQRARDCPSYSMPTLLACPLNPAPELQSEQEFSQRFAHVIRGVIDFAGMIPGFQLLTQDDKFTLLKAGLFDALFVRLICMFDSSINSIICLNGQVMRRDAIQNGANARFLVDSTFNFAERMNSMNLTDAEIGLFCAIVLITPDRPGLRNLELIEKMYSRLKGCLQYIVAQNRPDQPEFLAKLLETMPDLRTLSTLHTEKLVVFRTEHKELLRQQMWSMEDGNNSDGQQNKSPSGSWADAMDVEAAKSPLGSVSSTESADLDYGSPSSSQPQGVSLPSPPQQQPSALASSAPLLAATLSGGCPLRNRANSGSSGDSGAAEMDIVGSHAHLTQNGLTITPIVRHQQQQQQQQQIGILNNAHSRNLNGGHAMCQQQQQHPQLHHHLTAGAARYRKLDSPTDSGIESGNEKNECKAVSSGGSSSCSSPRSSVDDALDCSDAAANHNQVVQHPQLSVVSVSPVRSPQPSTSSHLKRQIVEDMPVLKRVLQAPPLYDTNSLMDEAYKPHKKFRALRHREFETAEADASSSTSGSNSLSAGSPRQSPVPNSVATPPPSAASAAAGNPAQSQLHMHLTRSSPKASMASSHSVLAKSLMAEPRMTPEQMKRSDIIQNYLKRENSTAASSTTNGVGNRSPSSSSTPPPSAVQNQQRWGSSSVITTTCQQRQQSVSPHSNGSSSSSSSSSSSSSSSSSTSSNCSSSSASSCQYFQSPHSTSNGTSAPASSSSGSNSATPLLELQVDIADSAQPLNLSKKSPTPPPSKLHALVAAANAVQRYPTLSADVTVTASNGGPPSAAASPAPSSSPPASVGSPNPGLSAAVHKVMLEA

SEQ ID NO: 30 - DNA, 3750 bp, Artificial Sequence
  Description of Artificial Sequence; note = synthetic construct
agtcaccgtcgcagtcgcagcagttgaggttcgctctcctcgatttcgggcaaatccgataccatatagcacagcgtaccgcactctgggtatattcgtaacgcgctttggcttttacagttagtcgcgttcgagaccttgtcgagttttgtcatgttagccagcgatccgcgggatccgaaataagccaagaatcacaacgcgagtgcggcagttgccagcagtaactacaccaatatttatattaattaaaataaattaaatgaaacaacatgctgattaatgccaatgaatgttaaatgcaattgttaatgtgaagaaaagtcgaccaagtctccccaaaacaacacttattcaacatccactacacactcgcctttctggattacgcgcccaaaaaaaaacaaaaattaaaaattaaaccaaaccaacaactaatttatttgctaaatattccaaaaattcaatcaatgtgaaaagcaagcaaacaaagttcctctcacaacaaaacagcagttaattaaaatatctaaccgagataaagtgcaaagaagataacaagtttctcaagcaaacatccatatgtacctgagtaccaaccaaaaagctgtgtgtgtgccaaaaaccgaagaggaattatccaaaaatatttaatgagcaagctcaactgagtggttgatgtgccccccaagggaaaagtgaccaagtcaagatattttgtcaaatcgaacacagaaaacacaaaaatgggcgaagaactcccgatattgaagggcatacttaaaggcaacgtcaactatcacaatgcgcctgtgcgttttggacgcgtgccgaagcgcgaaaaggcgcgtatcctggcggccatgcaacagagcacccagaatcgcggccagcagcgagccctcgccaccgagctggatgaccagccacgcctcctcgccgccgtgctgcgcgcccacctcgagacctgtgagttcaccaaggagaaggtctcggcgatgcggcagcgggcgcgggattgcccctcctactccatgcccacacttctggcctgtccgctgaaccccgcccctgaactgcaatcggagcaggagttctcgcagcgtttcgcccacgtaattcgcggcgtgatcgactttgccggcatgattcccggcttccagctgctcacccaggacgataagttcacgctcctgaaggcgggactcttcgacgccctgtttgtgcgcctgatctgcatgtttgactcgtcgataaactcaatcatctgtctaaatggccaggtgatgcgacgggatgcgatccagaacggagccaatgcccgcttcctggtggactccaccttcaatttcgcggagcgcatgaactcgatgaacctgacagatgccgagataggcctgttctgcgccatcgttctgattacgccggatcgccccggtttgcgcaacctggagctgatcgagaagatgtactcgcgactcaagggctgcctgcagtacattgtcgcccagaataggcccgatcagcccgagttcctggccaagttgctggagacgatgcccgatctgcgcaccctgagcaccctgcacaccgagaaactggtagttttccgcaccgagcacaaggagctgctgcgccagcagatgtggtccatggaggacggcaacaacagcgatggccagcagaacaagtcgccctcgggcagctgggcggatgccatggacgtggaggcggccaagagtccgcttggctcggtatcgagcactgagtccgccgacctggactacggcagtccgagcagttcgcagccacagggcgtgtctctgccctcgccgcctcagcaacagccctcggctctggccagctcggctcctctgctggcggccaccctctccggaggatgtcccctgcgcaaccgggccaattccggctccagcggtgactccggagcagctgagatggatatcgttggctcgcacgcacatctcacccagaacgggctgacaatcacgccgattgtgcgacaccagcagcagcaacaacagcagcagcagatcggaatactcaataatgcgcattcccgcaacttgaatgggggacacgcgatgtgccagcaacagcagcagcacccacaactgcaccaccacttgacagccggagctgcccgctacagaaagctagattcgcccacggattcgggcattgagtcgggcaacgagaagaacgagtgcaaggcggtgagttcggggggaagttcctcgtgctccagtccgcgttccagtgtggatgatgcgctggactgcagcgatgccgccgccaatcacaatcaggtggtgcagcatccgcagctgagtgtggtgtccgtgtcaccagttcgctcgccccagccctccaccagcagccatctgaagcgacagattgtggaggatatgcccgtgctgaagcgcgtgctgcaggctccccctctgtacgataccaactcgctgatggacgaggcctacaagccgcacaagaaattccgggccctgcggcatcgcgagttcgagaccgccgaggcggatgccagcagttccacttccggctcgaacagcctgagtgccggcagtccgcgacagagtccagtcccgaacagtgtggccacgcccccgccatcggcggccagcgccgccgcaggtaatcccgcccagagccagctgcacatgcacctgacccgcagcagccccaaggcctcgatggccagctcgcactcggtgctggccaagtctctcatggccgagccgcgcatgacgcccgagcagatgaagcgcagcgatattatccaaaactacttgaagcgcgagaacagcacagcagccagcagcaccaccaatggcgtgggcaaccgcagtcccagcagcagctccacaccgccgccatcggcggtccagaatcagcagcgttggggcagcagctcggtgatcaccaccacctgccagcagcgccagcagtccgtgtcgccgcacagcaacggttccagctccagttcgagctctagctccagctccagttcgtcatcctcctccacatcctccaactgcagctccagctcggccagcagctgccagtatttccagtcgccgcactccaccagcaacggcaccagtgcaccggcgagctccagttcgggatcgaacagcgccacgcccctgctggaactgcaggtggacattgctgactcggcgcagcctctcaatttgtccaagaaatcgcccacgccgccgcccagcaagctgcacgctctggtggccgccgccaatgccgttcaaaggtatcccacattgtccgccgacgtcacagtgacagcctccaatggcggtcctccgtcggcggcggcgagtccggcgcccagcagcagtccgccggcgagtgtgggctcccccaatccgggcctgagcgccgccgtgcacaaggtaatgctggaggcgtaagagcgggaggaggtaggtggttttacgcggagaagtgggagagacagagactgggagtggcagttcagcgaagcaggaagcaggatcacttggagcggcgggagttgaattaaattattttaccatttaattgagacgtgtacaaagtttgaaagcaaaaccaacatgcatgcaatttaaaactaatatttaaagcaacaacaaacaaaacaactacaagttattaatttaaaaaacaaacaaacaaacaaacaacaaaaaacccaagcttgaatggtattac

SEQ ID NO: 31 - protein, 392 aa, Artificial Sequence
  Description of Artificial Sequence; note = synthetic construct
MHPSHLQQQQQQHLLQQQQQQQHQPQLQQHHQLQQQPHVSGVRVKTPSTPQTPQMCSIASSPSELGGCNSANNNNNNNNNSSSGNASGGSGVSVGVVVVGGHQQLVGGSMVGMAGMGTDAHQVGMCHDGLAGTANELTVYDVIMCVSQAHRLNCSYTEELTRELMRRPVTVPQNGIASTVAESLEFQKIWLWQQFSARVTPGVQRIVEFAKRVPGFCDFTQDDQLILIKLGFFEVWLTHVARLINEATLTLDDGAYLTRQQLEILYDSDFVNALLNFANTLNAYGLSDTEIGLFSAMVLLASDRAGLSEPKVIGRARELVAEALRVQILRSRAGSPQALQLMPALEAKIPELRSLGAKHFSHLDWLRMNWTKLRLPPLFAEIFDIPKADDEL

SEQ ID NO: 32 - DNA, 3341 bp, Artificial Sequence
  Description of Artificial Sequence; note = synthetic construct
aagcattaacgaaagaactgcgcacaaagtagggaggcaataattacatatgtacatggctgggaaaggccttaactaaacttagcaaactaataaatagaaaaaaggaaatattggccaaatattatagtattgggaatattaggttacttgatatcaaaaattaatgtctattttatacacttattcttagacttaatgttaacttatcgtacttattatgattggtttttcaagattaccagaacttgatagattggtctagcttttgaaatcggatagcattttctttaaaggactttgccatatgctaaagcctaacttcttttttcaattcagccacagctgacaaaagcgaagaaaatttgaaagaccgtgaatccttttgaaacgccctctccggattcctcattaagtgcaaaagatataacatcgcagagatttcccataaaaatgctgatcaggcgccctcgcaggttgccaacgtcgatttccgccagcaggacgatgatgaagatgatggatgcccatctcaccgattcgatccgagcaacatggatgtataccaaatagagctggaggaacaggcacaaatccgctccaaactgctggtcgaaacctgtgtgaagcactcgtcttcggagcagcagcagctccaagttaagcaggaggacctcatcaaggatttcactcgggacgaggaggaacagccaagcgaagaggaggcggaggaagaggacaacgaagaggacgaggaagaagaaggcgaagaagaagaggaggacgaggacgaggaagccctgctgccggtagtcaattttaatgcaaattcagactttaatttgcatttctttgacacaccggaggactcgtccacccaaggggcctacagtgaggccaatagcttggaatccgagcaggaagaggagaagcaaacacagcagcatcagcagcagaagcagcatcaccgggatttggaggattgcctaagtgccattgaagctgatccattgcagttgttgcattgcgacgacttctatagaacatcagccctagcagagagtgttgcagccagtctaagcccacagcagcagcagcaacggcagcacacccaccagcaacaacagcaacagcagcagcagcagcaacaccctggacagcagcaacatcagctcaactgcacgctgagcaatggtggaggtgctttgtacaccatcagcagtgtgcatcagttcggtccggccagcaaccacaacaccagcagcagctccccctcctccagcgccgcccactcttcgccggacagcggctgctcgtcggcctcctcctccggatcttcgcgatcctgcggatcctcctctgcatcctcctcctcgtcagcggtcagcagcaccatcagcagcggccgcagcagcaacaacagcgtcgtcaaccccgcagcaacatcttcatctgttgcgcatctgaacaaagagcaacagcagcagccactgccgacgacacagctgcaacagcagcagcagcaccagcagcagttgcaacacccgcagcagcagcaatcttttggcctagcagacagcagcagcagcaacggcagcagcaacaacaacaacggtgtctcctcgaaatcatttgtgccctgcaaagtctgtggcgacaaggcatcgggataccactatggtgtaacctcctgcgagggttgcaagggattctttcgtcgcagtatccagaagcaaatcgaatatcgctgtttgcgggacggcaagtgcctggtcatcagactgaaccgcaatcgctgccagtactgccgcttcaagaaatgcctttccgctggcatgagccgcgattccgtacgttatggtcgcgttcccaagcgttcccgtgagctgaacggagcggccgcctcctccgccgccgctggagctcctgcctccctcaatgtggatgactctaccagcagcacactgcacccgagtcacctacagcagcagcagcaacagcatctactacagcagcaacagcagcagcaacatcagccacagctgcagcaacaccaccaactgcaacagcagccgcatgtaagcggcgtacgtgtgaagaccccgagtactccacaaacgccacaaatgtgttcgatcgcctcctcgccatcggagctgggcggttgcaatagtgccaataacaataacaataataacaacaacagtagcagcggtaatgccagcggtggcagcggcgtgagcgtcggcgttgttgttgtgggcggacaccagcaactggtgggaggcagcatggtgggaatggcgggcatgggcacggatgcccaccaggtgggcatgtgtcacgacggcttggcgggaacggcaaacgagctgaccgtctacgatgtcatcatgtgcgtgtcgcaggcgcaccgcctcaactgctcctacacggaggaactgaccagagagctcatgcgtcgtcccgtgacggtgccacaaaatgggattgccagcacagtggccgagagtctggagttccagaagatctggctgtggcaacagttctcggccagggtgacgcctggcgttcagcggattgtggagtttgcgaaacgcgtacctggcttctgtgatttcacccaagatgaccagcttatactaataaagctgggcttcttcgaggtctggttgacccatgtggcccggttgatcaatgaggcgacattgacactggacgatggtgcctacctgacgcgccagcagcttgagatactctacgattctgactttgtcaacgccttgctgaactttgccaacacgctgaacgcctacgggctgagtgacaccgaaatcggactcttctcggccatggtgctgcttgcctcggatcgagctggactcagcgagcccaaggtgatcggcagggccagggaactggtggccgaggcgctgcgcgtacagatcctgcgttcgcgggcaggatccccacaggcgctgcagctgatgccggcgctggaagccaagatacccgagctgagatccttgggggccaagcacttctcacacctagactggctacggatgaactggaccaagctgcgcctgccgcccctcttcgccgagatcttcgacatcccgaaggctgacgatgagctgtaggatgtggagccaaccccgcgattccagggccgtgcaaagcaaaccgcaacaagaacagaatattctaccacttgtaggcttaagcaacgtagctatagatcgaaatgggagggccgcagatcagatacacgtctactcagcattaccggagagatagtccactaagcctatatgcatactactatactagcagtgtta

SEQ ID NO: 33 - protein, 878 aa, Artificial Sequence
  Description of Artificial Sequence; note = synthetic construct
MKRRWSNNGGFMRLPEESSSEVTSSSNGLVLPSGVNMSPSSLDSHDYCDQDLWLCGNESGSFGGSNGHGLSQQQQSVITLAMHGCSSTLPAQTTIIPINGNANGNGGSTNGQYVPGATNLGALANGMLNGGFNGMQQQIQNGHGLINSTTPSTPTTPLHLQQNLGGAGGGGIGGMGILHHANGTPNGLIGVVGGGGGVGLGVGGGGVGGLGMQHTPRSDSVNSISSGRDDLSPSSSLNGYSANESCDAKKSKKGPAPRVQEELCLVCGDRASGYHYNALTCEGCKGFFRRSVTKSAVYCCKFGRACEMDMYMRRKCQECRLKKCLAVGMRPECVVPENQCAMKRREKKAQKEKDKMTTSPSSQHGGNGSLASGGGQDFVKKEILDLMTCEPPQHATIPLLPDEILAKCQARNIPSLTYNQLAVIYKLIWYQDGYEQPSEEDLRRIMSQPDENESQTDVSFRHITEITILTVQLIVEFAKGLPAFTKIPQEDQITLLKACSSEVMMLRMARRYDHSSDSIFFANNRSYTRDSYKMAGMADNIEDLLHFCRQMFSMKVDNVEYALLTAIVIFSDRPGLEKAQLVEAIQSYYIDTLRIYILNRHCGDSMSLVFYAKLLSILTELRTLGNQNAEMCFSLKLKNRKLPKFLEEIWDVHAIPPSVQSHLQITQEENERLERAERMRASVGGAITAGIDCDSASTSAAAAAAQHQPQPQPQPQPSSLTQNDSQHQTQPQLQPQLPPQLQGQLQPQLQPQLQTQLQPQIQPQPQLLPVSAPVPASVTAPGSLSAVSTSSEYMGGSAAIGPITPATTSSITAAVTASSTTSAVPMGNGVGVGVGVGGNVSMYANAQTAMALMGVALHSHQEQLIGGVAVKSEHSTTA

SEQ ID NO: 34 - DNA, 5586 bp, Artificial Sequence
  Description of Artificial Sequence; note = synthetic construct
tagtatttttttggactttgttgttaacggttgttcgctcgcacgtacgaagcccgatcgcgttcgtcaaaaaacaagatacaaaatacagcacacacaattgaaaacgacaacctaacagtacggtttcccaaagcaccttacatttcaaaaccgaaaacccccaaaatgttgtaaccaaataatgtttaaatcacatatacacctacatatatttatgaaaaattgttagacaaatcccaaataataccagttcccccaacaaccgcaacaaacacaagtgcaattcatcggcaaaaattaatataaagtgcaaatgcattgtagctgaaactcaaacaatagtaaaaatacatacataagtggtgaagaagcaaaaggaaatagttcttaaaataacgcaaatcgagagcatatattcatatttgtacagatattatatggcggctgcatagtgcaaactgcggctgagggaatacagcggtatcgaaatgtaaataggaaacaacgaagccagaactcgaaatcaaacatcagcaacgtgacacacagacataagacgcccgtctagtcgtggtctgtggaacgctagctccgctttgccaggagccggagactttttccgcatccacaatattacatatgtacatatatcgaagatagtgcgcgagtgagtgagggatttgtgccgtggatcccgatccccttacatatatataaaggtagtgaaaagattttactcaacattccaaatagtgctttgtcaactggaataccttttgttcaaatacgcagtgggcccatggatacttgtggattagtagcagaactggcgcactatatcgacgcatatgctctgattgtttcccgcactaaatgagcagggattcgggcgaaaatgtattttgaacgcaaacaagtgcgcaaaaaatactagctccaccacgaaactgcacaaaacaccgccagaagcgagcagaacctcgggccgcacgaccgagcttcgtaaagcaacagaggatcttaccaggagatagctcttctccacatagaccaactgccagggacaagctccttgtccccagccgacgctaagtgaacggaaaacggccacaaaacggcgactatcggctgccagaggatgaagcggcgctggtcgaacaacggcggcttcatgcgcctaccggaggagtcgtcctcggaggtcacgtcctcctcgaacgggctcgtcctgccctcgggggtgaacatgtcgccctcgtcgctggactcgcacgactattgcgatcaggacctttggctctgcggcaacgagtccggttcgtttggcggctccaacggccatggcctaagtcagcagcagcagagcgtcatcacgctggccatgcacgggtgctccagcactctgcccgcgcagacaaccatcattccgatcaacggcaacgcgaatgggaatggaggctccaccaatggccaatatgtgccgggtgccactaatctgggagcgttggccaacgggatgctcaatgggggcttcaatggaatgcagcaacagattcagaatggccacggcctcatcaactccacaacgccctcaacgccgaccaccccgctccaccttcagcagaacctggggggcgcgggcggcggcggtatcgggggaatgggtattcttcaccacgcgaatggcaccccaaatggccttatcggagttgtgggaggcggcggcggagtaggtcttggagtaggcggaggcggagtgggaggcctgggaatgcagcacacaccccgaagcgattcggtgaattctatatcttcaggtcgcgatgatctctcgccttcgagcagcttgaacggatactcggcgaacgaaagctgcgatgcgaagaagagcaagaagggacctgcgccacgggtgcaagaggagctgtgcctggtttgcggcgacagggcctccggctaccactacaacgccctcacctgtgagggctgcaaggggttctttcgacgcagcgttacgaagagcgccgtctactgctgcaagttcgggcgcgcctgcgaaatggacatgtacatgaggcgaaagtgtcaggagtgccgcctgaaaaagtgcctggccgtgggtatgcggccggaatgcgtcgtcccggagaaccaatgtgcgatgaagcggcgcgaaaagaaggcccagaaggagaaggacaaaatgaccacttcgccgagctctcagcatggcggcaatggcagcttggcctctggtggcggccaagactttgttaagaaggagattcttgaccttatgacatgcgagccgccccagcatgccactattccgctactacctgatgaaatattggccaagtgtcaagcgcgcaatataccttccttaacgtacaatcagttggccgttatatacaagttaatttggtaccaggatggctatgagcagccatctgaagaggatctcaggcgtataatgagtcaacccgatgagaacgagagccaaacggacgtcagctttcggcatataaccgagataaccatactcacggtccagttgattgttgagtttgctaaaggtctaccagcgtttacaaagataccccaggaggaccagatcacgttactaaaggcctgctcgtcggaggtgatgatgctgcgtatggcacgacgctatgaccacagctcggactcaatattcttcgcgaataatagatcatatacgcgggattcttacaaaatggccggaatggctgataacattgaagacctgctgcatttctgccgccaaatgttctcgatgaaggtggacaacgtcgaatacgcgcttctcactgccattgtgatcttctcggaccggccgggcctggagaaggcccaactagtcgaagcgatccagagctactacatcgacacgctacgcatttatatactcaaccgccactgcggcgactcaatgagcctcgtcttctacgcaaagctgctctcgatcctcaccgagctgcgtacgctgggcaaccagaacgccgagatgtgtttctcactaaagctcaaaaaccgcaaactgcccaagttcctcgaggagatctgggacgttcatgccatcccgccatcggtccagtcgcaccttcagattacccaggaggagaacgagcgtctcgagcgggctgagcgtatgcgggcatcggttgggggcgccattaccgccggcattgattgcgactctgcctccacttcggcggcggcagccgcggcccagcatcagcctcagcctcagccccagccccaaccctcctccctgacccagaacgattcccagcaccagacacagccgcagctacaacctcagctaccacctcagctgcaaggtcaactgcaaccccagctccaaccacagcttcagacgcaactccagccacagattcaaccacagccacagctccttcccgtctccgctcccgtgcccgcctccgtaaccgcacctggttccttgtccgcggtcagtacgagcagcgaatacatgggcggaagtgcggccataggacccatcacgccggcaaccaccagcagtatcacggctgccgttaccgctagctccaccacatcagcggtaccgatgggcaacggagttggagtcggtgttggggtgggcggcaacgtcagcatgtatgcgaacgcccagacggcgatggccttgatgggtgtagccctgcattcgcaccaagagcagcttatcgggggagtggcggttaagtcggagcactcgacgactgcatagcaggcgcagagtcagctccaccaacatcaccaccacaacatcgacgtcctgctggagtagaaagcgcagctgaacccacacagacataggggaaatggggaagttctctccagagagttcgagccgaactaaatagtaaaaagtgaataattaatggacaagcgtaaaatgcagttatttagtcttaagcctgcaaatattacctattattcatacaaattaacatataatacagcctattaacaattacgctaaagcttaattgaaaaagcttcaacaacaattggacaaacgcgttgaggaaccgggagaaaatttaagaaaaaaaaaaccattgaaaattatgaaatttagtatacattttttttgggtggatgtatgtcgcatcagactcacgatcaattctcgaattttgttaactaaattgatcctccaaactgcatgcgaaacagatcagaaaagagaacagacagtagggcgtgaacagagggaagagagaagagaataaagattgtttatatttaaaaaatatataaaataataattactaactctaaacgtaatgaaagcaactgtataatatctaactataactataaattcgtactgtagggaagtgagaaaatctgttaaatgaaacaaaaataatgataataacattatcatccaccataattaaaatcatttaaagtaattaaaaacaaaacacttttaaaacacgcaaaacttggactgattttataaatattttttaatcataaagaaaggcaacctgaaaaaaatattacaaaaacaaataacaacatattttattatgacacccttatatgttttcaaaacgagaatttaaattcttagattcttataatttcatccaaaaatattagccagcaaaaacctttattattggcattgtttttagacatgttttcaaaaaaaactttgatattgaaactaaacaaaggataatgaaatgaaagtgattggagtcttactcaaaaaccaaaaggcatcaaaaggtattaaattaaaaatataatctaatttcgagttcaagaaacactttttggtggaaaatagttttcaatcactttgataaaaaccacacaaattaataaatacatgcatacaccaaaagacttcaatatatatttttaaaatttacattgataattcgaaatttgaataagaatcacatccatctaatttggctaaatcaaaatttttatgaaagccacacaaaaaacgtgcaaatttgattactttggcaatttttatgttatacaaaatttatgcaattgattttcaaaataatttttattagattgtattagtttcattttgctttgggatgtacattttaaataaattttactttaaattgttggccttattttaacttaaatcaaatttattctaattttagtaaaaaaaaatgtgtttaaaattgaaaataagaacactgtaaaatattaataaaaaattaaagtttaaagtgattcttttattatgtaaaaagaagacaaaaaatatcttacgtagctttctacttgaattgtgcaattttttacttttactactaatcctaatttaaatataatttacacacacgcctacacatccagccacatatttttaattttaagtcaacctaatttataaatatgaatttgtataatgacgaactaaaattagcatgacatcatggacatacttggaaataactctatcaaacgagctaaatgcattgaagaagaaaattcttgttaaatatagtctgcacttcgacaaacgaaaatcagtgaatt

SEQ ID NO: 35 - protein, 808 aa, Artificial Sequence [sequence omitted]

SEQ ID NO: 36 - DNA, 4841 bp, Artificial Sequence
  Description of Artificial Sequence; note = synthetic construct
actaacaaaacaaacattttgctacttcgtcgcaggcgggactgtgttgcgtcgtgtgatcgctagagcggttgtggaatcggattcgagcgcaaaacaccgttcatgctgtgagcgaaaaagagtggtagcgcctacagtggcatatgtagttaaatccgtgaataagtgaaaaatccgatatttgtcgtgcaataatttcctcgattggcatcaagtggcttccagtcgggtacatattgcacaagaaatgttatacgcataatgtgcacgcaaattaaacgaattctctatgaaaatgtgactagaatgtgagtcgaacaaaacgagtaaaacgtgaaatcccaactggcttttgggtaacaaatcttatcaacacagcaacggaaatacattaaaatcttgatagactgagaaagggacaattggaatacttttagttatttttaaatgttttacaacacaatggaactgcatcaacgacacctctcaaacttttacaaattgcacaactgagaaatagtctttgataaataaataaaatataagaaatcgctactgaaacaagatgccaaacatgtccagcatcaaagcggagcagcaaagcggtcctcttggaggaagtagcggctatcaagtaccggtcaacatgtgcaccaccacagtcgcgaatacgacgaccactttgggaagctccgccgggggagccactggctcccggcacaacgtctccgtgacaaacatcaagtgcgaactagacgaactaccgtcaccgaacggcaacatggtgccggttatcgcaaactacgttcacggtagcttgcgcattccactcagtggacattcaaatcatagggagtccgattcggaggaggagctggcaagtattgagaacttgaaggttcggcgaaggacggcggcggacaaaaatggtcctcgtccaatgtcctgggagggcgagctgagcgatactgaggtcaacgggggcgaagagctgatggaaatggagccaacaattaagagtgaggtggtccctgctgttgcacccccacaacccgtctgcgcactacaaccgataaaaacagagctagagaacattgcaggcgagatgcagattcaagagaagtgttacccccagtccaacacacaacatcacgctgccacaaaattaaaagtggccccgacgcaaagtgatccgatcaatctcaagttcgaaccgcctctgggagacaattctccgctactggctgcacgtagcaagtccagcagtggaggccacctaccactgccaacgaatcccagtcccgactccgccatacattccgtctacacgcacagctccccctcgcagtcgcctctgacgtcgcgccacgccccctacactccgtctctgagccgcaacaacagcgacgcctcgcacagtagctgctacagctatagctccgaattcagtcccacacactcgcccattcaagcgcgtcatgccccacccgccggcacgctctatggcaaccaccatggtatttaccgccagatgaaggtggaagcctcatccactgtgccgtccagtgggcaggaggcgcagaacctgagtatggactctgcctctagcaatctggatacagtgggcttaggatcttcgcaccccgcatctccggcgggcatatcacgtcagcagttgatcaactcgccctgccccatctgcggtgacaagatcagcggatttcattacgggattttctcctgcgagtcttgcaagggcttcttcaagcgcaccgtgcaaaatcgcaagaactacgtgtgcgtgcgtggtggaccatgtcaggtcagcatttccacgcgcaagaaatgtccagcctgccgcttcgagaagtgtctgcagaagggaatgaaactagaagcgattcgggaggaccgaacccgtggcggccgctccacataccagtgctcctacacgctgcccaactcaatgcttagtccgctgcttagtcctgatcaagcggcagcagctgccgccgcagcagcagtggcaagtcagcagcagccgcaccagcgactacatcaactaaatggatttggaggtgtacccattccctgctctacttctcttccagccagccctagtttggcaggaacttcggtcaagtcggaagagatggcggagacgggcaagcaaagcctccgaacgggaagcgtaccaccactactgcaggaaatcatggatgtagagcatctgtggcagtacaccgatgcagagctggcccgcatcaaccaaccactgtccgcattcgcctctggcagctcttcgtcgtcgtcatcgtcaggtacatcctcaggcgcccatgcacaactcaccaatccactactggctagtgctggtctctcgtccaatggcgagaatgccaatcctgatcttatcgctcatctctgcaacgtggctgatcaccgtctttataaaatcgtcaaatggtgcaagagcttgccgctttttaagaacatttcgatcgatgaccaaatctgcttgctcattaactcgtggtgcgagctgttgctcttctcctgctgttttagatcaattgatactcctggagagattaaaatgtcacaaggcaggaagataaccctatcgcaggccaaatcaaatggcttgcagacttgcattgaacggatgctcaacctaacagatcacctgaggcgattgcgcgttgatcgctacgaatatgttgccatgaaagttattgtgctgttgcagtcagatacgacagagttacaggaagcggtaaaggtgcgcgagtgtcaggaaaaagctttgcagagcttgcaagcttacaccctggcgcattatcctgacacgccatccaagtttggggagcttttgctacgcattcctgatttgcagcgaacgtgccagcttggcaaggagatgttgacgatcaagactcgcgatggagctgatttcaatttgctaatggagcttttgcgcggagagcattgacaattgataactaagacggaaatcttttaccattggcaaaacaagtttcacatatttagtattagatatatatattctatagataagatccttactgtaagttctgaaaacatgtgcctaaaaaccaaagccacgatagcagtcacatcaggcccactggtcgagattaaatccaagagcaagattgccaaatttttacaccaatatatattttgatatgagccatgtgcagggcctcagatcgctgttgttgtcggctaaagtttcagtaagaaaagtatatattgattttgctatttatacatatttgacttatgtatagtgtaaactaaagcacacatggaaaatgaaaagactaaacaaatttatttaaagattacttttactattatagaaaaaggggaaaaataaaaaacacaaaggcagagaagaaaatttagttacaacaggtagcgacatttttatattttcttatataaggaaatattcaatgtattttaaatataaagccaaacccgatttggtttgggaaagagctactgaaatttttgatatctatatattcatcactagaagacgaatgaatgtatccaatgtttaaatgttgtagcgtttagttttagtgcaatttcacacatgtctacatacatgaatattcagcgagatatgtttgcaaactattataaagcaaaagaccactcgaaatcgccatcactgggttggctaagactattccagttatgctgtttgttgcataaaaaaccacaactacgtacatcaataaaatgtataattttttattggagttttagatttgtattaacttcttccttataattacgattattattattattactaattttatgaatattgtgtaacactgacttaaatagctgaaaaaatcctgcaacaggatttaaaacacctgaatacacaaaacattataacatgaatacattttgcttatggcctagatagtttgatatgtactttgcatatgtatgcatgtgtctatatgtgagtacgtaccatacaaattcctgtcccaccagaaaaatcacacgcaataaaaaattccaaaatactaagctcgtatctacaaagaaagattaaaagacaaattgatgaataggaatatgttgccggaagtccaagagatttggctgaaagtatcgacaaattttcaacacatcgttcatggatattgtgctaacactctcagtttgaaaatcattttctgttaaactttctatataataagttctccattcgattttgtatttacaatttgtttctttaattttcctttatcagttgtatctatgaaacatgaggatctcagttcatattgatcgtgttcttctgccgtacaccgcttctgtccgttaatgtaaaccataagtataaatgaaattagttaaatgtttatttataaataaagcgctataataaatttcaatacatttatcatagttaactgattaagaccactgaaatcaaaaatattttatttactaagcaaagcacacgcaaacaatttataatgtttattacgttaacaacaaactcatttttaataattctttatgaatacacaaagttacgcaattttccctctaggcgcattgcttaaatagttaaagaaaaataataaacccatagcgcaatatttaatgtaaaacagttttccttgcgtgtgatgtttgctctagctacgtacaaattcatcatttattaaatttaaaactcaattttgcttttaaataaatttaataagtaaaattcaacaataattgatatacaattgtcaatgcaatattttgtaataaaaatgcgaaaaatc

SEQ ID NO: 37 - DNA, 7555 bp, Artificial Sequence
  Description of Artificial Sequence; note = synthetic construct
gggccccccctcgaggtcgacggtatcgataagcttgccggtggcggagaaggtgtatccgtgattaagaaagagccagccgatgagaagcagccacagccacatgaccacggtgcgtccgtacacaagatggcactgctggtgccgtttcgagaccgatttgaggaactcctccagttcgtcccccacatgaccgcctttttgaagcggcagggcgtggcgcaccacatctttgtgctgaaccaggtggacaggttccgcttcaatcgcgcctctctcatcaacgtgggtttccagtttgccagcgatgtgtacgattacattgccatgcacgacgtagacttgctgcccttgaatgacaatctgctctatgagtatcccagcagcttgggaccactgcacatcgccggaccgaagctacatcccaaataccactatgataacttcgttggaggaatattactggtgcgacgcgagcactttaagcagatgaacggcatgtcgaaccagtactggggctggggattagaggacgacgagttcttcgtgcgcatccgggatgcaggactgcaggtgacgcggccgcagaacattaagactggcactaatgatacattcaggtgagaccagtgctccggatttcgcaactagacgtgactactaataattattgtcattcaacctcagccatattcacaaccgctatcatcgtaagcgggacacccagaagtgcttcaaccagaaggagatgacccgcaagcgggaccacaagacgggcctggacaacgtgaagtacaaaatacttaaggtgcatgagatgctcattgaccaggtgccggtgaccatcctcaacattttgctcgattgtgatgttaataaaacgccttggtgcgactgctccggaacggcagcggctgcatcggcggtacaaacctgatgggttgtgttaaaccaaagatcctatgtttatttcgctattatagtgtgttgtattgtataaatgcgctaatacacgtgcaccatgccatagaggaatgtccagaagagcacgtaggtgcaaaggccgcccatgaactgattggtcagcagatttctgcggttaatgaaaaacttgcgccactgggtgcccgatttcacgagcaccagaatccagagcacgaacacggacaggaagtagaaaaggaatcccagcgtaccactcaggcccaaaatacctgcgaattggtgggacattaactaagttggttcaccatcaattggagccaattacccgcagcgcagcccgagatggcagccatcgatgtgcgacagtattccacggcggatatgttgttccggatggcgccctcgctgtaggcgattatttcgccagtcttggactgcgtggtcttcactcgattcattttatttaattaaattctactttaatttctagcaaaaatattcctaggctgtgaacttcgattgtgtgccgattgtgttatcgattggtgccgataactatgcactgtaaaaattcactagcggtttttgcaggataaatagtttttgtaaattttccgagataaacttgacgagctgtttaatgttaaataatgaagtttaatacaatatcaaatatatttgctgaagtgtatatttattctcaccgctctgtgcttcgatggctcacaattgcgtttgccattcgcccgggcacgtagattgttgttattgggattggcctggagcactcggacggacagtaattcattaaaatatgtggtgataacgcgagctgccgaatctgcgtgcaattcgtgcgtttgacgtgggtactaactgctatgctgtcgcgcggacagttgttctgatacgcagagttcctgcctcaccacacacgaccacctccattaaaaccagccaccccccccagcgcctcctccaccgacagcagctgctccaccgcaccaccaggagaggggcaattaaaaaatcaatcagagggcccatcacttgcttgtaaccgccgaagaactgcgcggtgtgcggggacaaggctctgggctacaacttcaatgcggtcacctgcgagagctgcaaggcgttcttccgacggaacgcgctggccaagaagcagttcacctgccccttcaaccaaaactgcgacatcactgtggtcactcgacgcttctgccagaaatgccgcctgcgcaagtgcctggatatcgggatgaagagtgaaaacattatgtccgaggaggacaagctgatcaagcggcgcaagatcgagaccaaccgggccaagcgacgcctcatggagaacggcacggatgcgtgcgacgccgatggcggcgaggaaagggatcacaaagcgccggcggatagcagcagcagcaaccttgaccactactcggggtcacaggactcgcagagctgcggctcggcggacagcggggccaatgggtgctccggcagacaggccagttcgccgggcacacaggtcaatccgcttcagatgacggccgagaagatagtcgaccagatcgtatccgacccggatcgagcctcgcaggccatcaaccggttgatgcgcacgcagaaagaggctatatcggtgatggagaaggtaatcagctcacaaaaggacgccttaaggctggtgtcgcatttgatcgactatccaggtgggtgcagacaagatttcatcgtttagccttatccgctcacctatgaacgacttgaatctttacaggcgacgcactcaagatcatttcaaagtttatgaactcgccctttaacgcgctgacaggttagagttttaaaatttgtggttttaaacttaatttcacattccttgttaatttaaatacgcagtattcaccaaattcatgagctcacccacggacggcgttgaaattatctcaaagatagttgattcgcccgcggacgtggtggagttcatgcagaacttgatgcactcgccagaggacgccatcgatataatgaacaagttcatgaataccccagcggaggcgctgcgcattcttaaccgaatcctaagcggcggaggagcgaacgcagcccagcagacagcagaccgcaagccattgctggacaaggagccggcggtgaagcctgcagcgccagcggagcgagctgatactgtcattcaaagcatgctgggcaacagtccgccaatttcgccacatgatgctgccgtggatctgcagtaccactcgcccggtgtcggggagcagcccagtacatcgagtagccaccccttgccttacatagccaactcgccggacttcgatctgaagaccttcatgcagaccaactacaacgacgagcccagtctggacagtgattttagcattaactcaatcgaatcggtgctatccgaggtgatccgcattgagtaccaggccttcaatagcatacaacaagcggcatcgcgcgtaaaggaggagatgtcctacggcactcagtctacgtacggtggatgcaattcggctgcaaacaatagccagccgcacctgcagcaacccatctgcgccccatccacccagcagttggatcgcgagctaaacgaggcggagcaaatgaagctgcgggagctgcgactggccagcgaggctctttatgatcccgtggacgaggacctcagcgccctgatgatgggcgatgatcgcattaaggtaacccgctagggataacagggtaataacagtccacggtattagcctataggtctttctacatttatagctccaacaccacggcttatctaatcagagtgtgcgagctgcgatatatgtacacacggcacctggcactttttagccattcggtgattcagtgcgtctctcgatgttggcccacgggccgtatcttcgtcagccagtttctgggttcccagcaatgctcgcctaccaaatgtaaacacactttttaatggggtggctcaaagtttttgatttcccaagagctttggtcgagtaaaagaaaattgatcgaaccagataagctattttcccccagagggttaaagaatttgaagtcatgcgactgggtctagttaagatatttgattacgaaaattggcctttaattaagaccctaaacgtgacaaacttccattctatatacttcttgatgagtatttaaacaaatatggctattttcggaacaaatcgggcactcatttatatctttagctttatctttattttttaagatgtgtccacacctttgatcgacctctagttcccctggagaaatgatttggaattatccaataatgattcatcacttccacgaattgttgtcccattaatcgagccaccctagctttcatgcaatcagaacgtctggtctgccaagaaggagcagacagcggctttatcagcctctgggcgtgccaattgtgacactatcaaccattatcaagagtaccagcaggcgctcatgagtctccagccagccgtcgatttgggcgtttattgtcgcttagactgtttaccgattttgcctcatcgcaattagcacatttcagtattgttaattgggaaaaacgatacaattttgacgaaatatatggagcagccaggtgttgggcgctatgataagcagtgctccgccattcgattgagtcaccttccagggagaagcctttacgattatggcgataataatggccaccaaagagaacatgggcaacatacgcactgacctgctcaagtttgccgaaggcaatatctacgaggagcaccaaaagttcatcacaacgtttgacgagaagtggcgcatggacgagaacataatcctgatcatgtgtgccattgtcctttttacctcggctcgatcgcgagtgatacacaaagacgtgattagattggaacaggtgagtaagcacttgataccatactgcagtattactaactttctttcattcgatagaattcctactattatcttctgcgaagatatctggagagtgtttattctggctgtgaggcgagaaacgcgtttatcaagctaatccaaaagatttcagatgtggagcgtctgaacaagttcataattaatgtctatttgaatgttaacccatcccaggtggagcccttgctgcgtgaaatattcgatttgaaaaatcactagacaaccgatgcgtgtcgggcatttaatgcctatgttgatgcccaatgatgaatggtcaacaagctgtagttgttgttgttgttgatgtctgttttatcttgtcgcttgtaatgttagattttaatcgaatgtgattgttagatttgcatatactgcatagattttatatttctacatcaaagagagcatatttaggataccaagtgcaaagcaacacaatctatatgtaatgtacaccgtttacctagtttcaaataaactagacgataatgcaataactaacttggaagcgtgggttctgtgcaaaaaggaaaaaagacaaaaaaaataaactgactttgagaaccagtggtaataaaatgtctcgtattcttttctactcgaatgaatttcgaaccctccaggacaaattacgcaaacgagtgattttgaacaacaatccaaaataatttaattccgaaagtcacaaaataaaaattcgaagtaggaaaaaacaaataagatgtttggaaaccaacgagagatgtgcttcgttaaagcatcaacccggggaaacaccacagcaaccgcgcatgtgtacccgcgaccagtcctcagaaatccacgtcgtgtacgtatccgcagccagcgtatgtgtccgcatctgccgaccccgtcttacatagtcatttatgtataatgtaggtaatataatagctcgagctcgctccgcaccaccaatgtgcgtcgtgcaagtccattccaattgttatccggtccactcgccgcgcaaatcggctttcaggttgattcgcggcaatccttggcccattgcagaaactcatccaacgcgctgacggccaaattgcgagaaagagccttcacacgcagattacgatcggttgtaatgagcaccaattccgtttgaatgaaacacttgccatctgcaaaagagttttagttagaaatgctatcaggaaggacatttaacggaagcagctcacctgtgcattgttcggtttttgccgttttcgagacagccattgccgttgccagaatcttgtcgtcattggacaaatattcctcctcaactagggcaaacaccgatgcattgacaaacgagccctttgtggtagcacatctagaaaagaaatcaataaggtattattgatcagcaggaaaagctttcctgaacaactattactactgatttaaaagtaaaatttcaatacattatcaggaaacttttatctatctcaatagcaaccaatgaattagacagaattataaatagctaatcgctagtaaaccctttatcagatatcagtaataaaggaactatgagctgacgcgcggaatataattaacaatagcttacttcacattgcctttggccgacttgatgaactctaacgactttttggcccgcgacgacacctcgtcaaagtggtggatgcgctgcgtctgcttcgaactgcggtacgagtccaacttaacgcccttggagaggccatccagttccttaaccactgtcagtggtataataagtgtgtagcgtttaaactccgtggacagtttttcaaagtcttcaaggcagtcgataaagcagttggtatccggtagaagatagcgcggtcgcacctcgatgtatattttcgtgtccacgaacttgagaatgtcctccaacttgctggtgcatatctgcttaaccctagccttggcctcaagctctttcttcagcttgcacagctcggaaacatcggaatcagttgaacgacacaacaattcctcaacggctttacattgaatctttgaaacgttcgccgcaagaccacaactttcagcatcatagttttccaatgcgttcttcatcgctgcgtttaggtgctcgacagtgaagcttgattccaacggctgctggagagcctcttgatgctggacgtagtattcctggaactgaccaatcctacgaacacgttcgaaaaactgaagacgttcggatcctcttttgagatagtccatgctcagcgtttcacgacccaacggagtgaaacctctaagggccacatcctcatccaacagtatttcgtttttctcaagtttgtgcttccccattaagcattcaatgtactcaaaaaggattgttagctcagcccagcaatcgatgaaagagtgtctgcaatattggagttaatgaaaaactaataaaaggcattcaatttatacatactcttccgatctgacgggttcccacacgtccaaactgatgctcaaccaacgtacataaacattcacgaactgtaggtatgtgttcacagtcgcaaagctgaaataaaagattaattagcaataataaataaacaaggcgaattttagcttactcttcttctgggagacactggacatttgtagaatcctctagatctactagtcc

SEQ ID NO: 38 - DNA, 545 bp, Artificial Sequence
  Description of Artificial Sequence; note = synthetic construct
gaagcaagcctctagaaagatgaagctactgtcttctatcgaacaagcatgcgatatttgccgacttaaaaagctcaagttcgcgatggcggcgaggaaagggatcacaaagcgccggcggatagcagcagcagcaaccttgaccactactcggcagaaagaggctatatcggtgatggagaaggtaatcagctcacaaaaggacgccttaacagaggacgccatcgatataatgaacaagttcatgaataccccagctcgcccggtgtcggggagcagcccagtacattctacgtacggtggatgcaatctgaagttcatcacaacgtttgacgagaagtggcgcatggacgagaacataatcctgatcatgtgtgccattgtcctttaatgtctatttgaatgttaacccatcccaggtggagcccttgctgcgtgaaatattcgatcaaagagagcatatttaggataccaagtgcaaagcaacacaatctataagacgataatgcaataactaacttggaagcgtgggttctgtgcaaacc

SEQ ID NO: 39 - DNA, 1119 bp, Artificial Sequence
  Description of Artificial Sequence; note = synthetic construct
tggcgaatgggacgcgccctgtagcggcgcattaagcgcggcgggtgtggtggttacgcgcagcgtgaccgctacacttgttagggtgatggttcttaatacaacctattaatttcccctcgtcaaaaataaggttatcaagtgagaaatcaccatgagtgacgactaaccggcgcaggaacactgccagcgcatcaacaatattttcacctgaatcaggatatgcttcccatacaatcgatagattgtcgcacctgattgcccgacagatcttcttgagatcctttttttctgcgcgttggcgataagtcgtgtcttggtagtgagcgaggaagcggaagagcgcctgatgcggtattttctccttacgcatctgtgcggtatttcacaccgcagggagctgcatgtgtcagaggttttcaccgtcatcaccgaaacgcgcgaggcagctgcggcgatgaaacgagagaggatgctcacgatacgggttactgatgatgaaacggaaaccgaagaccattcatgttgttgctcagaagattccgaataccgcaagcgctcactgtcttcggtatcgtcgtatcccactaccgagatatccgcaccaacgcgcagcccggactcggtaatggcgcgcattgccgagacagaacttaatgggcccgctaacagcgcgatttgctggtgacccaatgcgaccagatcgctttacaggcttcgacgccgcttcgttctaccatcgacaccaccacgcttcaccacgcgggaaacggtctgataagagacaccggaaggagatggcgcccaacagtccctctagaaataaaaccttgaccactactcggggtcacaggactcgcagagctgcggctcggcggacagcggggccaatgggtgctccggcaccttaaggctggtgtcgcatttgatcgactatccaggcgacgcactcaagatcatttcaaagtttagctgcgcattcttaaccgaatcctaagcggcggaggagcgaacgcagcccagctacatagccaactcgccggacttcgatctgaagaccttcaagcaacccatctgcgccccatccacccagcattccgtgacaaactatatccggat

SEQ ID NO: 40 - DNA, 30 bp, Artificial Sequence
  Description of Artificial Sequence; note = synthetic construct
gagagatgtgcttcgttaaagcatcaaccc

SEQ ID NO: 41 - DNA, 44 bp, Artificial Sequence
  Description of Artificial Sequence; note = synthetic construct
ggactagtagatctagaggattctacaaatgtccagtgtctccc

SEQ ID NO: 42 - DNA, 27 bp, Artificial Sequence
  Description of Artificial Sequence; note = synthetic construct
ccattattatcgccataatcgtaaagg

SEQ ID NO: 43 - DNA, 46 bp, Artificial Sequence
  Description of Artificial Sequence; note = synthetic construct
attaccctgttatccctagcgggttaccttaatgcgatcatcgccc

SEQ ID NO: 44 - DNA, 30 bp, Artificial Sequence
  Description of Artificial Sequence; note = synthetic construct
ggaaagcttttcctgctgatcaataatacc

SEQ ID NO: 45 - DNA, 41 bp, Artificial Sequence
  Description of Artificial Sequence; note = synthetic construct
tgggcccatcacttgcttgtaaccgccgaagaactgcgcgg

SEQ ID NO: 46 - DNA, 47 bp, Artificial Sequence
  Description of Artificial Sequence; note = synthetic construct
cgctagggataacagggtaataacagtccacggtattagcctatagg

SEQ ID NO: 47 - DNA, 47 bp, Artificial Sequence
  Description of Artificial Sequence; note = synthetic construct
cgattatggcgataataatggccaaagagaacatgggcaacatacgc

SEQ ID NO: 48 - DNA, 26 bp, Artificial Sequence
  Description of Artificial Sequence; note = synthetic construct
gaagcaagcctctagaaagatgaagc

SEQ ID NO: 49 - DNA, 39 bp, Artificial Sequence
  Description of Artificial Sequence; note = synthetic construct
cgtgccgttctccatcgatacagtcaactgtctttgacc

SEQ ID NO: 50 - DNA, 23 bp, Artificial Sequence
  Description of Artificial Sequence; note = synthetic construct
gcctggatagtcgatcaaatgcg

SEQ ID NO: 51 - DNA, 20 bp, Artificial Sequence
  Description of Artificial Sequence; note = synthetic construct
atggagaacggcacggatgc

SEQ ID NO: 52 - DNA, 40 bp, Artificial Sequence
  Description of Artificial Sequence; note = synthetic construct
tacattctagagaccaactacaacgacgagcccagtctgg

SEQ ID NO: 53 - DNA, 41 bp, Artificial Sequence
  Description of Artificial Sequence; note = synthetic construct
cattcatccggacattaattatgaacttgttcagacgctcc

SEQ ID NO: 54 - DNA, 39 bp, Artificial Sequence
  Description of Artificial Sequence; note = synthetic construct
gggcatcaactccggaattaaatgcccgacacgcatcgg

SEQ ID NO: 55 - DNA, 42 bp, Artificial Sequence
  Description of Artificial Sequence; note = synthetic construct
gtctcacgacgttttgaacccagaaatcgagctcgcccgggg

SEQ ID NO: 56 - DNA, 36 bp, Artificial Sequence
  Description of Artificial Sequence; note = synthetic construct
cacgaattccaaactgtctcacgacgttttgaaccc

SEQ ID NO: 57 - DNA, 44 bp, Artificial Sequence
  Description of Artificial Sequence; note = synthetic construct
gagagctagcatgccggctagatctcgagatcggccggcctagg

SEQ ID NO: 58 - DNA, 30 bp, Artificial Sequence
  Description of Artificial Sequence; note = synthetic construct
gaactgcagctcgagagctagcatgccggc

SEQ ID NO: 59 - DNA, 32 bp, Artificial Sequence
  Description of Artificial Sequence; note = synthetic construct
ggagatatacatatggctagcatgactggtgg

SEQ ID NO: 60 - DNA, 31 bp, Artificial Sequence
  Description of Artificial Sequence; note = synthetic construct
tgctcgaagcttcgcagaagataatagtagg

(19) United States
(12) Patent Application Publication
(10) Pub. No.: US 2009/0215859 A1
(43) Pub. Date: Aug. 27, 2009

(54) Compositions and methods for modulating dhr96

(76) Inventors: Carl S. Thummel, Salt Lake City, UT (US); Kirst King-Jones, Edmonton (CA); Michael Horner, Salt Lake City, UT (US); Geanette Lam, Holiday, UT (US)

Correspondence Address: Ballard Spahr Andrews & Ingersoll, LLP, SUITE 1000, 999 PEACHTREE STREET, ATLANTA, GA, 30309-3915, US

(21) Appl. No.: 10/585,841

(22) PCT Filed: Jan. 13, 2005
(86) PCT No.: PCT/US2005/001218

§ 371 Date: Mar. 17, 2009

Related U.S. Application Data
(60) Provisional application No. 60/536,337, filed on Jan. 13, 2004.

(51) Int. Cl. A01N 43/90; C07H 21/02; C12N 5/10; C12Q 1/68; A01P 7/04

(52) U.S. Cl. 514/44 A; 536/24.5; 435/348; 435/6

(57) ABSTRACT

Disclosed are compositions and methods for modulating DHR96 activity and identifying molecules that modulate DHR96 activity.

### I. BACKGROUND

The control of insects with toxins pesticides) is one of the largest industries in the world. Insects have evolved many methods to deal with pesticides, most of which act through a xenobiotic detoxification pathway. The regulation of the xenobiotic pathway represents an attractive target for pesticides. Disclosed herein, DHR96, a Drosophila gene is shown to regulate the xenobiotic pathway, and inhibition of the DHR96 gene expression or activity decreases the ability of Drosophila to adapt to toxins, including pesticides, such as DDT.

### II. SUMMARY

Disclosed are methods and compositions related to compositions and methods for regulating DHR96 and increasing the effect of existing any toxins to control insects are disclosed.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows DHR96 is closely related to the PXR/CAR/VDR subfamily of xenobiotic receptors. An alignment using the programs PHYLIP and CLUSTALW is depicted of the DHR96, DAF-12, PXR, CAR, and NHR-8 nuclear receptors, showing the percent identical amino acids within either the DNA binding domain or ligand binding domain.

FIG. 2 shows DHR96 is expressed in organs involved in nutrient absorption, metabolism, and excretion. Organs were dissected from wandering third instar larvae, fixed in 25% formaldehyde and stained with affinity-purified antibodies to detect DHR96 protein. In wild type larvae, nuclear DHR96 protein is detected in the fat body, in salivary glands and regions of the digestive tract including the gastric caece and the Malpighian tubules. Only background staining is detected in other tissues, including the imaginal discs and brain. No expression was detectable in fat bodies dissected from DHR96^{E25} mutant larvae, demonstrating the specificity of the antibody stains.

FIG. 3 shows a strategy for targeted mutagenesis of the DHR96 locus. Δ1 depicts the start methionine deletion and A2 depicts the deletion of the fourth exon/intron of DHR96. A transgene containing the targeting construct and the GFP marker was circularized by FLP recombinase and subsequently cut with I-SceI. Homologous pairing between the targeting construct and the endogenous DHR96 locus results in the generation of a tandem duplication by ‘ends-in’ recombination. To generate a single copy insertion, the tandem duplication was reduced by means of homologous recombination by inducing a DNA double stranded break with I-CreI.

FIG. 4 shows DHR96 mutants are more sensitive than wild type flies to the pesticide DDT. A time course is shown. 20 wild type or DHR96^{E25} mutant flies were treated with a high concentration of DDT (100 ng/μl) and assayed for survival every hour up to 10 hours. Each assay (A+B) was done in triplicate to determine the standard deviation as shown by the error bars.

FIG. 5 shows an alignment of Drosophila nuclear hormone receptor DNA-binding domains. An alignment of the DNA-binding domains of known Drosophila nuclear hormone receptor superfamily members reveals two regions of conserved amino acids flanking a central unique region. The conserved amino acids were used to design PCR primers for amplifying fragments of Drosophila receptors: F3, F4, F5, R4, R5, R6 and R8. The unique region was used to design gene-specific oligonucleotide probes to eliminate previously identified family members from further study.

FIG. 6 shows alignments of DNA-binding domain sequences. The DNA-binding domain sequence of each gene was used to search the PIR/Swiss Prot/GenBank databases. An alignment of each sequence with representative matches from the databases is presented. Shaded boxes indicate identity with the new protein sequence, and the percent identity is shown to the right of each sequence.

FIG. 7 shows temporal profiles of DHR38, DHR78, and DHR96 transcription during the onset of metamorphosis. Northern blots containing RNA samples isolated from staged third instar larvae and prepupae collected at 2 hr intervals were probed to detect DHR38, DHR78, and DHR96 mRNAs. These blots have been used previously for detailed studies of 20E-regulated gene transcription ((Andres, A. J., Fletcher, J. C., Karim, F. D. & Thummel, C. S. (1993). Dev. Biol. 160, 388-404) One set of blots was sequentially stripped and hybridized with probes from each gene, in order to allow direct comparison of transcription patterns. The blots were also hybridized to detect rp49 mRNA, as a control for equal loading (data not shown)). Developmental times are shown at the top as hours after egg laying for third instar larval development, and as hours after puparium formation for prepupal and pupal development. Landmark 20E-triggered developmental transitions are shown at the top.

FIG. 8 shows a time course of DHR38, DHR78, and DHR96 transcription in cultured larval organs treated with 20E. Mass-isolated late third instar larval organs were treated with 5×10-7 M 20E for the times shown, as described (Thummel, C. S., Burtis, K. C. & Hogness, D. S. (1990). Cell 61, 101-111) Equal amounts of total RNA isolated from each time point were fractionated by formaldehyde agarose gel electrophoresis, transferred to a nylon membrane, and hybridized with probes to detect DHR38, DHR78, DHR96 and rp49 mRNA. One northern blot was sequentially stripped and hybridized with a probe from each gene, in order to allow direct comparison of transcription patterns. Detection of DHR38 transcripts required the use of an antisense RNA probe.

FIG. 9 shows the DNA-binding specificities of DHR38, DHR78, and DHR96 protein. Each protein was overproduced in E. coli, purified, and tested for its ability to bind to eight oligonucleotides using electrophoretic mobility shift assays. The names of each oligonucleotide are shown at the top. In all cases, binding could be competed by the addition of an excess of the appropriate unlabelled oligonucleotide. FIG. 10 shows that no DHR96 protein was detectable in DHR96 mutants. Total protein was isolated from wild type control flies (w1118) DHR96E25 mutants, DHR9616A mutants, or 1/50 the amount of protein from heat-induced hs-DHR96 transformants that overexpress DHR96 protein were analyzed on a Western blot using DHR96 antibodies. The mutants shown in the center two lanes had no detectable DHR96 protein.

FIG. 10 shows DHR96E25 mutants are sensitive to phenobarbital and tebufenozide. Control Canton S adult flies (CanS), original DHR96E25 mutants (DHR96E25), and the outcrossed DHR96E25 mutant (outcross 1) were exposed to either DDT (FIG. 11A) or phenobarbital (FIG. 11B) for 23 hours and then scored for viability or motility, respectively. A dose response curve is shown. Twenty wild type or DHR96^{E25} mutant flies were exposed to eight DDT concentrations, from 0.78 to 100 ng/μl, and then scored for survival 10 hours later. A similar test was conducted for sensitivity to tebufenizide (FIG. 11C) using larvae raised on food supplemented with the drug. In parallel experiments, the original DHR96^{16A} stock showed responses similar to the original DB96E25 mutant.

FIG. 11 shows that DHR96 regulates members of all four classes of insect detoxification genes. The top genes that are down-regulated upon ectopic DHR96 overexpression are listed. Total RNA was extracted and purified to allow probe generation. Affymetrix microarray chips were hybridized with the probes and scanned. Raw data was analyzed with dCHIP, and filtering was performed in MS ACCESS. The expression levels in control (WWPHS) and hs-DHR96 (96WPHS) animals are shown, along with the fold change in gene expression. Members of gene families known to be involved in detoxification in insects are also shown.

FIG. 12 shows a schematic representation of the GAL4-LBD activation assay. A gene fusion of the GAL4 DNA binding domain (DBD) and DHR96 ligand binding domain (LBD) is expressed upon heat-induction of the hsp70 promoter. The resultant fusion protein can bind to GAL4 response elements (UAS) on a seperate transgenic construct, but will only activate lacZ transcription in the presence of an appropriate ligand and/or co-factors (a ligand is shown). β-galactosidase expression is detected as the substrate from an Xgal staining reaction.

FIG. 13 shows GAL4-DHR96 is activated by tebufenozide. Third instar larvae were heat-treated to induce GAL4-DHR96 expression, dissected, and organs were cultured in the presence of 1×10⁻⁵ M tebufenozide. UAS-lacZ reporter gene expression was detected by Xgal staining. Control animals were either from a non-transgenic control line or GAL4-DHR96 transgenic animals that were not treated with tebufenozide.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. COMPOSITIONS AND METHODS

Four lines of evidence show that DHR96 plays a central role in coordinating insect xenobiotic responses. First, this gene is a member of the nuclear receptor subclass that includes the PXR, SXR, VDR, and NHR-8 xenobiotic receptors. Second, DHR96 protein is expressed specifically in tissues that are involved in absorption, metabolism, and excretion of toxic compounds. Third, a DHR96 mutant is sensitive to phenobarbital and tebufenozide. Finally, members of all four classes of known insect detoxification genes can be regulated by ectopic DHR96 expression.

Higher organisms neutralize environmental toxins or xenobiotics through enzymes that include cytochrome p450 monooxygenases, glutathione transferases, carboxylesterases, and UDP-glucuronosyl transferases. In mammals, some of these detoxification enzymes are directly regulated by the nuclear receptors PXR and CAR, which in turn are activated by a broad spectrum of xenobiotics including prescription drugs, plant toxins and other contaminants. In contrast, there is little understanding of how similar xenobiotic responses might be controlled in insects. Herein it is shown that mutants in the DHR96 nuclear receptor of Drosophila are viable and fertile under standard laboratory conditions, as are flies that widely express double stranded DHR96RNA (RNAi) from a transgene. However, when exposed to a pesticide like DDT, mutant animals are less resistant to the insecticide challenge, dying more rapidly and at lower concentrations than control animals. Unlike many other nuclear receptors, widespread ectopic expression of DHR96 has no effect on the viability of larvae or flies, suggesting that activation of DHR96 is ligand-dependent.

Disclosed herein, DHR96 is expressed in tissues that have been associated with the detoxification process, including the gastric caeca, the major site of absorption in Diptera, and the fat body, the insect equivalent of the liver. Microarray studies disclosed herein show that overexpression of DHR96 results in the downregulation of members of all four classes of the detoxification machinery, supporting the proposal that DHR96 functions as a xenobiotic regulator in Drosophila. These findings demonstrate how detoxification enzymes are activated in insects upon challenge with an insecticide. Given that this receptor has been highly conserved in the distant insect species, Anopheles gambiae, it is likely that it exerts a similar function in all insects. Also disclosed are methods for the identification of specific compounds or peptides that affect DHR96 activity and can act as effective synergists that, for example, enhance the lethality of pesticides for insect control.

Disclosed are mutants of the DHR96 gene which have reduced DHR96 activity in the xenobiotic pathway. These mutants can be used in a variety of methods for isolating new molecules that inhibit the xenobiotic pathway, by for example, being used as controls in methods that are testing the xenobiotic activity of a particular compound. The mutants can also be used as stock for production of other mutant flies. The mutants can also be used as seed genetic backgrounds to change a given population of flies to insecticide sensitive flies, by introducing the mutant backgrounds into the populations, through fly breeding.

Also disclosed are compositions which are capable of inhibiting DHR96 protein function or gene function, and which in turn inhibit the xenobiotic effect of the DHR96 protein. For example, disclosed are iRNA molecules which inhibit the function of DHR96 and inhibit the xenobiotic effect of DHR96.

Also disclosed are methods of inhibiting insect growth by administering an inhibitor of DHR96 to an insect, such as a fly.

Also disclosed are methods of identifying molecules that inhibit DHR96, and inhibit the xenobiotic activity in an insect, such as a fly, comprising for example, testing compounds for inhibition activity of DHR96 and/or inhibition of xenobiotic activity and, then for example, comparing the activity of these molecules to the disclosed inhibitors of DHR96, such as the mutants or the disclosed iRNA molecules.

1. The Xenobiotic Response

Virtually every organism faces a fundamental challenge when exposed to potentially harmful environmental substances called xenobiotics, which may include pharmaceuticals, plant toxins, pollutants, pesticides, hormones and fatty acids. Exposure to xenobiotics can occur either directly by physical contact, inhalation, or ingestion of nutrients or indirectly when an organism generates toxic metabolites from less harmful precursors. The mechanisms by which toxic compounds are removed and/or neutralized fall into two broad categories. Usually as a result of extreme selective pressures, organisms may develop adaptive processes that are highly specific to a particular substance, as can be observed in many insect species that become resistant to pesticides (Wilson, T. G. (2001). Annu Rev Entomol 46, 545-571) or that have evolved the ability to utilize hazardous plant species as a food source (Danielson, P. B. et al. (1997). Proc Natl Acad Sci USA 94, 10797-10802; Fogleman, J. C. (2000). Chem Biol Interact 125, 93-105.). In contrast to this highly specific response, all metazoan species appear to have a general machinery that allows the efficient detoxification of a vast range of chemicals. The general detoxification mechanisms display a surprising flexibility, which is mainly achieved by two factors. First, at least three enzyme classes comprising more than 160 proteins in the mosquito and the fruit fly are responsible for metabolizing lipophilic toxins into less harmful substances (Ranson, H., et al. (2002). Science 298, 179-181). Second, some enzymes appear to have an immense range of substrate specificity. For instance, Cyp3A4, a member of the cytochrome p450 monooxygenase family, is capable of neutralizing an estimated 50% of all existing prescription drugs (Maurel, P. (1996). (Boca Raton, CRC Press), pp. 241-270). Cytochrome p450 enzymes are often referred to as phase I enzymes, because they catalyze the first step in the detoxification process by adding oxygen groups to lipophilic chernicals, thus resulting in more water-soluble compounds, which in turn facilitates efficient excretion. Other enzyme families like glutathione transferases, carboxylesterases and UDP-glucuronosyl transferases are classified as phase II enzymes, as their role is to catalyze subsequent detoxification steps.

In insects, pesticide resistance is most often the result of mutations that affect the general detoxification pathway. For example, the overexpression of a single gene, Cyp6g1, a member of the cytochrome p450 family, is sufficient to confer DDT resistance in Drosophila melanogaster (Daborn, P. B. et al. (2002), Science 297, 2253-2256). The same study demonstrated that Cyp6g1 is hypertranscribed in over 20 DDT-resistant Drosophila strains of worldwide origin, but further analysis suggested that this finding could be traced back to a single event, since all alleles harbor the same Accord transposon in their 5′ regulatory region.

In the past decade considerable progress in the field has revealed the mechanisms that allows an organism to sense a wide range of toxic substances and to understand how xenobiotic sensing translates into the induction of highly specific sets of detoxifying enzymes. It quickly became apparent that certain members of the so-called nuclear receptor superfamily are the central players in this process. Nuclear receptors are ligand-activated transcription factors that play important roles in diverse physiological processes such as cell growth and differentiation, embryonic development, and cholesterol metabolism (Francis, G. A. et al. (2003) Annu Rev Physiol 65, 261-311; Mangelsdorf, D. J., et al. (1995). Cell 83, 835-839; Tontonoz, P., and Mangelsdorf, D. J. (2003). Mol Endocrinol 17, 985-993) Of the 48 nuclear receptors encoded by the human genome ˜26 have identified ligands (Kliewer, S. A. (2003) J Nutr 133, 2444S-2447S), but only three have been associated with xenobiotic activity, namely PXR, CAR and VDR (Maglich, J. M., et al. (2002) Mol Pharmacol 62, 638-646; Makishima, M., et al. (2002). Science 296, 1313-1316). These three closely related receptors are not only able to sense and bind lipophilic xenobiotic substances directly, but once activated by such a ligand, they can regulate the expression of enzymes that will neutralize the very compound that had activated these nuclear receptors in the first place, thus creating feedback loop. Disclosed is an analogous mechanism that exists in the fruit fly, Drosophila melanogaster. The disclosed mechanism involves an insect nuclear receptor, the Drosophila DHR96 nuclear receptor.

### (1) Nuclear Receptors

Members of the nuclear receptor superfamily have been one of the most productive targets for drug development by the pharmaceutical industry. Efforts along these lines have resulted in drugs that have had a major impact on human health, including cancer treatments, fertility control, and cholesterol reduction. Nuclear receptors are ligand-activated transcription factors, but can have many regulatory functions aside from this ligand activated function. Nuclear receptors have been organized in a phylogeny-based nomenclature (Nuclear Receptors Nomenclature Committee, (1999) Cell 97, 1-3.) of the form NRxyz, where x is the sub-family, y is the group and z the gene. For a review see, Robinson-Rechavi, M., et al., Journal of Cell Science, Cell Science at a Glance, 116(4):585-586 and poster insert, (2003), which is herein incorporated by reference at least for material related to nuclear receptors).

Nuclear receptors lend themselves to drug intervention because their activity can be modulated by small lipophilic compounds that can be easily delivered to animals in a stable format. Compounds can be developed that either constitutively activate their cognate receptor, called agonists, or constitutively inactivate the receptor, called antagonists. The use of these compounds in animals provides a means of tightly regulating nuclear receptor activity in vivo, with resultant effects on growth and development.

Surprisingly, no similar effort has been made by the agricultural industry to target insect nuclear receptors as a means of pest control. This is largely because the mechanism of action of most insect nuclear receptors has remained undefined. Disclosed herein it was shown that an insect nuclear receptor, encoded by DHR96, is required for resistance to toxic compounds in Drosophila. Also disclosed are molecules that inhibit the DHR96 function and that inhibiting the function of DHR96 makes DHR96 have decreased resistance to pesticides and toxins. Also disclosed are methods utilizing DHR96 to identify compounds that modulate its function, such as inhibit its function. Molecules that inhibit DHR96 render the insect more susceptible and sensitive to pesticides.

The Drosophila genome encodes 18 nuclear receptors that have a classical DNA-binding and ligand-binding domain and, of those, just two have identified ligands. In the nematode C. elegans, it was shown that a mutation in the nuclear receptor nhr-8 gene causes a reduced resistance to colchicine and chloroquine, suggesting that this gene is involved in the xenobiotic pathway (Lindblom, T. H., et al. (2001). Curr Biol 11, 864-868, which is herein incorporated by reference at least for material related to nuclear receptors and their activity, and for material related to NHR8). Disclosed herein DHR96 mutants are viable under normal conditions, but exhibit a significantly lower resistance to DDT when compared to wild type flies. Additionally, microarray analysis of animals that overexpress DHR96 indicate that this nuclear receptor regulates genes which primarily encode detoxification enzymes.

Disclosed herein insecticide function in insects can be reviewed from a different perspective. Disclosed are methods for identifying DHR96 antagonists and agonists. Also disclosed are methods related to the identification of the DHR96 target gene network. Also disclosed is a class of pesticides that targets the regulatory pathways that control the detoxification machinery.

(a) Classes of Nuclear Receptors

Retinoid, vitamin D, steroid, and thyroid hormones are small hydrophobic ligands that initiate a diverse array of developmental and metabolic responses. The receptors that mediate these responses form the basis of the nuclear hormone receptor superfamily (see Tsai, M.-J. & O'Malley, B. W. (1994). Annu. Rev. Biochem. 63, 451-486, for a review). This family is defined by a characteristic protein domain structure including a conserved DNA-binding domain and a ligand binding/dimerization domain. Members of this superfamily can be divided into three classes based on their ligand-binding and DNA-binding properties. Steroid receptors, including the estrogen and glucocorticoid receptors, form homodimers that bind to an inverted repeat of 6 bp consensus half-sites (Tsai, M.-J. & O'Malley, B. W. (1994). Annu. Rev. Biochem. 63, 451-486, Gronemeyer, H. (1992). FASEB J. 6, 2524-2529). The second class includes the retinoid receptors, RAR and RXR, as well as receptors for thyroid hormone and vitamin D. These receptors can bind to direct repeats of AGGTCA half-sites as homodimers or heterodimers (Stunnenberg, H. G. (1993). BioEssays 15, 309-315). The third and largest class are referred to as orphan receptors since their potential ligands are unknown. At least some of these receptors, including Rev-Erb and NGFI-B, can bind to a single AGGTCA half-site (Harding, H. P. & Lazar, M. A. (1993). Mol. Cell. Biol. 13, 3113-3121; Wilson, T. E., et al., (1993). Mol. Cell. Bio. 13, 5794-5804). Although extensive studies have provided significant insights into the mechanisms by which nuclear hormone receptors regulate the transcription of target genes, we still know little about how these changes in gene expression result in specific and diverse developmental responses.

(b) Drosophila Nuclear Receptors

There are 18 canonical nuclear receptor genes in the complete genome of the fly Drosophila melanogaster (Adams et al., (2000) Science 287, 2185-2195, which is herein incorporated by reference at least for material related to nuclear receptors). The 18 members of the nuclear hormone receptor superfamily identified in Drosophila are: EcR, usp, tll (Pignoni, F., et al., (1990). Cell 62, 151-163), svp (Mlodzik, M., et al., (1990). Cell 60, 211-224), dHNF-4 (hong, W., et al., (1993). EMBO J. 12, 537-544), E75 (Segraves, W. A. & Hogness, D. S. (1990). Genes Dev. 4, 204-219), E78 (Stone, B. L. & Thummel, C. S. (1993). Cell 75, 307-320), FTZ-F1 (Lavorgna, G., et al., (1991). Science 252, 848-851), DHR3 (Koelle, M. R., et al., (1992). Proc. Natl. Acad. Sci. USA 89, 6167-6171), DHR4 (Weller J, Sun G C, Zhou B, Lan Q, Hiruma K, Riddiford I M. Isolation and developmental expression of two nuclear receptors, MHR4 and betaFT-F1, in the tobacco hornworm, Manduca sexta. Insect Biochem Mol. Biol. 2001 Jun. 22; 31(8):827-37.; King-Jones, K. Charles, J.-P., & C.S. Thummel, The DHR4 orphan nuclear receptor is required for Drosophila growth and metamorphosis, manuscript in prep; Adams et al., (2000) Science 287, 2185-2195) and DHR39 (Ohno, C. K. & Petkovich, M. (1992). Mech. Dev. 40, 13-24; Ayer, S., et al., (1993). Nuc. Acids Res. 21, 1619-1627), DHR38, DHR78 (Fisk and Thummel, (1995), PNAS, Proc Natl Acad Sci USA. 1995 Nov. 7; 92(23):10604-8), DHR83 (King-Jones, K. and C. S. Thummel (2003) Drosophila nuclear receptors. In “Handbook of Cell Signaling,” Vol. 3, (Bradshaw, R. and Dennis, E., eds.), Academic Press, New York, pp. 69-73; Adams et al., (2000) Science 287, 2185-2195), DHR96 (Fisk and Thummel, 1993), dsf (Finley, K. D., et al. (1998). “dissatisfaction encodes a Tailless-like nuclear receptor expressed in a subset of CNS neurons controlling Drosophila sexual behavior.” Neuron 21, 1363-1374), dERR (King-Jones, K. and C. S. Thummel (2003) Drosophila nuclear receptors in “Handbook of Cell Signaling,” Vol. 3, (Bradshaw, R. and Dennis, E., eds.) Academic Press, New York, pp. 69-73; Adams et al., (2000) Science 287, 2185-2195), and dFAX-1 (King-Jones, K. and C. S. Thummel (2003) Drosophila nuclear receptors. In “Handbook of Cell Signaling,” Vol. 3, (Bradshaw, R. and Dennis, E., eds.), Academic Press, New York, pp. 69-73; Adams et al., (2000) Science 287, 2185-2195) At least seven of these genes appear to contribute to the 20E regulatory hierarchies that direct the onset of metamorphosis—E75, E78, βFTZ-F1, DHR3, DHR39, EcR, and usp (Richards, G. (1992). Current Biology 2, 657-659; Horner, M., et al., (1995). Dev. Biol. 168, 490-502; Woodard, C. T., et al., (1994). Cell 79, 607-615).

Table 5 provides a list of Drosophila nuclear receptors.

TABLE 5

| | | | | | |
|---|---|---|---|---|---|
| probe set | CG | CT | Accession | Description | SEQ ID NO |

| | | | | | |
|---|---|---|---|---|---|
| 144004_at | CG16902 | CT37504 | FBgn0023546 | sym = Hr4 | SEQ ID NO: 1 |
| | | | | orEG:133E12.2 | |
| | | | | /name = DHR4 | |
| 154699_at | CG4059 | CT13432 | FBgn0001078 | sym = ftz/name = ftz | SEQ ID NO: 3 |
| | | | | transcription factor 1 | |
| 143123_at | CG11823 | CT11367 | FBgn0000448 | sym = Hr46 or DHR3 | SEQ ID NO: 5 |
| | | | | /name = Hormone receptor-like in 46 | |
| 152580_at | CG11783 | CT33046 | FBgn0015240 | sym = Hr96 or | SEQ ID NO: 7 |
| | | | | DHR96/name = Hormone | |
| | | | | receptor-like in 96 | |
| 143535_at | CG9310 | CT40906 | FBgn0004914 | sym = Hnf4 | SEQ ID NO: 9 |
| | | | | /name = Hepatocyte | |
| | | | | nuclear factor 4 | |
| 143768_at | CG1864 | CT5732 | FBgn0014859 | sym = Hr38 or DHR38 | SEQ ID NO: 11 |
| | | | | /name = Hormone receptor-like in 38 | |
| 149398_at | CG10296 | CT28911 | FBgn0037436 | sym = CG10296 or | SEQ ID NO: 13 |
| | | | | DHR83/name = Hr83 | |
| 143372_at | CG11502 | CT12919 | FBgn0003651 | sym = svp/name = seven up | SEQ ID NO: 15 |
| | | | | /prod = nuclear receptor | |
| | | | | NR2F3 | |
| 143379_at | CG1378 | CT3134 | FBgn0003720 | Sym = til/name = tailless | SEQ ID NO: 17 |
| | | | | /prod = nuclear receptor | |
| | | | | NR2E2 | |
| 143805_at | CG9019 | CT25922 | FBgn0015381 | sym = dsf | SEQ ID NO: 19 |
| | | | | /name = dissatisfaction/ | |
| | | | | prod = /func = receptor | |
| 147244_at | CG16801 | CT37351 | FBgn0034012 | sym = CG16801/name = FAX-1 | SEQ ID NO:21 |
| | | | | /prod = nuclear hormone | |
| | | | | receptor-like | |
| 153072_at | CG7404 | CT22787 | FBgn0035849 | sym = CG7404 /name = ERR | SEQ ID NO: 23 |
| | | | | /prod = /func = steroid hormone | |
| | | | | receptor | |
| 152160_at | CG7199 | CT22217 | FBgn0015239 | sym = Hr78 or | SEQ ID NO: 25 |
| | | | | DHR78/name = Hormone- | |
| | | | | receptor-like in 78 | |
| 153675_at | CG4380 | CT14272 | FBgn0003964 | sym = usp/name = ultraspiracle | SEQ ID NO: 27 |
| | | | | /prod = nuclear receptor | |
| | | | | NR2B4 | |
| 153197_at | CG8127 | CT24290 | FBgn0000568 | sym = Eip75B or | SEQ ID NO: 29 |
| | | | | E75/name = Ecdysone-induced | |
| | | | | protein 75B | |
| 143525_at | CG18023 | CT40336 | FBgn0004865 | sym = Eip78C or | SEQ ID NO: 31 |
| | | | | E78/name = Ecdysone-induced | |
| | | | | protein 78C | |
| 154377_at | CG1765 | CT5200 | FBgn0000546 | sym = ECR/name = Ecdysone | SEQ ID NO: 33 |
| | | | | receptor/prod = ecdysone | |
| | | | | receptor | |
| 155094_at | CG8676 | CT5296 | FBgn0010229 | sym = ECR/name = Ecdysone | SEQ ID NO: 35 |
| | | | | receptor/prod = ecdysone | |
| | | | | receptor | |

While there are 18 nuclear receptors in flies, there are 48 in humans (Robinson-Rechavi et al., (2001) Trends Genet. 17, 554-556), 49 in the mouse with the addition of FXRβ, (Robinson-Rechavi and Laudet, 2003, Methods Enzymol. 2003; 364:95-118) and more than 270 genes in the nematode worm Caenorhabditis elegans (Sluder et al., (1999). Genome Research 9, 103-120.

(c) Role of 20-hydroxyecdysone (20E) in Drosophila

20E is involved in the metamorphosis of the fruit fly, Drosophila melanogaster through steroid hormone receptors. A high titer 20E pulse at the end of third instar larval development triggers puparium formation, followed 10 hrs later by an 20E pulse that triggers head eversion and the onset of pupal development (Pak, M. D., & Gilbert, L. I. (1987). J. Liq. Chrom. 1.0, 2591-2611; Richards, G. (1981). Mol. Cell. Endocrin. 21, 181-197). The 20E receptor is encoded by two members of the nuclear hormone receptor superfamily, EcR (Koelle, M. R., et al., (1991). Cell 67, 59-77) and usp (Henrich, V. C., et al., (1990). Nuc. Acids Res. 18, 4143-4148; Shea, M. J., et al., (1990). Genes Dev. 4, 1128-1140; Oro, A. E., et al., (1990). Nature 347, 298-301). Usp is most closely related to the vertebrate RXR family and can heterodimerize with vertebrate thyroid and vitamin D receptors, as well as with EcR (Yao, T.; et al., (1992). Cell 71, 63-72; Thomas, H. E., et al., (1993). Nature 362, 471-475; Yao, T., et al., (1993). Nature 366, 476-479; Koelle, M. R. (1992) Ph.D. thesis, Stanford University). The ability of RXRs to function as promiscuous heterodimerization partners combined with the sequence similarity of many receptor binding sites raises the possibility that other members of the superfamily may function in transducing 20E signals, either by interacting directly with EcR and/or Usp, or by competing for receptor binding sites (Richards, G. (1992). Current Biology 2, 657-659).

### (d) General Structure of Nuclear Receptors

There are a number of domains in a nuclear receptor. From the N terminus to the C terminus there is the A/B domain, followed by a DNA binding domain (DBD, C), which contains the DNA sequence recognition domain called the P-box, which is followed by a less conserved region, D, which acts as a flexible hinge between the DBD and the ligand binding domain (LBD, E) and the D domain typically contains the nuclear localization signal, but this may overlap with the C domain, and finally some nuclear receptors contain a C-terminal F domain whose function is unknown.

The A/B domain and N terminal region in general is highly variable and can range in size from less than about 50 amino acids to more than about 500 amino acids. The A/B domain typically contains the transactivation domains which typically include at least one constitutively active domain, the AF-1 domain, and than typically one or more autonomous activation domains which can be regulated or not, called AD domains.

The DBD is typically the most conserved region. It contains the P-box, a six amino acid region that confers specificity for binding to particular target sites in the DNA. The P-box for DHR96 is ESCKA. An example of DHR96 is shown in SEQ ID NO:7. The DBD is also typically the site of homo- and hetero-dimerization. The 3D structure of the DBD shows that it contains contains two highly conserved zinc-fingers —C—X2-C-X13-C—X2-C and CX5—C—X9-C—X2-C—the four cysteines of each finger chelating one Zn₂₊ ion.

The LBD is typically the largest domain and is only moderately conserved, but the secondary structure is often conserved and contains 12 α-helixes. Many functions are associated with the E domain, including the AF-2 transactivation function, a strong dimerization interface, another NLS, and often a repression function. Typically the functions are ligand regulated.

(e) Dimerization of Nuclear Receptors.

Dimerization of nuclear receptors is very important to their function. The dimerization domains typically reside in the DBD and LBD. Many nuclear receptors heterodimerize with RXRs (USP in arthropods), such as DHR38 (NR4A4), NGFIB (NR4 μl), NURR1 (NR4A2), NOR1 (NR4A3), LXR and FXR subfamilies (LXRα, (NR1H3), LXRβ (NR1H2, HO), ECR(NR1H1), FXRα (NR1H4, HO), FXRβ (NR1H5, HO), the CAR1 and VDR subfamilies including, CAR1 (NR1I3), PXR (NR1I2), VDR(NR1L1) (NR1J1), the PPAR subfamily including, PPARγ (NR1C3), PPARα (NR1C1), AND PPARβ (NR1C2), the RAR subfamily including RARβ (NR1B2), RARα (NR1B1), and RARγ (NR1B3), and TRα (NR1A1), and TRβ (NR1A2), and possibly COUP-TF and FXRβ (for a review, see Robinson-Rechavi M, Escriva Garcia H, Laudet V., J Cell Sci. 2003 February 15; 116(Pt 4):585-6). DHR96 can also be found to dimerize with any other receptor, such as USB, or itself.

(f) Ligands for Nuclear Receptors

The superfamily includes receptors for many different types of molecules. For example, nuclear receptors bind hydrophobic molecules such as steroid hormones, such as estrogens, glucocorticoids, progesterone, mineralocorticoids, androgens, vitamin D3, ecdysone, oxysterols and bile acids. Certain nuclear receptors also bind retinoic acids, such as all-trans and 9-cis isoforms, thyroid hormones, fatty acids, leukotrienes and prostaglandins (Escriva et al., 2000, Bioessays 22, 717-727 and Robinson-Rechavi M, Escriva Garcia H, Laudet V., J Cell Sci. 2003 Feb. 15; 116(Pt 4):585-6).

### (g) How Nuclear Receptors Function

Nuclear receptors typically act in a stepwise fashion that starts with repression, moves to a state of derepression, and ends with transcription activation. (reviewed by Robinson-Rechavi M, Escriva Garcia H, Laudet V., J Cell Sci. 2003 Feb. 15; 116(Pt 4):585-6).

Repression typically occurs with corepressors, such as the histone deacetylase activity (HDAC) (for example, the apo-nuclear receptor). Usually ligand binding results in derepression, caused by the disassociation of the receptor from the corepressors. Also ligand binding typically causes the recruitment of coactivators, such as histone acetyltransferase (HAT) activity, which causes chromatin decondensation, which is believed to be necessary but not sufficient for activation of the target gene. After the HAT complex dissociates, typically a second coactivator complex is assembled (TRAP/DRIP/ARC), which is able to establish contact with the basal transcription machinery, and thus results in transcription activation of the target gene, but many other transcription co-activators can be associated with the nuclear receptor and these coactivators can provide activation discrimination. This general scheme does not apply for all nuclear receptors, as for example, some nuclear receptors can activate without ligand and some may bind DNA without ligand and some may repress with or without ligand.

### (2) DHR96 Gene

DHR96 maps to 96B12-14 in the polytene chromosomes of Drosophila. The DHR96 gene was cloned and sequenced and its sequence is set forth in SEQ ID NO: 1. (Fisk and Thummel (1995) Proc. Natl. Acad. Sci. USA, 92: 10604-10608, herein incorporated by reference at least for material related to the DHR96 gene and its sequence including the specific sequence).

DHR96 is highly conserved in Anopheles gambiae, a distant (˜250 M years) dipteran species (see Table 4). Similarly, many other Drosophila nuclear receptors are conserved in even more distant insects and, when examined, their regulatory functions appear to be conserved as well (Swevers L, Iatrou K. The ecdysone regulatory cascade and ovarian development in lepidopteran insects: insights from the silknoth paradigm. Insect Biochem Mol. Biol. 2003 December; 33(12):1285-97; Riddiford L M, Hiruma K, Zhou X, Nelson C A. Insights into the molecular basis of the hormonal control of molting and metamorphosis from Manduca sexta and Drosophila melanogaster. Insect Biochem Mol Biol. 2003 December; 33(12):1327-38). This is consistent with the role of detoxification via DHR96 being conserved through evolution. Thus, inactivation of DHR96 function in known insect pests provides a novel mode of intervention. It is understood that DHR96 homologs in other insects, insect orders, insect families and other insect specifies are considered disclosed and that they function in a manner similar to DHR96 in Drosophila. There is significant homology within the order Diptera and within the class of insects in general for nuclear receptors, and there is shown in Table 4, that there is a high degree of homology between DHR96 in other insects, such as the mosquito.

Disclosed are DHR96 variants that have at least 60%, 65%, 70%, 75%, 80%, 81%, 82%, 83%, 84%, 85%, 86%, 87%, 88%, 89%, 90%, 91%, 91%, 92%, 93%, 94%, 95%, 96%, 97%, 98%, or 99% identity or homology as discussed herein in to the LBD of DHR96, DBD of DHR96, or full length DHR96, or of fragments of DHR96, functional or otherwise.

Among the C. elegans receptors, DHR96 is most similar to DAF-12, which is a gene involved in dauer larva formation in C. elegans (68% identity DBD; 29% identity LBD). The match with NHR-8 in C. elegans is weaker (60%; 25%). This is consistent with DHR96 having a role similar to DAF-12. DAF-12 reads signals from TGFbeta and insulin and decides when the worm should enter diapause to survive difficult conditions. Diapause is similar to pupal stages in many ways (indeed many insects diapause during metamorphosis). Disclosed herein, mutants of DHR96 did not have any effects on metamorphosis—and they survived. Thus it was expected that DHR96 would have a function similar to DAF-12. DAF-12 is a gene involved in dauer larva formation in C. elegans. DAF-12 reads signals from TGFbeta and insulin and decides when the worm should enter diapause to survive difficult conditions. Diapause is similar to pupal stages in many ways (indeed many insects diapause during metamorphosis). However, as disclosed herein, mutants of DHR96 did not have any effects on metamorphosis—as they survived.

Disclosed are systems that assay for effects of drugs that alter DH96—and thus one can assay for effects on target gene transcription and relate that expression to the ability of an animal, such as an insect, to resist toxins.

TABLE 4

| | DBD | | LBD amino |
|---|---|---|---|
| | amino acids | | acids 501-723 |
| species | 7-72 identity | p-box | identity |
| anopholes gambiae | 86% | same | 65% |
| | % | | % |
| c. elegans daf-12 | 69% | same | 26% |
| strongyloides stercoralis- | 67% | different | 27% |
| parasitic worm | | | |
| c. elegans nhr-48 | 66% | same | |
| | % | | |
| VDR-zebrafish | 65% | different | 27% |
| VDR-bastard halibut | 63% | different | 27% |
| mouse vdr | 62% | different | 23% |
| human vdr | 62% | different | 24% |
| c. elegans nhr-8 | 60% | same | 25% |
| mouse pxr | 59% | different | 23% |
| human pxr | 59% | different | 22% |
| human car | 56% | different | 19% |
| AamEcRA1-tick | 54% | different | |
| ecdysone receptor-locusta | 53% | different | |
| migratoris-locust | | | |
| ecdysone receptor-calliphor | 53% | different | |
| vicina-insect | | | |
| EcR- tenebrio molitor- | 53% | different | |
| yellow mealworm | | | |
| EcR- d. melanogaster | 51% | different | |
| EcR- aedes albopictus- | 51% | different | |
| mosquito mouse car | 51% | different | 20% |

Table 4 shows the percent identical amino acids within the DNA binding domain and ligand binding domain for DHR96 and the best matches in the public databases (Genbank). Shown is the mosquito DHR96 gene, and it is the orthologous receptor in mosquito. (anopholes gambiae) (85% and 65% identity—very high). Also listed is whether the sequence within the P box, is either the same as DHR96 or different. This sequence directs the DNA binding specificity of the receptor. DHR96DNA binding is predicted to be similar to that of all three nematode homologs (daf-12, nhr-48 and nhr-8), but none of the vertebrate ones.

In certain embodiments homologs of DHR96 in other insect species can have at least 50% identity in the DBD and 25% identity in the LBD.

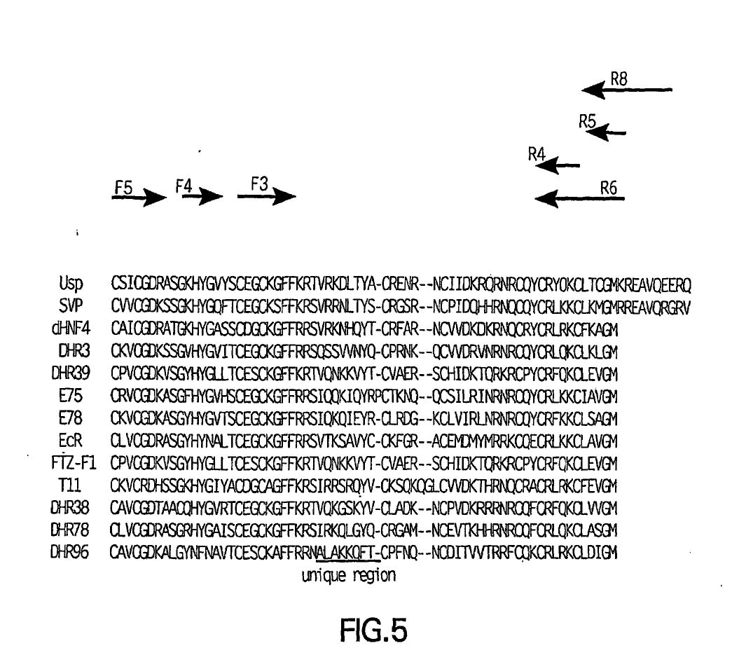

An alignment of the Drosophila nuclear hormone receptor DNA-binding domains reveals a central region of 8-9 unique amino acids flanked by highly conserved regions that each contain a C₂C₂ zinc finger (FIG. 5).

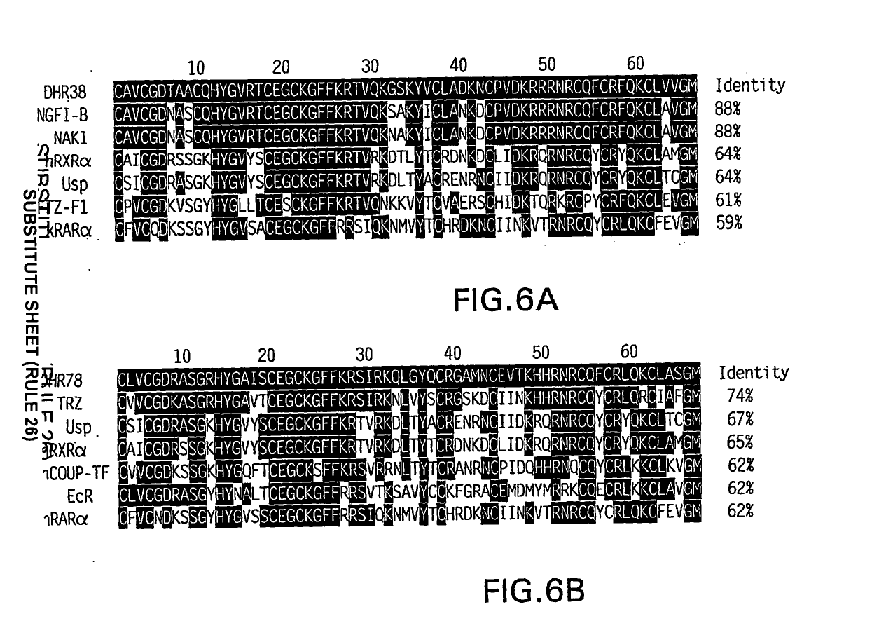

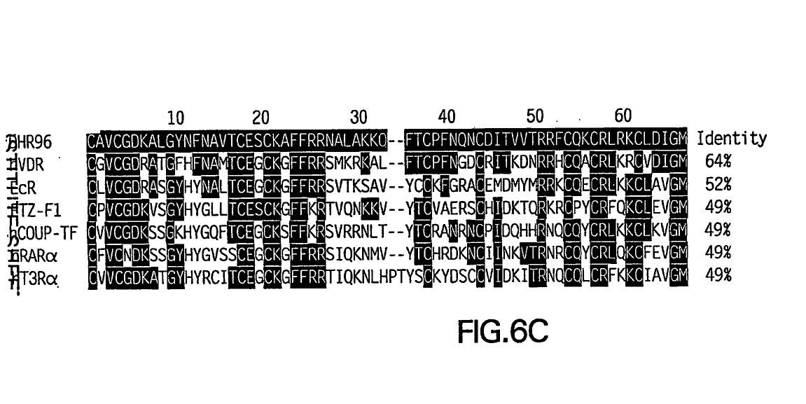

The DNA-binding domain of DHR96 is 64% identical to the human vitamin D receptor and 52% identical to EcR (FIG. 6C). The DHR96 ligand binding domain (amino acids 501-723) is most similar to that of thyroid hormone receptor, with 23% identity.

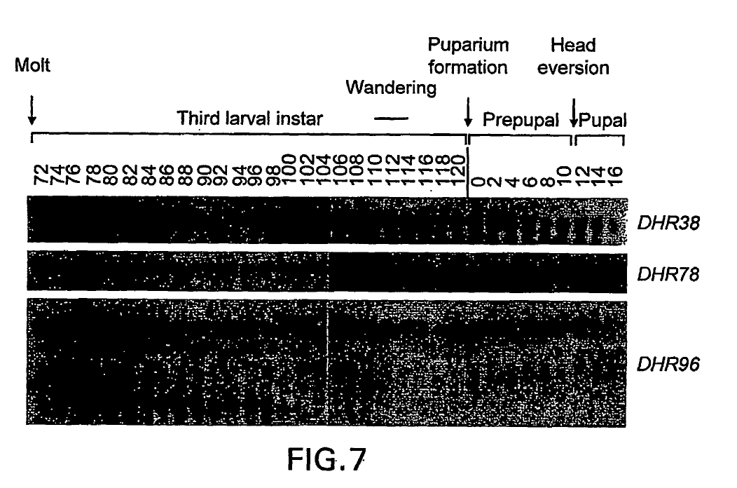

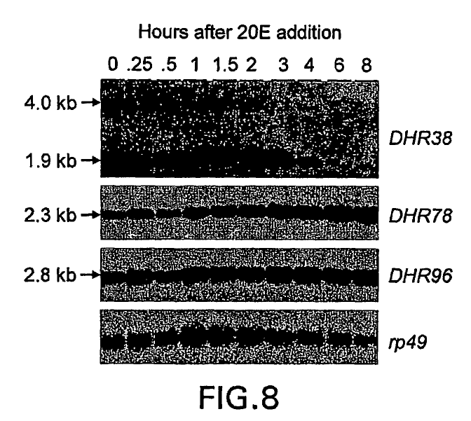

DHR96 encodes a 2.8 kb transcript that is expressed throughout third instar larval and prepupal development, with distinct increases in abundance at 106 hrs after egg laying (FIG. 7). The temporal patterns of DHR96 transcription most closely resemble those of the genes encoding the 20E receptor. EcR and usp mRNAs can be detected throughout third instar larval and prepupal development (Andres, A. J., et al., (1993). Dev. Biol 160, 388-404; 36; Henrich, V. C., et al., (1994). Dev. Biol. 165, 38-52).

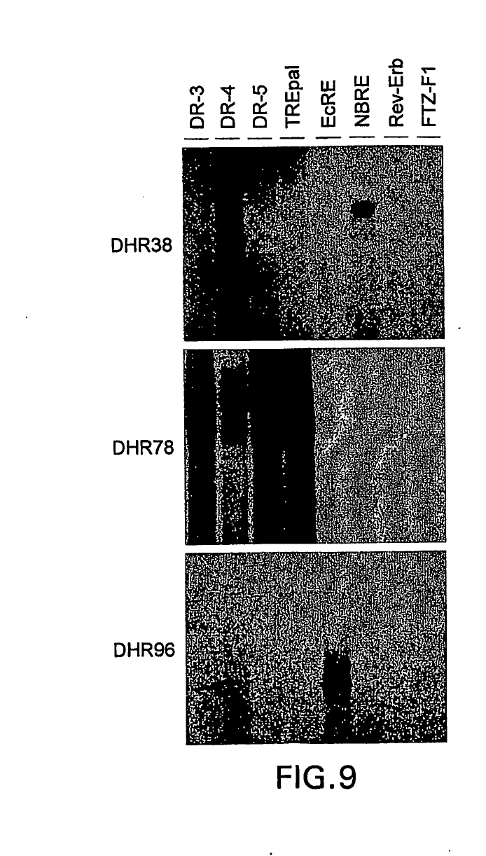

The hsp27 EcRE is the only oligonucleotide bound by DHR96, albeit it a weak interaction (FIG. 9). The EcRE consists of a palindromic arrangement of the imperfect half-sites AGtgCA and gGtTCA. DHR78 and DHR96 recognize distinct sequences that can also be bound by the EcR/Usp heterodimer (Horner, M., et al., (1995). Dev. Biol. 168, 490-502). These distinct binding specificities are consistent with the P-box sequences of the DHR78 and DHR96 proteins. The DHR78P-box, EGCKG, like that of DHR38, directs binding to an AGGTCA half-site sequence (Tsai, M.-J. & O'Malley, B. W. (1994). Annu. Rev. Biochem. 63, 451-486). In contrast, DHR96 contains a unique P-box sequence that is only present in its three C. elegans homologs (see Table 4 above)—ESCKA The binding of the hsp27 EcRE by DHR96 is very weak. An optimal DNA binding site can be identified by further experimentation.

It will be of interest to determine whether DHR78 or DHR96 can heterodimerize with EcR, Usp, or any of the Drosophila orphan receptors.

(a) DHR96 Functions in the Xenobiotic Pathway

Several lines of evidence support the conclusion that DHR96 acts in a xenobiotic pathway. First, the protein is selectively expressed in tissues involved in nutrient absorption (gastric cacae), metabolism (fat body), and excretion (Malpighian tubules)—tissues that should play a primary role in detoxification and elimination of both endobiotic and xenobiotic compounds. Second, DHR96 mutants, like null mutants in the mouse PXR and CAR xenobiotic nuclear receptors; are viable and fertile, indicating no critical role in normal development. Third, DHR96 mutants are more sensitive to the pesticide DDT. Fourth, the most highly repressed genes in response to DHR96 overexpression comprise members of all four classes of insect detoxifying genes.

The effect of the mutants can be confirmed by the expression of wild type DHR96 (from a heat-inducible DHR96 transgene, for example) in a homozygous mutant background, and test for DDT sensitivity. This experiment should rescue the sensitivity back to wild type levels. In addition, DHR96 function was reduced by RNAi and this results in levels of DDT sensitivity that are similar to those of DHR96 mutants.

The decreased resistance to DDT in DHR96 mutants can be confirmed as related to the inability to neutralize toxins rather than a general lack of fitness by demonstrating that sensitivity of DHR96 mutants occurs for toxic compounds. It can also be confirmed by showing that detoxifying genes fail to be induced in DHR96 mutants treated with toxic compounds, by for example, microarray analysis, with the mutants in the presence or absence of a toxin. These results could be compared to the microarray data disclosed herein. Two toxins that could be used for this are DDT and phenobarbital because the latter was shown to induce a number of cytochrome P450 genes in Drosophila (Danielson, P. B. et al. (1998) Mol Gen Genet. 259, 54-59).

The expression of DHR96 and its activation level can be assayed to determine if it is directly activated by toxic compounds, similar to the ability of xenobiotics to bind to human PXR xenobiotic nuclear receptor. This can be done using transformed Drosophila that express a fusion of the yeast GAl4 DNA binding domain to the ligand binding domain of DHR96. When combined with a GAL4-dependent lacZ reporter gene, the expression of β-galactosidase will only occur when the DHR96 ligand binding domain is in an active conformation. This could be caused by a direct interaction between DHR96 and the toxin. Larval organs that carry these constructs can be cultured in the presence of various xenobiotic inducers, testing for induction of lacZ reporter gene activity. Furthermore, target gene promoters can be identified which can also demonstrate a direct interaction between DHR96 and the expression of a detoxifying enzyme.

In the disclosed microarray study, DHR96 was overexpressed and it was found that this resulted in repression of a significant number of members of the major detoxification gene families. Repression of cuticle proteins was also observed, consistent with a role for cuticle formation in inhibiting pesticide toxicity (Wilson, T. G. (2001). Annu Rev Entomol 46, 545-571). The observation that these target genes are repressed suggests that DHR96 might function as a repressor in the absence of ligand. This is consistent with the action of other nuclear receptors, for example, both Endocrine receptor (EcR) and thyroid receptor (TR) are known to function in this manner. Very strict filtering criteria were used in the disclosed microarray experiments further strengthening the results.

The microarray studies allow the identification of the direct targets of DHR96. This will allow the identification of the genetic hierarchy that is regulated by this nuclear receptor. Once target genes have been identified, it will be possible to construct reporter genes that are inducible by endogenous DHR96. Such a system can then be utilized to screen for drugs or combinations of drugs that activate or repress these reporter genes, in both a wild type and DHR96 mutant background. This can further confirm that DHR96 can directly regulate the expression of detoxifying genes. This system would also provide a direct readout of DHR96 activity that would be useful for further studies of DHR96 function and for the development of appropriate inhibitors of DHR96 function. The mutants of DHR96 can be used to identify and confirm other factors that can act as xenobiotic receptors in insects, and test whether these act in a partially redundant manner with DHR96.

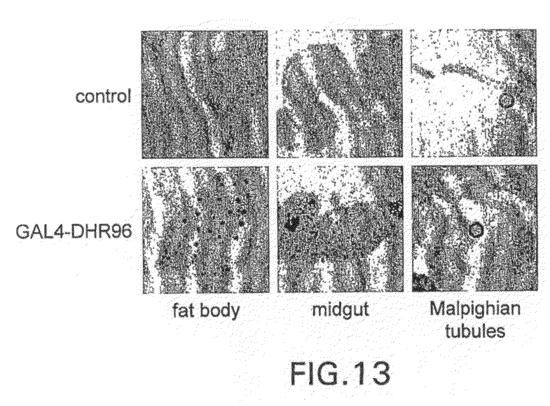

As disclosed herein, PXR and DHR96 are highly homologous. PXR transactivation and binding assays have been developed into high-throughput assays (Zhu et al., J Biomol Screen. 2004 September; 9(6):533-40; Kliewer et al., Endocrine Rev. 2002 23(5):687-702 herein incorporated by reference in its entirety for its teaching concerning PXR, transactivation assays, and binding assays.) Zhu et al. found a good correlation between the results of the transactivation and binding assays. An example of an antagonist of PXR is ecteinascidin-743. Furthermore, several compounds can activate DHR96, such as tebufenozide (RH-5992, FIG. 13) (Dinan et al. 1997 Biochem J. 327:643-50). This compound is both an ecdysteroid agonist and a lepidopteran insecticide.

The steroid and xenobiotic receptor (SXR) is another nuclear receptor with a high degree of homology with DHR96. SXR is a nuclear receptor that regulates drug clearance in the liver and intestine via induction of genes involved in drug and xenobiotic metabolism. The α, β, Δ, and γ tocotrienols specifically bind to and activate SXR (Zhou et al. Drug Metab Dispos. 2004 October; 32(10):1075-82, herein incorporated by reference for its teaching concerning SXR). Many other compounds also activate SXR and can be activators of DHR96 as well (Blumberg et al. Genes Dev. 1998 October 15 12(20):3195-205, herein incorporated by reference in its entirety for its teaching regarding nuclear receptor modulators.)

Nuclear receptors, such as DHR96, SXR, and PXR, contain a lypophilic ligand binding pocket. This pocket can be bound by compounds that affect the activity of the nuclear receptor, and therefore act as selective modulators of the nuclear receptor. These selective modulators can act as either agonists or antagonists, and modulators of one nuclear receptor can act as modulators of another.

### (3) Mutants of the DHR96 Gene

Various DHR96 mutant alleles were made. A series of studies to characterize the DHR96 mutant alleles were performed. These included Southern, Northern and Western blotting, tissue stains, sequencing of PCR products, and genetic mapping to validate the mutations in the different DHR96 alleles. Validation of these alleles was particularly important because flies homozygous for DHR96 mutations are viable and fertile. At least one of the alleles generated, DHR96^{16A}, is a protein null, because the translation start site was deleted and no protein was detectable in Western blots or tissue stains of homozygous mutant animals.

Gene targeting (Rong, Y. S., and Golic, K. G. (2000). Science 288, 2013-2018) was used to generate mutations in DHR96 because no deficiencies or P elements were known in this region of the genome. (see Example 1). Using these methods any mutations of the DHR96 gene can be made, such as mutations at or around the start site; mutations at or around the splice sites; mutations which prevent or render inactive complete or partial exon sequences; mutations which render inactive or remove the complete or partial DBD or LBD or any of the domains of DHR96 discussed herein that it contains as a nuclear receptor.

The DHR96 gene resides on the third chromosome. When mutations are made in certain embodiments the mutations of the DHR96 gene are made such that there is only a single copy of the mutant and no copies of the wildtype gene in the insect, such as the fly. This is done, for example, by using vectors for the mutation generation, which have sites built in that allow for recombination and excision of the site, and fly stocks containing a single copy can be selected. (see for example, Rong, Y. et al., (2002) Genes Dev 16, 1568-1581).

Disclosed are null mutants of the DHR96 gene. A null mutant is defined herein as a mutant that lacks functional DHR96 protein product.

A null mutant disclosed herein is DHR96^{16A} which is mutant having two specific deletions, one removing the start codon for translation and the second removing intron/exon 4, deleting a critical portion of the LBD.

Another null mutant disclosed herein is the mutant DHR96^{E25} which carries a tandem duplication of the DHR96 gene in place of the single wild type copy. One of these mutant DHR96 genes is identical to the DHR96^{16A} allele described above, missing both the start codon and intron/exon 4. The other mutant DHR96 gene is lacking only intron/exon 4. Western blot analysis indicates that both DHR96^{E25} mutants, as well as DHR96^{16A} mutants, produce no detectable DR96 protein. Thus, both alleles can be considered as null mutations.

One way to functionally test the mutants is in a viability assay based on different nutritional backgrounds. Disclosed herein, DHR96 mutants will have a decreased ability to grow on instant fly food, such as Carolina 424. If yeast is restored to the instant food, viability is restored to within wildtype levels, indicating that DHR96 mutants are sensitive to the absence of yeast in their food source. In contrast, mutants such as DHR96^{E25} or DHR96^{16A} are viable when grown on standard cornmeal medium.

Disclosed are insects, such as flies, containing the mutant DHR96 gene, as well as any of their developmental stages, such as larvae, eggs, or pupae. These flies can be used, for example, to be crossed with other strains of flies to make new strains harboring the DHR96 mutants. These strains could also be used, for example, as a type of insect inhibitor themselves, by being released in the wild to cross with wildtype insects creating mutant insects. For this purpose, mutations that create a dominant negative phenotype are preferred, such as those that have non-functional LBD, but retain their ability to heterodimerize, thus, interacting with and reducing the effect of native proteins in the insect.

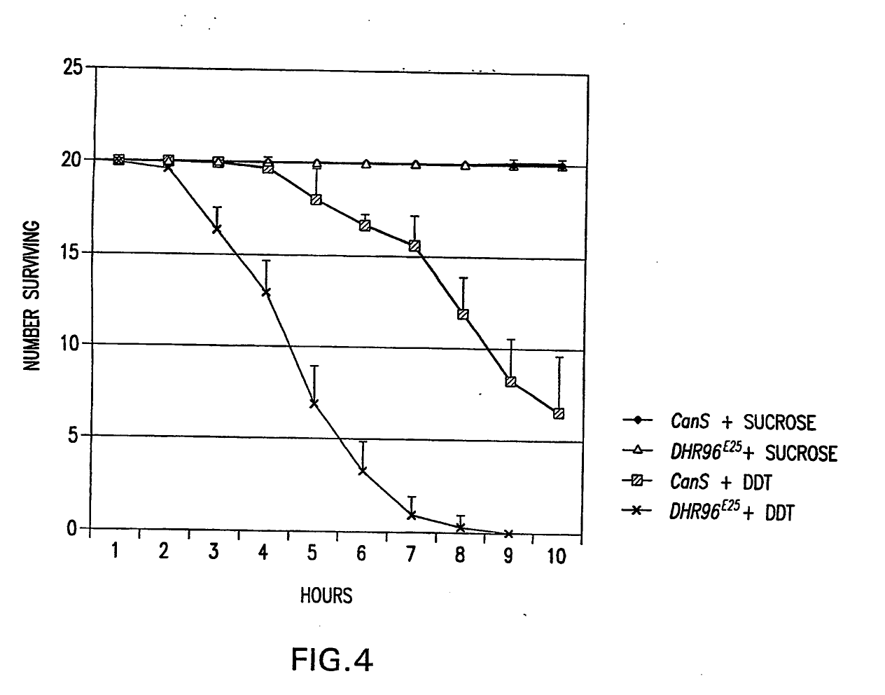

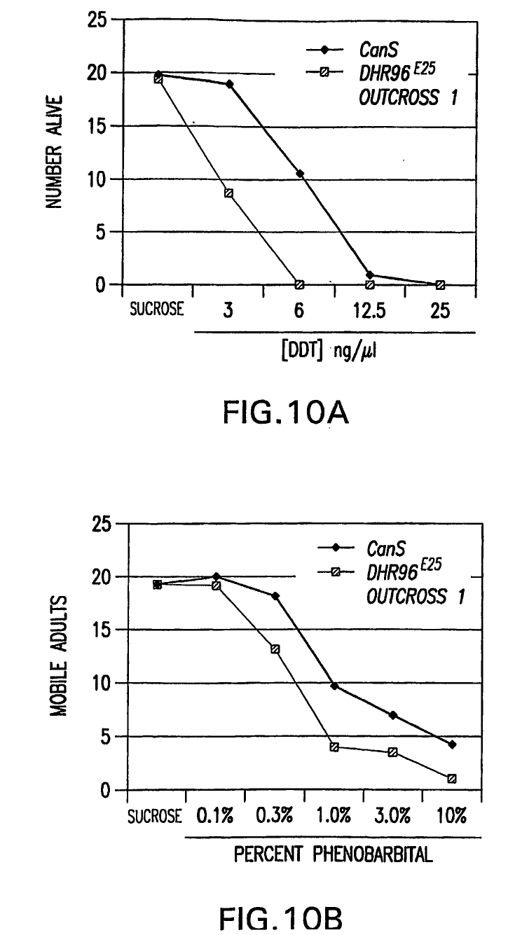

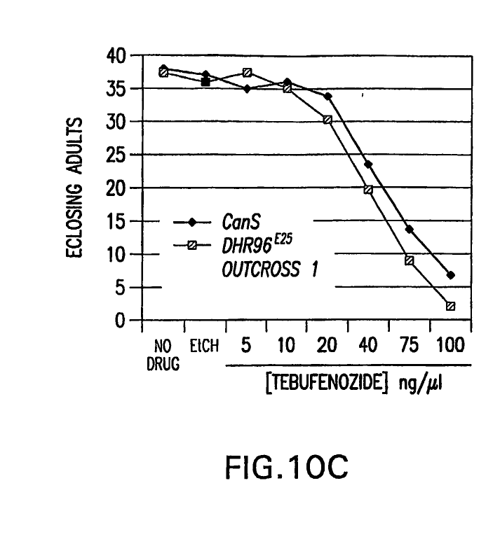

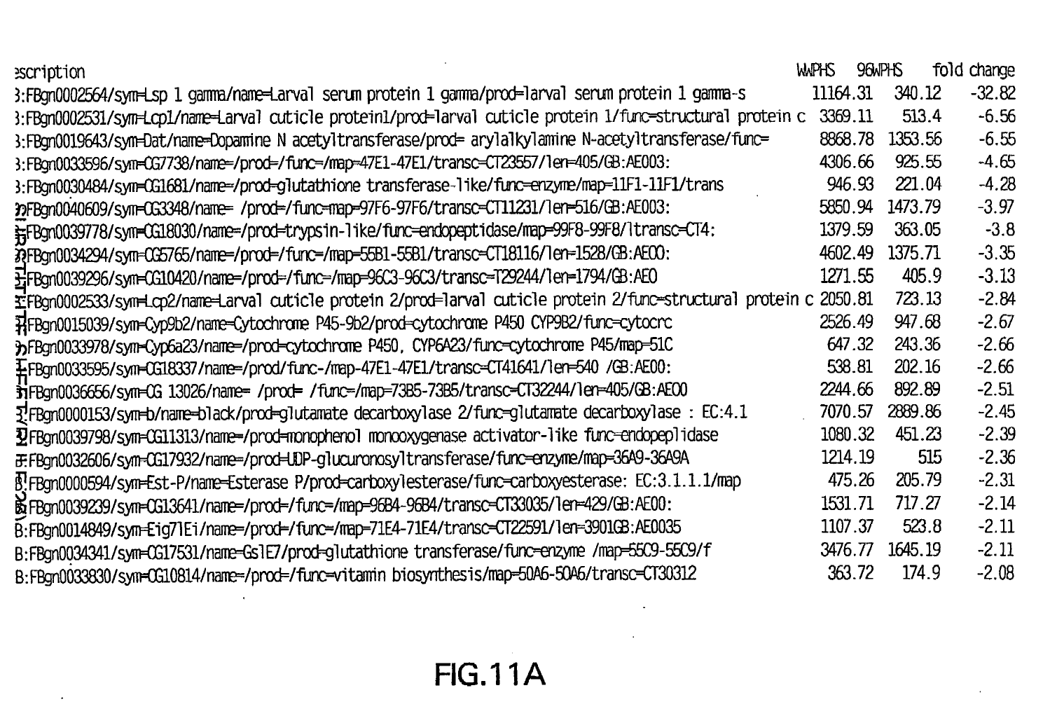

The disclosed mutants cause a decrease in the insect's ability to react to toxins or pesticides, such as DDT. The disclosed mutants, such as DHR96^{16A} or DHR96^{E25} insects, such as flies, were more sensitive to DDT and died at lower concentrations of DDT compared to control animals (FIG. 4). In addition, when challenged with a fixed concentration of DDT, DHR96 homozygotes died more rapidly than wild type flies (FIG. 10).

Also disclosed are mutants which have a defect in for example, activation with and without retention of dimerization ability, defects in ligand binding, and defects in DNA binding with and without loss of dimerization ability.

Also disclosed are mutants that, when overexpressed, fail to modulate genes in the xenobiotic pathway, such as genes in the four major detoxification families, cytochrome P450s, carboxylesterases, glutathione S-transferases, and UDP-glucuronosyltransferases (Oakeshott J G, Home I, Sutherland T D, Russell R J. The genomics of insecticide resistance. Genome Biol. 2003; 4(1):202). In Table 3, two are P450s (Cyp genes), two are glutathione S-transferases, and one each of the carboxylesterases and UDP-glucuronosyltransferases were identified by microarray analysis. These represent the function of these proteins. Also denoted in Table 3 are the names of the genes. These are the gene names according to FlyBase (http://flybase.bio.indiana.edu/) They are either a proper name, like black or Lcpl, or the CG number, which is a numerical designation given to each fly gene. The CG number is usually used when the gene is new or of unknown function. This can be determined using microarrays as disclosed herein.

### (4) Compounds that Modulate DHR96 Activity

Disclosed are compounds that modulate DHR96 activity. These compounds can, for example, modulate the activity of the protein through binding with the protein of DHR96, or through binding the mRNA of DHR96, and inhibiting the mRNA, through, for example, degradation or prevention of translation. The compositions can be any type of molecule, including, for example, proteins, small peptides, antibodies, functional nucleic acids, such as aptamers, antisense, ribozymes, dsRNA for RNAi or siRNA, or small molecules, such as those found in various combinatorial chemistry libraries or natural product libraries.

For example, disclosed are compounds that function by, for example, binding to the ligand binding domain of DHR96 and inactivating its function or turning it into a constitutive repressor, or mimicking the normal cofactors that mediate nuclear receptor signaling to the general transcription machinery. These compounds, such as peptides, would render the receptor incapable of directing proper target gene transcription, blocking the detoxification response. The disclosed compounds can act in combination with known or any pesticide by increasing the effectiveness of the pesticide by decreasing the insect's ability to react to the pesticide. The compositions could be added to pre-existing pesticide formulations, increasing their effectiveness. Moreover, resistant lines of insects that respond poorly to a particular pesticide may be made more sensitive by adding compounds that affect DHR96 function. DHR96 is a target for pest control, capable of regulating insect populations. The compositions could also prevent or reduce the translation or expression of the DHR96 mRNA, by for example, through RNAi or antisense mechanisms.

(a) Functional Nucleic Acids

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include RNAi, antisense molecules, aptamers, ribozymes, triplex forming molecules, and external guide sequences. The functional nucleic acid molecules can act as affectbrs, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of DHR96 or variants or fragments or the genomic DNA of DHR96 or variants or fragments or they can interact with the polypeptide DHR96 or variants or fragments. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Disclosed are molecules that inhibit DHR96 activity that are based on RNA interference (RNAI) or small interfering RNA (SiRNA). It is thought that RNAi involves a two-step mechanism for RNA interference (RNAi): an initiation step and an effector step. For example, in the first step, input double-stranded (ds) RNA is processed into small fragments (siRNA), such as 21-23-nucleotide ‘guide sequences’. RNA amplification appears to be able to occur in whole animals. Typically then, the guide RNAs can be incorporated into a protein RNA complex which is cable of degrading RNA, the nuclease complex, which has been called the RNA-induced silencing complex (RISC). This RISC complex acts in the second effector step to destroy mRNAs that are recognized by the guide RNAs through base-pairing interactions. RNAi involves the introduction by any means of double stranded RNA into the cell which triggers events that cause the degradation of a target RNA. RNAi is a form of post-transcriptional gene silencing. Disclosed are RNA hairpins that can act in RNAi.

RNAi has been shown to work in a number of cells, including mammalian and invertebrate cells. In certain embodiments the RNA molecules which will be used as targeting sequences within the RISC complex are shorter. For example, less than or equal to 50 or 40 or 30 or 29, 28, 27, 26, 25, 24, 23, 22, 21, 20, 19, 18, 17, 16, 15, 14, 13, 12, 11, or 10 nucleotides in length. These RNA molecules can also have overhangs on the 3′ or 5′ ends relative to the target RNA which is to be cleaved. These overhangs can be at least or less than or equal to 1, 2, 3, 4, 5, 6, 7, 8, 9, 10, 15, or 20 nucleotides long.

Methods of RNAi and SiRNA are described in detail in Hannon et al. (2002), RNA Interference, Nature 418:244-250; Brummelkamp et al. (2002), A System for Stable Expression of Short Interfering RNAs in Mammalian Cells, Science 296:550-508; Paul et al. (2002), Effective expression of small interfering RNA in human cells, Nature Biotechnology 20: 505-508, which are each incorporated by reference in their entirety for methods of RNAi and SiRNA and for designing and testing various oligos useful therein.

RNA interference (RNAi) and gene targeting were used to disrupt DHR96 function because no existing mutants were available. The effects of DHR96 RNAi were analyzed by generating transgenic lines that express snapback RNA under the control of a heat-inducible promoter. Three independent lines showed strong reduction of DHR96 mRNA in northern blots when treated with a single heat-shock, but displayed no discernable phenotype. Using a variety of heat-shock regimens, e.g. longer single and double treatments or 12 hr repetitions, did not affect the outcome of this observation. These findings suggest that DHR96 mRNA is not necessary for viability under standard conditions, indicating either that DHR96 protein is very stable or dispensable for survival, and is consistent with the studies of DHR96 null mutants.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d}) less than or equal to 10⁻⁶, 10⁻⁸, 10⁻¹², or 10⁻¹². A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with k_{d}s from the target molecule of less than 10⁻¹² M. It is preferred that the aptamers bind the target molecule with a k_{d} less than 10⁻⁶, 10⁻⁸, 10⁻¹⁰, or 10⁻¹². Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a k_{d} with the target molecule at least 10, 100, 1000, 10,000, or 100,000 fold lower than the k_{d} with a background binding molecule. It is preferred when doing the comparison for a polypeptide for example, that the background molecule be a different polypeptide. For example, when determining the specificity of aptamers to DHR96 protein or fragments or variants, the background protein could be serum albumin. Representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos. 5,595,873 and 5,652,107). There are also a number of ribozymes that are not found in natural systems, but which have been engineered to catalyze specific reactions de novo (for example, but not limited to the following U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶, 10⁻⁸, 10⁻¹⁰, or 10⁻¹². Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukarotic cells. (Yuan et al., Proc. Natl. Acad. Sci. USA 89:8006-8010 (1992); WO 93/22434 by Yale; WO 95/24489 by Yale; Yuan and Altman, EMBO J. 14:159-168 (1995), and Carrara et al. Proc. Natl. Acad. Sci. (USA) 92:2627-2631 (1995)). Representative examples of how to make and use EGS molecules to facilitate cleavage of a variety of different target molecules be found in the following non-limiting list of U.S. Pat. Nos. 5,168,053, 5,624,824, 5,683,873, 5,728,521, 5,869,248, and 5,877,162.

(b) Antibodies

Disclosed are monoclonal and polyclonal as well as chimeric variants of these, that bind DHR96 or variants or fragments thereof. Also disclosed are monoclonal and polyclonal antibodies that bind DHR96 or variants or fragments thereof that inhibit DHR96 activity in, for example, the xenobiotic pathways disclosed herein. Various assays are disclosed herein that can be used to identify these antibodies, such as the nutritional viability assay disclosed herein or the sensitivity to toxins assay disclosed herein.

As used herein, the term “antibody” encompasses, but is not limited to, whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V(H)) followed by a number of constant domains. Each light chain has a variable domain at one end (V(L)) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (k) and lambda (l), based on the amino acid sequences of their constant domains. Depending on the amino acid sequence of the constant domain of their heavy chains, immunoglobulins can be assigned to different classes. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “variable” is used herein to describe certain portions of the variable domains that differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a b-sheet configuration, connected by three CDRs, which form loops connecting, and in some cases forming part of, the b-sheet structure. The CDRs in each chain are held together in close proximity by the FR regions and, with the CDRs from the other chain, contribute to the formation of the antigen binding site of antibodies (see Kabat E. A. et al., “Sequences of Proteins of Immunological Interest,” National Institutes of Health, Bethesda, Md. (1987)). The constant domains are not involved directly in binding an antibody to an antigen, but exhibit various effector functions, such as participation of the antibody in antibody-dependent cellular toxicity.

As used herein, the term “antibody or fragments thereof” encompasses chimeric antibodies and hybrid antibodies, with dual or multiple antigen or epitope specificities, and fragments, such as F(ab′)2, Fab′, Fab and the like, including hybrid fragments. Thus, fragments of the antibodies that retain the ability to bind their specific antigens are provided. For example, fragments of antibodies which maintain binding activity to the DHR96 or variants or fragments thereof are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies) as described, for example, in U.S. Pat. No. 4,704,692, the contents of which are hereby incorporated by reference.

Optionally, the antibodies are generated in other species and “humanized” for administration in humans. Humanized forms of non-human (e.g., murine) antibodies are chimeric immunoglobulins, immunoglobulin chains or fragments thereof (such as Fv, Fab, Fab′, F(ab′)2, or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature, 332:323-327 (1988); and Presta, Curr. Op. Struct. Biol., 2:593-596 (1992)).

Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence, from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important in order to reduce antigenicity. According to the “best-fit” method, the sequence of the variable domain of a rodent antibody is screened against the entire library of known human variable domain sequences. The human sequence which is closest to that of the rodent is then accepted as the human framework (FR) for the humanized antibody (Sims et al., J. Immunol., 151:2296 (1993) and Chothia et al., J. Mol. Biol., 196:901 (1987)). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding (see, WO 94/04679, published 3 Mar. 1994).

Transgenic animals (e.g., mice) that are capable, upon immunization, of producing a full repertoire of human antibodies in the absence of endogenous immunoglobulin production can be employed. For example, it has been described that the homozygous deletion of the antibody heavy chain joining region (J(H)) gene in chimeric and germ-line mutant mice results in complete inhibition of endogenous antibody production. Transfer of the human germ-line immunoglobulin gene array in such germ-line mutant mice will result in the production of human antibodies upon antigen challenge (see, e.g., Jakobovits et al., Proc. Natl. Acad. Sci. USA, 90:2551-255 (1993); Jakobovits et al., Nature, 362:255-258 (1993); Bruggemann et al., Year in Immuno., 7:33 (1993)). Human antibodies can also be produced in phage display libraries (Hoogenboom et al., J. Mol. Biol., 227:381 (1991); Marks et al., J. Mol. Biol., 222:581 (1991)). The techniques of Cote et al. and Boerner et al. are also available for the preparation of human monoclonal antibodies (Cole et al., Monoclonal Antibodies and Cancer Therapy, Alan R. Liss, p. 77 (1985); Boerner et al., J. Immunol., 147(1):86-95 (1991)).

Disclosed are hybidoma cells that produces the monoclonal antibody. The term “monoclonal antibody” as used herein refers to an antibody obtained from a substantially homogeneous population of antibodies, i.e., the individual antibodies comprising the population are identical except for possible naturally occurring mutations that may be present in minor amounts. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired activity (See, U.S. Pat. No. 4,816,567 and Morrison et al., Proc. Natl. Acad. Sci. USA, 81:6851-6855 (1984)).

Monoclonal antibodies may be prepared using hybridoma methods, such as those described by Kohler and Milstein, Nature, 256:495 (1975) or Harlow and Lane. Antibodies, A Laboratory Manual. Cold Spring Harbor Publications, New York, (1988). In a hybridoma method, a mouse or other appropriate host animal, is typically immunized with an immunizing agent to elicit lymphocytes that produce or are capable of producing antibodies that will specifically bind to the immunizing agent. Alternatively, the lymphocytes may be immunized in vitro. Preferably, the immunizing agent comprises DHR96 or variants or fragments thereof. Traditionally, the generation of monoclonal antibodies has depended on the availability of purified protein or peptides for use as the immunogen. More recently DNA based immunizations have shown promise as a way to elicit strong immune responses and generate monoclonal antibodies. In this approach, DNA-based immunization can be used, wherein DNA encoding a portion of DHR96 or variants or fragments thereof expressed as a fusion protein with human IgG1 is injected into the host animal according to methods known in the art (e.g., Kilpatrick K E, et al. Gene gun delivered DNA-based immunizations mediate rapid production of murine monoclonal antibodies to the Flt-3 receptor. Hybridoma. 1998 December; 17(6):569-76; Kilpatrick K E et al. High-affinity monoclonal antibodies to PED/PEA-15 generated using 5 microg of DNA. Hybridoma. 2000 August; 19(4):297-302, which are incorporated herein by referenced in full for the methods of antibody production) and as described in the examples.

An alternate approach to immunizations with either purified protein or DNA is to use antigen expressed in baculovirus. The advantages to this system include ease of generation, high levels of expression, and post-translational modifications that are highly similar to those seen in mammalian systems. Use of this system involves expressing domains of antibodies to DHR96 or variants or fragments thereof as fusion proteins. The antigen is produced by inserting a gene fragment in-frame between the signal sequence and the mature protein domain of the antibodies to DHR96 or variants or fragments thereof nucleotide sequence. This results in the display of the foreign proteins on the surface of the virion. This method allows immunization with whole virus, eliminating the need for purification of target antigens.

Generally, either peripheral blood lymphocytes (“PBLs”) are used in methods of producing monoclonal antibodies if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, “Monoclonal Antibodies: Principles and Practice” Academic Press, (1986) pp. 59-103). Immortalized cell lines are usually transformed mammalian cells, including myeloma cells of rodent, bovine, equine, and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells. Preferred immortalized cell lines are those that fuse efficiently, support stable high level expression of antibody by the selected antibody-producing cells, and are sensitive to a medium such as HAT medium. More preferred inunortalized cell lines are murine myeloma lines, which can be obtained, for instance, from the Salk Institute Cell Distribution Center, San Diego, Calif. and the American Type Culture Collection, Rockville, Md. Human myeloma and mouse-human heteromyeloma cell lines also have been described for the production of human monoclonal antibodies (Kozbor, J. Immunol., 133:3001 (1984); Brodeur et al., “Monoclonal Antibody Production Techniques and Applications” Marcel Dekker, Inc., New York, (1987) pp. 51-63). The culture medium in which the hybridoma cells are cultured can then be assayed for the presence of monoclonal antibodies directed against DHR96 or variants or fragments thereof. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art, and are described further in the Examples below or in Harlow and Lane “Antibodies, A Laboratory Manual” Cold Spring Harbor Publications, New York, (1988).

After the desired hybridoma cells are identified, the clones may be subcloned by limiting dilution or FACS sorting procedures and grown by standard methods. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, protein G, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies can be readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as simian COS cells, Chinese hamster ovary (CHO) cells, plasmacytoma cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567) or by covalently joining to the immunoglobulin coding sequence all or part of the coding sequence for a non-immunoglobulin polypeptide. Optionally, such a non-immunoglobulin polypeptide is substituted for the constant domains of an antibody or substituted for the variable domains of one antigen-combining site of an antibody to create a chimeric bivalent antibody comprising one antigen-combining site having specificity for DHR96 or variants or fragments thereof and another antigen-combining site having specificity for a different antigen.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994, U.S. Pat. No. 4,342,566, and Harlow and Lane, Antibodies, A Laboratory Manual, Cold Spring Harbor Publications, New York, (1988). Papain digestion of antibodies typically produces two identical antigen binding fragments, called Fab fragments, each with a single antigen binding site, and a residual Fc fragment. Pepsin treatment yields a fragment, called the F(ab′)2 fragment, that has two antigen combining sites and is still capable of cross-linking antigen.

The Fab fragments produced in the antibody digestion also contain the constant domains of the light chain and the first constant domain of the heavy chain. Fab′ fragments differ from Fab fragments by the addition of a few residues at the carboxy terminus of the heavy chain domain including one or more cysteines from the antibody hinge region. The F(ab′)2 fragment is a bivalent fragment comprising two Fab′ fragments linked by a disulfide bridge at the hinge region. Fab′-SH is the designation herein for Fab′ in which the cysteine residue(s) of the constant domains bear a free thiol group. Antibody fragments originally were produced as pairs of Fab′ fragments which have hinge cysteines between them. Other chemical couplings of antibody fragments are also known.

An isolated immunogenically specific paratope or fragment of the antibody is also provided. A specific immunogenic epitope of the antibody can be isolated from the whole antibody by chemical or mechanical disruption of the molecule. The purified fragments thus obtained are tested to determine their immunogenicity and specificity by the methods taught herein. Immunoreactive paratopes of the antibody, optionally, are synthesized directly. An immunoreactive fragment is defined as an amino acid sequence of at least about two to five consecutive amino acids derived from the antibody amino acid sequence.

One method of producing proteins comprising the antibodies is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the antibody, for example, can be synthesized by standard chemical reactions. For example, a peptide or polypeptide can be synthesized and not cleaved from its synthesis resin whereas the other fragment of an antibody can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY. Alternatively, the peptide or polypeptide is independently synthesized in vivo as described above. Once isolated, these independent peptides or polypeptides may be linked to form an antibody or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide-alpha-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site. Application of this native chemical ligation method to the total synthesis of a protein molecule is illustrated by the preparation of human interleukin 8 (IL-8) (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

Also disclosed are fragments of antibodies which have bioactivity. The polypeptide fragments can be recombinant proteins obtained by cloning nucleic acids encoding the polypeptide in an expression system capable of producing the polypeptide fragments thereof, such as an adenovirus or baculovirus expression system. For example, one can determine the active domain of an antibody from a specific hybridoma that can cause a biological effect associated with the interaction of the antibody with DHR96 or variants or fragments thereof. For example, amino acids found to not contribute to either the activity or the binding specificity or affinity of the antibody can be deleted without a loss in the respective activity. For example, in various embodiments, amino or carboxy-terminal amino acids are sequentially removed from either the native or the modified non-immunoglobulin molecule or the immunoglobulin molecule and the respective activity assayed in one of many available assays. In another example, a fragment of an antibody comprises a modified antibody wherein at least one amino acid has been substituted for the naturally occurring amino acid at a specific position, and a portion of either amino terminal or carboxy terminal amino acids, or even an internal region of the antibody, has been replaced with a polypeptide fragment or other moiety, such as biotin, which can facilitate in the purification of the modified antibody. For example, a modified antibody can be fused to a maltose binding protein, through either peptide chemistry or cloning the respective nucleic acids encoding the two polypeptide fragments into an expression vector such that the expression of the coding region results in a hybrid polypeptide. The hybrid polypeptide can be affinity purified by passing it over an amylose affinity column, and the modified antibody receptor can then be separated from the maltose binding region by cleaving the hybrid polypeptide with the specific protease factor Xa. (See, for example, New England Biolabs Product Catalog, 1996, pg. 164.). Similar purification procedures are available for isolating hybrid proteins from eukaryotic cells as well.

The fragments, whether attached to other sequences or not, include insertions, deletions, substitutions, or other selected modifications of particular regions or specific amino acids residues, provided the activity of the fragment is not significantly altered or impaired compared to the nonmodified antibody or antibody fragment. These modifications can provide for some additional property, such as to remove or add amino acids capable of disulfide bonding, to increase its bio-longevity, to alter its secretory characteristics, etc. In any case, the fragment must possess a bioactive property, such as binding activity, regulation of binding at the binding domain, etc. Functional or active regions of the antibody may be identified by mutagenesis of a specific region of the protein, followed by expression and testing of the expressed polypeptide. Such methods are readily apparent to a skilled practitioner in the art and can include site-specific mutagenesis of the nucleic acid encoding the antigen. (Zoller M J et al. Nucl. Acids Res. 10:6487-500 (1982).

A variety of immunoassay formats may be used to select antibodies that selectively bind with a particular protein, variant, or fragment. For example, solid-phase ELISA immunoassays are routinely used to select antibodies selectively immunoreactive with a protein, protein variant, or fragment thereof. See Harlow and Lane. Antibodies, A Laboratory Manual. Cold Spring Harbor Publications, New York, (1988), for a description of immunoassay formats and conditions that could be used to determine selective binding. The binding affinity of a monoclonal antibody can, for example, be determined by the Scatchard analysis of Munson et al., Anal. Biochem., 107:220 (1980).

Also provided is an antibody reagent kit comprising containers of the monoclonal antibody or fragment thereof and one or more reagents for detecting binding of the antibody or fragment thereof to DHR96 or variants or fragments thereof. The reagents can include, for example, fluorescent tags, enzymatic tags, or other tags. The reagents can also include secondary or tertiary antibodies or reagents for enzymatic reactions, wherein the enzymatic reactions produce a product that can be visualized.

(c) Compositions Identified by Screening with Disclosed Compositions/Combinatorial Chemistry

(i) Combinialorial Chemistry

The disclosed compositions can be used as targets for any combinatorial technique to identify molecules or macromolecular molecules that interact with the disclosed compositions in a desired way. The nucleic acids, peptides, and related molecules disclosed herein, such as DHR96 or variants or fragments thereof, can be used as targets for the combinatorial approaches. Also disclosed are the compositions that are identified through combinatorial techniques or screening techniques in which the compositions, such as DHR96 or variants or fragments thereof, or portions thereof, are used as the target in a combinatorial or screening protocol.

It is understood that when using the disclosed compositions in combinatorial techniques or screening methods, molecules, such as macromolecular molecules, will be identified that have particular desired properties such as inhibition or stimulation or the target molecule's function. The molecules identified and isolated when using the disclosed compositions, such as, DHR96 or variants or fragments thereof, are also disclosed. Thus, the products produced using the combinatorial or screening approaches that involve the disclosed compositions, such as, DHR96 or variants or fragments thereof, are also considered herein disclosed.

It is understood that the disclosed methods for identifying molecules that inhibit the interactions between, for example, DHR96 or variants or fragments thereof, can be performed using high through put means. For example, putative inhibitors can be identified using Fluorescence Resonance Energy Transfer (FRET) to quickly identify interactions. The underlying theory of the techniques is that when two molecules are close in space, ie, interacting at a level beyond background, a signal is produced or a signal can be quenched. Then, a variety of experiments can be performed, including, for example, adding in a putative inhibitor. If the inhibitor competes with the interaction between the two signaling molecules, the signals will be removed from each other in space, and this will cause a decrease or an increase in the signal, depending on the type of signal used. This decrease or increasing signal can be correlated to the presence or absence of the putative inhibitor. Any signaling means can be used. For example, disclosed are methods of identifying an inhibitor of the interaction between any two of the disclosed molecules comprising, contacting a first molecule and a second molecule together in the presence of a putative inhibitor, wherein the first molecule or second molecule comprises a fluorescence donor, wherein the first or second molecule, typically the molecule not comprising the donor, comprises a fluorescence acceptor; and measuring Fluorescence Resonance Energy Transfer (FRET), in the presence of the putative inhibitor and the in absence of the putative inhibitor, wherein a decrease in FRET in the presence of the putative inhibitor as compared to FRET measurement in its absence indicates the putative inhibitor inhibits binding between the two molecules. This type of method can be performed with a cell system as well.

Combinatorial chemistry includes but is not limited to all methods for isolating small molecules or macromolecules that are capable of binding either a small molecule or another macromolecule, typically in an iterative process. Proteins, oligonucleotides, and sugars are examples of macromolecules. For example, oligonucleotide molecules with a given function, catalytic or ligand-binding, can be isolated from a complex mixture of random oligonucleotides in what has been referred to as “in vitro genetics” (Szostak, TIBS 19:89, 1992). One synthesizes a large pool of molecules bearing random and defined sequences and subjects that complex mixture, for example, approximately 10¹⁵ individual sequences in 100 μg of a 100 nucleotide RNA, to some selection and enrichment process. Through repeated cycles of affinity chromatography and PCR amplification of the molecules bound to the ligand on the column, Ellington and Szostak (1990) estimated that 1 in 10¹⁰ RNA molecules folded in such a way as to bind a small molecule dyes. DNA molecules with such ligand-binding behavior have been isolated as well (Ellington and Szostak, 1992; Bock et al, 1992). Techniques aimed at similar goals exist for small organic molecules, proteins, antibodies and other macromolecules known to those of skill in the art. Screening sets of molecules for a desired activity whether based on small organic libraries, oligonucleotides, or antibodies is broadly referred to as combinatorial chemistry. Combinatorial techniques are particularly suited for defining binding interactions between molecules and for isolating molecules that have a specific binding activity, often called aptamers when the macromolecules are nucleic acids.

There are a number of methods for isolating proteins which either have de novo activity or a modified activity. For example, phage display libraries have been used to isolate numerous peptides that interact with a specific target. (See for example, U.S. Pat. Nos. 6,031,071; 5,824,520; 5,596,079; and 5,565,332 which are herein incorporated by reference at least for their material related to phage display and methods relate to combinatorial chemistry)

A preferred method for isolating proteins that have a given function is described by Roberts and Szostak (Roberts R. W. and Szostak J. W. Proc. Natl. Acad. Sci. USA, 94(23)12997-302 (1997). This combinatorial chemistry method couples the functional power of proteins and the genetic power of nucleic acids. An RNA molecule is generated in which a puromycin molecule is covalently attached to the 3′-end of the RNA molecule. An in vitro translation of this modified RNA molecule causes the correct protein, encoded by the RNA to be translated. In addition, because of the attachment of the puromycin, a peptidyl acceptor which cannot be extended, the growing peptide chain is attached to the puromycin which is attached to the RNA. Thus, the protein molecule is attached to the genetic material that encodes it. Normal in vitro selection procedures can now be done to isolate functional peptides. Once the selection procedure for peptide function is complete traditional nucleic acid manipulation procedures are performed to amplify the nucleic acid that codes for the selected functional peptides. After amplification of the genetic material, new RNA is transcribed with Puromycin at the 3′-end, new peptide is translated and another functional round of selection is performed. Thus, protein selection can be performed in an iterative manner just like nucleic acid selection techniques. The peptide which is translated is controlled by the sequence of the RNA attached to the puromycin. This sequence can be anything from a random sequence engineered for optimum translation (i.e. no stop codons etc.) or it can be a degenerate sequence of a known RNA molecule to look for improved or altered function of a known peptide. The conditions for nucleic acid amplification and in vitro translation are well known to those of ordinary skill in the art and are preferably performed as in Roberts and Szostak (Roberts R. W. and Szostak J. W. Proc. Natl. Acad. Sci. USA, 94(23)12997-302 (1997)).

Another preferred method for combinatorial methods designed to isolate peptides is described in Cohen et al. (Cohen B. A., et al., Proc. Natl. Acad. Sci. USA 95(24):14272-7 (1998)). This method utilizes and modifies two-hybrid technology. Yeast two-hybrid systems are useful for the detection and analysis of protein:protein interactions. The two-hybrid system, initially described in the yeast Saccharomyces cerevisiae, is a powerful molecular genetic technique for identifying new regulatory molecules, specific to the protein of interest (Fields and Song, Nature 340:245-6 (1989)). Cohen et al., modified this technology so that novel interactions between synthetic or engineered peptide sequences could be identified which bind a molecule of choice. The benefit of this type of technology is that the selection is done in an intracellular environment. The method utilizes a library of peptide molecules that attached to an acidic activation domain. A peptide of choice, for example, of DHR96 or variants or fragments thereof, is attached to a DNA binding domain of a transcriptional activation protein, such as Gal 4. By performing the two-hybrid technique on this type of system, molecules that bind DHR96 or variants or fragments thereof can be identified.

Using methodology well known to those of skill in the art, in combination with various combinatorial libraries, one can isolate and characterize those small molecules or macromolecules, which bind to or interact with the desired target. The relative binding affinity of these compounds can be compared and optimum compounds identified using competitive binding studies, which are well known to those of skill in the art.

Techniques for making combinatorial libraries and screening combinatorial libraries to isolate molecules which bind a desired target are well known to those of skill in the art. Representative techniques and methods can be found in but are not limited to U.S. Pat. Nos. 5,084,824, 5,288,514, 5,449,754, 5,506,337, 5,539,083, 5,545,568, 5,556,762, 5,565,324, 5,565,332, 5,573,905, 5,618,825, 5,619,680, 5,627,210, 5,646,285, 5,663,046, 5,670,326, 5,677,195, 5,683,899, 5,688,696, 5,688,997, 5,698,685, 5,712,146, 5,721,099, 5,723,598, 5,741,713, 5,792,431, 5,807,683, 5,807,754, 5,821,130, 5,831,014, 5,834,195, 5,834,318, 5,834,588, 5,840,500, 5,847,150, 5,856,107, 5,856,496, 5,859,190, 5,864,010, 5,874,443, 5,877,214, 5,880,972, 5,886,126, 5,886,127, 5,891,737, 5,916,899, 5,919,955, 5,925,527, 5,939,268, 5,942,387, 5,945,070, 5,948,696, 5,958,702, 5,958,792, 5,962,337, 5,965,719, 5,972,719, 5,976,894, 5,980,704, 5,985,356, 5,999,086, 6,001,579, 6,004,617, 6,008,321, 6,017,768, 6,025,371, 6,030,917, 6,040,193, 6,045,671, 6,045,755, 6,060,596, and 6,061,636.

Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. Nos. 6,017,768 and 5,821,130), amide alcohols (U.S. Pat. No. 5,976,894), hydroxy-amino acid amides (U.S. Pat. No. 5,972,719) carbohydrates (U.S. Pat. No. 5,965,719), 1,4-benzodiazepin-2,5-diones (U.S. Pat. No. 5,962,337), cyclics (U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofurans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5,856,496), imidazol-pyrido-indole and imidazol-pyrido-benzothiophenes U.S. Pat. No. 5,856,107) substituted 2-methylene-2,3-dihydrothiazoles (U.S. Pat. No. 5,847,150), quinolines (U.S. Pat. No. 5,840,500), PNA (U.S. Pat. No. 5,831,014), containing tags (U.S. Pat. No. 5,721,099), polyketides (U.S. Pat. No. 5,712,146), morpholino-subunits (U.S. Pat. Nos. 5,698,685 and 5,506,337), sulfamides (U.S. Pat. No. 5,618,825), and benzodiazopines U.S. Pat. No. 5,288,514);

As used herein combinatorial methods and libraries included traditional screening methods and libraries as well as methods and libraries used in interative processes.

(ii) Computer Assisted Drug Design

The disclosed compositions can be used as targets for any molecular modeling technique to identify either the structure of the disclosed compositions or to identify potential or actual molecules, such as small molecules, which interact in a desired way with the disclosed compositions. The nucleic acids, peptides, and related molecules disclosed herein, such as DHR96 or variants or fragments thereof, can be used as targets in any molecular modeling program or approach.

It is understood that when using the disclosed compositions in modeling techniques, molecules, such as macromolecular molecules, will be identified that have particular desired properties such as inhibition or stimulation or the target molecule's function. The molecules identified and isolated when using the disclosed compositions, such as, DHR96 or variants or fragments thereof, are also disclosed. Thus, the products produced using the molecular modeling approaches that involve the disclosed compositions, such as, DHR96 or variants or fragments thereof, are also considered herein disclosed.

Thus, one way to isolate molecules that bind a molecule of choice is through rational design. This is achieved through structural information and computer modeling. Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

Examples of molecular modeling systems are the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen, et al., 1988 Acta Pharmaceutica Fennica 97, 159-166; Ripka, New Scientist 54-57 (Jun. 16, 1988); McKinaly and Rossmann, 1989 Annu. Rev. Pharmacol. Toxiciol. 29, 111-122; Perry and Davies, OSAR: Ouantitative Structure-Activity Relationships in Drug Design pp. 189-193 (Alan R. Liss, Inc. 1989); Lewis and Dean, 1989 Proc. R. Soc. Loud. 236, 125-140 and 141-162; and, with respect to a model enzyme for nucleic acid components, Askew, et al., 1989 J. Am. Chem. Soc. 111, 1082-1090. Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. Although these are primarily designed for application to drugs specific to particular proteins, they can be adapted to design of molecules specifically interacting with specific regions of DNA or RNA, once that region is identified.

Although described above with reference to design and generation of compounds which could alter binding, one could also screen libraries of known compounds, including natural products or synthetic chemicals, and biologically active materials, including proteins, for compounds which alter substrate binding or enzymatic activity.

### (5) Insects that can be Targeted

Arthropods include Crustacea, which are things like prawns, crabs and woodlice; Myriapoda, which are centipedes, millipedes and such; Chelicerata (Arachnida), which are spiders, scorpions and harvestmen etc., and Uniramia (Insecta), which are things like beetles, bees and flies.

Insects are found in the phylum Arthorpoda, Subphylum Insecta (also often called a class), Class Hexapoda, and Subclasses Apterygota, Exopterygota, and Endopterygota. The Apterygota includes the orders Protura, Collembola (Springtails), Thysanura (Silverfish), Diplura (Two Pronged Bristle-tails). The Exopterygota includes the orders Ephemeroptera (Mayflies), Odonata (Dragonflies), Plecoptera (Stoneflies), Grylloblatodea, Orthoptera, Phasmida (Stick-Insects), Dermaptera (Earwigs), Embioptera (Web Spinners), Dictyoptera (Cockroaches and Mantids), Isoptera (Termites), Zoraptera, Psocoptera (Bark and Book Lice), Mallophaga (Biting Lice), Siphunculata (Sucking Lice), Hemiptera (True Bugs) Thysanoptera, The Endopterygota includes the orders Neuropter (Lacewings), Coleoptera (Beetles), Strepsiptera (Stylops), Mecoptera (Scorpionflies), Siphonaptera (Fleas), Diptera (True Flies which are unusual in that they only have one pair of functional wings. The other pair is reduced to a pair of knoblike organs, called halteres, which play a part in stabilizing these insects during flight. True flies include house flies and bluebottles, mosquitoes, horseflies, midges, and antler-headed flies), Lepidoptera (Butterflies and Moths), Trichoptera (Caddis Flies), and Hymenoptera (Ants Bees and Wasps).

### (6) Exemplary Pesticides that can be Used in Combination

The disclosed compositions, such as DHR96 inhibitors can be combined with any pesticide or class of pesticides. For example, the DHR96 inhibitors can be combined with a pesticide that invokes the xenobiotic pathway. The DHR96 inhibitors can also be combined with any pesticide that effects the expression of a gene in the following four familes, cytochrome P450s, carboxylesterases, glutathione S-transferases, and UDP-glucuronosyltransferases When it is unknown which xenobiotic genes are affected by the pesticide, this can be determined by observing whether the pesticide turns on one or more genes that are in the xenobiotic pathway, by for example, microarray technology, or any other technology that determines gene expression, such as RT-PCR. In certain embodiments, when a particular gene product is specifically overexpressed in a resistant line of insects, that gene product can be considered a xenobiotic gene. Other examples, such as cuticle proteins and a serum carrier protein, were seen in the microarray experiments as well. In other embodiments any encoded protein that confers resistance to a toxic compound can be considered a xenobiotic compound.

There are many different pesticides that are relatively common chemicals, such as arsenicals, petroleum oils, nicotine, pyrethrum, rotenone, sulfur, hydrogen cyanide gas, and cryolite. However, most pesticides are non-natural chemically synthesized compounds. For example, there are different classes and subclasses of pesticides, such as organochlorines, examples of which are diphenyl aliphatics, hexchlorocyclohexane (HCH) or benzenehexachloride (BHC), Cyclodienes, Polychloroterpenes, organophosphates (OPs) examples of whch are esters of phosphorus, organosulfers, carbamates, formamidines, dinitrophenols, oganotins, pyrethroids, nicotinoids (also known as nitro-quanidines, neonicotinyls, neonicotinoids, chloronicotines, or chloronicotinyls), spinosyns, fiproles (or Phenylpyrazoles), pyrroles, pyrazoles, pyridazinones, quinazolines, benzoylureas, botanicals, (natural insecticides), synergists or activators, antibiotics, fumigants, insect repellants, and inorganics.

Another way of classifying insecticides is by their mode of action, for example, sodium and/or potassium channel inhibitors, buerotoxins, GABA (gamma-aminobutyric acid) receptor modulators, such as inhibitors and activators, cholinesterase (ChE) inhibitors, aliesterase inhibitors, monoamine oxidase inhibitors, oxidative phosphorylation couplers or uncouplers, adenosine triphosphate (ATP) formation inhibitors, dinitrophenol uncoupling inhibitors, axionic poisons, inhibition of postsynaptic nicotinergic acetylcholine receptors, inhibiting of binding of acetylcholine in nicotinic acetylcholine receptors at the postsynaptic cell, inhibition of gamma-aminobutyric acid-(GABA) regulated chloride channels in neurons, inhibitors of mitochondrial electron transport at the NADH-COQ reductase site, general inhibitors of mitochondrial electron transport at Site 1, insect growth regulators (IGR, inhibitors of various life cycles and stages in the insect), chitin synthesis inhibitors, inhibitors of exoskeleton development, respiratory enzyme inhibitors, inhibitors of the interaction between NAD+ and coenzyme Q, inhibitors of molting, inhibitors of the biosynthesis or metabolism of ecdysone, synergists, such as inhibitors of cytochrome P450 dependent polysubstrate monooxygenases (PSMOs), and narcotics, calcium channel inhibitors, and repellants.

Examples of organochlorines are (chlorinated hydrocarbons, chlorinated organics, chlorinated insecticides, and chlorinated synthetics) Diphenyl Aliphatics, such as DDT, DDD, dicofol, ethylan, chlorobenzilate, and methoxychlor, Hexchlorocyclohexanes (HCH) or benzenehexachloride (BHC), which are typically gamma isomers, such as lindane, Cyclodienes, such as chlordane, aldrin and dieldrin, heptachlor, endrin, mirex, endosulfan, and chlordecone (Kepone®), and Polychloroterpenes, such as toxaphene and strobane.

Examples of organophosphates (OPs) examples of which are esters of phosphorus, (also called organic phosphates, phosphorus insecticides, nerve gas relatives, and phosphoric acid esters) derived from phosphorus acids, such as sarin, soman, and tabun, subclasses included phosphates, phosphates, phosphorothioates, phosphorodithioates, phosphorothiolates and phosphoramidates. There are also aliphatic, phenyl, and heterocyclic derivatives. The aliphatics include TEPP, malathion, trichlorfon (Dylox®), monocrotophos (Azodrin®), dimethoate (Cygon®), oxydemetonmethyl Ieta Systox®), dimethoate (Cygon®), dicrotophos (Bidrin®), disulfoton (Di-Syston®), dichlorvos (Vapona®), mevinphos (Phosdrin®), methamidophos (Monitor®), and acephate (Orthene®). The Phenyl derivatives parathion (ethyl parathion), methyl parathion, profenofos (Curacron®), sulprofos (Bolstar®), isofenphos (Oftanol®, Pryfon®), fenitrothion (Sumithion®), fenthion (Dasanit®), famphur (Cyflee® and Warbex®). The Heterocyclic derivatives include diazinon, azinphos-methyl (Guthion®), azinphos-ethyl (Acifon®, Gusathion®), chlorpyrifos (Dursban®, Lorsban®; Lock-On®), methidathion (Supracide®), phosmet (Imidan®), isazophos (Brace®., Triumph®), and chlorpyrifos-methyl (Reldan®).

Examples of organosulfers typically contain two phenyl rings, resembling DDT, with sulfur in place of carbon as the central atom, and include tetradifon (Tedion®), propargite (Omite®, Comite®), and ovex (Ovotran®).

Examples of carbamates are derivatives of carbamic acid and include carbaryl (Sevin®), methomyl (Lannate®), carbofuran (Furadan®), aldicarb (Temik®), oxamyl (Vydate®), thiodicarb (Larvin®), methiocarb (Mesurol®), propoxur (Baygon®), bendiocarb (Ficam®), carbosulfan (Advantage®), aldoxycarb (Standak®), promecarb (Carbamult®), and fenoxycarb (Logic®, Torus®).

Examples of formamidines include chlordimeform (Galecron®&, Fundal®), forinetanate (Carzol®), and amitraz (Mitac®, Ovasyn®.

Examples of dinitrophenols include binapacryl (Norocide®) and dinocap (Karathane®).

Examples of oganotins include cyhexatin (Plictran®) and Fenbutatin-oxide (Vendex®).

Examples of pyrethroids natural pyrethrum and synthetic pyrethroids including allethrin (Pynamin®), tetramethrin (Neo-Pynamin®) (1965), resmethrin (Synthrin®), bioresmethrin, Bioallethrin®, phonothrin (Sumithrin®), fenvalerate (Pydrin®, Tribute®, & Belhmark®), permethrin (Ambush®, Astro®, Dragnet®, Flee®, Pounce®, Prelude®, Talcord® & Torpedo®), bifenthrin (Capture®, Talstar®), lambda-cyhalothrin (Demand®, Karate®, Scimitar® & Warrior®), cypermethrin (Ammo®, Barricade®, Cymbush®, Cynoff® & Ripcord®&), cyfluthrin (Baythroid®, Countdown®, Cylense®, Laser® & Tempo®), deltamethrin (Decis®) esfenvalerate (Asana®, Hallmark®), fenpropathrin (Danitol®), flucytbrinate (Cybolt®, Payoff®), fluvalinate (Mavrik®, Spur®), prallethrin (Etoc®), tau-fluvalinate (Mavrik®) tefluthrin (Evict®, Fireban®, Force® & Raze®), tralomethrin (Scout X-TRA®, Tralex®), and zeta-cypermethrin (Mustang® & Fury®), acrinathrin (Rufast®), and imiprothrin (Pralle®.

Examples of nicotinoids (also known as nitro-quanidines, neonicotinyls, neonicotinoids, chloronicotines, or chloronicotinyls) including Imidacloprid (Admire®, Confidor®, Gaucho®, Merit®&, Premier®, Premise® and Provado®), acetamiprid (Mospilan®), thiamethoxam (Actara®, Platinum®), and nitenpyram (Bestguard®).

Examples of spinosyns include (Success®, Tracer Naturalyte®).

Examples of fiproles (or Phenylpyrazoles) include Fipronil ((Regent®, Icon®, Frontline®).

Examples of pyrroles include Chlorfenapyr ((Alert®, Pirate®.

Examples of pyrazoles include tebufenpyrad (Pyranica®, Masai®) and fenpyroximate (Acaban®, Dynamite®).

Examples of pyridazinones include Pyridaben ((Nexter®, Sannite®).

Examples of quinazolines fenazaquin ((Matador®).

Examples of benzoylureas include triflumuron (Alsystint®), chlorfluazuron (Atabron®, Helix®), followed by teflubenzuron Nomolt® &, Dart®), hexaflumuron (Trueno®, Consult®), flufenoxuron (Cascade®), flucycloxuron (Andalin®), flurazuron, novaluron, diafenthiuron, Lufenuron (Axor®), and diflubenzuron ((Dimilin®, Adept®, Micromite®).

Examples of botanicals, (natural insecticides) include sulfur, tobacco, pyrethrum, derris, hellebore, quassia, camphor, and turpentine, and Pyretrum, alkaloids, such as nicotine, caffeine (coffee, tea), quinine (cinchona bark), morphine (opium poppy), cocaine (coca leaves), ricinine (a poison in castor oil beans), strychnine (Strychnos nux vomica), coniine (spotted hemlock, the poison used by Socrates), and LSD (a hallucigen from the ergot fungus attacking grain), rotenone, Limonene or d-Limonene, neem, Azadirachtin (Azatin® is marketed as an insect growth regulator, and Align® and Nemix®).

Examples of synergists or activators are not insecticides per se, but rather enhance the activity of insecticides having a primary insecticidal effect. Examples include, piperonyl butoxide, and contain the methylenedioxyphenyl moiety (found in sesame seed oil (sesainin)).

Examples of antibiotics include avermectins, Abamectin, Clinch®, Emamectin benzoate (Proclaim®, Denim®).

Examples of fumigants typically contain one or more halogens, such as methyl bromide (Aspelin and Grube 1998), ethylene dichloride, hydrogen cyanide, sulfuryl fluoride (Vikane®)), Vapam®, Telone® II, D-D®; chlorothene, ethylene oxide, napthalene crystals, paradichlorobenzene crystals, Phosphine gas (PH₃) produced by aluminum or magnesium phosphide pellets.

Examples of insect repellants include dimethyl phthalate, Indalone®, Rutgers 612®, dibutyl phthalate, various MGK® repellents, benzyl benzoate, the military clothing repellent (N-butyl acetanilide), dimethyl carbate (Dimelone®) and diethyl toluamide (DEET, Delphene®).

Examples of inorganics include sulfur, mercury, boron, thallium, arsenic, antimony, selenium, and fluoride, arsenicals, including copper arsenate, Paris green, lead arsenate, and calcium arsenate, inorganic fluorides such as sodium fluoride, barium fluosilicate, sodium silicofluoride, and cryolite (Kryocide®), Boric acid, Sodium borate (disodium octaborate tetrahydrate) (Tim-Bor®, Bora-Care®), silica gels or silica aerogels, such as Dri-Die®, Drianone®, and Silikil Microcel®.

Other compounds not easily categorized include cyromazine (Larvadex®, Trigard®), a triazine, pyriproxyfen (Knack®, Esteem®, Archer®), insect growth inhibitors such as buprofezin (Applaud®) and thiadiazines, tetrazines, such as clofentezine (Apollo®, Acaristop®), Enzone®, sodium tetrathiocarbonate, and Clandosan®.

Also used are Veratrum Alkaloids, such as sabadilla, veratridine, and cevadine.

Also used are ryanoids, such as ryanodine, 10-(O-methyl)-ryanodine, 9,21-dehydroryanodine, ryanodol, and 9,21-dehydroryanodine.

Also used are octopamines mimics, such as Amitraz® and chlordimeform.

Also included are respiration inhibitors, such as fenazaquin, pyridaben, amidinohydrazone, hydramethylnon and the perfluorooctanesulfonamide, and sulfluramid.

Also included are juvenile hormone mimics, such a juvenile hormone III, methoprene, and fenoxycarb.

Also included are toxins produced by Bacillus thuringiensis, such as Dipel®, Javelin®, Agree®.

### C. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular DHR96 or variants or fragments thereof is disclosed and discussed and a number of modifications that can be made to a number of molecules including the DHR96 or variants or fragments thereof are discussed, specifically contemplated is each and every combination and permutation of DHR96 or variants or fragments thereof and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C—F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

1. Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general; variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2:482 (1981), by the homology alignment algorithm of Needleman and Wunsch, J. Mol. Biol. 48:443 (1970), by the search for similarity method of Pearson and Lipman, Proc. Natl. Acad. Sci. U.S.A. 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

2. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 100 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

3. Nucleic Acids

There are a variety of molecules disclosed herein that are nucleic acid based, including for example the nucleic acids that encode, for example DHR96 or variants or fragments thereof, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantagous that the antisense molecule be made up of nucleotide analogs that reduce the degradation of the antisense molecule in the cellular environment.

a) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to the base moiety would include natural and synthetic modifications of A, C, G, and T/U as well as different purine or pyrimidine bases, such as uracil-5-yl (.psi.), hypoxanthin-9-yl (I), and 2-aminoadenin-9-yl. A modified base includes but is not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and

2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Additional base modifications can be found for example in U.S. Pat. No. 3,687,808, Englisch et al., Angewandte Chemie, International Edition, 1991, 30, 613, and Sanghvi, Y. S., Chapter 15, Antisense Research and Applications, pages 289-302, Crooke, S. T. and Lebleu, B. ed., CRC Press, 1993. Certain nucleotide analogs, such as 5-substituted pyrimidines, 6-azapyrimidines and N-2, N-6 and O-6 substituted purines, including 2-aminopropyladenine, 5-propynyluracil and 5-propynylcytosine. 5-methylcytosine can increase the stability of duplex formation. Often time base modifications can be combined with for example a sugar modification, such as 2′-O-methoxyethyl, to achieve unique properties such as increased duplex stability. There are numerous such United States patents as U.S. Pat. Nos. 4,845,205; 5,130,302; 5,134,066, 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, which detail and describe a range of base modifications. Each of these patents is herein incorporated by reference.

Nucleotide analogs can also include modifications of the sugar moiety. Modifications to the sugar moiety would include natural modifications of the ribose and deoxy ribose as well as synthetic modifications. Sugar modifications include but are not limited to the following modifications at the 2′ position: OH; F; O-, S-, or N-alkyl; O-, S-, or N-alkenyl; O—, S- or N-alkynyl; or O-alkyl-O-alkyl, wherein the alkyl, alkenyl and alkynyl may be substituted or unsubstituted C₁ to C₁₀, alkyl or C₂ to C₁₀ alkenyl and alkynyl. 2′ sugar modifications also include but are not limited to —O[(CH₂)ₙO]ₘ CH₃, —O(CH₂), OCH₃, —O(CH₂), NH₂, —O(CH₂)ₙ CH₃, —O(CH₂)ₙ—ONH₂, and —O(CH₂)ₙON[(CH₂)ₙCH₃)]₂, where n and m are from 1 to about 10.

Other modifications at the 2′ position include but are not limited to: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars would also include those that contain modifications at the bridging ring oxygen, such as CH₂ and S, Nucleotide sugar analogs may also have sugar mimetics such as cyclobutyl moieties in place of the pentofuranosyl sugar. There are numerous United States patents that teach the preparation of such modified sugar structures such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

Nucleotide analogs can also be modified at the phosphate moiety. Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

It is understood that nucleotide analogs need only contain a single modification, but may also contain multiple modifications within one of the moieties or between different moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

Nucleotide substitutes are nucleotides or nucleotide analogs that have had the phosphate moiety and/or sugar moieties replaced. Nucleotide substitutes do not contain a standard phosphorus atom. Substitutes for the phosphate can be for example, short chain alkyl or cycloalkyl internucleoside linkages, mixed heteroatom and alkyl or cycloalkyl internucleoside linkages, or one or more short chain heteroatornic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference. (See also Nielsen et al., Science, 1991, 254, 1497-1500).

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety (Letsinger et al., Proc. Natl. Acad. Sci. USA, 1989,

86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937. Numerous United States patents teach the preparation of such conjugates and include, but are not limited to U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

b) Sequences

There are a variety of sequences related to the DHR96 gene, and these sequences and others are herein incorporated by reference in their entireties as well as for individual subsequences contained therein.

One particular sequence set forth in SEQ ID NO:7 and having Genbank accession number NM_{—}079769 is used herein, as an example, to exemplify the disclosed compositions and methods. It is understood that the description related to this sequence is applicable to any sequence related to DHR96 or any other sequences disclosed herein, unless specifically indicated otherwise. Those of skill in the art understand how to resolve sequence discrepancies and differences and to adjust the compositions and methods relating to a particular sequence to other related sequences (i.e. sequences of DHR96 or variants or fragments thereof). Primers and/or probes can be designed for any DHR96 sequence given the information disclosed herein and known in the art.

c) Primers and Probes

Disclosed are compositions including primers and probes, which are capable of interacting with the genes disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the nucleic acid or region of the nucleic acid or they hybridize with the complement of the nucleic acid or complement of a region of the nucleic acid.

4. Delivery of the Compositions to Cells

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 8.15-818, (1991) Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

a) Nucleic Acid Based Delivery Systems

The term “transgene” is used herein to describe genetic material which is artificially inserted into the genome of an invertebrate cell. The transgene encodes a product that, when expressed in embryos, gives rise to a specific phenotype. A transgene can encode a transcription factor or mimetic thereof having the desired result. A recombinant DNA molecule or vector containing a heterologous protein gene expression unit can be used to transfect invertebrate cells (U.S. Pat. Nos. 4,670,388 and 5,550,043, herein incorporated by reference in their entirety.) A gene expression unit can contain a DNA coding sequence for a selected protein or for a derivative thereof. Such derivatives can be obtained by manipulation of the gene sequence using traditional genetic engineering techniques, e.g., mutagenesis, restriction endonuclease treatment, ligation of other gene sequences including synthetic sequences and the like (T. Maniatis et al, Molecular Cloning, A Laboratory Manual., Cold Spring Harbor Laboratory, Cold Spring Harbor, N.Y. (1982).

Expression of the transgene can be targeted to occur in a non-adult stage of the animal, the transgene can be stably integrated into the genome of the animal in a manner such that its expression is controlled both spatially and temporally to the desired cell type and the correct developmental stage, i.e. to expression in embryonic neuroblasts. Specifically, the subject transgene can stably integrated into the genome of the animal under the control of a promoter that provides for expression. The transgene may be under the control of any convenient promoter that provides for this requisite spatial and temporal expression pattern, where the promoter can be endogenous or exogenous. A suitable promoter is the promoter located in the Drosophila melanogaster genome at position 86E1-3.

Another suitable promoter of the Drosophila origin includes the Drosophila metallothionein promoter (Lastowski-Perry et al, J. Biol. Chem., 260:1527, 1985). This inducible promoter directs high-level transcription of the gene in the presence of metals, e.g., CuSO4. Use of the Drosophila metallothionein promoter results in the expression system of the invention retaining full regulation even at very high copy number. This is in direct contrast to the use of the mammalian metallothionein promoter in mammalian cells in which the regulatory effect of the metal is diminished as copy number increases. In the Drosophila expression system, this retained inducibility effect increases expression of the gene product in the Drosophila cell at high copy number.

The Drosophila actin 5C gene promoter (B. J. Bond et al, Mol. Cell. Biol., 6: 2080, 1986) is also a desirable promoter sequence. The actin 5C promoter is a constitutive promoter and does not require addition of metal. Therefore, it is better-suited for use in a large scale production system, like a perfusion system, than is the Drosophila metallothionein promoter. An additional advantage is that the absence of a high concentration of copper in the media maintains the cells in a healthier state for longer periods of time.

Examples of other known Drosophila promoters include, e.g., the inducible heatshock (Hsp70) and COPIA LTR promoters. The SV40 early promoter gives lower levels of expression than the Drosophila metallothionein promoter.

The transgene may be integrated into the fly genome in a manner that provides for direct or indirect expression activation by the promoter, i.e. in a manner that provides for either cis or trans activation of gene expression by the promoter. In other words, expression of the transgene may be mediated directly by the promoter, or through one or more transactivating agents. Where the transgene is under direct control of the promoter, i.e. the promoter regulates expression of the transgene in a cis fashion, the transgene is stably integrated into the genome of the fly at a site sufficiently proximal to the promoter and in frame with the promoter such that cis regulation by the promoter occurs.

In other embodiments where expression of the transgene is indirectly mediated by the endogenous promoter, the promoter controls expression of the transgene through one or more transactivating agents, usually one transactivating agent, i.e. an agent whose expression is directly controlled by the promoter and which binds to the region of the transgene in a manner sufficient to turn on expression of the transgene. Any convenient transactivator may be employed. The GAL4 transactivator system an example of such a system.

The GAL4 encoding sequence can be stably integrated into the genome of the aniimal in a manner such that it is operatively linked to the endogenous promoter that provides expression in the appropriate location. The GAL4 system consists of the yeast transcriptional activator GAL4 and its target the upstream activating sequence (UAS) located within the P-element. Initially, GAL4 and UAS are in separate lines. The UAS is mobilized to generate new UAS insertion lines which remain silent until a source of GAL4 is made available. Under the control of a promoter, the expression of GAL4 is directed in a particular pattern. Specialized promoters can be used to drive expression of GAL4 in tissue and cell specific manners. The GAL4 containing line is then crossed to the UAS containing line. The UAS in the presence of GAL4 directs the expression of any genes adjacent to its insertion site. When the insertion site is located upstream from the coding region over- or ectopic expression occurs.

Flies of line 31-1 (also referred to as 1822), as disclosed in Brand & Perrimon, Development (1993) 118: 401-415 express GAL4 in this manner, and are known to those of skill in the art. The transgene is stably integrated into a different location of the genome, generally a random location in the genome, where the transgene is operatively linked to an upstream activator sequence, i.e. UAS sequence, to which GAL4 binds and turns on expression of the transgene. Transgenic flies having a UAS: GAL4 transactivation system are known to those of skill in the art and are described in Brand & Perrimon, Development (1993) 118: 401-415; and Phelps & Brand, Methods (April 1998) 14:367-379.

A desirable gene expression unit or expression vector for the protein of interest cal also be constructed by fusing the protein coding sequence to a desirable signal sequence. The signal sequence functions to direct secretion of the protein from the host cell. Such a signal sequence may be derived from the sequence of tissue plasminogen activator (tPA). Other available signal sequences include, e.g., those derived from Herpes Simplex virus gene HSV-I gD (Lasky et al, Science, 233:209-212 1986).

The DNA coding sequence can also be followed by a polyadenylation (poly A) region, such as an SV40 early poly A region. The poly A region which functions in the polyadenylation of RNA transcripts appears to play a role in stabilizing transcription. A similar poly A region can be derived from a variety of genes in which it is naturally present. This region can also be moditied to alter its sequence provided that polyadenylation and transcript stabilization functions are not significantly adversely affected.

The recombinant DNA molecule may also carry a genetic selection marker, as well as the protein gene functions. The selection marker can be any gene or genes which cause a readily detectable phenotypic change in a transfected host cell. Such phenotypic change can be, for example, drug resistance, such as the gene for hygromycin B resistance (i.e., hygromycin B phosphotransferase).

Alternatively, a selection system using the drug methotrexate, and prokaryotic dihydrofolate reductase (DHFR) gene, can be used with Invertebrate cells. The endogenous eukaryotic DHFR of the cells is inhibited by methotrexate. Therefore, by transfecting the cells with a plasmid containing the prokaryotic DHFR which is insensitive to methotrexate and selecting with methotrexate, only cells transfected with and expressing the prokaryotic DHFR will survive. Unlike methotrexate, selection of transformed mammalian and bacterial cells, in the Drosophila system, methotrexate can be used to initially high-copy number transfectants. Only cells which have incorporated the protective prokaryotic DHFR gene will survive. Concomitantly, these cells have the gene expression unit of interest.

The subject transgenic flies can be prepared using any convenient protocol that provides for stable integration of the transgene into the fly genome in a manner sufficient to provide for the requisite spatial and temporal expression of the transgene, i.e. in embryonic neuroblasts. A number of different strategies can be employed to obtain the integration of the transgene with the requisite expression pattern. Generally, methods of producing the subject transgenic flies involve stable integration of the transgene into the fly genome. Stable integration is achieved by first introducing the transgene into a cell or cells of the fly, e.g. a fly embryo. The transgene is generally present on a suitable vector, such as a plasmid. Transgene introduction may be accomplished using any convenient protocol, where suitable protocols include: electroporation, microinjection, vesicle delivery, e.g. liposome delivery vehicles, and the like. Following introduction of the transgene into the cell(s), the transgene is stably integrated into the genome of the cell. Stable integration may be either site specific or random, but is generally random.

Where integration is random, the transgene is typically integrated with the use of transposase. In such embodiments, the transgene can be introduced into the cell(s) within a vector that includes the requisite P element, terminal 31 base pair inverted repeats. Where the cell into which the transgene is to be integrated does not comprise an endogenous transposase, a vector encoding a transposase can also be introduced into the cell, e.g. a helper plasmid comprising a transposase gene, such as pTURBO (Steller & Pirrotta, Mol. Cell. Biol. 6:1640-1649, 1986). Methods of random integration of transgenes into the genome of a target Drosophila melanogaster cell(s) are disclosed in U.S. Pat. No. 4,670,388, the disclosure of which is herein incorporated by reference.

Transcription and expression of the heterologous protein coding sequences can be monitored. For example, Southern blot analysis can be used to determine copy number of the gp120 gene. Northern blot analysis provides information regarding the size of the transcribed gene sequence. The level of transcription can also be quantitated. Expression of the selected protein in the recombinant cells can be further verified through Western blot analysis, for example.

In those embodiments in which the transgene is stably integrated in a random fashion into the fly genome, means are also provided for selectively expressing the transgene at the appropriate time during development of the fly. In other words, means are provided for obtaining targeted expression of the transgene. To obtain the desired targeted expression of the randomly integrated transgene, integration of particular promoter upstream of the transgene, as a single unit in the P element vector may be employed. Alternatively, a transactivator that mediates expression of the transgene may be employed. Of particular interest is the GAIA system described in Brand & Perrimon, Development (1993) 118: 401-415; and Phelps & Brand, Methods (April 1998) 14:367-379.

In one embodiment, the subject transgenic flies are produced by: (1) generating two separate lines of transgenic flies: (a) a first line that expresses GAL4; and (b) a second line in which the transgene is stably integrated into the cell genome and is fused to a UAS domain; (2) crossing the two lines; and (3) screening the progeny for the desired phenotype, i.e. adult onset neurodegeneration. Each of the above steps are well known to those of skill in the art (Brand & Perrimon, Development 118: 401-415, 1993; and Phelps & Brand, Methods 14:367-379, April 1998.)

b) Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed compositions or vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci. USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem. 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other speciifc cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

c) In Vivo/Ex Vivo

As described above, the compositions can be administered in a pharmaceutically acceptable carrier and can be delivered to the subject=s cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

5. Peptides

a) Protein Variants

As discussed herein there are numerous variants of the DHR96 protein that are known and herein contemplated. In addition, to the known functional DHR96 strain variants there are derivatives of the DHR96 protein which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| Amino Acid | Abbreviations |
|---|---|
| alanine | AlaA |
| allosoleucine | AIle |
| arginine | ArgR |
| asparagine | AsnN |
| aspartic acid | AspD |
| cysteine | CysC |
| glutamic acid | GluE |
| glutamine | GlnK |
| glycine | GlyG |
| histidine | HisH |
| isolelucine | IleI |
| leucine | LeuL |
| lysine | LysK |
| phenylalanine | PheF |
| proline | ProP |
| pyroglutamic acidp | Glu |
| serine | SerS |
| threonine | ThrT |
| tyrosine | TyrY |
| tryptophan | TrpW |
| valine | ValV |

TABLE 2
Amino Acid Substitutions

Original Residue Exemplary Conservative Substitutions,
others are known in the art.
Alaser
Arglys, gln
Asngln; his
Aspglu
Cysser
Glnasn, lys
Gluasp
Glypro
Hisasn; gln
Ileleu; val
Leuile; val
Lysarg; gln;
MetLeu; ile
Phemet; leu; tyr
Serthr
Thrser
Trptyr
Tyrtrp; phe
Valile; leu

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SEQ ID NO:8 sets forth a particular sequence of DHR96 cDNA and SEQ ID NO:7 sets forth a particular sequence of a DHR96 protein. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2:482 (1981), by the homology alignment algorithm of Needleman and Wunsch, J. Mol. Biol. 48:443 (1970), by the search for similarity method of Pearson and Lipman, Proc. Natl. Acad. Sci. U.S.A. 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. For example, one of the many nucleic acid sequences that can encode the protein sequence set forth in SEQ ID NO:7 is set forth in SEQ ID NO:8. It is also understood that while no amino acid sequence indicates what particular DNA sequence encodes that protein within an organism, where particular variants of a disclosed protein are disclosed herein, the known nucleic acid sequence that encodes that protein in the particular organism from which that protein arises is also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); lbba, Biotechnology & Genetic Enginerring Reviews 13:197-216 (1995), Cahill et al., TIBS, 14(10):400-403 (1989); Benner, TIB Tech, 12:158-163 (1994); Tbba and Hennecke, Bio/technology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂—, —CH═CH—(cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CHH₂—S); Hann J. Chem. Soc Perkin Trans. 1307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

6. Pharmaceutical Carriers/Delivery of Pharamceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconijugate Chem., 2:447-451, (1991); Bagshawe, K. D., Br. J. Cancer 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research 49:6214-6220, (1989); and Litzinger and Huang, Biochimica et Biophysica Acta 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

Effective dosages and schedules for administering the compositions may be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

7. Chips and Micro Arrays

Disclosed are chips where at least one address is the sequences or part of the sequences set forth in any of the nucleic acid sequences disclosed herein. Also disclosed are chips where at least one address is the sequences or portion of sequences set forth in any of the peptide sequences disclosed herein.

Also disclosed are chips where at least one address is a variant of the sequences or part of the sequences set forth in any of the nucleic acid sequences disclosed herein. Also disclosed are chips where at least one address is a variant of the sequences or portion of sequences set forth in any of the peptide sequences disclosed herein.

8. Computer Readable Mediums

It is understood that the disclosed nucleic acids and proteins can be represented as a sequence consisting of the nucleotides of amino acids. There are a variety of ways to display these sequences, for example the nucleotide guanosine can be represented by G or g. Likewise the amino acid valine can be represented by Val or V. Those of skill in the art understand how to display and express any nucleic acid or protein sequence in any of the variety of ways that exist, each of which is considered herein disclosed. Specifically contemplated herein is the display of these sequences on computer readable mediums, such as, commercially available floppy disks, tapes, chips, hard drives, compact disks, and video disks, or other computer readable mediums. Also disclosed are the binary code representations of the disclosed sequences. Those of skill in the art understand what computer readable mediums. Thus, computer readable mediums on which the nucleic acids or protein sequences are recorded, stored, or saved.

Disclosed are computer readable mediums comprising the sequences and information regarding the sequences set forth herein. Also disclosed are computer readable mediums comprising the sequences and information regarding the sequences set forth herein wherein the sequences do not include SEQ ID Nos: 37, 38, 39, 40, 41, and 42.

9. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended.

### D. METHODS OF MAKING THE COMPOSITIONS

The compositions disclosed herein and the compositions necessary to perform the disclosed methods can be made using any method known to those of skill in the art for that particular reagent or compound unless otherwise specifically noted.

1. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold. Spring Harbor Laboratory Press, Cold Spring Harbor, N.Y., 1989) Chapters 5, 6) to purely synthetic methods, for example, by the cyanoethyl phosphoramidite method using a Milligen or Beckman System lPlus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzylnol., 65:610-620 (1980), (phosphotriester method). Protein nucleic acid molecules can be made using known methods such as those described by Nielsen et al., Bioconjug. Chem. 5:3-7 (1994).

2. Peptide Synthesis

One method of producing the disclosed proteins, such as SEQ ID NO:23, is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides may be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

3. Processes for Making the Compositions

Disclosed are processes for making the compositions as well as making the intermediates leading to the compositions. For example, disclosed are nucleic acids and proteins in SEQ ID NOs:1-60. There are a variety of methods that can be used for making these compositions, such as synthetic chemical methods and standard molecular biology methods. It is understood that the methods of making these and the other disclosed compositions are specifically disclosed.

Disclosed are nucleic acid molecules produced by the process comprising linking in an operative way a nucleic acid comprising the sequence set forth herein and a sequence controlling the expression of the nucleic acid.

Also disclosed are nucleic acid molecules produced by the process comprising linking in an operative way a nucleic acid molecule comprising a sequence having 80% identity to a sequence set forth in herein, and a sequence controlling the expression of the nucleic acid.

Disclosed are nucleic acid molecules produced by the process comprising linking in an operative way a nucleic acid molecule comprising a sequence that hybridizes under stringent hybridization conditions to a sequence set forth herein and a sequence controlling the expression of the nucleic acid.

Disclosed are nucleic acid molecules produced by the process comprising linking in an operative way a nucleic acid molecule comprising a sequence encoding a peptide set forth in SEQ ID NO:7 and a sequence controlling an expression of the nucleic acid molecule.

Disclosed are nucleic acid molecules produced by the process comprising linking in an operative way a nucleic acid molecule comprising a sequence encoding a peptide having 80% identity to a peptide set forth in herein and a sequence controlling an expression of the nucleic acid molecule.

Disclosed are nucleic acids produced by the process comprising linking in an operative way a nucleic acid molecule comprising a sequence encoding a peptide having 80% identity to a peptide set forth in herein, wherein any change from the herein are conservative changes and a sequence controlling an expression of the nucleic acid molecule.

Disclosed are cells produced by the process of transforming the cell with any of the disclosed nucleic acids. Disclosed are cells produced by the process of transforming the cell with any of the non-naturally occurring disclosed nucleic acids.

Disclosed are any of the disclosed peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the non-naturally disclosed nucleic acids.

Disclosed are animals and invertebrates produced by the process of transfecting a cell within the animal or invertebrate with any of the nucleic acid molecules disclosed herein. Disclosed are animals or invertebrates produced by the process of transfecting a cell within the animal any of the nucleic acid molecules disclosed herein, wherein the animal is a mammal invertebrate is an insect, such as Drosophila. Also disclosed are animals produced by the process of transfecting a cell within the animal any of the nucleic acid molecules disclosed herein, wherein the mammal is mouse, rat, rabbit, cow, sheep, pig, or primate.

Also disclose are animals produced by the process of adding to the animal any of the cells disclosed herein.

### E. METHODS OF USING THE COMPOSITIONS

1. Methods of Using the Compositions as Research Tools

The disclosed compositions can be used in a variety of ways as research tools. For example, the disclosed compositions, such as molecules disclosed herein can be used to study the interactions between the molecules, and for example, their ligands or other compounds, by for example acting as inhibitors of binding.

The compositions can be used for example as targets in combinatorial chemistry protocols or other screening protocols to isolate molecules that possess desired functional properties related to inhibiting DHR96 activity, for example.

The disclosed compositions can be used as discussed herein as either reagents in micro arrays or as reagents to probe or analyze existing microarrays. The disclosed compositions can be used in any known method for isolating or identifying single nucleotide polymorphisms. The compositions can also be used in any method for determining allelic analysis of for example, DHR96, particularly allelic analysis as it relates to xenobiotic pathway functions. The compositions can also be used in any known method of screening assays, related to chip/micro arrays. The compositions can also be used in any known way of using the computer readable embodiments of the disclosed compositions, for example, to study relatedness or to perform molecular modeling analysis related to the disclosed compositions.

### F. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1 The DHR96 Nuclear Receptor is Required for Xenobiotic Responses in Drosophila

### a) Materials and Methods

(1) Construction of the DHR96 Targeting Fragment

A 7.55 kb DNA fragment that contains a mutated version of the Drosophila melanogaster DHR96 gene was generated by introducing two deletions: (1) deleting sequences harboring the start site (26 bp) and (2) deleting the fourth exon and intron (331 bp) from the wild type sequence. In addition, a recognition site for the restriction enzyme I-Sce I was inserted into the center (cuts between position 3699 and 3700) of the 7.55 kb fragment (see fig. M1). To obtain a genomic clone DNA of the P1 clone 26-95 that harbored the complete DHR96 gene was isolated (provided by BDGP: http://www.fruitfly.org/). The assembly of the 7.55 kb targeting sequence was achieved by fusing three fragments:

(a) Fragment 1 A 1.958 Ab Apa I-Hind III Fragment

This was isolated by cutting P1 26-95 with Hind III and isolating a 6.599 kb Hind m fragment, which then was cut with Apa I and Sgr AI. The 1.958 kb Apa 1—Hind III fragment was cloned into Litmus 38 (New England BioLabs) (cut with Apa I and Hind III).

(b) Fragment 2 A 4.325 kb Fragment

This fragment contains the actual mutations and forms the core of the targeting construct. It was generated by using three pairs of PCR primers (for sequences, see oligos): (1) FAPA96 and R96EX3Sce, (II) F96Int3Sce and R96Int3, (III) F96Ex5Int3 and R96EndHind. The P1 26-95 genomic clone served as a template. Primer pair (I) produced a 1724 bp fragment, primer pair (II) a 993 bp fragment and primer pair (III) a 1650 bp fragment. The 993 bp and the 1650 bp fragments were fused in a PCR reaction using the primers F96Int3Sce and R96EndHind, generating a 2.62 kb fragment. Likewise, the 1724 bp and the 993 bp fragments were fused using the FAPA96 and R96Int3 primers to form a 2.70 kb fragment. In a final step, the 2.70 and the 2.62 kb fragments were fused using the primers FAPA96 and R96EndHind to form the aforementioned 4.325 kb fragment, which was cloned into PCR TOPO 2.1 (Invitrogen).

(c) Fragment 3 A 1.86 kb PCR Fragment

Fragment 3 was generated using the primers F96Xma and R96SpeBgl, with the P1 26-95 clone as a template. The fragment was eluted and cut directly with Xma I and Spe I.

The 1.86 kb PCR fragment was cloned into the PCR Topo 2.1 vector (Invitrogen) containing the 4.325 kb, which was cut with Xma I and Spe I. The resulting clone was cut with Apa I and Spe I and fused to the 1.958 kb fragment, which had been previously isolated from Litmus 38 (New England Biolabs) with Apa I and Spe I. The resulting clone is the 7.55 kb targeting fragment. A sequence printout and annotation of this fragment is included (SEQ ID NO:37).

(2) Construction of the hs-Gal4-DEOR96 Fusion Gene

A fusion of the Gal4 DNA binding domain (amino acids 1 to 147) and the DHR96 hinge region and ligand binding domain (LBD) (amino acids 99 to 723) was generated to create a Gal4-LBD fusion protein. Two PCR fragments were generated: (I) a 475 bp fragment using the primers FGALXB and RGAL96 and a Gal4 containing plasmid as a template. (II) F96BEG and R96/936 generate a 372 bp fragment from pLF20N, which contains the DHR96 cDNA (Fisk and Thummel, 1995). Fragments (I) and (II) possess a 15 bp overlap that was then utilized to fuse them by PCR. The resulting 832 bp fragment was cut with Xba I and Age I and cloned into pLF20N, which had been cut with the same enzymes to remove the DHR96DNA-binding domain. The resulting plasmid is termed pGAL96. To obtain the final transformation vector, the Gal4-DHR96 fusion gene was isolated from pGAL96 with Not I and Nhe I and ligated to pCASPER hs-act cut with Xba I and Not I (SEQ ID NO:38, (see Seq 2 for the sequence of the insert in this vector, encoding the Gal4-LBD fusion).

(3) Construction of the hs-DHR96RNAI Vector

An inverted repeat sequence that corresponds to a part of the coding region for the DHR96 ligand-binding domain (each repeat corresponds to nucleotides 1444-2371 of the DHR96 plasmid pLF20N; Fisk and Thummel, 1995) was generated. The repeats are separated by a unique spacer region of 101 bp that corresponds to nucleotides 2372-2472 of the same DHR96 cDNA. Two primer pairs were used: (I) F96XbaI and R96BspE1 and (II) F96XbaI and R96BspE2. Both fragments were cut with Bsp E1 and ligated. The ligated fragment was purified and cut with Xba I and cloned into Litmus 28 (New England Biolabs) cut with Xba I. After the cloned fragment (1956 bp) was verified by restriction analysis, it was excised with Xba I and inserted into pCasper hs-act cut with Xba I.

(4) Construction of the hs-DHR96 Vector and Fly Transformation

This vector produces wild type DHR96 protein under the control of an hsp70 promoter in a transgenic animal. A full length cDNA was excised from the plasmid pLF20N with the restriction enzymes Not I and NheI and cloned it into pCasper hs-act vector cut with Not I and Xba I. Transformant flies were isolated using standard methods (Rubin G M, Spradling A C. Genetic transformation of Drosophila with transposable element vectors. Science. 1982 October 22; 218(4570):348-53).

(5) Construction of pET24c-DHR96

To generate antibodies, DHR96 antigen was produced from a 1.8 kb EcoRV fragment (597 amino acids), which includes most of the cDNA, but excludes the DNA binding domain. The 1.8 kb Eco RV fragment was isolated from pLF20, a plasmid that contains a full length DHR96 cDNA (pLF20 differs from pLF20N in the following: pLF20 was cut with HindIII, filled in, and religated to create a unique Nhe I site. The new plasmid was termed pLF20N). pET24c (Novagen) was cut with Bam HI and Xho I and blunt ends were generated by fill-in, and subsequently the Eco RV fragment was cloned into this vector. Orientation was tested using restriction analysis. A sequence printout of this clone is included (SEQ ID NO:39Seq. 3).

(6) Construction of pMAL-DHR96

To purify antisera, soluble DHR96 protein was produced by fusing the original antigen to the Maltose-binding protein. To subclone the Eco RV fragment of DHR96 (the original antigen coding section) into pMAL-c2X (New England Biolab), a fragment from pET24c-DHR96 was PCR amplified by using the primer pair F96ANhe and R96AHind. The fragment was cut directly with Nhe I and HindIII and cloned into pMAL-c2X cut with Xba I and Hind”.

(7) Oligonucleotides

Oligonucleotides

| | | |
|---|---|---|
| SEQ ID | F96Xma | 5′-GAGAGATGTGCTTCGTTAAAGCATCAACC |
| NO:40 | | C |
| SEQ ID | R96SpeBgl | 5′-GGACTAGTAGATCTAGAGGATTCTACAAA |
| NO:41 | | TGTCCAGTGTCTCCC |
| SEQ ID | R961nt3 | 5′-CCATTATTATCGCCATAATCGTAAAGG |
| NO:42 | | |
| SEQ ID | R96EX3SCE | 5′-ATTACCCTGTTATCCCTAGCGGGTTACCT |
| NO:43 | | TAATGCGATCATCGCCC |
| SEQ ID | R96endhind | 5′-GGAAAGCTTTTCCTGCTGATCAATAATAC |
| NO:44 | | C |
| SEQ ID | FAPA96 | 5′-TGGGCCCATCACTTGCTTGTAACCGCCGA |
| NO:45 | | AGAAGTGCGCGG |
| SEQ ID | F96TNT3SCE | 5′-CGCTAGGGATAACAGGGTAATAACAGTCC |
| NO:46 | | ACGGTATTAGCCTATAGG |
| SEQ ID | F96EX5Int3 | 5′-CGATTATGGCGATAATAATGGCCAAAGAG |
| NO:47 | | AACATGGGCAACATACGC |
| SEQ ID | FGALXB | 5′-GAAGCAAGCCTCTAGAAAGATGAAGC |
| NO:48 | | |
| SEQ ID | RGAL96 | 5′-CGTGCCGTTCTCCATCGATACAGTCAACT |
| NO:49 | | GTCTTTGACC |
| SEQ ID | R961936 | 5′-GCCTGGATAGTCGATCAAATGCG |
| NO:50 | | |
| SEQ ID | F96BEG | 5′-ATGGAGAACGGCACGGATGC |
| NO:51 | | |
| SEQ ID | F96XBA1 | 5′-TACATTCTAGAGACCAACTACAACGACGA |
| NQ:52 | | GCCCAGTCTGG |
| SEQ ID | R96BspE1 | 5′-CATTCATCCGGACATTAATTATGAACTTG |
| NO:53 | | TTCAGACGCTCC |
| SEQ ID | R96BspE2 | 5′-GGGCATCAACTCCGGAATTAAATGCCCGA |
| NO:54 | | CACGCATCGG |
| SEQ ID | RPAXCRE-AN | 5′-GTCTCACGACGTTTTGAACCCAGAAATCG |
| NO:55 | | AGCTCGCCCGGGG |
| SEQ ID | RPAXCRECO | 5′-CACGAATTCCAAACTGTGTCACGACGTTT |
| NO:56 | | TGAACCC |
| SEQ ID | FPAXFSE-AN | 5′-GAGAGCTAGCATGCCGGCTAGATCTCGAG |
| NO:57 | | ATCGGCCGGCCTAGG |
| SEQ ID | EPAXPOLY | 5′-GAACTGCAGCTCGAGAGCTAGCATGCCGG |
| NO:58 | | C |
| SEQ ID | F96ANhe | 5′-GGAGATATACATATGGCTAGCATGACTGG |
| NO:59 | | TGG |
| SEQ ID | R96AHind | 5′-TGCTCGAAGCTTCGCAGAAGATAATAGTA |
| NO:60 | | GG |

(8) DHR96 Gene Targeting

The 7.55 kb genomic fragment containing a mutated DR96 gene (see above) was inserted into the Drosophila genome as described (Rong Y S, Golic K G. Gene targeting by homologous recombination in Drosophila. Science. 2000 Jun. 16; 288(5473):2013-8). w; [hsp70-FLP]4 [hsp70 I Sce I]2b Sco/S2 CyO females were crossed to w; [<(96TG GFP+>w+] males that carried the targeting fragment on the second chromosome. Larvae were heat shocked during the third larval instar to trigger targeting events in the germline of females. [hsp70-FLP]4 [hsp70 I Sce I]2b Sco/[<(96TG GFP+>w+] females were then collected and crossed them to w; Ser1/TM6B, Tb males. 918 vials of such crosses (5 males and 10 females) were set up which generated approximately 150,000 flies that were screened for GFP+, but white-eyed individuals. These flies were crossed to w1118; Ly/TM6C Tb Sb, and stocks were subsequently established from a single chromosome. The DHR96E25 allele was isolated from one of these stocks.

(9) Reduction of the DHR96 Targeted Event to a Single Copy by I-CreI

Males carrying the tandem duplication allele (w118/Y; DHR96E25/DHR96E25) were mated to v hsp70 CreI; Sb/TM6 females in mass. After 3 days at 25° C., the parental flies were removed and the progeny were heat-treated at 36° C. for one hour to induce CreI recombinase. Males that eclosed were individually mated to w1118; Ly/TM6C females. One male progeny (w1118/Y; DHR96Cre reduced/TM6C) that had lost GFP expression (indicating a recombination event had occurred) was selected from each vial and individually mated to w1118; Ly/TM6C females to establish a stock containing the reduced allele (Rong and Golic 2002). Mutant strains were characterized by Southern blotting, PCR, and DNA sequencing using standard methods. The DHR9616A mutant stock was selected for further characterization.

(10) Tissue Antibody Stains

Wandering third instar larval tissues were dissected and fixed as previously described (Boyd, L., O'Toole, E. and Thummel, C. S. (1991). Patterns of E74A RNA and protein expression at the onset of metamorphosis in Drosophila. Development 112, 981-995). DHR96 protein was detected with anti-DHR96 antibodies diluted 1:100 and incubated overnight at 4° C. Donkey anti-rabbit CY3 secondary antibodies (Jackson) were used at a 1:200 dilution as a secondary antibody. The stains were visualized on a Biorad confocal laser scanning microscope.

(11) Western Blots Analysis

Protein from adult flies was extracted by grinding flies in SDS sample buffer and boiling. The equivalent of approximately one adult fly was loaded in each lane of an 8% polyacrylamide gel, separated by electrophoresis and transferred to PVDF membrane. Ectopically expressed DHR96 protein was produced by heat-treating flies at 37.5° C. for 30 minutes followed by a three hour recovery at room temperature before the extraction procedure. DHR96 protein was detected by incubating the membrane first with a 1:500 dilution of anti-DHR96 affinity purified antibodies followed by a 1:1000 dilution of goat anti-rabbit HRP secondary antibody (Pierce). A supersignal chemiluminescence kit was used to develop the signal (Pierce).

(12) Toxicity Assays

Adult flies were raised on standard cornmeal/agar food and starved overnight under humid conditions at 25 0 C before treatment with DDT. A DDT stock solution was prepared by dissolving crystalline DDT (Sigma) in 100% ethanol. Appropriate DDT dilutions were made by diluting the DDT stock with 5% sucrose and pipetting 275 μl of the solution onto a strip of Whatman filter paper inside a small glass scintillation vial. Twenty adult flies were placed in each vial which was plugged with cotton. Mortality was scored 10 hours later at room temperature. For each DDT concentration, three replicates, each of twenty adult flies, were used. For the time course assay, 100 ng/μl of DDT was used and mortality scored every hour for 10 hours.

### b) Results

(1) DHR96 is Closely Related to Known Xenobiotic Receptors

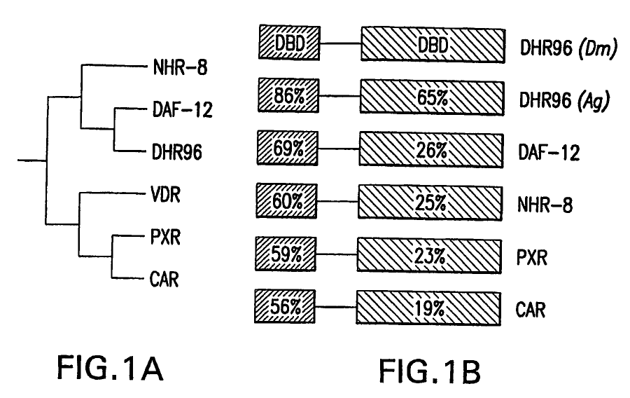

The phylogenetic relationship of DHR96 to other nuclear receptors was investigated for information related to function. When performing a BLASTP search, the closest homolog to DHR96 in vertebrates is the Vitamin D3 Receptor (VDR). The Pregnane X Receptor (PXR) as well as the Constitutively Androstane Receptor (CAR) comprise other high scoring homologs. (FIG. 1).

(2) DHR96 is Expressed in the Alimentary Canal, the Salivary Glands and the Fat Body

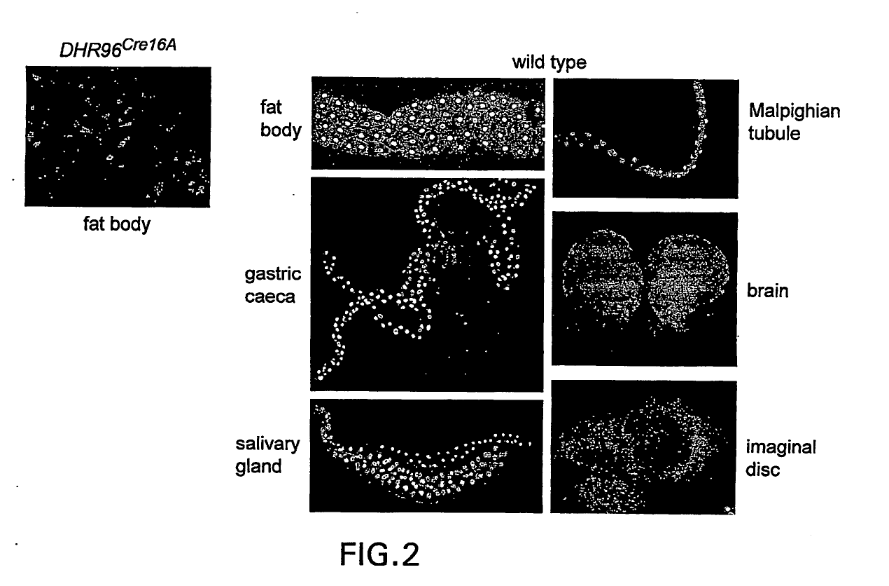

Antibody stains of third instar larvae were used to analyze whether DR96 would be expressed in tissues that function in detoxification. DHR96 antibodies strongly stain tissues of the alimentary canal (FIG. 2). In particular, the gastric caeca, the major site of absorption in Diptera, show a much stronger staining than the remainder of the midgut, which also plays a role in nutrient absorption. Strong expression in the Malpighian tubules, the principal excretory organ in insects, was also observed. The excretory system maintains homeostasis, controlling salt levels and osmotic pressure, but is primarily responsible for the removal of harmful metabolites such as nitrogenous wastes derived from purine metabolism, or toxic compounds that were absorbed from the food. Outside the alimentary canal, strong staining in the salivary gland and the fat body were detected. The insect fat body is the functional equivalent of the mammalian liver, because it is the principal site of intermediary metabolism and detoxification. Taken together, the finding that DHR96 expression is tightly associated with tissues known to be involved in detoxification provides strong support for the proposal that DHR96 functions in a xenobiotic pathway.

(3) DER96 Function is Dispensable Under Standard Conditions

RNA interference (RNAi) and gene targeting were used to disrupt DHR96 function because no existing mutants were available. The effects of DHR96RNAi were analyzed by generating transgenic lines that express snapback RNA under the control of a heat-inducible promoter. Three independent lines showed strong reduction of DHR96 mRNA in northern blots when treated with a single heat-shock, but displayed no discernable phenotype. Using a variety of heat-shock regimens, e.g. longer single and double treatments or 12 hr repetitions, did not affect the outcome of this observation. These findings suggest that DHR96 mRNA is not necessary for viability under standard conditions, indicating either that DHR96 protein is very stable or dispensable for survival.

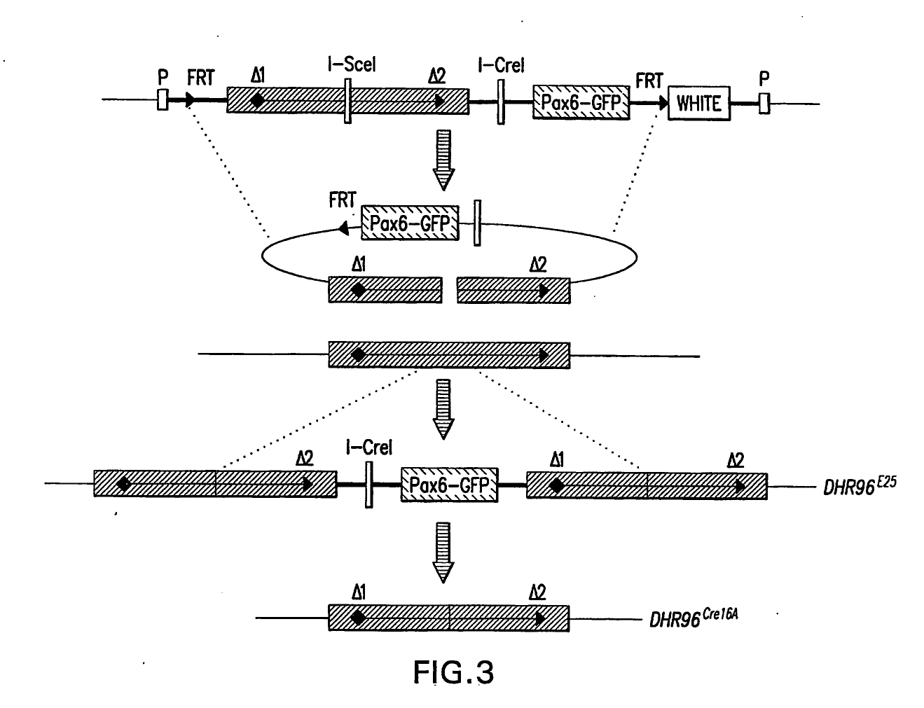

Gene targeting (Rong, Y. S., and Golic, K. G. (2000). Science 288, 2013-2018) was used to generate mutations in DHR96 because no deficiencies or P elements were known in this region of the genome. As a first step, the gene targeting procedure requires classical P-element transformation in order to generate transgenes that harbor the targeting sequence flanked by FRT sites. The targeting DNA is then mobilized and turned into a linear, recombinogenic molecule in vivo by activating the FLP recombinase and the endonuclease I Sce I. As a consequence of this targeting technique, which is based on an “ends-in” mechanism, the resulting mutation is basically a replacement of the original gene with a tandem duplication of two mutant copies (FIG. 3). Mutations were engineered in such a way that both copies would result in non-functional gene products. In particular, a region around the translation start site (25 bp), and the complete sequence of exon four was deleted, the downstream intron, and the splice acceptor site at exon 5 (together ˜300 bp). These mutations should lead to a block in translation initiation as well as removal of most of the ligand binding domain of the receptor. We constructed a targeting vector that contained two eye markers: pax6-EGFP and mini-white. Once mobilized by the FLP recombinase, the EGFP gene separates physically from the mini-white gene, which lies outside the FRT sites. Consequently, the subsequent strategy employed to identify potential targeting events is based on the presence of the EGFP marker and the simultaneous absence of the mini-white marker in the eye.

In a screen of ˜150,000 flies, a total of 42 events were detected. Of these, 18 mapped to the third chromosome, which harbors the DHR96 gene. At least one of the 18 events was identified as a targeting event in the DHR96 gene, and we termed this allele DHR96^{E25}. To avoid problems that might arise from the truncated protein in the DHR96^{E25} mutant, we decided to reduce the existing duplication to one mutant copy by utilizing the I Cre I site that was built into the targeting vector, essentially following the procedure described by (Rong, Y. et al., (2002) Genes Dev 16, 1568-1581). This procedure yielded a new DHR96 allele, DHR96^{169A}, which, based on sequence and western analysis, constitutes a protein null. Several lines of evidence suggest that these alleles represent specific targeting events in the DHR96 gene. First, genomic Southern blots of animals homozygous for the targeting events displayed the predicted fragment patterns of a tandem duplication (DHR96^{E25}) or a reduced single copy (DHR96^{16A}). Second, northern analysis revealed the absence of the wild type mRNA in the mutant animals. Third, antibody stains and Western analysis show a strong reduction or absence of the DH96 protein in DHR96^{16A} or DHR96^{E25} flies (add fig for this). Fourth, Southern blot hybridization and sequencing of PCR products demonstrated that exon/intron 4 of wild type DHR96 is absent in homozygous DHR96^{16A} or DHR96^{E25} animals.

Flies homozygous for DHR96^{E25} or DHR96^{16A} are viable and fertile when grown on standard cornmeal food. However, when placed on instant food (Carolina 424) in the absence of yeast, viability decreases to about 1%, whereas wild type flies do comparably well with a survival rate of ˜35% compared to standard food. Interestingly, the addition of yeast restores viability to 100%. This suggests that either DHR96 is required for the proper execution of certain nutritional pathways, or that DHR96^{E25} larvae fail to neutralize toxic metabolites that are produced when animals are reared on nutritionally poor media To test the possibility that DHR96 mutants have a decreased tolerance for toxins, it was determined whether DHR96 is expressed in tissues that are known to play critical roles in the detoxification process.

(4) DHR96 Mutants Display Reduced Viability in the Presence of DDT

As a test of DHR96 acting in a xenobiotic pathway, DHR96 mutants were tested for sensitivity to the pesticide DDT. Adult wild type flies (Canton S) and DHR96^{16A} were exposed or DHR96^{E25} flies to varying concentrations of DDT and recorded survival rates after a fixed time. The findings showed that DHR96 mutants were more sensitive to DDT and died at lower concentrations of DDT compared to control animals (FIG. 4A). In addition, when challenged with a fixed concentration of DDT, DHR96 homozygotes died more rapidly than wild type flies (FIG. 4B). Taken together, these results indicated that DHR96 is required for natural resistance levels to the pesticide DDT, and that DHR96 functions in a xenobiotic response pathway.

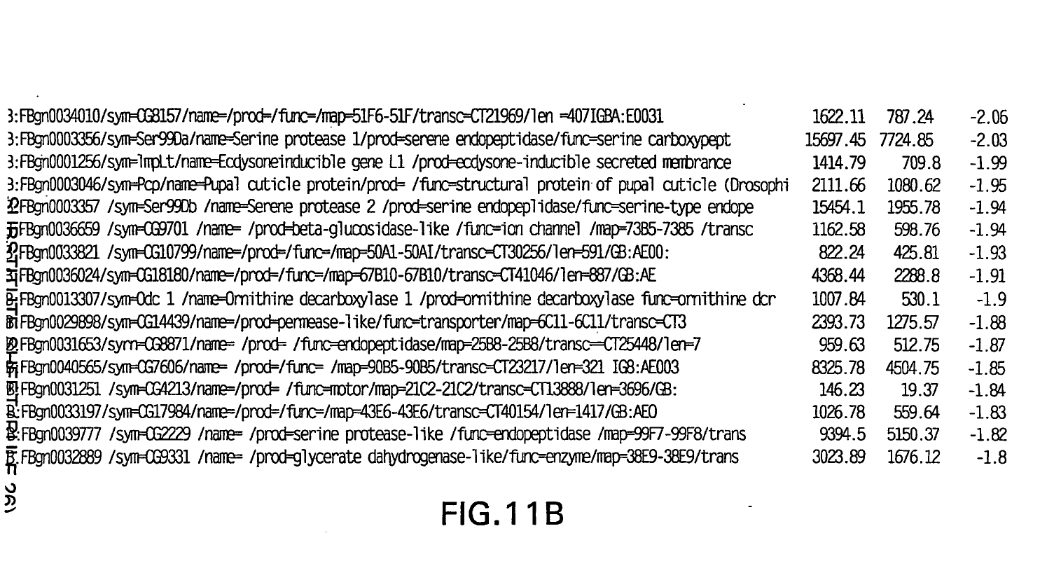

In addition to DDT, the outcrossed lines were tested for sensitivity to phenobarbital (a well characterized cytochrome P450 agonist), and tebufenozide (an insect growth regulator that is widely used in agricultural applications). The adult Canton S flies and the DHR96E25 outcrossed lines were exposed to varying concentrations of drug and recorded effects after a fixed time (FIG. 11). DDT was assayed by starving young healthy adult flies overnight and then transferring them to vials, in three groups of 20 flies each, with filter paper soaked with 5% sucrose alone or 5% sucrose and DDT at different concentrations. The number of living flies was scored after 23 hours. Phenobarbital was tested in the same way, except that the number of actively moving flies was scored after 23 hours. Tebufenozide was administered to larvae in the food, and the number of surviving adult flies was scored. These studies showed that, whereas the original DHR96E25 mutant line is more sensitive than Canton S to DDT treatment, this sensitivity must be due to a difference in genetic background since the outcrossed line showed no such sensitivity to this compound (FIG. 11A). In contrast, both the original and outcrossed DHR96E25 mutant lines are more sensitive to phenobarbital than Canton S, indicating that the genetic background did not contribute to this effect (FIG. 11B). Treatment with tebufenozide resulted in a slight sensitivity of the outcrossed DHR96E25 mutant to this compound (FIG. 11C). Taken together, these results indicate that DHR96 is required for natural resistance levels, showing it acts in a xenobiotic response pathway.

### (5) Overexpression of DHR1R96 has no Effect on Viability

Most nuclear receptors cause lethality when overexpressed, indicating that these proteins do not require an obligatory ligand for some or even all of their functions. To analyze whether DHR96 would disrupt essential pathways and cause lethality when expressed ectopically, a transgenic line that harbored a full-length DHR96 cDNA under the control of a heat-inducible promoter was produced. Western and Northern analysis showed that heat-treated larvae and flies carrying this construct generated at least 100 times more DHR96 mRNA and protein than wild type flies lacking the transgene. Nevertheless, overexpression of this protein did not result in any visible effect, suggesting two possible scenarios: (I) DHR96 activity requires binding to a ligand or a protein partner, or (II) DHR96 target genes do not function in vital pathways, at least not under standard laboratory conditions. Naturally, both possibilities may be true. Microarray experiments were used to dissect how DHR96 might function on the molecular level.

c) Microarray Experiments

As a first step toward identifying target genes regulated by DHR96, the protein was overexpressed in larvae and analyzed its effects on gene expression by microarray analyzed. Affymetrix oligonucleotide chips designed to detect ˜13,200 genes (the majority in the fly genome) were used, the raw data with dCHIP (Li C, Wong W H. Model-based analysis of oligonucleotide arrays: expression index computation and outlier detection. Proc Natl Acad Sci USA. 2001 Jan. 2; 98(1):31-6; Li, C., and Wong, W. H. (2001) Genome Biol 2, 0032.1-0032.11; http://www.dchip.org/) was analyzed, and filtering with Microsoft Access was performed. After rigorous filtering, only 72 genes remained that had a higher than 1.8-fold change when compared to the controls. Interestingly, of the top 20 reduced genes, six are members of all four major detoxification gene families, which comprise a total of 198 members in Drosophila. This represents a highly significant result (p=2.8×10⁻²⁷, based on χ²), because the chances of picking 6 of these genes in a random sample of 20 genes are more than 20-fold lower than the observed number. Interestingly, no such concentration of genes encoding detoxifying enzymes exists on the list of induced genes, suggesting that DHR96 may repress these genes in the absence of suitable ligands.

Further examination of this list reveals other genes that can contribute to a xenobiotic response pathway. The top down-regulated gene (25-fold by dChip) encodes Lspl-g, which is synthesized by the fat body and constitutes one of the most abundant proteins in the insect hemolymph. This protein is thought to act as a storage reservoir for nutrients during metamorphosis although it has also been proposed to transport small hydrophobic compounds within the circulatory system. The remaining down-regulated genes include three cuticle genes and one gene involved in cuticle tanning (black), consistent with the known role for cuticle deposition in toxin defense (Wilson et al. Ann. Rev. Entomol. 46:545-71, 2001). Other genes include a disproportionately large number that encode enzymes, such as a carboxylesterase, seven serine proteases, ornithine decarboxylase-1, dopamine N-acetyltransferase, an oxidoreductase, a g-butyrobetaine dioxygenase, a putative glucosidase, a chitin binding protein, and a transporter. Many genes that are up-regulated upon ectopic DHR96 expression) also have functions consistent with detoxification, including two cytochrome P450 genes (Cyp4p1, Cyp12d1-d). Only four families of cytochrome P450s are known to play a role in pesticide resistance: Cyp4, Cyp6, Cyp9, and Cyp12, each of which are represented in our microarray results (Ranson et al. Science, 298:179-81, 2002; Hemingway et al. Insect Biochem Mol Biol, 34:653-65, 2004). A range of enzyme-encoding genes were also detected, including the neuralized ubiquitin-protein ligase gene, phr DNA repair enzyme, eTrypsin, mitochondrial carnitine palmitoyltransferase I, a phosphatidate phosphatase gene (wunen-2), a oxidoreductase-encoding gene, a lysosomal transport gene, the drosomycin-2 defense response gene, a glycine dehydrogenase gene, two genes encoding chitin binding proteins (CG10140, CG7714), and, interestingly, SCAP, which encodes the fly ortholog of the mammalian protein that releases sterol regulatory element binding-protein (SREBP) from intracellular membranes in response to sterol depletion. This set of 72 DHR96-regulated genes appears to represent a coordinated genomic response to xenobiotics.

### 2. Example 2

### a) GAL4-DHR96/LBD Experiments

To determine if DHR96 is activated by the pesticide DDT the methods disclosed herein can be used. Flies containing two different transgenes will be mated together allowing us to directly assay for DHR96LBD activation in vivo (for detailed methods and description of vectors see: (Kozlova, T., and C. S. Thummel (2003) Methods to characterize Drosophila nuclear receptor activation and function in vivo. In: “Methods in Enzymology. Nuclear Receptors, Vol. 364 (Russell, D. W., and Mangelsdorf, D. J., eds.), Acadernic Press, New York, pp. 475-490.)). One transgene is under the control of a heat-inducible promoter and contains the GAL4 DNA binding domain fused to the DHR96 ligand binding domain. The second transgene contains a GAL4-dependent GFP or lacZ reporter gene (Kozlova, T., and C. S. Thummel (2003) Methods to characterize Drosophila nuclear receptor activation and function in vivo. In: “Methods in Enzymology. Nuclear Receptors, Vol. 364 (Russell, D. W., and Mangelsdorf, D. J., eds.), Academic Press, New York, pp. 475-490.)). Upon heat induction, GAL4-DHR96LBD protein can bind to the UAS-GFP or UAS-lacZ reporter. In the absence of a ligand, the reporter will not be activated; however, in the presence of a ligand, the GAL4 DHR96LBD protein can be switched into an active conformation and induce reporter gene expression (Kozlova, T., and C. S. Thummel (2003) Methods to characterize Drosophila nuclear receptor activation and function in vivo. In: “Methods in Enzymology. Nuclear Receptors, Vol. 364 (Russell, D. W., and Mangelsdorf, D. J., eds.), Academic Press, New York, pp. 475-490.); Kozlova, T. and Thummel, C. S. (2002). Spatial patterns of ecdysteroid receptor activation during the onset of Drosophila metamorphosis. Development 129, 1739-1750).

To determine if drugs, such as DDT, can activate the DHR96 GAL4-LBD construct, two developmental stages will be tested. First, organs from late third instar larvae that have both transgenes will be dissected and cultured in the presence of several different concentrations of drug and assayed for reporter gene expression. Second, if activation of the GAI4-LBD construct by drug requires either ingestion of the toxin or contact with the cuticle of the fly, adults will be heat-shocked to induce the GAL4-LBD construct, placed in scintillation vials containing drug, as previously above in the toxicity assays, and assayed for induction of reporter gene expression in adult tissues. Changes in the activity of the reporter gene in the presence, but not the absence, of drug will be an indication that that compound is having a direct effect on the activity state of the DHR96 LBD.

Disclosed are systems that can identify ligands, such as hormones, for nuclear receptors, such as drosophila nuclear receptors. There are many members of the nuclear receptor superfamily for which there is no known ligand—the so called orphan nuclear receptors. It is desirable to link these receptors to a ligand if it exists.

One way of identifying ligands for nuclear receptors involves expressing a fusion of the GAL4 DNA binding domain to a nuclear receptor ligand binding domain (LBD), in combination with a GAL4-responsive reporter gene. The fusion protein is inactive unless its hormone is present, allowing it to switch into an active conformation and turn on the GAL4-responsive reporter, such as a lacZ report giving a color readout. In one variation of this method, which has been widely exploited by pharma companies for high throughput screens, stably transfected tissue culture cells of different cell types are used for the cell background to perform the assay. One way to do this assay would be use every tissue in the animal as a context for screening for hormones, not just a tissue culture cell where the appropriate cofactors or partner transcription factors might be missing, because presumably every cell has a different molecular background.

One method used to get around this problem in mice is disclosed in WO 00/17334 for “Analysis of ligand activated nuclear receptors (in vivo)” by Solomon et al. (See also, Solomin, L., et al., (1998). Nature 395, 398-402). This system was designed for the mouse, because the GAL4 system of linking the GAL4 DBD to a particular LBD works poorly in mouse.

Disclosed herein is a system for Drosophila for identifying ligands for nuclear receptors, where the GAL4 system works very well for driving tissue- and stage-specific ectopic gene expression. The system typically utilizes a heat-inducible promoter to widely express the GAL4-LBD fusion proteins, but any inducible promoter can be used. This allows monitoring of activation in all tissues both spatially and temporally. The pattern of lacZ expression in animals so transformed allows visualization of where and when a particular LBD is active during development, guiding one towards possible sources of hormone.

This has been used to show the patterns of GAL4-EcR and GAL4-USP activation during the onset of metamorphosis accurately reflect what would be expected for regulation of EcR/USP by its hormone, 20-hydroxyecdysone (Kozlova, T. and Thummel, C. S. (2002). Spatial patterns of ecdysteroid receptor activation during the onset of Drosophila metamorphosis. Development 129, 1739-1750). Spatial patterns of ecdysteroid receptor activation during the onset of Drosophila metamorphosis. Development 129, 1739-1750). This system has also been used to show that an orphan nuclear receptor, DHR38, is activated by a unique set of ecdysteroids in the animal (Baker, K. D., et al., (2003). The Drosophila orphan nuclear receptor DHR38 mediates an atypical ecdysteroid signaling pathway. Cell 113, 731-742).

Disclosed herein are hsp70-GAL4-LBD transformants for all 18 Drosophila nuclear receptors. The activation patterns of these constructs have been characterized during embryogenesis and the onset of metamorphosis. These constructs can be used with a UAS-GFP reporter to simplify the readout of activation, paving the way for compound screens.

These constructs can be used to screen compounds for ligand activity. For example, a collection of pesticides can be found in the Agro plate (see http://www.msdiscovery.com). Other plates can also be found at Micro Source Discovery, and are herein incorporated by reference at least for compound libraries and their contents. They also list plates of available collections of natural compounds.

### 3. Example 3

### Effective Assays for Studying Drug Sensitivity in DHR96 Mutants

Two contact poisons, DDT and tebufenozide, as well as the GABA agonist, Phenobarbital, have been tested. This set of compounds can be expanded to include the major classes of pesticides used for insect control, all of which have been compromised to some extent by adaptive resistance in pest species. These major classes include organochlorines, organophosphates, carbamates, pyrethroids, nicotinoids, and insect growth regulators. Representative compounds from these classes are shown in Table 3, along with their solubility. They include several compounds that have been used in studies of C. elegans and vertebrate xenobiotic responses, as well as paraquat to test responses to oxidative stress. Methyl parathion can also be tested, which is a weak insecticide, but which becomes a potent acetylcholinesterase inhibitor (methyl paraoxon) upon metabolism. DHR96 mutants can be less sensitive to this compound than wild type. Imidacloprid, a nicotinoid that that is one of the most widely used insecticides worldwide, fipronil which has both pet and agricultural applications and acts as a GABA antagonist, or additional pyrethroids can also be tested.

TABLE 4
List of compounds:

| Compound | Description | Solubility |
|---|---|---|
| DDT | Organochlorine, contact poison, | ethanol |
| | thought to target sodium channels | |
| Phenobarbital | GABA mimetic, causes paralysis | water |
| Permethrin | Pyrethroid, blocks voltage gated | comes as |
| | sodium channels | liquid |
| Sodium | Carbamate, cholinesterase inhibitor | water |
| diethyldithiocarbamate | | |
| trihydrate | | |
| Carbaryl | Carbamate, cholinesterase inhibitor | water |
| Methyl parathion | Organophosphate, contact poison | acetone |
| Malathion | Organophosphate, contact poison | comes as |
| | | liquid |
| Propetamphos | Organophosphate contact poison, | comes as |
| | cholinesterase inhibitor | liquid |
| Tebufenozide | Contact poison, ecdysone agonist | ethanol |
| Nicotine | Contact poison | water |
| Nithiazine | Neonicotinoid, used on plant | water |
| | sucking insects | |
| Methoprene | JH mimetic, insect growth regulator | ethanol |
| PCN | Synthetic hormone that induces | DMSO |
| | P450s in vertebrates | |
| Rifampicin | Antibiotic that inhibits RNA | DMSO |
| | polymerase, used in vertebrate | |
| | xenobiotic studies | |
| Colchicine | Alkaloid that inhibits mitosis, | ethanol |
| | used in vertebrate xenobiotic studies | |
| Paraquat | Generates oxygen radicals, inducing | water |
| | stress and decreasing life span, | |
| | induces GSTs which can provide | |
| | resistance to oxidative stress | |

The key to defining the sensitivity of DHR96 mutants to toxic compounds is the development of effective and reproducible assays for drug delivery. To feed compounds to adult insects, the method for administering the mutagen ethylmethane sulfonate (EMS) (Lewis et al. Dros Info. Serv. 43:193, 1968) can be used. Young adult flies, within the first five days of their life, are starved overnight in an empty vial and then transferred to a vial that contains 5% sucrose and different concentrations of the drug to be tested. The flies congregate on the filter paper to drink the sugar solution along with the drug. This method of application also provides significant surface contact as well as possible fumigant modes of entry through the trachael system. This assay has not resulted in detectable differences in the behavior of wild type and DHR96 mutant flies, indicating that there are no obvious differences in taste reception, or eating and drinking behavior that might result in different doses of drug between mutant and control. For all of our drug treatment studies, the highest concentration of vehicle alone is tested to determine that it does not have an effect on the experiment. An initial dose-response curve using 10-fold changes in drug concentration for either 10 or 24 hours can be used. Treatment with each drug concentration is performed in triplicate, with 20 adult flies per vial. These numbers can be increased as well, although this has not had a significant effect on experimental variability in past studies. These initial dose-response curves result in the identification of a concentration at which most animals survive as well as a higher concentration that kills most animals. The study is then repeated using 2- to 3-fold differences in dose spanning this critical range of concentrations. This provides us with a lethality curve, error bars for each data point, and an LD50 that can be compared between mutant and wild type. If desired, a time course study at a fixed concentration of pesticide can also be conducted using a similar assay.

A method used in other insects to assay contact toxins in Drosophila can also be used (Daborn et al. Mol Genet Genomics, 266:556-63, 2001). Different amounts of the compound to be tested are mixed with 200 μl acetone and added to a glass scintillation vial. The vial is rolled so that the liquid contacts all glass surfaces. This is continued until the acetone has evaporated, leaving the toxin evenly distributed inside the vial. Groups of 20 young adult flies are transferred to each vial and lethality is scored after a fixed time. Alternatively, a fixed compound concentration is tested over a range of times. The determination of appropriate doses and treatment times is similar to that described above for the adult feeding assay. This method has been used successfully in to generate a lethality curve for Canton S wild type animals treated with DDT.

The above assays are for adult toxicity studies, scoring the number of dead flies resulting from exposure. Not all compounds, however, result in lethality. For example, phenobarbital increases the chloride current from the GABA receptor, enhancing the effects of this inhibitory neurotransmitter (Barber et al., Proc R Soc Lond B Biol Sci 206:319-27, 1979). This compound is used clinically in humans as an anticonvulsant. At high doses in insects, it results in ataxia and, eventually, lethality. The experiment depicted in FIG. 11B shows that DHR96 mutants display a significant sensitivity to this compound relative to the Canton S control, a result we have seen reproducibly. Standardized assays have been developed to characterize behavioral defects in Drosophila (ainton et al., Curr Biol 10: 187-94, 2000; Rival et al. Curr Biol 14:599-605, 2004). Several of these can be employed to quantitate the effects of phenobarbital and similar drugs that result in abnormal behavior. First, running ability can be tested by transferring eight young adult flies, either DHR96 mutants or Canton S control, into a 10 ml plastic pipette. Both ends are sealed with paraflim and one half of the pipette will is inserted into a hole in a black foam block such that the pipette is held horizontally, allowing the flies to run along its length. A fiber optic lamp is placed at the opposite end of the pipette to create a clear gradient from dark to light, to stimulate a phototactic response. For each test, the flies are knocked into the dark half of the pipette and then returned to the horizontal test position. The time is recorded at which the first six flies enter the light half of the pipette. Four trials will be done for each set of eight adults tested. The resulting times are used to calculate mean performance coefficients, as described (Palladino et al. Genetics 161:1197-208, 2002). Statistical analysis of the data can be performed using a Student's t-test.

The second behavioral assay is a flight ability assay, performed essentially as described (Benzer et al. Sci Am 229:2437, 1973). Twenty young adult mutant or wild type flies are dumped into a glass funnel placed on top of a 500 ml graduated cylinder, such that they are released into the cylinder near the 500 ml mark on top. The glass cylinder is coated with paraffin oil to provide a sticky surface to which flies will adhere. Healthy animals initiate flight immediately and thus tend to become caught near the opening of the funnel. Weaker flying animals, in contrast, fall farther toward the bottom before being caught. Performance coefficients are calculated for the population added to the cylinder by assigning a numerical score for the distance fallen by each fly, as described (Palladino et al). Statistical analysis of the data can be performed using a Student's t-test.

Finally, the most widely used behavioral assay for measuring locomotor activity, called a climbing assay or negative geotaxis assay is used. Twenty young adult flies are placed in a 250 ml graduated cylinder and the top is sealed with parafilm. The flies are knocked gently to the bottom of the cylinder and then allowed to climb for one minute. The number of flies in the top, middle, or bottom one-third is determined and recorded. This can be further subdivided if necessary. Three trials are performed with one population of flies, and the results are averaged. The mean number of flies in each region of the cylinder can be calculated as a fraction of the total population of flies, and a performance index is determined as described (Rival et al.). Statistical analysis of the data will be performed using a Student's t-test. A more general motility assay can also be used in which flies are treated with drug and then transferred to a regular vial without food. The flies are gently banged into the bottom of the vial, the top is removed from the vial, and the flies are allowed to escape for a fixed period of time before the top is resealed. The number of remaining flies is then scored and an average is calculated from several repeated tests of the same population.

An advantage to non-lethal drugs such as phenobarbital is that they allow for the testing of a different ability of DHR96 mutant flies—their ability to recover from drug treatment. If, indeed, DHR96 mutants express lower levels of detoxifying enzymes than wild type flies, a slower rate of recovery for mutant flies exposed to a drug should be seen. This test requires treating young adult flies with sub-lethal doses of a drug and then scoring the time it takes for those animals to regain normal behavior following transfer back to normal food. The choice of assay to measure behavior depends on the type of drug being tested, as described above. The advantage of a recovery test is that it may uncover more subtle effects on detoxification gene expression than could be detected by the acute tests described above. For example, whereas mutant and wild type flies might show a small difference in negative geotaxis when challenged with a particular drug, assaying for the ability of these two stocks to recover from drug treatment may significantly increase this difference.

The above assays are for testing the effect of xenobiotics on adult flies. Compounds can also be tested for their larvicidal effects by administering them in the food to staged populations of larvae (Grant et al. Bull. Envir. Contam. Tox. 69:35-40, 2002). DHR96 and Canton S control flies are maintained on normal cornmeal/molasses agar supplemented with yeast. Egg lays are collected overnight from these stocks and used to innoculate fresh vials of food supplemented with a specific concentration of the drug to be tested. The drug are mixed with either Instant Drosophila Medium (Formula 4-24, Carolina Biological Supply) or added to a defined growth medium for Drosophila (Sang et al.). The Instant Medium is a flake formulation that is simply mixed with water before use. Drugs at different concentrations can be easily added to each vial and mixed into an even suspension for oral delivery. The defined medium is in an agar base and thus the drug needs to be added as the food is being prepared. The advantage of the former is its ease of use. The advantage of the latter is its defined constitution of specific amino acids, vitamins, and other essential nutrients. The use of the Carolina Instant medium with drugs such as tebufenozide (FIG. 11C) has already been tested.

All studies described above are conducted with a DHR96 mutant stock that has been outcrossed for 10 generations to the Canton S control stock. As a further test of specificity, toxin sensitivity rescue can be tested by using a wild type DHR96 transgene in a DHR96 mutant background. Two transgenes are used for this propose. First, the heat-inducible hsp70-DHR96 fusion gene described above can be used. This construct has been established in transformed flies and used to overexpress wild type DHR96 protein (FIG. 10). This transgene has been crossed into a DHR96 mutant background and expressed DHR96 protein with a 30 minute 37° C. heat treatment. Western blots reveal that DHR96 protein can be easily detected at 24 hours after heat induction, at levels comparable to endogenous expression, indicating that the protein is relatively stable (FIG. 10). This hsp70-DHR96 transgene can be crossed into the tenth outcross stock of the DHR96^{E25} mutant and DHR96 expression induced by a single 30 minute 37° C. heat treatment in larvae or adult flies tested with the drug. DHR96 mutant and Canton S control animals are subjected to an identical heat treatment regime to control for any effects due to temperature. The appropriate drug and assay can then be used, as described above, to determine how the transgene affects the DHR96 mutant phenotype. Thus, for example, while DHR96 mutant flies might show sensitivity to a particular drug under conditions in which Canton S flies are relatively normal, this sensitivity can be rescued by heat-induced DHR96 expression, essentially recovering wild type function.

A second rescue construct can be used that does not depend on heat-induced expression. A 11.8 kb fragment, extending from 2.5 kb 5′ of the wild type DHR96 gene to 2.8 kb 3′ of the gene, can be excised from a P1 genomic clone and inserted into the Carnegie 4 fly transformation vector (Rubin et al., Nucleic Acids Res 11:6341-51, 1983). This DHR96 rescue fragment is introduced into the fly genome using standard methods for transformation, and crossed into the DHR96^{E25} mutant background. Western blot analysis of this stock can reveal a recovery of wild type levels of DHR96 protein, indicating that the transgene is functioning as expected. This rescued stock, along with the DHR96 mutant and Canton S control, can then be tested using an appropriate drug assay. Both the Canton S and rescued stock can show a similar wild type response while the DhR96 mutant shows a defective Response, indicating that the phenotype seen in the mutant can be specifically ascribed to the DHR96 locus.

Finally, it can be determined whether DHR96 overexpression in a wild type genetic background has any effects on xenobiotic sensitivity. The hsp70-DHR96 transgene is crossed into a Canton S background to ensure that no phenotypic differences between these stocks are due to genetic background. Heat-induced hsp70-DHR96 transformants are then tested with a range of compounds, using assays as described above, comparing their sensitivity to heat-treated Canton S controls. This gain-of-function genetic test complements the loss-of-function genetics described above.

### 4. Example 4

### A Role for DHR96 in the Regulation of Specific Detoxifying Genes

Genes that are expressed in response to xenobiotic challenge can be identified, and it can be determined what role DHR96 might play in mediating this regulation. The observation that DHR96 mutants display a reproducibly increased sensitivity to phenobarbital (FIG. 11B) can be used. This compound has been used extensively in vertebrates for inducing xenobiotic responses and studying the transcriptional functions of the PXR and CAR xenobiotic receptors (Sueyoshi et al. Annu Rev Pharmacol Toxicol 41:123-43, 2001). Phenobarbital is also the most widely used inducer of xenobiotic gene transcription in insects. In Drosophila, it has been shown to have a significant effect on Cyp6a2, Cyp6a8, Cyp6a9, and Cyp28 transcription, genes that are proposed to have xenobiotic activity. Northern blot hybridizations have been used to study the effects of phenobarbital on Cyp6a2 and Cyp6a8 transcription in wild type and DHR96 mutant adult flies treated with 0.3%, 1%, and 3% phenobarbital. These results showed a dramatic induction of Cyp transcription in wild type animals, although no change in expression was seen in the DHR96 mutant. As many potential detoxifying genes as possible can be considered. Canton S wild type and DHR96^{E25} mutant adult flies, of identical genetic background and age, can be treated with either sucrose alone, or sucrose and 0.3% phenobarbital. This concentration is the lowest one at which DHR96 mutants show a clear and reproducible sensitivity to the drug relative to wild type (FIG. 11B). It is also one that has been used in published studies of phenobarbital induced genes in Drosophila (Dunkov et al. DNA Cell Biol. 16:1345-56, 1997; Brun et al. Insect Biochem Mol Biol 26:697-703, 1996). Each treatment is done in triplicate. RNA is extracted from each set of animals, purified by TRIzol extraction (Gibco BRL) followed by RNeasy column chromatography (Qiagen), and ethanol precipitation. The RNA is then labeled and hybridized to Affymetrix GeneChip® Drosophila Genome 2.0 arrays designed to detect 18,500 Drosophila transcripts. Data is then analyzed using DChip 1.3 (http://biosun1.harvard.edu/complab/dchip/) and Significance Analysis of Microarrays (SAM). The data is scanned for changes in Cyp6a2 and Cyp6a8 mRNA levels, to confirm that phenobarbital treatment has had the expected effect in both wild type and DHR96 mutant animals. Cyp6a9 and Cyp28 induction in wild type animals based on published data can also be seen (Danielson et al., Proc Natl Acad Sci 94: 19797-802, 1997). Additional attention is paid to the genes that were identified by DHR96 overexpression as potential regulatory targets.

There are two sets of data that emerge from this study. First, the data from untreated and treated Canton S controls identifies, for the first time, the genomic response to a xenobiotic compound in a wild type insect. This data can be analyzed to identify as many known detoxification genes as possible, focusing on the four main classes. Comparisons can be made with previous microarray studies that examined Drosophila genes involved in oxidative stress, to identify common stress response pathways (Landis et al. Proc Natl Acad Sci, 101:7663-8, 2004; Girardot BMC Genomics, 5:74, 2004). Gene ontology listings of array data can also be examined to identify new players in the xenobiotic response pathway (Misra et al. Genome Biol. 3:83, 2002). The second set of data to emerge from this microarray study allows for the determination of how DHR96 might contributes to xenobiotic transcriptional responses in Drosophila. By comparing the set of genes regulated by phenobarbital in Canton S animals to those same genes in the DHR96 mutant, it can be determined whether DHR96 is required for this transcriptional response. Some genes can change their expression in wild type animals treated with phenobarbital will respond differently in DHR96 mutants. The number and type of these gene changes provides insights into why DHR96 mutants are more sensitive to phenobarbital than Canton S control animals. In addition, this experiment provides possible direct targets of DHR96 transcriptional control, providing a foundation for the experiments described below.

Genes that change their regulation in Canton S animals treated with phenobarbital, and genes that are affected by the DHR96 mutant, are validated by northern blot analysis. Collections of adult animals fed phenobarbital, as described above, can be used along with dose-response and time-course studies to understand the mechanisms of xenobiotic gene regulation. Validation can be conducted on selected genes, covering the different classes of detoxification pathways as well as new players that identified. Similar microarray studies using at least two other compounds, depending on which compounds show an effect in the viability and behavioral assays. It will be confirmed that wild type Canton S flies show a response to DDT using Cyp12d1 and other P450 genes as probes for northern blot hybridization. One experiment showed a low level of Cyp6g1 induction by DDT in Canton S. Provided that a response can be detected, the survey can be conducted of DDT-regulated genes by performing microarray studies similar to those reported above for phenobarbital. Alternatively, it can be determined whether senita cactus alkaloids, compounds that have been shown to regulate the three Cyp28 genes in Drosophila mettleri, also regulate these genes in D. melanogaster (Danielson et al. Proc Natl Acad Sci 94: 10797-802, 1997). Other pesticides can also be surveyed for effects on a select group of Cyp gene targets to identify other compounds for use in comparative microarray profiling. The genomic response to these compounds can be determined and compared with the phenobarbital response, as well as determine how DHR96 impacts these regulatory pathways. Determining the transcriptional response to more than one xenobiotic compound can provide an initial impression of how insects respond to different toxins in their environment. It is possible that a common core defense response can be activated in response to a range of drugs. Alternatively, the genetic response may be fine-tuned to combat specific xenobiotic compounds.

### 5. Example 5

### DHR96 Activation by Xenobiotic Compounds

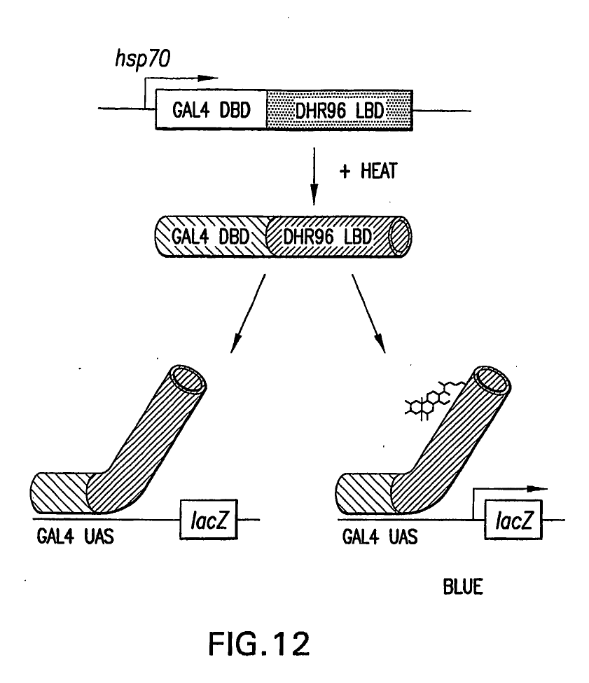

The human PXR xenobiotic nuclear receptor can directly bind xenobiotic compounds in its ligand binding pocket (Watkins et al., Science, 292:2329-2333, 2001), triggering induction of PXR targets, including the CYP3A detoxifying gene (Jones et al. Mol Endocrinol 14:27-39, 2000). This defines a positive feedback loop in which toxic compounds directly induce the expression of detoxifying genes through the PXR receptor. It can be determined whether DHR96 (the fly homolog of PXR, FIG. 1), acts in a similar manner. Several lines of evidence suggest that DHR96 might require a ligand for its activity. First, it is constitutively expressed throughout development, indicating that any temporal or spatial specificity for activation would have to be conferred post-transcriptionally. Second, ectopic overexpression of DHR96 has no effects on growth or development, unlike the majority of Drosophila orphan nuclear receptors that appear to act as constitutive transcriptional regulators (Thummel, Cell 83:871-7, 1995). Third, ectopic overexpression of DHR96 represses target genes, as shown by the microarray study (FIG. 12), similar to unliganded nuclear receptors such as the thyroid hormone receptor (Hu et al. Trends Endocrinol Metab 11:6-10, 2000). Finally, good evidence exists that the close relative of DHR96, the C. elegans DAF-12 receptor (FIG. 1A), is regulated by a steroid ligand (Matyash et al. PloS Biol. 2, e280, 2004, Gerisch et al. Development 129:1739-50, 2004).

DHR96 activation can be assayed for by using a method established to follow the activation status of a nuclear receptor ligand binding domain (LBD) in a developing animal. This method uses transformed Drosophila that carry the hsp70 heat-inducible promoter upstream from the coding region for the yeast GAL4 DNA binding domain fused to the coding region for the DHR96LBD (FIG. 13). These hs-GAL4-DHR96 transformants are crossed with flies that carry a GAI4-dependent promoter driving a lacZ reporter gene that expresses nuclear β-galactosidase (UAS-lacZ). Expression of β-galactosidase can be detected by histochemical staining using X-gal as a substrate, generating a blue dye (FIGS. 13, 14). A UAS-GFP reporter has also been used to detect GAL4-LBD activation in living animals, although this assay is somewhat less sensitive than that provided by β-galactosidase detection. The hsp70 promoter was selected in order to provide precise temporal control, reducing potential lethality that might be caused by overexpression of the GAL4-LBD fusion protein (similar fusions to nuclear receptors have been shown to function as dominant negatives). In addition, the hsp70 promoter should direct widespread expression of the GAL4-DHR96 protein upon heat induction, allowing for the assay for activation throughout the animal. Activation by this fusion protein, however, should only occur at times and in places where the appropriate hormonal ligand and/or co-factors are present. This method thus provides a visual readout of where and when and LBD can be activated in the context of an intact developing animal, providing a powerful tool for defining nuclear receptor signaling pathways. This system has been used to characterize the activation patterns of the Drosophila EcR and USP nuclear receptors, which act as a heterodimeric-receptor for the steroid hormone ecdysone (Kozlova et al. 129:1739-1750, 2002). More recently, all 18 canonical Drosophila nuclear receptors have been used, defining their activation patterns during both embryogenesis and metamorphosis. These experiments have shown that GAL4-DHR96 is not normally active in wild type animals.

To test that, like its vertebrate counterparts, DHR96 is activated by xenobiotic compounds, thereby inducing the expression of detoxification target genes, activation of the GAL4-DHR96 fusion protein by xenobiotic compounds using three different means of compound delivery: (1) adding xenobiotic compounds to cultured third instar larval organs, (2) feeding larvae with xenobiotic compounds, and (3) feeding adult flies with xenobiotic compounds.

An advantage of the GAL4-LBD system is that it can be used in tissues dissected from transgenic larvae to test specific compounds for their ability to activate the fusion protein. Thus, for example, the steroid hormone 20-hydroxyecdysone is a potent activator of the GAL4-USP fusion protein, and this response is dependent on its EcR partner, as expected (Kozlova et al. Development 129:1739-50, 2002). Similarly, tests of several compounds using the GAL4-LBD system in cultured larval organs revealed that the Drosophila NGFI-B ortholog, DHR38, can be activated by α-ecdysone and 3-epi-20-hydroxyecdysone, but not 20-hydroxyecdysone. A similar assay can be used to test the ability of xenobiotic compounds to activate the GAL4-DHR96 fusion protein in cultured larval organs, using either UAS-lacZ or UAS-GFP as a readout. A few compounds have been tested in this manner in an initial effort to determine whether this approach will work as desired with the GAL4-DHR96 fusion. Of the compounds tested (DDT, phenobarbital, and tebufenozide), tebufenozide showed a reproducible and distinct pattern of activation. Control tissues dissected from heat-induced UAS-lacZ larvae treated with either vehicle alone or tebufenozide, or heat-induced hs-GAL4-DHR96; UAS-lacZ larvae treated with vehicle alone, gave a low background pattern of activation (control in FIG. 14). In contrast, larval organs dissected from hs-GAL4-DHR96; UAS-lacZ larvae and treated with tebufenozide gave a reproducible pattern of activation (GAL4-DHR96 in FIG. 14). Interestingly, this pattern is similar to that of endogenous DHR96 protein: in the fat body, midgut (but not restricted to the gastric caeca), and Malpighian tubules (but not salivary glands).

Organs isolated from other stages of development can be tested for their ability to direct GAL4-DHR96 activation by tebufenozide, to control for the possibility that a critical co-factor for DHR96 activation can be temporally restricted. The stage used for the experiment depicted in FIG. 14 is not ideal as mid- and late third instar larvae stop feeding in preparation for metamorphosis. Actively feeding stages during the second and early third instar can therefore be tested. Finally, it can be determined whether a natural form of compound delivery is more effective at revealing GAL4-DHR96 activation than using an in vitro organ culture system. Providing compounds to the animal in their growth medium allows for entry through the digestive system, epidermis, and/or tracheal system. Compounds added in this way can then have either a direct effect on the GAL4-DHR96 reporter or an indirect effect, with LBD activation occurring via a metabolic product of the compound being tested. Compounds are fed to control UAS-lacZ larvae and hs-GAL4-DHR96; UAS-lacZ larvae using either Instant Drosophila Medium (Formula 4-24, Carolina Biological Supply) or the defined growth medium. These animals are then be heat-treated, allowed to recover for 4-6 hours, and the patterns of lacZ expression are determined by Xgal assays (or fluorescence can be used to detect GFP for the UAS-GFP reporter gene). The methods described above can also be used to provide xenobiotics to adult Drosophila, feeding with a sucrose solution or using a contact assay. Taken together, these assays should provide a list of compounds that can activate the GAL4-DHR96LBD fusion protein in an intact animal, providing a basis for determining whether these compounds directly activate the DHR96 receptor as well as a means of understanding how xenobiotic compounds are sensed in insects.

While the GAL4-LBD system can be used to identify compounds that activate the LBD, it does not indicate the mechanism by which this activation is achieved. This effect could be obtained by direct binding of the compound to the LBD, as is the case for the EcR/USP heterodimer in Drosophila, or it could be due to the recruitment of protein co-factors or any post-transcriptional modification that could provide a transcriptional activation function. Accordingly, compounds that are scored as positive by our GAL4-DHR96 assay act directly on the D1R96LBD are tested.

### 6. Example 6

### Conserved Regulatory Sequences in Detoxification Target Promoters

The studies described above provide insights into how xenobiotics are sensed by insects and how the animal reprograms its gene expression to detoxify these compounds. Biochemical techniques can be used to determine whether DHR96 functions as a monomer, homodimer, or heterodimer with USP, and determine its DNA binding specificity. Second, the sequences bound by DHR96 can be tested in vivo, using chromatin immunoprecipitation (CHIP) and antibody stains of the larval salivary gland polytene chromosomes. Comparison of this data with the in vitro DNA binding results should provide an understanding of how DHR96 contacts target genes and identify potential regulatory targets in the genome for further characterization. Third, the regulatory sequences of coordinately expressed detoxification genes can be compared, as determined by the microarray studies, to identify common sequence elements. It can be determined which of these sequence elements are bound by DHR96 and which might be bound by other regulatory factors. Taken together with the functional studies described herein, this work can provide a strong foundation for understanding how insects reprogram-their patterns of gene expression to respond to toxic compounds in their environment.

DHR96 contains a novel P box sequence within its DNA binding domain: ESCKA (Fisk et al. Proc Natl Acad Sci, 92:10604-8, 1995). This P box is shared by only three other nuclear receptors in any organism—the three C. elegans homologs of DHR96: DAF-12, NHR-8, and NHR-48—suggesting that DHR96 regulates a unique set of target genes in the insect genome. Consistent with this observation, it was found that DHR96 protein fails to bind to most canonical nuclear receptor response elements, except for weak binding to a pallindromic ecdysone response element (EcRE). A recent paper has determined the DNA sequences bound by DAF-12, providing initial insights into the binding specificity of this receptor subfamily (Shostak et al. Genes Dev 18:2529:44, 2004). They identified a direct repeat of two distinct hexanucleotide sequences (AGGACA and AGTGCA), separated by five nucleotides (DR5), as a functional DAF-12 binding site and response element. The authors proposed that DAF-12 would contact these sequences as a homodimer, although no experiments were done to address this issue. The DNA sequences bound by DH96 can be determined. As a first step toward this goal, we will determine whether DHR96 acts as a monomer, a homodimer, or forms a heterodimer with USP, the fly ortholog of vertebrate retinoid X receptor (RXR). The vertebrate DHR96 homologs, PXR, CAR, and VDR, all act as heterodimers with RXR, suggesting that this interaction may have been conserved through evolution, Like vertebrate RXR, USP heterodimerizes with multiple nuclear receptor partners, including EcR and DHR38, indicating that it has relatively broad regulatory functions. GST-tagged USP protein are overexpressed in bacteria and purified by glutathione chromatography. All tags are added to the amino-terminal ends of the proteins, distant from the C-terminal dimerization sequences within the LBD. GST-USP is mixed with either FLAG-EcR or FLAG-DHR96, purified by glutathione chromatography, fractionated by gel electrophoresis, and FLAG-tagged proteins that are bound by GST-USP can be detected by Western blot analysis using anti-FLAG antibodies. Detection of the EcR/USP heterodimer acts as a positive control for this study. Results from this experiment can be confirmed by performing protein-protein interaction studies using either radiolabeled or unlabeled DHR96 and USP proteins synthesized in vitro, and our anti-DHR96 antibodies or AB11 mouse monoclonal antibodies directed against USP for immunoprecipitation. Again, detection of the EcR/USP heterodimer can be used as a positive control. These studies are directed at determining if DHR96 can heterodimerize with USP. To test if DHR96 can homodimerize, co-express GST-tagged DHR96 and FLAG-tagged DHR96 by in vitro translation. Protein is purified by using affinity beads for one of the two tags, and the presence of the other tag is assayed by gel electrophoresis followed by Western blot analysis, using antibodies directed against GST or anti-FLAG antibodies (both are commercially available).

To facilitate our identification of DHR96 regulatory targets, it can be determined which DNA sequences are preferentially bound by this transcription factor. DHR96 protein can be overexpressed and purified. This protein can be used either alone or in equimolar combination with purified USP, depending on whether it forms a USP heterodimer. USP is purified from an overproducing strain of baculovirus, generously provided by M. Arbeitman and D. S. Hogness (Arbietman et al. Cell 101:67-77, 2000). The selected and amplified binding site assay (SAAB) developed originally by Blackwell and Weintraub can be used. This method has been used widely to determine the optimal recognition sequences for DNA binding proteins. By using PCR to amplify each round of oligonucleotides that are selected for their ability to bind to DHR96, multiple random positions in the DNA sequence can be used, and thus better determined which sequences are optimally recognized by the protein. One choice of oligonucleotide sequences for this study can be informed by our earlier determination of how DHR96 contacts DNA, as a monomer, homodimer, or USP heterodimer. A pallindromic arrangement of random hexanucleotide sequences can also be tested, based on the identification of weak binding to the pallindromic EcRE, as well as a DR5 arrangement of hexanucleotide sequences based on the DAF-12 binding site. This analysis provides a set of ideal high affinity DHR96 binding sites, allowing for the determination of an optimal consensus recognition sequence. Although such ideal sites are rarely used in vivo, they nonetheless provide an invaluable guide for identifying bonefide binding sites within cis-acting regulatory sequences. For example, the determination of an optimal E74A ETS-domain DNA binding site by random oligonucleotide selection greatly facilitated the identification of downstream target genes (Umess et al. EMBO J. 14:6239-46).

DHR96 binding sites used in vivo can also be used, and, by comparing them with the above biochemical data, define a set of potential direct regulatory targets in the genome. Two methods are used to determine where DHR96 protein is bound—antibody stains of the giant larval salivary gland polytene chromosomes and chromatin immunoprecipitation (ChIP). The giant larval salivary gland polytene chromosomes provide a unique and powerful tool for defining gene regulatory circuits in Drosophila. The fortuitous expression of DHR96 in the salivary glands of late third instar larvae provides an ideal opportunity to map its natural binding sites along the length of the giant polytene chromosomes. Since the cytological location of genes on the chromosomes has been well defined and correlated with the Drosophila genome sequence, DHR96 polytene binding sites can be matched to specific regions of DNA (Flybase Consortium, 2003 Nul Acid Res. 31:172-5). A similar genome-wide study of the in vivo binding sites of transcription factors has been conducted by using antibody stains of the polytene chromosomes, and these results have been used to predict direct regulatory targets which, in turn, have been confirmed at the molecular level. An advantage of this approach is that it is rapid, easy, and provides a complete survey of the genome. A clear shortcoming, however, is that this method only allows a resolution of several hundred kilobases of genomic DNA. To overcome this problem, the search can be focused on binding sites on candidate genes that encode detoxification enzymes. Polytene binding data can be cross-referenced with the results of the microarray studies described above to identify likely DHR96 gene targets. These genes can be scanned for clusters of DHR96 binding sites, as determined by the biochemical studies described above. Finally, in vivo binding of DHR96 to specific sequences by ChIP is determined, as described below.

ChIP has been widely used to identify in vivo binding sites for DNA binding proteins, in many different organisms (Weinmann et al. Methods 26:37-47, 2002). Moreover, ChIP protocols are available for cultured cells, intact tissues, Drosophila embryos, or Drosophila adults, facilitating the use of this method (Cavalli et al., Damjanovski et al., Schwartz et al.). Two third instar larval tissues can be focused on, the fat body and salivary glands, both of which contain high levels of nuclear DHR96 protein. Crosslinking is performed using 0.3% formaldehyde, chromatin is fragmented by sonication, and aliquots are flash frozen in liquid nitrogen for subsequent chromatin immunoprecipitation. Efficient sonication of chromatin is tested by gel electrophoresis of purified DNA. DHR96 antibodies are used as a means of purifying chromatin fragments that are crosslinked to DHR96 protein. Antibodies effectively immunoprecipitate purified DHR96, and thus can work well for chromatin IP. If the antibodies fail to work as desired, affinity-purified and tested DHR96 antibodies from the antisera of two other rabbits can be used. Alternatively, if all antibodies fail, ectopically expressed tagged DHR96 can be used for chromatin IP. PCR can then be used to assay for the enrichment of DNA sequences that encompass potential DHR96 binding sites, as determined by biochemical studies described above as well as our polytene chromosome binding data. Attention can also be paid to promoters that are regulated by DHR96 as determined by microarray studies. Finally, potential DHR96 binding sites can be tested that are identified by bioinformatics, as described below.

In parallel with the above studies that are aimed at defining the DNA binding specificity of DHR96, conserved potential regulatory sequences can be determined within co-expressed target genes identified by the microarray studies. The microarray experiments described above generate two gene lists for each compound tested—one list showing which genes change their level of expression in response to a xenobiotic compound in wild type animals, and a second list showing which of those genes require DHR96 for that regulatory response. These gene lists can be used to scan for clustered regulatory elements that are conserved between multiple co-regulated genes using several bioinformatic approaches. This effort can identify novel DHR96 binding sites in the genome. In addition, other conserved regulatory elements can be determined that expands the understanding of detoxification gene expression beyond DHR96.

Bioinformatics is a rapidly evolving area with a number of labs developing and improving algorithms for mapping and predicting transcription factor binding sites. One program to identify nuclear receptor binding sites is “cis-analyst” (http://rana.lbl.gov/cis-analyst/). This is a web-based visualization tool that scans a given genomic region for the presence of a specific binding site consensus sequence, allowing the user to establish a cutoff point for eliminating weak binding sites. It searches for sequences of a specified length that contain a minimum number of predicted binding sites, allowing the detection of binding site clusters. This provides an ideal computational tool to enhance for functional sites rather than orphan binding sites that one might encounter on a random basis. The program generates a readily analyzed visual output that depicts binding sites on the DNA, along with genome annotation (Berman et al. Proc Natl Acad Sci, 99:757-62, 2002). Cis-analyst has been used to identify novel clustered binding sites for five well characterized Drosophila transcription factors, and these new regulatory targets have been validated by in vivo studies in transgenic animals Matlnspector and Patch can also be used to look for binding sites of known transcription factors in Drosophila promoters of interest (http://www.gene-regulation.com/pub/programs.html), and Improbizer to scan for sequences that occur with an improbable frequency in a given segment of DNA (http://www.cse.ucsc.edu/˜kent/improbizer/improbizer.html). These or similar programs can be used to analyze the promoter sequences of co-regulated genes identified by the microarray studies.

In order to determine whether the sequences identified above are likely to have functional significance, it can be determined if they have been conserved through Drosophila evolution. Evolutionary conservation has been widely used as a means of parsing regulatory sequences to identify true functional elements. This is particularly powerful in Drosophila, where the genome sequences of eight different species is becoming available. The first such sequence, that of Drosophila pseudoobscura (which diverged from D. melanogaster ˜45 million years ago), was available earlier this year (http://www.hgsc.bcm.tmc.edu/projects/Drosophila/). This has now been supplemented with the ongoing genomic analysis of six other species, including Drosophila virilis, which diverged from D. melanogaster ˜60 million years ago (http://www.genome.gov/11008080; http://rana.lbl.gov/Drosophila/multipleflies.html). The cis-regulatory sequences can be analyzed from selected detoxification target genes using as many of these species as possible in order to determine whether DHR96 binding sites, or the binding sites of potential new transcriptional regulators, have been conserved through Drosophila evolution. Although confirmatory, this, is an important step in determining whether the sequences we identify by informatics are likely to be functional in vivo.

### 7. Example 7

### The Molecular Mechanisms of Detoxification Gene Expression

The functional significance of these elements using both biochemical and genetic approaches can be determined. Nuclear extracts are prepared from larval fat bodies using published protocols (Lehmann et al. EMBO J. 14:716-26, 1995; Antoniewski et al. Mol. Cell. Biol 14:4465-74, 1994; von Kalm et al. EMBO J. 13:3505-16, 1994). The choice of fat bodies derives from its functional equivalence to the mammalian liver as well as the abundant expression of DHR96 in this tissue. Sequences that encompass prospective DHR96 binding sites, or the binding sites of other potential regulators, are amplified by PCR and tested for their ability to be bound by factors in the fat body nuclear extracts. Protein binding to these fragments will be is monitored by electrophoretic mobility shift assays (EMSAs). The specificity of potential DHR96 interactions is determined by competition experiments using an oligonucleotide with an idealized DHR96 binding site, as well as by using DHR96 antibodies to supershift the complex. Antibodies directed against USP can be used to determine whether the binding complex also contains this potential heterodimer partner. Competition assays and antibody supershift experiments can be used to identify factors that bind to other conserved regulatory elements. The identity of some of these transcription factors, for example GAGA factor or C/EBP, should be predictable based on their DNA binding specificity (Lehmann et al., Park et al. DNA Cell Biol. 15:693-701, 2004). Other potential regulators can be found based on the sequences of oligonucleotides that efficiently compete for binding in nuclear extracts, and confirm this deduction by using appropriate antibodies for supershift studies. This approach has been used to identify ecdysone-regulated transcription factors that control glue gene transcription in Drosophila salivary glands as well as characterize ecdysone-inducible Fbp-1 transcription in fat bodies.

The above studies confirms the presence of functional DHR96 binding sites in target promoters as well as allows for the identification of other potential trans-acting regulators of detoxification gene expression. The corresponding sequences in the target promoters are disrupted by site-directed mutagenesis using PCR. The resultant mutated fragments are tested by DNA sequencing to ensure that only the desired base changes have occurred. These fragments are then be tested by EMSA to confirm that the mutations have disrupted binding to the corresponding transcription factor. The mutated fragments are then be used in combination with wild type sequences to reassemble target promoters for functional studies in transgenic animals.

Studies can also be conducted in transgenic animals as a means of determining the functional significance of specific transcription factor binding sites. 2-3 target promoters can be defined in the preceding specific aim, but can include other promoters to test specific hypotheses regarding possible transcription factor interactions that arise. Each of the target promoters can be fused to a lacZ reporter gene in the P element transformation vector pCaSpeR-AUG-βgal (Thummel et al. Dros. Info. Services 71:150, 1992). These are introduced into the fly genome using conventional methods and multiple independent insertions are isolated to control against the effects of flanking sequences on reporter gene expression. Each promoter-lacZ fusion transgene is crossed into wild type and DHR96 mutant genetic backgrounds to establish permanent stocks. These animals are exposed to either regular food or food supplemented with a xenobiotic, after which dissected tissues are tested for β-galactosidase expression using X-gal staining. Responses to phenobarbital can be testedbased on earlier studies which showed that several hundred base pairs of the Cyp6a2 or Cyp6a8 promoter is sufficient to mediate phenobarbital-inducible transcription of a reporter gene in transgenic wild type Drosophila. Little or no β-galactosidase expression can be seen in tissues dissected from untreated wild type animals, and high levels of β-galactosidase expression in tissues from wild type animals exposed to phenobarbital. X-gal assays are performed on tissues dissected from DHR96 mutant animals.

The wild type promoter sequences in the transgene vectors can be replaced with the mutated fragments described above, and introduce these P elements into the genome of both wild type and DHR96 mutant animals. As before, multiple independent transgenic lines can be established to control against the effects of flanking sequences on reporter gene expression. The regulation conferred by the mutant promoter fragment will bise tested in trangenic animals after exposure to phenobarbital or other xenobiotics, depending on our earlier studies. If a reduction or absence of lacZ transcription is seen, then the regulatory interaction disrupted by the promoter mutation is of functional significance. Alternatively, no effect on lacZ transcription indicates that the binding site is not essential for proper promoter regulation. In this case, additional transgenic lines will be is established that carry multiple binding site mutations for that transcription factor, to determine whether they act in a redundant manner. Similarly, the contributions of individual binding sites are tested in other transgenic lines.

The effects of mutations in DHR96 binding sites should confirm the studies of the wild type transgene in DHR96 mutant animals. That is, if the wild type promoter is unable to respond to a xenobiotic in a DHR96 mutant background, then that same promoter carrying mutated DHR96 binding sites should show defective xenobiotic responses in wild type animals. A similar approach can be used to test the functional significance of other transcription factor binding sites, crossing wild type promoter-lacZ fusion transgenes into stocks that carry mutations in putative trans-acting regulators, combined with studies of promoter transgenes that carry mutations in the corresponding binding sites. Such a demonstration of both cis and trans effects can be taken as a good indication that the corresponding transcription factor is involved in the observed regulatory interaction. Methods are available that allow us to create clones of mutant tissue, so that the effects of otherwise lethal transcription factor mutations can be studied. Taken together, these studies of wild type and mutated promoter-lacZ transgenes should allow for the decoding of the mechanisms of detoxification gene expression. It can be determined which binding sites are critical for the activity of a specific detoxification gene promoter, and which binding sites mediate xenobiotic-inducible transcription. In addition, it can be determined which transcription factors act through these sequences as well as how these transcription factors might interact to control the xenobiotic response.

Disclosed are methods for screening for the presence of xenobiotic receptor ligands using the constructs and methods disclosed herein, such as those for the GAL4-DHR96 fusions.

### G. REFERENCES

Boyd, L., O'Toole, E. and Thummel, C. S. (1991). Patterns of E74A RNA and protein expression at the onset of metamorphosis in Drosophila. Development 112, 981-995.
Kozlova, T. and Thummel, C. S. (2002). Spatial patterns of ecdysteroid receptor activation during the onset of Drosophila metamorphosis. Development 129, 1739-1750.
Kozlova, T., and C. S. Thummel (2003) Methods to characterize Drosophila nuclear receptor activation and function in vivo. In: “Methods in Enzymology. Nuclear Receptors, Vol. 364 (Russell, D. W., and Mangelsdorf, D. J., eds.), Academic Press, New York, pp. 475-490.).
Rong, Y. K., Titen, S. W., Xie, H. B., Golic, M. M., Bastiani, M., Bandyopadhyay, P., Olivera, B. M., Brodsky, M., Rubin, G. M., and Golic, K. G. (2002). Targeted mutagenesis by homologous recombination in D. melanogaster. Genes and Development 16, 1568-1581.
Daborn, P. J., Yen, J. L., Bogwitz, M. R., Le Goff, G., Feil, E., Jeffers, S., Tijet, N., Perry, T., Heckel, D., Batterham, P., et al. (2002). A single p450 allele associated with insecticide resistance in Drosophila. Science 297, 2253-2256.
Danielson, P. B., Foster, J. L., McMahill, M. M., Smith, M. K., and Fogleman, J. C. (1998). Induction by alkaloids and phenobarbital of Family 4 Cytochrome P450s in Drosophila: evidence for involvement in host plant utilization. Mol Gen Genet. 259, 54-59.
Danielson, P. B., Macintyre, R. J., and Fogleman, J. C. (1997). Molecular cloning of a family of xenobiotic-inducible drosophilid cytochrome p450s: evidence for involvement in host-plant allelochemical resistance. Proc Natl Acad Sci USA 94, 10797-10802.
Fogleman, J. C. (2000). Response of Drosophila melanogaster to selection for P450-mediated resistance to isoquinoline alkaloids. Chem Biol Interact 125, 93-105.
Francis, G. A., Fayard, E., Picard, F., and Auwerx, J. (2003). Nuclear receptors and the control of metabolism. Annu Rev Physiol 65, 261-311.
Kliewer, S. A. (2003). The nuclear pregnane X receptor regulates xenobiotic detoxification. J Nutr 133, 2444S-2447S.
Li, C., and Wong, W. H. (2001). Model-based analysis of oligonucleotide arrays: model validation, design issues and standard error application. Genome Biol 2, RESEARCH0032.
Lindblom, T. H., Pierce, G. J., and Sluder, A. E. (2001). A C. elegans orphan nuclear receptor contributes to xenobiotic resistance. Curr Biol 11, 864-868.
Maglich, J. M., Stoltz, C. M., Goodwin, B., Hawkins-Brown, D., Moore, J. T., and Kliewer, S. A. (2002). Nuclear pregnane x receptor and constitutive androstane receptor regulate overlapping but distinct sets of genes involved in xenobiotic detoxification. Mol Pharmacol 62, 638-646.
Makishima, M., Lu, T. T., Xie, W., Whitfield, G. K., Domoto, H., Evans, R. M., Haussler, M. R., and Mangelsdorf, D. J. (2002). Vitamin D receptor as an intestinal bile acid sensor. Science 296, 1313-1316.
Mangelsdorf, D. J., Thummel, C., Beato, M., Herrlich, P., Schutz, G., Umesono, K., Blumberg, B., Kastner, P., Mark, M., Chambon, P., and et al. (1995). The nuclear receptor superfamily: the second decade. Cell 83, 835-839.
Maurel, P. (1996). Cytochromes P450: Metabolic and Toxicological Aspects. In Cytochromes P450: Metabolic and Toxicological Aspects, C. Ioannnides, ed. (Boca Raton, CRC Press), pp. 241-270.
Ranson, H., Claudianos, C., Ortelli, F., Abgrall, C., Hemingway, J., Sharakhova, M. V., Unger, M. F., Collins, F. H., and Feyereisen, R. (2002). Evolution of supergene families associated with insecticide resistance. Science 298, 179-181.
Rong, Y. S., and Golic, K. G. (2000). Gene targeting by homologous recombination in Drosophila. Science 288, 2013-2018.
Rong, Y. S., Titen, S. W., Xie, H. B., Golic, M. M., Bastiani, M., Bandyopadhyay, P., Olivera, B. M., Brodsky, M., Rubin, G. M., and Golic, K. G. (2002). Targeted mutagenesis by homologous recombination in D. melanogaster. Genes Dev 16, 1568-1581.
Robinson-Rechavi M, Escriva Garcia H, Laudet V., The nuclear receptor superfamily, J Cell Sci. 2003 Feb. 15; 116(Pt 4):585-6
Tontonoz, P., and Mangelsdorf, D. J. (2003). Liver X receptor signaling pathways in cardiovascular disease. Mol Endocrinol 17, 985-993.
Wilson, T. G. (2001). Resistance of Drosophila to toxins. Annu Rev Entomol 46, 545-571.

### H. SEQUENCES

```
1. SEQ ID NO: 1
Accession No. NM_130611 Drosophila melanogaster
CG16902-PA
MTLSRGPYSELDKMSLFQDLKLKRRKIDSRCSSDGESIADTSTSSPDLLA
PMSPKLCDSGSAGASLGASLPLPLALPLPMALPLPMSLPLPLTAASSAVT
VSLAAVVAAVAETGGAGAGGAGTAVTASGAGPCVSTSSTTAAAATSSTSS
LSSSSSSSSSTSSSTSSASPTAGASSTATCPASSSSSSGNGSGGKSGSIK
QEHTEIHSSSSAISAAAASTVMSPPPAEATRSSPATPEGGGPAGDGSGAT
GGGNTSGGSTAGVAINEHQNNGNGSGGSSRASPDSLEEKPSTTTTTGRPT
LTPTNGVLSSASAGTGISTGSSAKLSEAGMSVIRSVKEERLLNVSSKMLV
FHQQREQETKAVAAAAAAAAAGHVTVLVTPSRIKSEPPPPASPSSTSSTQ
RERERERDRERDRERERERDRDREREREQSISSSQQHLSRVSASPPTQLS
HGSLGPNIVQTHHLHQQLTQPLTLRKSSPPTEHLLSQSMQHLTQQQAIHL
HHLLGQQQQQQQASHPQQQQQQQHSPHSLVRVKKEPNVGQRLHLSPHHQQ
QSPLLQHHQQQQQQQQQQQQHLHQQQQQQQHHQQQPQALALMHPASLALR
NSNRDAAILFRVKSEVHQQVAAGLPHLMQSAGGAAAAAAAAVAAQRMVCF
SNARINGVKPEVIGGPLGNLRPVGVGGGNGSGSVQCPSPHPSSSSSSSQL
SPQTPSQTPPRGTPTVIMGESCGVRTMVWGYEPPPPSAGQSHGQHPQQQQ
QSPHHQPQQQQQQQQQQSQQQQQQQQQQSLGQQQHCLSSPSAGSLTPSSS
SGGGSVSGGGVGGPLTPSSVAPQNNEEAAQLLLSLGQTRIQDMRSRPHPF
RTPHALNMERLWAGDYSQLPPGQLQALNLSAQQQQWGSSNSTGLGGVGGG
MGGRNLEAPHEPTDEDEQPLVCMICEDKATGLHYGIITCEGCKGFFKRTV
QNRRVYTCVADGTCEITKAQRNRCQYCRFKKCIEQGMYLQAVREDRMPGG
RNSGAVYNLYKVKYKKHKKTNQKQQQQAAQQQQQQAAAQQQHQQQQQHQQ
HQQHQQQQLHSPLHHHHHQGHQSHHAQQQHHPQLSPHHLLSPQQQQLAAA
VAAAAQHQQQQQQQQQQQQQAKLMGGVVDMKPMFLGPALKPELLQAPPMH
SPAQQQQQQQQQQQQQQASPHLSLSSPHQQQQQQQGQHQNHHQQQGGGGG
GAGGGAQLPPHLVNGTILKTALTNPSEIVHLRLDSAVSSSKDRQISYEHA
LGMIQTLIDCDAMEDIATLPHFSEFLEDKSEISEKLCNIGDSIVHKLVSW
TKKLPFYLEIPVEIHTKLLTDKWHEILILTTAAYQALHGKRRGEGGGSRH
GSPASTPLSTPTGTPLSTPIPSPAQPLHKDDPEFVSEVNSHLSTLQTCLT
TLMGQPIAMEQLKLDVGHMVDKMTQITIMFRRIKLKMEEYVCLKVYILLN
KGTWFDLQNPFIQGSCYLLVRFVNPAEVELESIQERYVQVLRSYLQNSSP
QNPQARLSELLSHIPEIQAAASLLLESKMFYVPFVLNSASIRORIGIN
2. SEQ ID NO: 2
Accession No. NM_130611 Drosophila melanogaster
CG1 6902-PA
      1 atgacactga gccgtggccc gtacagcgag ctcgataaaa tgagcctttt tcaagacctc
    61 aaactcaaac ggcgcaaaat cgattcgcga tgcagcagtg acggcgagtc catagcggac
  121 acgtccacct cgtcgccgga cctgctggcg cccatgtcgc cgaagctctg cgacagcggc
  181 tcggcggggg cgtcgctggg ggcatcgctg cccctgccgc tggccctgcc cctgccaatg
  241 gccctgccae tgcccatgtc gctgcccctg cccctcacgg cggcatcttc ggcggtcacc
  301 gtttcgctgg cagcggtcgt ggccgcggtg gccgagacgg gtggcgcggg cgcgggagga
  361 gctgggacag cagtaacagc gtcgggagca ggaccatgcg tctccacgtc gtctacgacg
  421 gcagcggcag ccacatcctc gacctcctcg ctctcgtcct cctcctcttc gtcatcctcc
  481 acgtcctcca gcacttcctc cgcctcgccg acagctggag cctcctccac ggccacctgc
  541 cccgccagca gcagcagcag cagtggaaac ggaagtgggg gcaaaagtgg tagcatcaag
  601 caggagcaca cggagataca ctcgtcgagc agtgcgattt cggcggccgc cgcctcaacg
  661 gtgatgtcac cgccgcccgc tgaggcgacg agatccagtc cagccacgcc cgagggaggc
  721 ggaccagctg gcgacggaag tggagcaacg ggaggcggaa acacgagcgg cggatcaacg
  781 gctggagtgg ccattaatga acaccaaaac aatggcaatg gcagcggcgg gagcagtcga
  841 gcctctcccg attcgctgga agagaagccc tctaccacaa cgaccacagg tcgtccaacg
  901 ctcacgccca cgaatggggt gctgtcctcc gcctcggcgg gcacggggat ttccacagga
  961 agcagcgcca agctgagcga ggctggtatg agtgtgatac ggtccgtgaa ggaggagcgc
1021 ttgctcaacg tatccagcaa gatgctggtg ttccatcagc agcgggagca agagaccaaa
1081 gcagtggcgg ctgcagcagc agcagcagcg gcgggccatg tgacggttct agtgacgcca
1141 tcgcgcatca aatcggagcc accgccgccg gcttcaccct cctctacatc cagcacacaa
1201 agggaaaggg aacgggaacg cgatcgagag agggatcgcg aaagggaacg cgagcgggac
1261 cgggaccggg aacgggaacg ggaacagtcc atcagctcct cgcagcagca cctaagtcgg
1321 gtctccgcca gtccacccac tcagctgtcc cacggcagcc tgggacccaa cattgtgcag
1381 acgcaccatc ttcaccagca actcacacag ccgctgacgc tgcgcaagag cagcccgccc
1441 acagagcacc tgctcagtca gtccatgcaa catctcacac agcagcaggc gatccacctg
1501 catcacctac ttggccagca gcagcagcag cagcaggcgt cgcatcccca gcagcaacag
1561 cagcagcaac actcgcccca ctccctggtg cgggtgaaaa aggaaccgaa tgttggtcag
1621 cggcacttat cgccgcatca ccaacaacag tcgccactcc tgcagcacca ccaacagcag
1681 cagcagcagc aacaacaaca gcaacagcat ctgcatcagc aacagcaaca gcagcagcat
1741 caccagcagc agccccaggc actggccctg atgcatccgg cttccctggc gctaaggaac
1801 agcaatcggg atgcggccat tctgtttcgg gtgaagagcg aagtgcacca gcaggtggcc
1861 gccgggctgc cgcatctgat gcagtccgct ggtggggcag cggccgccgc cgcagcagct
1921 gtggccgctc agcgaatggt atgcttcagc aatgccagga tcaatggcgt taagccggag
1981 gtgattggag gaccgctggg caacctgcgg cccgtgggcg tcggtggcgg aaacggaagt
2041 ggctccgtgc agtgcccctc gccgcatcca tcctcctcgt cgtcatcctc gcagctgtcg
2101 ccgcagacgc cctcccagac gccgccccga ggcacgccca ccgtcataat gggcgagagc
2161 tgcggggtgc gcaccatggt ctggggctac gagcctccgc caccctcggc gggccagtcc
2221 cacggccagc acccgcaaca gcaacagcag tcgccccacc accagccgca acaacaacag
2281 cagcagcaac aacagcagtc gcagcagcaa cagcaacagc agcagcaaca gtcgctgggc
2341 cagcagcagc actgcctctc ctcgccgtcg gcgggatcgc tgacgccctc ctcttcgtcc
2401 ggcggtggtt cggtatctgg cggcggagtg ggcggaccac tcacaccctc ctcggtggcg
2461 ccgcagaata acgaggaggc cgcccaactc ctgctctccc tgggacagac acgcatccag
2521 gacatgagat cacggccaca ccccttccgc acaccgcacg cccttaatat ggagcggctg
2581 tgggcgggag actactcgca attgccgccc ggccagctgc aggctctgaa tctcagtgcc
2641 caacagcagc agtggggcag cagcaactcc acgggtcttg gtggcgtagg cggcggcatg
2701 ggcggacgca acctggaggc gccgcacgag ccgaccgacg aggacgaaca gccgctcgtt
2761 tgcatgatct gcgaggacaa ggccaccggc ctgcactacg gcatcatcac ctgcgagggg
2821 tgcaagggct tcttcaagcg gacggtgcag aaccgacgag tctacacctg cgtggcggac
2881 ggcacctgcg agataaccaa agcacagcgc aaccgttgtc agtattgtcg atttaagaag
2941 tgcatcgagc agggcatggt gctgcaagcc gttcgcgagg atcgcatgcc gggcggtcgc
3001 aacagtggcg ccgtctacaa tttgtacaag gtgaagtaca agaagcacaa gaagaccaat
3061 cagaagcagc agcagcaggc cgcccagcag cagcagcagc aggcggcggc gcagcagcag
3121 caccagcaac agcagcagca tcaacagcac cagcaacatc agcaacagca gttgcactcg
3181 ccgctccacc atcaccacca ccagggccac cagtcgcacc acgcgcagca gcagcaccac
3241 ccacagctgt cgccgcacca cctgctgtcg ccgcagcagc agcaacttgc cgccgcggtg
3301 gcagcagctg cgcagcacca acagcaacag caacaacagc agcaacagca gcagcaggcc
3361 aagctgatgg gcggcgtggt ggacatgaag cccatgttcc tcggccccgc tttgaagccg
3421 gagttgctgc aagcaccccc catgcacagt ccggcccagc aacaacaaca gcagcagcag
3481 cagcagcagc aacagcaggc ctcgccgcat ctctcgctta gctcaccgca ccagcagcag
3541 cagcagcagc agggacagca ccaaaaccac caccagcaac aaggtggggg tggcggagga
3601 gctggtggag gagctcaact gccgccgcac ctggtgaacg gaacgatact gaagacggcc
3661 ctaaccaatc ccagcgagat tgtacatctg cgccaccgcc tcgactcggc ggtcagttcg
3721 tccaaggacc gacagatctc gtacgagcac gccttaggca tgatccagac actgatcgac
3781 tgcgacgcga tggaggacat agccacactg ccgcacttca gcgagttcct tgaggacaag
3841 tcggagatta gcgagaaact gtgcaacatc ggcgattcca tagtccacaa gctggtgtcg
3901 tggacaaaaa agttgccctt ctacctggag atcccggtgg agatacatac caaactactg
3961 acggacaagt ggcacgagat ccttatcctg accacggccg cctaccaggc gttgcatggc
4021 aagcggcgtg gcgagggagg aggcagcagg catggttcgc cggcgtcaac gccactgagc
4081 acgcccactg gtacgccgtt gagcacaccg ataccctcgc ccgcccagcc actgcacaag
4141 gacgacccgg agtttgtcag cgaggtgaac tcgcacctga gcacactgca aacctgcttg
4201 accacgctaa tgggccagcc gatagcgatg gagcagctga agctggacgt cgggcacatg
4261 gtggacaaga tgacccagat caccatcatg ttccggcgaa tcaagctcaa gatggaggag
4321 tacgtctgcc tgaaggttta catactgcta aacaaaggta cgtggttcga tttgcaaaac
4381 ccattcatac agtgctcatg ttaccttctc gttcgttttg taaatccagc agaagtggaa
4441 ctggagagca tccaggagcg gtacgtccag gtgctgcgct cctacctgca aaactcctcg
4501 ccgcagaatc cgcaggcgag gctcagtgaa ctgctctccc acataccaga gatccaggct
4561 gcggctagcc tgctgctcga gagcaagatg ttctatgtgc ccttcgtgct caactcggcg
4621 agcataaggtag
3. SEQ ID NO: 3
Accession No. NM_168775 Drosophila melanogaster ftz
transcription factor 1 CG4059-PA
MLLEMDQQQATVQFISSLNISPFSMQLEQQQQPSSPALAAGGNSSNNAAS
GSNNNSASGNNTSSSSNNNNNNNNDNDAHLTKFEHEYNAYTLQLAGGGGS
GSGNQQHHSNHSNHGNHHQQQQQQQQQQQQHQQQQQEHYQQQQQQNIANN
ANQFNSSSYSYIYNFDSQYIFPTGYQDTTSSHSQQSGGGGGGGGGNLLNG
SSGGSSAGGGYMLLPQAASSSGNNGNFNAGHMSSGSVGNGSGGAGNGGAG
GNSGPGNPMGQTSATPGHGGEEVIDFKHLFEELCPVCGDKVSGYHYGLLT
CESCKGFFKRTVQNKKVYTCVAERSCHIDKTQRKRCPYCRFQKCLEVGMK
LEAVRADRMRGGRNKFGPMYKRDRARKLQVMRQRQLALQALRNSMGPDIK
PTPISPGYQQAYPNMIKQEIQIPQVSSLTQSPDSSPSPIAIALGQVNAST
GGVTPMNAGTGGSGGGGLNGPSSVGNGNSSNGSSNGNNNSSTGNGTSGGG
GGNNAGGGGGGTNSNDGLHRNGGNGNSSCHBAGIGSLQNTADSKLCFDSG
THPSSTADALIEPLRVSPMIREFVQSIDDREWQTQLFALLQKQTYNQVEV
DLFELMCKVLDQNLFSQVDWARNTVFFKDLKVDDQMKLLQHSWSDMLVLD
HLHHRIHNGLPDETQLNNGQVFNLMSLGLLGVPQLGDYFNELQNKLQDLK
FDMGDYVCMKFLILLNPSVRGIVNRKTVSEGHDNVQAALLDYTLTCYPSV
NDKFRGLVNILPEIHAMAVRGEDHYTKHCAGSAPTQTLLMEMLHAKRKG
4. SEQ ID NO: 4
Accession No. NM_168775 Drosophila melanogaster ftz
transcription factor 1 CG4059-PA
      1 ctacgcaaaa taaaacgtac atgaaatgtt attagaaatg gatcagcaac aggcgaccgt
    61 acagtttata tcgtcgctga atatatcgcc gttcagcatg cagctggagc agcagcagca
  121 gccctccagt cccgctctgg ccgccggtgg caacagcagc aacaacgcgg ccagcggtag
  181 caacaacaac agcgccagcg gcaacaacac cagcagcagc agcaacaaca acaacaacaa
  241 taacaacgac aatgatgcac acgttctaac gaaattcgag cacgaataca atgcctacac
  301 gttgcagttg gccggaggcg gtgggagtgg cagcggcaat cagcagcacc acagcaacca
  361 cagcaaccac ggcaaccacc accagcagca gcagcaacaa cagcaacagc agcagcaaca
  421 tcagcagcag cagcaagaac actaccagca gcaacagcaa cagaatatcg ccaacaatgc
  481 caatcaattc aactcctcgt cctactcgta tatatacaat ttcgattcac agtatatatt
  541 cccgacaggc taccaggaca ccacctcctc acactcgcaa cagagcggag gaggcggtgg
  601 cggcggcggt ggcaacctgc taaacggcag ctccggcggc agctccgccg gcggtggcta
  661 catgctgctc ccccaggcgg ccagctccag tggcaataat ggcaatccga atgccggcca
  721 catgtcctcc ggttccgtgg gcaatggcag cggaggcgct ggcaatggcg gagcgggcgg
  781 caactccggt cccggcaatc ccatgggcgg tacgagcgcc acgccgggac acggcggcga
  841 ggtgatcgac ttcaagcacc tgttcgagga gctttgcccc gtgtgtggcg acaaggtgag
  901 cggctaccac tacggcctgc tcacctgcga gtcctgcaag ggattcttca agcgcaccgt
  961 gcagaacaag aaggtctaca cctgcgtggc ggagcggtcg tgccacatcg acaagacgca
1021 gcgcaagcgg tgtccctact gccgattcca gaagtgcctc gaggtgggca tgaagctaga
1081 ggctgttcga gcggatagaa tgcgtggtgg acgcaacaaa ttcggaccca tgtacaaacg
1141 ggatcgcgcg cggaagttgc aagtgatgcg gcagcggcag ttggcgctgc aagcgctgcg
1201 caactcgatg ggtccggaca tcaagccaac gccgatctcg ccgggctacc agcaagcata
1261 tccaaatatg aacattaagc aggaaattca aatacctcag gtatcctcac tcacccaatc
1321 tccggactcg tcgcccagcc ccatagcaat tgcgttggga caggtgaacg cgagcacggg
1381 cggtgttata gccacgccca tgaacgccgg cactggcggc agtgggggcg gtggtctgaa
1441 cggaccaagt tccgtgggca acggcaatag cagcaacggc agcagcaacg gcaacaacaa
1501 cagcagcacg ggcaacggaa cgtccggagg aggaggtggc aataatgcgg gcggcggagg
1561 aggaggaacc aattccaacg atggcctgca tcgcaacggc ggcaatggca acagcagttg
1621 ccacgaggct ggaataggat ctctgcagaa cacggccgac tcgaaattgt gcttcgattc
1681 tggcacacat ccatcgagca cagccgacgc gctaatcgag ccattaagag tctcaccgat
1741 gattcgtgaa tttgtgcaat ctattgacga tcgggaatgg cagacgcaac tgtttgccct
1801 gctgcagaag caaacctaca accaggtgga agtggatctc ttcgagctga tgtgcaaagt
1861 gctcgaccag aatttgttct cgcaagtaga ctgggcacgg aacaccgtct tcttcaagga
1921 tctgaaggtc gacgaccaaa tgaagctgct gcagcattcc tggtcggaca tgcttgttct
1981 ggatcacctg catcatcgaa tccataacgg cctgcccgac gagacgcaac tgaacaatgg
2041 tcaggtgttc aatctgatga gtctgggttt gttgggagtg ccacagctgg gcgattactt
2101 caacgagctg cagaacaagc tgcaggacct gaaattcgat atgggcgact atgtctgcat
2161 gaaattccta atcctgttga atccaagtgt acggggtatt gtcaaccgga agaccgtctc
2221 cgagggacat gataatgtgc aagccgcttt gctggactac accctcacct gctatccgtc
2281 agtgaatgac aaattcagag ggctagttaa catcttaccg gaaatccatg ccatggccgt
2341 tcgcggcgag gatcacctgt acaccaagca ctgtgccggc agtgcgccca cccaaacgct
2401 gctcatggag atgctgcacg ccaagcgcaa gggatagagg ccgggagaac gtgacacgga
2461 atacttaatc atttatgaaa tgtaaataac aaggcgggaa ggccctcggg gcaaccgggt
2521 catggaaggc gaacgaagga tacagcagaa ttccgtatta tgaatatggg aatgcatcat
2581 cactactacc accaactatc acacctatac acacacatgc acacatttgt tgattcaatg
2641 ttaattatta ttacgtttac ggttaggtct agtttacgtt taactaatta attaatttgt
2701 cttaaattaa ttcgtgtttt atttgtagtc cctgataaag caattttaaa acacttgaac
2761 ctaaacgaga atatgtagta gatgtatgga tttaaattta aatacggcaa ggagaaacac
2821 acttttttag gcattacaaa acaaaagaag catgagaaat tttattttta tatacctata
2881 tgaatacgat acttatggat acaaatctat atatattttt atgtaaattg gcgtactttt
2941 agcgtcctac atatttttta attagaattt ggttatacta tagttttgaa attagtatcg
3001 ttcccacttg aagatcgatt cttgtatttt tttgcgccaa gtgtcttgca tagtatttgc
3061 gtctaatcta atggcaacaa aaaaaatatt ggaaaatcca tacaaagaaa atgaaaacaa
3121 agcaaattta ggtgttcatg gtatgaatgt atgtgtatat tataattgta atttcatcta
3181 agtgtaagaa aacaatgcaa acaactacct acaacaagat aatgaagagc aagaaattat
3241 ataaattaat aaaggtcgtg ttaaaaact
5. SEQ ID NO: 5
Accession No. NM_176123 Drosophila melanogaster
Hormone receptor-like in 46 CG33183-PA
MYTQRMFDMWSSVTSKLEAHANNLGQSNVQSPAGQNNSSGSIKAQIEIIP
CKVCGDKSSGVHYGVITCEGCKGFFRRSQSSVVNYQCPRNKQCVVDRVNR
NRCQYCRLQKCLKLGMSRDAVKFGRMSKKQREKVEDEVRFHRAQMRAQSD
AAPDSSVYDTQTPSSSDQLHHNNYNSYSGGYSNNEVGYGSPYGYSASVTP
QQTMQYDISADYYDSTTYEPRSTIIDPEFISHADGDINDVLIKTLAEAHA
NTNTKLEAVHDMFRKQPDVSRILYYKNLGQEELWLDCAEKLTQMIQNIIE
FAKLIPGFMRLSQDDQILLLKTGSFELAIVRMSRLLDLSQNAVLYGDVML
PQEAFYTSDSEEMRLVSRIFQTAKSIAELKLTETELALYQSLVLLWPERN
GVRGNTEIQRLFNLSMNAIRQELETNHAPLKGDVTVLDTLLNNIPNFRDI
SILHMESLSKFKLQHPNVYFPALYKELFSIDSQQDLT
6. SEQ ID NO: 6
Accession No. NM_176123 Drosophila melanogaster
Hormone receptor-like in 46 CG33183-PA
      1 gaattcattc aactgcaaag agcagccaaa ttgcgcatac gccgcgtatg gccgtcggtg
    61 tgagtgcccg tgttcatcag cggttgcatc aactgatacc aagtgtacat aactacagct
  121 acaattgcaa ctatttcacc aatcaacggc agcggcaaca acatcagcaa cagcaccggc
  181 aaacgtttga aacgtcacca aagcttcgca tttcccacta ataattatgt atacgcaacg
  241 tatgtttgac atgtggagca gcgtcacttc gaaactggaa gcacacgcaa acaatctcgg
  301 tcaaagcaac gtccaatcgc cggcgggaca aaacaactcc agcggttcca ttaaagctca
  361 aattgagata attccatgca aagtctgcgg cgacaagtca tccggcgtgc attacggagt
  421 gatcacctgc gagggctgca agggattctt tcgaagatcg cagagctccg tggtcaacta
  481 ccagtgtccg cgcaacaagc aatgtgtggt ggaccgtgtt aatcgcaacc gatgtcaata
  541 ttgtagactg caaaagtgcc taaaactggg aatgagccgt gatgctgtaa agttcggcag
  601 gatgtccaag aagcagcgcg agaaggtcga ggacgaggta cgcttccatc gggcccagat
  661 gcgggcacaa agcgacgcgg caccggatag ctccgtatac gacacacaga cgccctcgag
  721 cagcgaccag ctgcatcaca acaattacaa cagctacagc ggcggctact ccaacaacga
  781 ggtgggctac ggcagtccct acggatactc ggcctccgtg acgccacagc agaccatgca
  841 gtacgacatc tcggcggact acgtggacag caccacctac gagccgcgca gtacaataat
  901 cgatcccgaa tttattagtc acgcggatgg cgatatcaac gatgtgctga tcaagacgct
  961 ggcggaggcg catgccaaca caaataccaa actggaagct gtgcacgaca tgttccgaaa
1021 gcagccggat gtgtcgcgca ttctctacta caagaatctg ggccaagagg aactctggct
1081 ggactgcgcc gagaagctta cacaaatgat acagaacata atcgaatttg ctaagctcat
1141 accgggattc atgcgcctaa gtcaggacga tcagatatta ctgctgaaga cgggctcctt
1201 tgagctggcg attgttcgca tgtccagact gcttgatctc tcacagaacg cggttctcta
1261 cggcgacgtg atgctgcccc aggaggcgtt ctacacatcc gactcggaag agatgcgtct
1321 ggtgtcgcgc atcttccaaa cggccaagtc gatagccgaa ctcaaactga ctgaaaccga
1381 actggcgctg tatcagagct tagtgctgct ctggccagaa cgcaatggag tgcgtggtaa
1441 tacggaaata cagaggcttt tcaatctgag catgaatgcg atccggcagg agctggaaac
1501 gaatcatgcg ccgctcaagg gcgatgtcac cgtgctggac acactgctga acaatatacc
1561 caatttccgc gatatttcca tcttgcacat ggaatcgctg agcaagttca agctgcagca
1621 cccgaatgtc gtttttccgg cgctgtacaa ggagctgttc tcgatagatt cgcagcagga
1681 cctgacataa caagagcagc agccgttcct ggagacgacc gcggacgatg ttgccgagga
1741 tgcggctgcc gccggatgtg tcctgccgcc ggtggcgccc cctgccgggc agcaaccagc
1801 gctgctcgag gactgagggc cgcaggatgt ggcaacaata attatttgag taaacactgc
1861 actgcgcatg cagcagatac aagaacttta tcatgattta agctagcata caaccaagga
1921 tgtgatcctc gccaaggact cacttaaaaa gaactctatc tatatacata tatatattat
1981 atatgacaga gcggatgacg caaagggaag ggaaaatatt tcaaaaatat tgttaactca
2041 gttaagactt ttgcttcgta gagaaccgaa accgaaaccg attgcatttc gagcaagggg
2101 catcaaactg attttcgagg ttatactata catatataca cacaaacaca cacacacaca
2161 tatatatata tgtaacttcc aaactttcat atcctggccc gagcagatca gatcgtctaa
2221 gtacttaaaa ccaagcgaaa ttctctacac cgcacaaccc aggacccgta gaccccaata
2281 attcagttcg gttagtgtta accccagaaa gcccgattcc gatcccgcct aggttgtctt
2341 tgccttacgt tgtaactaaa gtatgtgtat tatatataca gcaaatgtat gtataactat
2401 gtcgtatcgg ttatatgcct aacaacatta ttttttgtaa acaacaaaat cgaatatctc
2461 ggaaaatgtg ttcttataat tatattgatt aatgcaatta caatatattt acaatttacc
2521 gttacgtttt tacattatac ataagacgca agagaaggaa acggaagttt aaggattaga
2581 aagctgaata agaaaaggct taaggacgag ctgagtagca gttaaagtga gcgagaaatc
2641 gaatgaatac cagaaaattt caagcaagca cataaaagta tgcaatattt tgtttaaaaa
2701 caacttttta ttagtttctt aaatataaca taattacgta catacacaca cgtatatata
2761 gggctatata tatctatata tatatatata tacatgatag acaaatccca atccggttcc
2821 aaggtttagt aaaaataaag agaaataaaa cgaaaaacaa aaacttttga tatgaaatcc
2881 tacgcataat taacaacttt tattgtttct aagacttaaa cttaattaaa atggaaacca
2941 aaacagactg acggaccgac cccgacagca tgccacgccc tcccccgccc caccctccac
3001 agatcctggc agaaatttca aaggagtttg atacacaaat cgagaaaaga aattttcaaa
3061 aaaataatat aaagacaagc aaacggcgac ttttttggtt gatacatttg aaaagaatat
3121 acaattaaat atctgactga ctatacaaag acgttacaca cacgcataca catacacaca
3181 catacacgca tacacacaca gcttacgata cataaattag ttaaacttag agtaaacaaa
3241 caacaacaaa cacattggat agtaggtgat aattggtgtg tcttaaataa accttaaccc
3301 ctccccgacc cccgcccact tgcttaatac ccaacgcccc aaaaagcccc acatttctac
3361 taaatgaaaa gcttaatcaa aacttttttg aaattattca agtgaaaatt tcagcaggca
3421 ggcataaata ttaattaaca ttaattatag caaggaaact tataaataaa atgtatacaa
3481 caaaactaca aaaattaaat aaattacatt ttgcaaattc cacaaaaaat aaaacatgat
3541 tttgcaaatt cacttaaaat cctttccctg aatccaagca aaaatattta cactagctta
3601 catagaactg ggacgaggac atgaatattt caattgagaa aaaaatctat gttaatgtaa
3661 tcgatcgatt tggacatatt taagttcgac atttttggcc ttacaaaaca aaaaacaaaa
3721 agaagaaacc taaagtactt tatatatata caaaccatat atacaatata gagaatacaa
3781 aactagtttt aatttataca aagcaaggga gcagctttca aactcaaaac aaaaatatcc
3841 ccgaaaaaaa caacaacttt gttaaaaact gcgcataata aagaaaataa taaacaaagt
3901 taatctataa tataaattga agttaagttg atttgagcgg tcgacaacaa gaacataaat
3961 gtatctttaa atgatatatg tattgttaaa tttgtatgct aagtttttag aaaggttaca
4021 tttttaaaga ataataacaa aagatcgcga actcgacaag gtgtaaaatg agtacattta
4081 aattaaaatt tagcatatat aatgcataaa tattatgtta cgatatttac atttatataa
4141 aacaaaacaa aaacactaaa gaaaaccgaa aaaacagaag tcccatatta aaaatgaaat
4201 aaaatgagca gaacctataa actgataagg gaattctgaa tattaaaaaa aaaaagaaaa
4261 ca
7. SEQ ID NO: 7
Accession No. NM_079769 Drosophila melanogaster
Hormone receptor-like in 96 CG11783-PA
MSPPKNCAVCGDKALGYNFNAVTCESCKAFFRRNALAKKQFTCPFNQNCD
ITVVTRRFCQKCRLRKCLDIGMKSENIMSEEDKLIKRRKIETNRAKRRLM
ENGTDACDADGGEERDHKAPADSSSSNLDHYSGSQDSQSCGSADSGANGC
SGRQASSPGTQVNPLQMTAEKIVDQIVSDPDRSQAINRLMRTQKEAISVM
EKVISSQKDALRLVSHLIDYPGDALKIISKFMNSPFNALTVFTKFMSSPT
DGVEIISKVIDSPADVVEFMQNLMHSPEDAIDIMNKFMNTPAEALRILNR
ILSGGGANAAQQTADRKPLLDKEPAVKPAAPAERADTVIQSMLGNSPPIS
PHDAAVDLQYHSPGVGEQPSTSSSHPLPYIANSPDFDLKTFMQTNYNDEP
SLDSDFSINSIESVLSEVIRIEYQAFNSIQQAASRVKEEMSYGTQSTYGG
CNSAANNSQPHLQQPICAPSTQQLDRELNEAEQMKLRELRLASEALYDPV
DEDLSALMMGDDRIKPDDTRHNPKLLQLINLTAVAIKRLIKMAKKITAFR
DMCQEDQVALLKGGCTEMMIMRSVMIYDDDRAAWKVPHTKENMGNIRTDL
LKFAEGNTYEEHQKFITTFDEKWRMDENIILIMCAIVLFTSARSRVIHKD
VIRLEQNSYYYLLRRYLESVYSGCEARNAFIKLIQKISDVERLNKFIINV
YLNVNPSQVEPLLREIFDLKNH
8. SEQ ID NO: 8
Accession No. NM_079769 Drosophila melanogaster
Hormone receptor-like in 96 CG11783-PA
      1 gttattggga ttggcctgga gcactcggac ggacagtaat tcattaaaat atgtggtgat
    61 aacgcgagct gccgaatctg cgtgcaattc gtgcgtttga cgtgggtact aactgctatg
  121 ctgtcgcgcg gacagttgtt ctgatacgca gagttcctgc ctcaccacac acgaccacct
  181 ccattaaaac cagccacccc ccccagcgcc tcctccaccg acagcagctg ctccaccgca
  241 coaccaggag aggggcaatt aaaaaatcaa tcagagggcc ctaattgaaa gctgccaccg
  301 tcgaaatgtc gccgccgaag aactgcgcgg tgtgcgggga caaggctctg ggctacaact
  361 tcaatgcggt cacctgcgag agctgcaagg cgttcttccg acggaacgcg ctggccaaga
  421 agcagttcac ctgccccttc aaccaaaact gcgacatcac tgtggtcact cgacgcttct
  481 gccagaaatg ccgcctgcgc aagtgcctgg atatcgggat gaagagtgaa aacattatgt
  541 ccgaggagga caagctgatc aagcggcgca agatcgagac caaccgggcc aagcgacgcc
  601 tcatggagaa cggcacggat gcgtgcgacg ccgatggcgg cgaggaaagg gatcacaaag
  661 cgccggcgga tagcagcagc agcaaccttg accactactc ggggtcacag gactcgcaga
  721 gctgcggctc ggcggacagc ggggccaatg ggtgctccgg cagacaggcc agttcgccgg
  781 gcacacaggt caatccgctt cagatgacgg ccgagaagat agtcgaccag atcgtatccg
  841 acccggatcg agcctcgcag gccatcaacc ggttgatgcg cacgcagaaa gaggctatat
  901 cggtgatgga gaaggtaatc agctcacaaa aggacgcctt aaggctggtg tcgcatttga
  961 tcgactatcc aggcgacgca ctcaagatca tttcaaagtt tatgaactcg ccctttaacg
1021 cgctgacagt attcaccaaa ttcatgagct cacccacgga cggcgttgaa attatctcaa
1081 agatagttga ttcgcccgcg gacgtggtgg agttcatgca gaacttgatg cactcgccag
1141 aggacgccat cgatataatg aacaagttca tgaatacccc agcggaggcg ctgcgcattc
1201 ttaaccgaat cctaagcggc ggaggagcga acgcagccca gcagacagca gaccgcaagc
1261 cattgctgga caaggagccg gcggtgaagc ctgcagcgcc agcggagcga gctgatactg
1321 tcattcaaag catgctgggc aacagtccgc caatttcgcc acatgatgct gccgtggatc
1381 tgcagtacca ctcgcccggt gtcggggagc agcccagtac atcgagtagc caccccttgc
1441 cttacatagc caactcgccg gacttcgatc tgaagacctt catgcagacc aactacaacg
1501 acgagcccag tctggacagt gattttagca ttaactcaat cgaatcggtg ctatccgagg
1561 tgatccgcat tgagtaccag gccttcaata gcatacaaca agcggcatcg cgcgtaaagg
1621 aggagatgtc ctacggcact cagtctacgt acggtggatg caattcggct gcaaacaata
1681 gccagccgca cctgcagcaa cccatctgcg ccccatccac ccagcagttg gatcgcgagc
1741 taaacgaggc ggagcaaatg aagctgcggg agctgcgact ggccagcgag gctctttatg
1801 atcccgtgga cgaggacctc agcgccctga tgatgggcga tgatcgcatt aagcccgacg
1861 acactcgcca caacccaaag ctattgcagc tgatcaatct gacggcggtg gccatcaagc
1921 ggcttatcaa aatggccaag aagattacag cattccgtga catgtgccag gaggaccagg
1981 tggccctact caaaggtggc tgcacagaaa tgatgataat gcgctccgta atgatttacg
2041 acgacgatcg cgccgcctgg aaggtacccc ataccaaaga gaacatgggc aacatacgca
2101 ctgacctgct caagtttgcc gaaggcaata tctacgagga gcaccaaaag ttcatcacaa
2161 cgtttgacga gaagtggcgc atggacgaga acataatcct gatcatgtgt gccattgtcc
2221 tttttacctc ggctcgatcg cgagtgatac acaaagacgt gattagattg gaacagaatt
2281 cctactatta tcttctgcga agatatctgg agagtgttta ttctggctgt gaggcgagaa
2341 acgcgtttat caagctaatc caaaagattt cagatgtgga gcgtctgaac aagttcataa
2401 ttaatgtcta tttgaatgtt aacccatccc aggtggagcc cttgctgcgt gaaatattcg
2461 atttgaaaaa tcactagaca accgatgcgt gtcgggcatt taatgcctat gttgatgccc
2521 aatgatgaat ggtcaacaag ctgtagttgt tgttgttgtt gatgtctgtt ttatcttgtc
2581 gcttgtaatg ttagatttta atcgaatgtg attgttagat ttgcatatac tgcatagatt
2641 ttatatttct acatcaaaga gagcatattt aggataccaa gtgcaaagca acacaatcta
2701 tatgtaatgt acaccgttta cctagtttca aataaactag acgataatgc aataactaac
2761 ttggaagcgt gggttctgtg caaaaaggaa aaaagacaaa aaaaataaac tgactttgag
2821 aaccagtggt aa
9. SEQ ID NO: 9
Accession No. NM_057539 Drosophila melanogaster
Hepatocyte nuclear factor 4 CG9310-PA
MMKHPQDLSVTDDQQLMKVNKVEKMEQELHDPESESHIMHADALASAYPA
ASQPHSPIGLALSPNGGGLGLSNSSNQSSENFALCNGNGNAGSAGGGSAS
SGSNNNNSMFSPNNNLSGSGSGTNSSQQQLQQQQQQQSPTVCAICGDRAT
GKHYGASSCDGCKGFFRRSVRKNHQYTCRFARNCVVDKDKRNQCRYCRLR
KCFKAGMKKEAVQNERDRISCRRTSNDDPDPGNGLSVISLVKAENESRQS
KAGAAMEPNINEDLSNKQFASINDVCESMKQQLLTLVEWAKQIPAFNELQ
LDDQVALLRAHAGEHLLLGLSRRSMHLKDVLLLSNNCVITRHCPDPLVSP
NLDISRIGARIIDELVTVMKDVGIDDTEFACIKALVFFDPNAKGLNEPHR
IKSLRHQILNNLEDYISDRQYESRGRFGELLILPVLQSITWQMIEQIQFA
KIFGVAHIDSLLQEMLLGGELADNPLPLSPPNQSNDYQSPTHTGNMEGGN
QVNSSLDSLATSGGPGSHSLDLEVQHIQALIEANSADDSFRAYAASTAAA
AAAAVSSSSSAPASVAPASISPPLNSPKSQHQHQQHATHQQQQESSYLDM
PVKHYNGSRSGPLPTQHSPQRMHPYQRAVASPVEVSSGGGGLGLRNPADI
TLNEYNRSEGSSAEELLRRTPLKIRAPEMLTAPAGYGTEPCRMTLKQEPE
TGY
10. SEQ ID NO: 10
Accession No. NM_057539 Drosophila melanogaster
Hepatocyte nuclear factor 4 CG9310-PA
      1 agttgaattc cagtgacgtt ggaagaaaca actgcaaaag gcaaaaacaa agacaatgtt
    61 tataagctgt atattccgct ttgattgata taaatgaata tatgcagtgc gccagttata
  121 caactgccct gcaaaagtca ctcattaaat aaaaaacgcc cgagatgaat ttcacagcgg
  181 cggcaacaag tgcaataata gtaaaaaatc aaaagccaaa caacgaaatc tctcccaaaa
  241 aaacgaagaa gcgtgtcgcg gtgccaaaaa gaaaacaaaa atagaaaaat acacaacaaa
  301 ataatacgga gaaacgttaa ttataacgag ccacaaaatc gcataaagaa atcaacaagt
  361 gtgtgtctgc ctttttttcc atattcgctt tcattcatgc ggtcaactca acaataacaa
  421 ctcaaaatag caacaacaac aataacaata tcaacaagag cagcagcagt cgctgataaa
  481 agccctgcag ctaaaacaac aacaaaacaa caaagatagt tagaaagaac atcgtctggc
  541 cattgagctt taattgccgg tcattacttc attactatgt gattggatct tcccgaccca
  601 cttgtaaata aaaagtaaaa atactggtta tgaagcatga tgaagcatcc gcaggatctg
  661 agtgtcacgg atgaccagca gttaatgaag gtgaacaagg tggagaagat ggagcaggag
  721 ttgcacgacc ccgaatcgga gagccacata atgcacgcgg atgccctggc ctctgcctat
  781 ccggctgcct cgcagcccca cagtccgatc ggcctcgccc tcagccccaa tggcggtggg
  841 ctgggactga gcaacagtag caaccagagc agcgagaact ttgcgctctg caacggaaac
  901 ggaaatgcgg gcagcgcagg aggcggaagt gccagcagtg gcagcaacaa caacaacagc
  961 atgttctcac ccaacaacaa cttgagcgga agcggaagtg ggactaacag cagtcagcag
1021 caattgcagc agcaacaaca acagcaatca ccgacggtct gcgccatttg tggagatcgg
1081 gcgacgggca aacattatgg agcctccagc tgcgacggct gcaaaggatt cttcaggagg
1141 agtgtcagga aaaatcatca gtacacttgc agatttgcgc gaaactgcgt tgtggacaag
1201 gacaaacgga atcagtgccg ctactgccgg ctgaggaagt gcttcaaggc gggcatgaag
1261 aaggaggcgg tgcaaaacga gcgggatcgc attagctgcc gccgcacctc caatgacgac
1321 ccggatccgg gcaatgggct gtctgtgatt tccttggtta aggcggagaa tgagtcgcgt
1381 cagtcgaagg caggcgctgc catggagcca aacattaacg aggacctctc caacaagcag
1441 ttcgcgagca tcaacgatgt ctgcgagtcg atgaagcagc agctgctgac cctggtggaa
1501 tgggctaagc agattccggc ctttaacgag ctgcagctgg atgaccaggt ggcactgcta
1561 cgcgcccatg ctggcgagca tttgctcctc ggcctgtctc gtcgttcgat gcacttgaag
1621 gatgttctcc tgctgagcaa caattgtgtg atcacaaggc actgtccaga tccccttgtg
1681 tcgccgaatt tggacatctc ccggatcggc gcccgtatca tcgatgaact ggtgacggtc
1741 atgaaggatg tgggtatcga tgacactgaa ttcgcttgca tcaaggccct agtcttcttc
1801 gatcccaatg ccaagggtct taatgaaccg catcgcatca aatcgctacg gcatcagata
1861 ctcaataatc tcgaggacta catatcagat cggcaatacg agtcgcgcgg tcgctttggc
1921 gagattctgc tcatcctgcc ggttctgcag tctattacct ggcagatgat cgagcagatc
1981 cagtttgcca agatctttgg agtggcccac attgattcat tactgcagga aatgttgttg
2041 ggaggagagt tggccgacaa tcctctgccg ctatcgccgc ccaatcagtc aaatgactac
2101 cagagtccca cccacacagg caacatggag ggcggtaatc aagttaactc ctctctggac
2161 tcgctggcca cgtccggtgg tcctggctcg catagtctgg acctggaggt gcagcacatt
2221 caggctctta tcgaggcgaa cagtgcggat gattccttcc gggcctacgc ggccagcact
2281 gcagcggcag ccgctgcagc cgtctcgtcc tcctcctctg cacccgcatc cgttgctcca
2341 gcctcgatct ctcctccgct caacagcccc aagtcacaac atcaacatca gcaacatgcg
2401 acgcatcagc aacaacagga gagctcctac ttggacatgc ccgtcaagca ctacaatggc
2461 agtcggtccg gaccgctgcc aacacagcac agtccccaga ggatgcatcc ctaccaaaga
2521 gcagtcgcct cgccggtcga agtgtccagc gggggcggcg gattgggtct gcgcaatcct
2581 gccgatatta cgctcaacga gtacaaccgg agcgagggta gcagtgccga ggagctgctg
2641 cgacgaactc cactgaagat ccgggctccc gagatgctaa ccgcacccgc tggttatgga
2701 acggaaccct gtcgcatgac acttaaacag gagccagaga ctggttacta gaagaataac
2761 gaacggtgca atatgcagtt tgcaatagga caccccttaa gcacacaacc catacacata
2821 caggccctct cttgctgtac tccccaccaa gtgctatata gagatgaaat tgaaatgaag
2881 aacttactta attgttatgc cttgaaccat tttgatactt tttattagtc ctaagtaggt
2941 attttggaaa ttgttgctta atttttaatg tttaacgcag ttgcaatata tttttggagt
3001 catattttgc tcaagaagtt tattatatac aattatacta tatatataca ccatttagca
3061 tgtactgagt ttgttggtta tttggttatc ttatacttgt gcgtggatca caaaacattc
3121 atataaggcc atgcaatata ttgttttagg ttagggtgtt gtctagatta tgctgaaagt
3181 gtaatatata tttaatttta aacaaagaac tatttttata tgaatatgta taatatacaa
3241 actatttc
11. SEQ ID NO: 11
Accession No. NM_176065 Drosophila melanogaster
Hormone receptor-like in 38 CG1864-PC
MDEDCFPPLSGGWSASPPAPSQLQQLHTLQSQAQMSHPNSSNNSSNNAGN
SHNNSGGYNYHGHFNAINASANLSPSSSASSLYEYNGVSAADNNFYGQQQ
QQQQQQSYQQHNYNSHNGERYSLPTFPTISELAAATAAVEAAAAATVSSP
SVGGPPPVRRASLPVQRTVSPAGSTAQSPKLAKITLNQRHSHAHAHALQL
NSAPNSAASSPASADLQAGRLLQAPSQLCAVCGDTAACQHYGVRTCEGCK
GFFKRTVQKGSKYVCLADKNCPVDKRRRNRCQFCRFQKCLVVGMVKEVVR
TDSLKGRRGRLPSKPKSPQESPPSPPISLITALVRSHVDTTPDPSCLDYS
HYEEQSMSEADKVQQFYQLLTSSVDVIDQFAEKIPGYFDLLPEDQELLFQ
SASLELFVLRLAYRARIDDTKLIFCNGTVLHRTQCLRSFGEWLNDIMEFS
RSLHNLEIDISAFACLCALTLITERHGLREPKKVEQLQMKIIGSLRDHVT
YNAEAQKKQHYFSRLLGKLPELRSLSVQGLQRIFYLKLEDLVPAPALIEN
MFVTTLPF
12. SEQ ID NO: 12
Accession No. NM_176065 Drosophila melanogaster
Hormone receptor-like in 38 CG1864-PC
      1 ctcgcccatt ggagggcccc tgtcctgtgg cagcagcttg cccagcttcc aggagaccta
    61 ctccttgaag tacaacagca gcagcggtag cagcccccag caggcgtcct cctcctccac
  121 cgccgccccc acgcccactg accaggtgct gaccctcaag atggacgagg actgcttccc
  181 gcctctgtcc ggcggctgga gtgccagtcc gcccgccccc tcccagctcc agcagctgca
  241 caccctgcag tctcaggccc agatgtcgca tcccaacagc agcaacaaca gcagcaacaa
  301 cgcgggcaac agccacaaca acagtggggg ctacaactac cacggccact tcaatgccat
  361 caatgccagc gccaatctgt cgcccagctc ctcggccagt tccctctacg aatataatgg
  421 tgtttccgca gcggacaact tctacggaca acagcagcag cagcaacagc aaagctatca
  481 gcaacataac tacaactcgc acaatggcga gcgttactcg ctgcccacgt ttcccacgat
  541 ttcggagctg gctgcggcca ctgctgctgt cgaagctgcg gcggcggcca cagtctcctc
  601 cccttcggtg ggcggtccgc cgccagtacg ccgagcatcg ctgccggttc agcgaaccgt
  661 ttcgccagcc ggctccacgg cgcagagccc caagctggcc aagatcacac tgaaccagcg
  721 gcactcccat gcccatgccc atgccctaca gctcaactcg gcacccaatt cggcggcaag
  781 ttcgccagcg agtgcggatc tgcaggcggg ccgtttgctc caggctccgt cgcagctgtg
  841 tgccgtttgt ggcgacaccg ccgcctgcca gcattatgga gtgcgaacct gcgagggatg
  901 caagggattc ttcaagcgga ccgtgcagaa gggctccaag tatgtctgcc tagcggacaa
  961 gaattgcccg gtggacaaga ggcgccgcaa ccgttgccag ttctgccggt tccagaagtg
1021 cctggtcgta ggcatggtca aggaagtggt gcgcacggac tcgttgaagg gtcgccgcgg
1081 gagactgccc tcaaaaccga aatcgcccca ggagtcgcca ccatcaccac ccatctcgtt
1141 gatcacggcc ctggttcgca gccatgtcga cacgactccg gatccctcgt gcctggacta
1201 cagccactat gaggagcagt cgatgagcga ggcagataag gtgcaacagt tttaccagct
1261 gctgaccagc tccgtggacg tgatcaagca gttcgccgag aagattcccg gctacttcga
1321 tctcctgccg gaggatcagg agctgctctt ccagagcgca tcgctggaac tgttcgtcct
1381 gcggctggcc tatcgcgcca ggatcgatga caccaagctg atcttctgca acggcacggt
1441 gctccaccgc acccagtgcc tgcgctcctt cggcgagtgg ctcaacgaca tcatggagtt
1501 cagccgcagc ctgcacaacc tggagatcga catctccgcc ttcgcctgcc tctgtgccct
1561 aaccctgatc acagaacgcc atggcctgcg ggagccgaag aaggtggagc agctccagat
1621 gaagatcatt ggcagtctgc gcgaccacgt cacctacaat gccgaggccc agaagaagca
1681 gcactacttc agccgcctgc tgggcaagct gccggagctg aggtccctga gtgtccaggg
1741 actgcagagg atcttctacc tgaagctgga ggacctggtg cccgcgccag ctctcatcga
1801 gaacatgttc gtcaccacat tgcccttcta gaggcgatca tcaagcgtat catcacaact
1861 tgcttcctta aactagcccc taagttatgc ctcctaggat atacagagaa aggaccccat
1921 aggacggacg caactagctt tagtagaacc ctgaaataaa taaatctcac aacagcaaaa
1981 acaaaaccga accgaacaga aatgaagcga atagcagacc caggccatat ctttagtgta
2041 gagctaggta gttagccgga cagccccggc tccttcgata attacggaca tgcatatttg
2101 agagggggtt tccagtgcac agcctatggc tcctgcgtga ctcgtcagca ccgcgagctc
2161 caacttgttg acgttaattg ttaaattgtt taatttcaac tgtcaaaacc ggaatcaacg
2221 gccgggcacg caatggcaac actttctatc cccggacttc gaagcctgct caacattcgg
2281 cactacggac ggacaaacaa cggacagaaa cagaactcac tcttgctctc ttgccttttg
2341 ctaacttcta gtcaattgat ttaggcgaat caaataaata aataaataaa ataagggcgt
2401 gcagcagtag tgttatataa tttctatgcc agaccccagc ggttctcttc aaggaaatcc
2461 cccaatgagt tgcacaaatt gggataaagt acgatagcct attattctta tatttctttt
2521 aaaagctcga agatagatga gaactgtgtg gaaatccact atcatatcat atagttgcta
2581 taagccgtgc ttgccctaag ctaagttaga cccgcataaa gttgatagcc caaccaagta
2641 tttcggttat ttcctagact aaggtcctaa tagttatagg ctaagactat tctgttcgat
2701 ttatcaatgc accaaacagt gcacaatgag agtataagta ccttcttgtg atgattgtgt
2761 ctgacacaga gagagttgca cacaagcaca caaactagcc gataagttac taaatacgat
2821 ctaatatcta atatatataa tataatataa tatatataag tccaagtatt cggaaatcca
2881 agaacccttg cataaccgca gttcgtacgt tccaaacgag aaaagaactt tatttaatcc
2941 tagaccactc catctaagtt ctcaaagaat cgtatgtgga tcgttggatc tgtctctcta
3001 tatatgtgtg tgtgttatct cgatagaaaa cccctctatg tgattttgtg atagattggc
3061 attgaactct atatattlat atatatatgt ctataatata tatacacgca taaatatata
3121 tttttatgtc taacttttgt atggtttatt ttatacgtac cacttttctt tgataacaaa
3181 aagtaaaaaa ctcgttagat agcaaatatt tcaaaggtat gttacgagga cttttcaaag
3241 taccagtctt tagcgacttt ccaattaacg ttcgtattaa cgaaagacag attttctatg
3301 tgttaaattg aagacttcta taactataac taaatgcaag ctaagagcaa aaacacaaat
3361 ccacaaatcc ccaaagtgaa taacatatct cttcaagctt tcgagtgcac ggaacacgta
3421 gaaccgaaac ccaagtgtta ctaaatccat ttaataatcg gcaagccggg ggcgtcggcg
3481 tggttaatac gttctcatta cctatacaat ttagatagat cattattaaa ttattgtaca
3541 tgtagcacat gaaatgttcg acaactagat tttgtaccat cttaaagaag aacctaggcc
3601 aagctaaact aagtataaac tatgatctgc atgcggctga gctgtagcta tgagaaatat
3661 acctgcgtgg atctaagtga aatgggacac tttgaattta gatatgaaac gttctaaacg
3721 cgacgtacta actctcccaa ctgcgaactc taccaattaa gagaaattcc cagaaaatgt
3781 gtcaggattt caaagcgtcc catctcactt gaacccaccc aatcaacaaa tacaaatcct
3841 agggaagttg agaggttcag caaccataga gcaatatttc ataagaaaac gcaccttaaa
3901 ttaccgaaaa acatagatta acctgatctt gtaacgtttg ggagcgataa taagccagga
3961 ttaaacagga acagttaggt gaccaaatca gttcgaaacg agatgataga taggttcggg
4021 ttcgaaaccc taaacgcgat gccattttag ccgttacaac attggatatc aaccatgcac
4081 atgaatatga atatgaatat gaatattata gagatatatc tagctatagg aacctacttt
4141 gtacctacac gacatggaaa catcaaacct acatgcatat ttacacacat atattttgaa
4201 tagagcgacg acttttacaa gttgcgtaca aagctatagc tatagcttga tatggccatc
4261 ccagagcgag catatacata tattttgggt tattgttctt ttgtaatttt ataaatgcat
4321 acatatttat tgtactacgt gaatgtcaag tgtggattca tatttttgag atacagctac
4381 aaaacgaaac aaaagaaaat aaaacaaaac agaagagtaa acgtgaaatt tttcgatgaa
4441 acaattttaa atgagaactt tttaatattg ctattaaagg atatacatat acacactaac
4501 atacatatat attttactat gtaacggata gaattaagct agatgcagcg cataaagctt
4561 tatacaacaa attgaaaagc aacagaagaa attggcacaa attaaattta tatagcataa
4621 ttagacgtcc ttcgcaagat aatgttattc gtaataagag cgtcaatcgg tacatcgggc
4681 gctatttccc actacacccc caaccacaca atagataacc taagctatgt atgtacatta
4741 gctatgtata tccagcccac ttatgcgcct actactagaa atgcagaaag cagaaagaga
4801 ggtgaaacct atagacgcta tcacaaatgt ctatctgata gacatcggta ctaccaatgc
4861 tatattgcca gttgtgtaat ttactcttat ttgatcgttt catttaccag ttaagaaccc
4921 aaatcatata agtgttatga tggaagaact ataacttgca attcaattaa ctctgcaata
4981 cgataacaag caaagcgaat catttcattt cgatttaatc tttaattata tatacttaaa
5041 cgatgtaagc ccaaaacaaa cgttttttct atatctgtct tttgagcaaa ttagttatac
5101 gcaaaaccaa accgtattta cataaatgta tacaaaacaa atcgtatatt ttcattggtt
5161 tgaaataaat acataaaaca a
13. SEQ ID NO: 13
Accession No. NM_141390 Drosophila melanogaster
CG10296-PA
MSNFSACAVCGDQSSGKHYGVSCCDGCSCFFKRSVRRGSSYACIALVGNC
VVDKARRNWCPSCRFQRCLAVGMNAAAVQEERGPRNQQVALYRTGRRQAP
PSQAAPSPTPHSQALHFQILAQILVTCLRQAKANEQFALLDRCQQDAIFQ
VVWSEIFVLRASHWSLDISAMIDGCGDEQLKRLICEAHQLRADVLELNFM
ESLILCRKELAINAEYAVILGSHSKAALISLARYTLQQSNYLRFGQLLLG
LRQLCLRRFDCALSCMFRSVVRDILK
14. SEQ ID NO: 14
Accession No. NM_141390 Drosophila melanogaster
CG10296-PA
      1 atgtcgaact tcagtgcctg cgcagtgtgc ggcgatcaga gctccgggaa gcactacggc
    61 gtgtcctgct gcgatgggtg ctcctgcttt ttcaagcgga gcgtgcggcg cgggagcagc
  121 tacgcctgca tcgctctggt cgggaactgt gtggtggaca aggcgcggcg gaactggtgt
  181 ccctcctgcc gcttccagcg atgcctggcc gtgggaatga acgctgctgc ggttcaggag
  241 gagcgcggtc cgcgcaacca gcaggtggct ctctaccgca ctggccggag acaagctccg
  301 ccatctcagg cggcgccatc cccgacgccc cactcccagg cgctgcactt ccagatcctc
  361 gcccagatcc ttgtcacgtg cctgcgccag gcgaaggcca acgagcagtt cgctctgttg
  421 gatcgctgcc aacaagacgc catctttcag gtggtgtgga gcgagatctt cgtcctgcga
  481 gcgtcccact ggtctctgga catcagcgcc atgatcgacg gctgcggcga tgagcagctc
  541 aaacggctca tttgcgaggc ccaccagcta agggccgacg tcctggaact caactttatg
  601 gagtccctaa tcctgtgcag aaaagaattg gccatcaatg cggagtatgc cgttatcctg
  661 ggaagccact ctaaagccgc cctgatctcc ttagcccgct acaccctgca gcaatccaac
  721 tacctgcggt tcggacaact gctccttggt ctgaggcagc tgtgcctgag gcgcttcgac
  781 tgcgcgcttt cttgtatgtt tcgcagcgtg gtcagggaca tcttaaaaac actttag
15. SEQ ID NO: 15
Accession No. NM_169459 Drosophila melanogaster
seven up CG11502-PC
MGMRREAVQRGRVPPTQPGLAGMHGQYQIANGDPMGIAGFNGHSYLSSYI
SLLLRAEPYPTSRYGQCMQPNNIMGIDNICELAARLLFSAVEWAKNIPFF
PELQVTDQVALLRLVWSELFVLNASQCSMPLHYAPLLAAAGLHASPMAAD
RVVAFMDHIRIFQEQVEKLKALHVDSAEYSCLKAIVLFTTDACGLSDVTH
IESLQEKSQCALEEYCRTQYPNQPTRFGKLLLRLPSLRTVSSQVIEQLFF
VRLVGKTPIETLIRDMLLSGNSFSWPYLPSM
16. SEQ ID NO: 16
Accession No. NM_169459 Drosophila melanogaster
seven up CG11502-PC
      1 ctaaattgtt gttttcaaaa gaaatgaatt tctttccact cctttcagaa ttcaagaata
    61 aatattgaag caatatggct tcccttgttc aaaccgatca atcgttgcaa atctttcttc
  121 aagcgctcgg tgcgacgtaa tctaacttac tcttgccgcg gcagcagaaa ctgtcccata
  181 gatcaacacc atcgcaatca atgtcaatat tgtcgattga agaagtgcct caaaatgggc
  241 atgagacgcg aagctgttca acgtggacgc gtaccaccca ctcagcccgg tctggccggc
  301 atgcatgggc agtaccagat tgccaacggg gatcccatgg gcattgccgg ctttaacggg
  361 cactcgtacc tcagttccta catctcgctc ctgctgcggg cggaaccgta tccgacttcg
  421 cgatatggcc agtgcatgca acccaacaac attatgggca tcgacaacat ctgcgaactg
  481 gccgcccgac tgctcttctc ggcggtcgag tgggccaaga acataccctt cttcccggag
  541 ctgcaggtga ccgaccaggt ggccctgctc cggctcgtct ggtcagagct cttcgtccta
  601 aacgccagcc agtgctccat gccgctccat gtggcgccac tgctggccgc cgccggactt
  661 catgcctccc cgatggccgc cgatcgtgtg gtggccttca tggaccacat ccgcatcttc
  721 caggagcagg tggagaagct gaaggcgctg catgtcgact ccgcggagta ctcctgcctc
  781 aaggcgatcg tgctcttcac caccgatgcc tgcggcctgt ccgatgtgac gcacattgaa
  841 tccctgcaag agaagtcgca gtgcgccctc gaggaatact gccggaccca gtatcccaac
  901 cagcccacga gattcggcaa gctgcttctc agactgccat cgctgcgaac ggtctcctca
  961 caagtcattg agcaattgtt ttttgtgcgt ctagtcggaa aaacgccaat tgaaacgctg
1021 atacgcgata tgctgctgag cggcaacagt ttctcctggc cctatctgcc ttcgatgtga
1081 cacacgatgt ggcgccaatt gacaacaact tgatcatcgg ccgcagctgt ggcggctgca
1141 acgctcaaca tcaattccgg cggaggcggc atcggcatcg gcggcggggg cagtggcagt
1201 ggcggtggcg gtagtggagg cggtggcgga gtcgttggat gtggcagcca caacgttgtc
1261 gctgccagtc atgaccagct cgccaatgtt gctgtcatgc agcaaacata cggcagcggc
1321 ggcagcagca gcagcagcat cagcggttgc cacaacggta acaacggcag cggcggcagc
1381 atttgcaatc agcagatcaa caactacggc aacaacagca acaacaatgt cggcaatcat
1441 atgagtgcag gcagtttttt cggtgggtcc aacaacagca tccacagtag tggcaatagc
1501 aataccgatt atatgaccac gccagccacc gcttatgcga caccagcgac agcagccaca
1561 tccacggtga acaccacaac gatgctgtct aattactgcg atgccgccac catgatgatg
1621 gccgctgctg cagtcaatgc aaatcaatgc ctgcagcaac atcaccagcg catgttgctc
1681 gcgggcagca gcaacagcag cagcaacaac agcagcagca acagcaaogg cgcagcagca
1741 atgccctcct catcctcgtc tggctcactg tcatctgcct catcgacccc aacagcaaca
1801 gcaactgcga ctgcaattgc aacagcaaca gcaactgcag cagcaacagc cgcgcagcaa
1861 caacagcaac aatcgccgcc aaatttaatc gatatcagcg aagttcctct cattgtggat
1921 gtcaagtagt gtaattattt atgcatctag aaatggggct ataaaccaac cttgtagata
1981 ccccgccccg cccccaccac taccacaaaa accataaaac cccaaaaaaa aaacaattga
2041 aaaatgtaaa aaaaaaaagt tggaggatga gcgccgcgta gcttaattga ctaattttcc
2101 atttgtagct tttgttgtaa ctttgtacat aactcctcga aaaattcaag tttttctcta
2161 ggccacccca gctgtgagca aaaccaatct cagctgacat atccaagaga acttcaaaag
2221 tgaagccccc aaaaaaagta agaaggcgcc aaaaaaacgt ctttacatat gaatgtgtat
2281 aatatttaaa tggcactgag ttctacttaa ttttagacca caaacacttg aaaaaatcaa
2341 tgaaaaaata agaattgtgg aaagagaaaa atccccccta acactttcaa aagacaaaac
2401 ataaagatag ttaaaatatt tatatatgta atgtagcata tacacgtata tagtacatat
2461 atgaatatat aaacgaaact ctactcccag tggtttgcag aaatatacca aaaattttaa
2521 gctatgttta cttgatgtgt ggcaattttt atgtgtgctt tagcaatttt atttttactt
2581 taagtaaaat ttaaaattta taaacattcg attctcgact ggtttttctc ggcggatgta
2641 tctcaaagat gcttctgtat gggaaggccg aattgttgaa atacgaatgc aaaatttagc
2701 gaatttttta tttagtaacc attacgagta aaaacacaaa atgttcagtg caagtttcag
2761 ttcttaaacg attttttcgt aagcttaagc attatcttat ttatgtgtat agagtatgaa
2821 aagttttcta tattttgtaa taataaaaat ttgcgtttat aatgaa
17. SEQ ID NO: 17
Accession No. NM_079857 Drosophila melanogaster
tailless CG1378-PA (til) inRNA
MQSSEGSPDMMDQKYNSVRLSPAASSRILYHVPCKVCRDHSSGKHYGIYA
CDGCAGFFKRSIRRSRQYVCKSQKQGLCVVDKTHTNQCRACRLRKCFEVG
MNKDAVQHERGPRNSTLRRHMAMKDAMMGAGEMPQIPAEILMNTAALTGF
PGVPMPGLPQRAGHHPAHMAAFQPPPSAAAVLDLSVPRVPHHPVHQGHHG
FFSPTAAYMNALATRALPPTPPLMAAEHIKETAAEHLFKNVNWIKSVRAF
TELPMPDQLLLLEESWKEFFILAMAQYLMPMNFAQLLFVYESENANREIM
GMVTREVHAFQEVLNQLCHLNIDSTEYECLRAISLFRKSPPSASSTEDLA
NSSILTGSGSPNSSASAESRGLLESGKVAAMHNDARSALHNYIQRTHPSQ
PMRFQTLLGVVQLMHKVSSFTIEELFFRKTIGDITIVRLISDMYSQRKI
18. SEQ ID NO: 18
Accession No. NM_079857 Drosophila melanogaster
tailless CG1378-PA (tll) mRNA
      1 gagtccacat cggagtaacc aaggatatat cgaatatatc acacaatccg caataccgcc
    61 gtccacccaa accgttaaaa caaaaatcca aaacgactca aagatacacc agtgccaagt
  121 gaaattcaat ttgtgcaagc gtttctacaa aaatcgccaa aattacgccc cacatcggta
  181 tgcagtcgtc ggagggttca ccagacatga tggatcagaa atacaacagc gtgcgtcttt
  241 cgccagcggc atcgagtcgc attctatacc atgtgccctg caaagtctgc agagatcaca
  301 gctccggcaa gcattacggc atctacgcct gtgatggctg cgccggattc ttcaagagga
  361 gcattcggag atcccggcag tatgtgtgca agtcgcagaa gcagggactc tgtgtggtgg
  421 acaagacgca caggaaccaa tgtagggctt gccgactgag gaagtgcttt gaggtcggaa
  481 tgaacaagga tgcagtgcag cacgagcggg gaccgcggaa ctccactctg cgtcgccaca
  541 tggccatgta caaggatgcc atgatgggcg ccggcgagat gccacaaata cccgccgaaa
  601 ttctgatgaa cacggctgcc ttgaccggct ttcctggagt accgatgccc atgcctggcc
  661 tgccccagag ggctggtcat catcctgctc acatggctgc cttccagccg ccaccatcgg
  721 ctgccgctgt cttggactta tccgtgccac gagtgcccca tcacccggtg caccaaggac
  781 accacggttt cttctcgccc accgccgcct acatgaatgc cctggccact cgggccctgc
  841 cccccactcc tccgctgatg gcagctgagc acatcaagga aaccgcggcg gaacacctat
  901 tcaagaacgt caactggatc aagagcgtac gggccttcac cgaactgccc atgccggatc
  961 agctgctcct gctggaggag tcctggaagg agttcttcat cctggccatg gcccagtacc
1021 taatgcccat gaatttcgcc cagctgctgt tcgtctacga gtccgagaat gccaaccggg
1081 agatcatggg catggtgacc cgcgaggtgc acgccttcca ggaggtgctg aaccaactgt
1141 gccatctgaa cattgacagc accgagtacg agtgtctgag ggctatttcg ctcttccgta
1201 agtcaccacc gtcggcaagt tctaccgagg atttagccaa cagctcaatc ctgacaggaa
1261 gcggcagccc gaactcctcg gcctctgctg aatccagggg tcttctggag tcgggaaaag
1321 tggcggccat gcacaacgat gcccggagtg cgctgcacaa ctacatccag aggacccatc
1381 cctcgcagcc catgcgattc cagacgctct tgggcgtggt gcagctgatg cacaaggtct
1441 caagcttcac catcgaggag ctgttcttcc gaaagaccat cggcgacatc accattgtgc
1501 gcctcatctc cgacatgtac agtcagcgca agatctgaaa agtatgtaga gcctagacta
1561 atcgccgcac tcgaagtgcc ttccaagtgc tgggaactgt gataatctcg gaagaagcgc
1621 tttggacaat actcgatcag tgaaatcaac gatttctcat atccaggagt cgagccttaa
1681 aatacgtaca caacactcac cttaatacct tacctaaaca gaactcgaag taatcttagc
1741 taaagtctct cagaccatcc agatgtgttt caaattgcat tcgcaaaagt ttcaactttg
1801 cctgttaaat acgtcaatcg tagttttaaa cactttagtt ttaagcgcat attattagct
1861 ttaggatttg gaaaaataat tattc
19. SEQ ID NO: 19
Accession No. NM_057792 Drosophila melanogaster
dissatisfaction CG9019-PA
MGTAGDRLLDIPCKVCGDRSSGKHYGIYSCDGCSGFFKRSIHRNRIYTCK
ATGDLKGRCPVDKTHRNQCRACRLAKCFQSMNKDAVQHERGPRKLHPQLH
HHHHHAAAAAAAAHHAAAAHHHHHHHHHAHAAAAHHAAVAAAAASGLHHH
HHAMPVSLVTNVSASFNYTQHISTHPPAPAAPPSGFHLTASGAQQGPAPP
AGHLHHGGAGHQHATAFHHPGHGHALPAPHGGVVSNPGGNSSAISGSGPG
STLPFPSHLLHHNLIAEAASKLPGITATAVAAVVSSTSTPYASAAQTSSP
SSNNHNYSSPSPSNSIQSISSIGSRSGGGEEGLSLGSESPRVNVETETPS
PSNSPPLSAGSISPAPTLTTSSGSPQHRQMSRHSLSEATTPPSHASLMIC
ASNNNNNNNNNNNNGEHKQSSYTSGSPTPTTPTPPPPRSGVGSTCNTASS
SSGFLELLLSPDKCQELIQYQVQHNTLLFPQQLLDSRLLSWEMLQETTAR
LLFMAVRWVKCLMPFQTLSKNDQHLLLQESWKELFLLNLAQWTIPLDLTP
ILESPLIRERVLQDEATQTEMKTIQEILCRFRQITPDGSEVGCMKAIALF
APETAGLCDVQPVEMLQDQAQCILSDHVRLRYPRQATRFGRLLLLLPSLR
TIRAATIEALFFKETIGNVPIARLLRDMYTMEPAQVDK
20. SEQ ID NO: 20
Accession No. NM_057792 Drosophila melanogaster
dissatisfaction CG9019-PA
      1 gtcagcccag gcgatccgca tttgcgtccg cagcaggttt ccgatttcag aactctgatt
    61 ccagcggcag cgaatcgcgt cggcatctga acatttgaaa ataatctaaa attgcaagtg
  121 actttgtgca ccggttacac taaaattgtt aacaaatcgc catatattct gaatttaaat
  181 ttaaagtgcg cagtgcggaa tataaatcag agcaaactgg atacgttagg gttcaaatac
  241 ttccatcaac ggaaaatggg cacagcgggc gatcgcctgt tggacattcc ctgcaaggtg
  301 tgtggcgatc gcagctccgg caagcactat ggaatctaca gctgcgatgg ctgctccggt
  361 tttttcaagc ggagcattca tcgcaatcgg atttacacct gtaaggccac cggcgatctc
  421 aagggtcgct gtccggtgga caagacccat cggaatcagt gtcgcgcctg tcgcctggcc
  481 aagtgcttcc agtcggccat gaacaaggat gctgtgcagc acgagcgcgg tcctaggaaa
  541 cccaagttgc acccgcaact gcatcatcat catcatcatg ctgctgccgc cgccgctgca
  601 gcgcatcatg cagcagccgc ccatcaccat caccatcatc accaccacgc ccacgcagcg
  661 gccgcccatc atgcggcagt ggctgcagcg gctgcctccg ggctgcatca ccaccaccac
  721 gccatgcccg tctcgctggt gaccaatgtc tcggcctcgt tcaactatac gcagcacatc
  781 tccacgcatc cgcctgctcc ggcggcgcca cccagtggct ttcacctgac ggccagtggc
  841 gcccagcagg gaccagctcc accagctggc cacctgcacc atgttggagc cggacatcag
  901 cacgccacgg ccttccacca tccgggacat ggacacgcgc tgcctgcccc acatggcggc
  961 gtcgtcagca atcccggcgg caactcgagc gcaatctccg gcagcggtcc cggctccacg
1021 ctgcccttcc cctcgcacct gctgcaccac aatctgatag cggaggcggc cagcaagctg
1081 ccgggcatca ctgccacagc cgttgcggcg gtggtgtcct ccactagcac gccctacgcc
1141 tcggcggccc agacgtcgtc gcctagtagc aacaaccaca actactcctc gccctcgccc
1201 agcaactcca tccagtccat ctcgagcatt ggatcgcgca gcggtggtgg cgaggagggc
1261 ctcagcctgg gcagcgagag tccgcgcgtc aatgtggaaa cggagacacc ttcgccatcg
1321 aactcgccgc cccttagtgc tggtagcatt tcgccagcgc ccacgttgac cacctcgtcg
1381 ggatcgccgc agcaccgcca gatgtcgcgg cacagcctca gtgaggcaac cacgccgccc
1441 agccacgcct ctctcatgat ttgcgccagc aacaataaca ataacaacaa taataataac
1501 aataatggag agcacaagca gtcgagctac acatccggat caccgacacc cacaacgccc
1561 acgccgccac cgccgcgttc tggtgtaggt tccacctgca acacggccag cagctccagc
1621 ggcttcctgg agctgctgct cagtccggac aagtgccagg agctcatcca gtaccaggtg
1681 cagcacaaca cgctgctctt cccgcaacag ctgttggact cgcggctgct ctcctgggag
1741 atgctgcagg agacgacggc gcgactgctc ttcatggcgg tgcgctgggt caagtgcctc
1801 atgcccttcc agacgctctc caagaacgac cagcatttgc tgctccagga atcctggaag
1861 gagctcttcc tgctcaacct cgcccaztgg actataccgc tggatctaac gcccatactg
1921 gaatcaccgc tcatccgcga acgggtgctg caggacgagg ccacacaaac ggagatgaag
1981 acgatccagg agatcctctg ccgcttccgc cagatcacac ccgacggcag cgaggtgggc
2041 tgcatgaagg ccatcgccct gttcgcaccc gaaaccgccg gcctgtgcga cgtgcagccg
2101 gtggagatgt tgcaggatca ggcgcagtgc atcctctccg accatgtgcg actgcgctac
2161 cctcgccaag caacccgctt cggcaggctg ctgctcctgc tgccctcgct gcgcaccatc
2221 cgggcggcca ccatcgaggc gctgttcttc aaggagacca tcggcaatgt gcccattgct
2281 cgactgctgc gcgacatgta caccatggaa ccggcacagg tggacaagtg aaccggccac
2341 gcatgacagt cgaaatgaaa tcaaaatcga ttccctagca cctaagcgcc acccatcggt
2401 cgtcgtcata tgcgaactta tttgtattcc aatgcgaccc gaatcctatt cagattcact
2461 gcggcaggag gcggtccaaa tgtggggcgg aagctgcaga tgctatggtt cgcaggacgc
2521 catgtaatgg aggcgtatgt actaaccgcg ctcctccatt ggcgatgcag tccgcgatga
2581 tggcgcactc ccacacccac acccgtaccc acaccttgat ttatcgccgg caatgcgtcg
2641 gagtctcctt actttcgctt cgttttctaa catttgtatc cttattttat ttcatctttt
2701 tccacggatt tttcgttttg actgcctggg cggcactctt tatttatctt tcattcgacg
2761 ttttgtcgtc gcttttctaa aaattcccca tgttatttca acctggcaag gacctcgcag
2821 tcccattccc gcgcccttac ttacaaatca cttcccatcc cacatccagc aattccgtgg
2881 tttgaattct ttcgtgcatt gactacgaaa taccctttaa tcagacaaat aaagaatatt
2941 agttgtaatt cttttttctg caatccagct ctaaaacggg tttcttaatc gaaatcgata
3001 aatgtaaaaa ttatacatat cctttaccaa cattgtttgc cta
21. SEQ ID NO: 21
NM_166092 Drosophila melanogaster CG16801-PA
MATGRSLLFRVPWYVCLCVCAESAEPGVYWRLRLRLGLPTLAGPHTNTLT
LTARTSSCRSIKKERIKASQQANAPPELPLKVSVDVNIIIAAHSQRRRIG
LVRFHQRESEDRPLAVASPRLQINMEPTAMNPKKLHSPQRHCYTPPPAPM
HGQMAPPPTSTGVAPPTQPPPPHPAAPNVPNGRLLSWNHSAAAAAAAAAA
QAAANSMNHSSAAEGSSMTRIKGQNLGLICVVCGDTSSGKHYGILACNGC
SGFFKRSVRRKLIYRCQAGTGRCVVDKAHRNQCQACRLKKCLQMGMNKDD
DSIDVTNDNEEPHAVSRSDSSFIMPQFMSPNLYTHQHETVYETSARLLFM
AVKWAKNLPSFARLSFRDQVILLEESWSELFLLNAIQWCIPLDPTGCALF
SVAEHCNNLENNANGDTCITKEELAADVRTLHEIFCKYKAVLVDPAEFAC
LKAIVLFRPETRGLKDPAQIENLQDQAHHTKTQFTAQIARFGRLLLMLPL
LRMISSHKIESIYFQRTIGNTPMEKVLCDMYKN
22. SEQ ID NO: 22
NM_166092 Drosophila melanogaster CG16801-PA
      1 atggcgaccg ggcgttctct gctctttcga gtgccttggt atgtgtgctt gtgtgtgtgc
    61 gcagagagcg cagagccggg tgtttattgg agattgcgat tgcggcttgg cttacccaca
  121 ctcgcagggc cgcacaccaa cacactaaca ctaacagcga ggacaagctc ctgccgcagc
  181 atcaagaagg aacgaatcaa agcaagccaa caagcaaatg cgccaccaga gttgccacta
  241 aaagtctccg ttgacgttaa catcatcatc gcggcacact cgcagcgccg tcggatcgga
  301 ttggttcggt ttcatcagcg ggaatcagag gaccgtccac ttgccgtcgc ctctccacga
  361 ttgcaaatta atatggagcc tactgcgatg aacccgaaaa aactccacag tccgcagcgg
  421 cattgctaca ctccgccgcc ggcgccgatg cacggacagg cgcctccacc tacatcaacg
  481 ggcgtggccc cgcccacaca gccaccgccc cctcatcccg ccgccccaaa cgtgcccaat
  541 ggtcgattgc tgagctggaa tcacagtgcc gctgcagctg ctgcggcggc ggcagcccaa
  601 gcggcagcca actccatgaa ccactcgtcg gcggcggagg gttcatcgat gacccggatt
  661 aagggtcaga acctgggcct catctgcgtg gtgtgcggcg acaccagctc gggaaagcac
  721 tacggaatcc tagcctgcaa tggctgctcc ggattcttca aacgcagcgt gcggcggaaa
  781 ctcatttatc gctgccaggc gggaacggga cgctgtgtgg tggacaaagc tcatcggaat
  841 caatgccagg cctgcaggct caagaagtgc cttcaaatgg gaatgaacaa ggacgacgac
  901 tccatagatg taaccaacga caacgaggag ccgcatgcag tcagcagatc ggattcgagt
  961 ttcattatgc cgcagttcat gtcgcccaat ctgtacaccc atcaacacga aacagtttac
1021 gagacaagtg cccggctgct cttcatggcc gtcaagtggg ccaagaacct gcccagcttt
1081 gcaagacttt cctttcggga tcaggtaatt ttgctggagg agtcctggtc ggagctgttc
1141 ctgctgaacg caatccaatg gtgcattccc ctggatccca ccggctgcgc cctcttctcg
1201 gtggcggagc actgcaataa tctagagaac aatgccaatg gcgacacttg cataacaaag
1261 gaggagctgg cggcggatgt gcgaacgctc cacgagatct tctgcaaata caaggcggtg
1321 ctggtggacc ccgctgaatt cgcgtgcctc aaggcgatag ttctcttCCg gccggaaacg
1381 cgcggactta aagatccggc gcagatagag aatcttcagg atcaggcgca ccacacaaag
1441 acgcagttca ccgcccagat agccagattc ggacgactcc ttctcatgct gccgttgctg
1501 cgcatgatca gctcccacaa gattgagtcc atctattttc agcgcactat tgggaacacg
1561 cccatggaaa aggtgctctg tgacatgtat aagaactag
23. SEQ ID NO: 23
Accession No. NM_168258 Drosophila melanogaster
estrogen-related receptor CG7404-PA (ERR)
MSDGVSILHIKQEVDTPSASCFSPSSKSTATQSGTNGLKSSPSVSPERQL
CSSTTSLSCDLHNVSLSNDGDSLKGSGTSGGNGGGGGGGTSGGNATNASA
GAGSGSVRDELRRLCLVCGDVASGFHYGVASCEACKAFFKRTIQGNIEYT
CPANNECEINKRRRKACQACRFQKCLLMGMLKEGVRLDRVRGGRQKYRRN
PVSNSYQTMQLLYQSNTTSLCDVKILEVLNSYEPDALSVQTPPPQVHTTS
ITNDEASSSSGSIKLESSVVTPNGTCIFQNNNNNDPNEILSVLSDIYDKE
LVSVIGWAKQIPGFIDLPLNDQMKLLQVSWAEILTLQLTFRSLPFNGKLC
FATDVWMDEHLAKECGYTEFYHCVQIAQRMERISPRREEYYLLKALLLAN
CDILLDDQSSLRAFRDTILNSLNDVVYLLRHSSAVSHQQQLLLLLPSLRQ
ADDILRRFWRGIARDEVITMKKLFLEMLEPLAR
24. SEQ ID NO: 24
Accession No. NM_168258 Drosophila melanogaster
estrogen-related receptor CG7404-PA (ERR)
      1 ccctggtcag gtctggttca ccaaaaaaga aaataaaatt acatttcaat ctttccaata
    61 tgcaaatatc tgcacgaaaa ccagcgagaa cagcatgctc acaataaaga gcccccaaac
  121 aatgtgactc gtatccgcgc agagtgacgt ttcgtgcctt gcccgagtgc caaatccaaa
  181 tcccaatcca ggcgcacaaa atcgatgcag atgctgtctg cattctcata gaaagtgcaa
  241 ctgaataacc gatggtcgcc aaaagccacg atgtccagta ataatgacca gtgaataaac
  301 aattatgact cgagcatcga aaaatgctga ggaacgaata cataagcaat aacaagaagg
  361 tgctcaactc ggaccaaaac aagtactaca tgctaacggt cgaggaggcc gatatgtatt
  421 gacgttgtta cagtggagct gattacacaa aagatcctca gaacgatttt atccaaggca
  481 cgaacatgtc cgacggcgtc agcatcttgc acatcaaaca ggaggtggac actccatcgg
  541 cgtcctgctt tagtcccagc tccaagtcaa cggccacgca gagtggcaca aacggcctga
  601 aatcctcgcc ctcggtttcg ccggaaaggc agctctgcag ctcgacgacc tctctatcct
  661 gcgatttgca caatgtatcc ttaagcaatg atggcgatag tctgaaagga agtggtacaa
  721 gtggcggcaa tggcggagga ggaggtggtg gtacgagtgg tggaaatgcg accaatgcga
  781 gtgccggagc tggatcggga tccgtcaggg acgagctccg ccgattgtgt ttggtttgtg
  841 gcgatgtggc cagtggattc cactatggtg tggcgagttg tgaggcttgc aaagcgttct
  901 ttaaacgcac catccaaggc aacatcgagt acacgtgtcc ggcgaacaac gagtgtgaga
  961 ttaacaagcg gagacgcaag gcctgccaag cgtgtcgctt ccagaaatgt ctactaatgg
1021 gcatgctcaa ggagggtgtg cgcttggatc gagttcgtgg aggacggcag aagtaccgaa
1081 ggaatcctgt atcaaactct taccagacta tgcagctgct ataccaatcc aacaccacct
1141 cgctgtgcga tgtcaagata ctggaggtgc tcaattcata tgagccggat gccttgagcg
1201 tccaaacgcc gccgccgcaa gtccacacga ctagcataac taatgatgag gcctcatcct
1261 cctcgggcag cataaaactg gagtccagcg ttgttacgcc caatgggact tgcattttcc
1321 aaaacaacaa caacaatgat cccaatgaga tactaagcgt ccttagtgat atttacgaca
1381 aggaattggt cagcgtcatt ggctgggcca agcagatacc tggctttata gatctgccac
1441 ttaacgacca gatgaagctt ctccaggtgt cgtgggcaga gatcctgacg ctccagctga
1501 ccttccggtc cctaccgttc aatggcaagt tatgcttcgc cacggatgtc tggatggatg
1561 aacatttggc caaggagtgc ggttacacgg agttctacta ccactgcgtc cagatcgcac
1621 agcgcatgga aagaatatcg ccacgaaggg aggagtacta cttgctaaag gcgctcctgc
1681 tggccaactg cgacattctg ctggatgatc agagttccct gcgcgcattt cgtgatacga
1741 ttcttaattc tctaaacgat gtggtctact tgctgcgtca ttcgtcggcc gtgtcgcatc
1801 agcaacaatt gctgcttttg ctgccttcgc tgcggcaggc ggatgatatc ctgcgaagat
1861 tttggcgtgg aattgcacgc gatgaagtca ttaccatgaa gaaactgttc ctcgagatgc
1921 tcgagccgct ggccaggtga aaaggattat gcgggcgccc aaactagttg atctagctga
1981 taagcaaagg tgcaaatata gtcttaggta tatatggatg tatactagag tagattaagc
2041 gtaggataag ccatgtatat aaatagtaaa atacttgtcg ggtaagatta gttcgcagaa
2101 aaaatctctt ttaatggact accaactaca gcaactggaa aaccctactt atcttctaga
2161 atcggggtgt gcttacactg gttaaaggcg catataggtg ttatgtgtct aaagttgtga
2221 gtcacagatc ttcaataatt tgttcaattc tcactggttc tgatatatgt atatgccgca
2281 accttctgat gtaacgtatg aatttgtggg cacttttaaa atacgatagt ggttctacaa
2341 tacaatggat tatactgttt ctaagtgtca tgtaacccag tgattctgtg tctatgtggt
2401 acacatgcgg tcaaaagaat agcaatgtcg tccgtgaata ataaaccgtt tgtaactgtt
2461 gtttccatac tccctaagtt ctgtattctt tggggatttt cttttcctaa acaaattcaa
2521 attagtttt
25. SEQ ID NO: 25
Accession No. NM_168908 Drosophila melanogaster
Hormone-receptor-like in 78 CG7199-PC
MDGVKVETFIKSEENRAMPLIGGGSASGGTPLPGGGVGMGAGASATLSVE
LCLVCGDRASGRHYGAISCEGCKGFFKRSIRKQLGYQCRGAMNCEVTKHH
RNRCQFCRLQKCLASGMRSDSVQHERKPIVDRKEGIIAAAGSSSTSGGGN
GSSTYLSGKSGYQQGRGKGHSVKAESAATPPVHSAPATAFNLNENIFPMG
LNFAELTQTLMFATQQQQQQQQQHQQSGSYSPDIPKADPEDDEDDSMDNS
STLCLQLLANSASNNNSQHLNFNAGEVPTALPTTSTMGLIQSSLDMRVIH
KGLQILQPIQNQLERNGNLSVKPECDSEAEDSGTEDAVDAELEHMELDFE
CGGNRSGGSDFAINEAVFEQDLLTDVQCAFHVQPPTLVHSYLNIHYVCET
GSRIIFLTIHTLRKVPVFEQLEAHTQVKLLRGVWPALMAIALAQCQGQLS
VPTIIGQFIQSTRQLADIDKIEPLKISKMANLTRTLHDFVQELQSLDVTD
MEFGLLRLILLFNPTLLQQRKERSLRGYVRVQLYALSSLRRQGGIGGGEE
RFNVLVARLLPLSSLDAEAMEELFFANLVGQMQMDALIPFILMTSNTSGL
26. SEQ ID NO: 26
Accession No. NM_168908 Drosophila melanogaster
Hormone-receptor-like in 78CG7199-PC
      1 attggaacaa ggagatttta ttgcgttaga aaaggttcaa aataggcaca aagtgcctga
    61 aaatatcgta actgaccgga agtaacataa ctttaaccaa gtgcctcgaa aaatagatgt
  121 ttttaaaagc tcaagaatgg tgataacaga cgtccaataa gaattttcaa agagccaaat
  181 gtttgggttt cagttattta tacagccgac gactattttt tagccgcctg ctgtggcgac
  241 aatggacggc gttaaggttg agacgttcat caaaagcgaa gaaaaccgag cgatgccctt
  301 gatcggagga ggcagtgcct caggcggcac tcctctgcca ggaggcggcg tgggaatggg
  361 agccggagca tccgcaacgt tgagcgtgga gctgtgtttg gtgtgcgggg accgcgcctc
  421 cgggcggcac tacggagcca taagctgcga aggctgcaag ggattcttca agcgctcgat
  481 ccggaagcag ctgggctacc agtgtcgcgg ggctatgaac tgcgaggtca ccaagcacca
  541 caggaatcgg tgccagttct gtcgactaca gaagtgcctg gccagcggca tgcgaagtga
  601 ttctgtgcag cacgagagga aaccgattgt ggacaggaag gaggggatca tcgctgctgc
  661 cggtagctca tccacttctg gcggcggtaa tggctcgtcc acctacctat ccggcaagtc
  721 cggctatcag caggggcgtg gcaaggggca cagtgtaaag gccgaatccg cggccacgcc
  781 tccagtgcac agcgcgccag caacggcctt caatttgaat gagaatatat tcccgatggg
  841 tttgaatttc gcagaactaa cgcagacatt gatgttcgct acccaacagc agcagcaaca
  901 acagcaacag catcaacaga gtggtagcta ttcgccagat attccgaagg cagatcccga
  961 ggatgacgag gacgactcaa tggacaacag cagcacgctg tgcttgcagt tgctcgccaa
1021 cagcgccagc aacaacaact cgcagcacct gaactttaat gctggggaag tacccaccgc
1081 tctgcctacc acctcgacaa tggggcttat tcagagttcg ctggacatgc gggtcatcca
1141 caagggactg cagatcctgc agcccatcca aaaccaactg gagcgaaatg gtaatctgag
1201 tgtgaagccc gagtgcgatt cagaggcgga ggacagtggc accgaggatg ccgtagacgc
1261 ggagctggag cacatggaac tagactttga gtgcggtggg aaccgaagcg gtggaagcga
1321 ttttgctatc aatgaggcgg tctttgaaca ggatcttctc accgatgtgc agtgtgcctt
1381 tcatgtgcaa ccgccgactt tggtccactc gtatttaaat attcattatg tgtgtgagac
1441 gggctcgcga atcatttttc tcaccatcca tacccttcga aaggttccag ttttcgaaca
1501 attggaagcc catacacagg tgaaactcct gagaggagtg tggccagcat taatggctat
1561 agctttggcg cagtgtcagg gtcagctttc ggtgcccacc attatcgggc agtttattca
1621 aagcactcgc cagctagcgg atatcgataa gatcgaaccg ttgaagatct cgaagatggc
1681 aaatctcacc aggaccctgc acgactttgt ccaggagctc cagtcactgg atgttactga
1741 tatggagttt ggcttgctgc gtctgatctt gctcttcaat ccaacgctct tgcagcagcg
1801 caaggagcgg tcgttgcgag gctacgtccg cagagtccaa ctctacgctc tgtcaagttt
1861 gagaaggcag ggtggcatcg gcggcggcga ggagcgcttt aatgttctgg tggctcgcct
1921 tcttccgctc agcagcctgg acgcagaggc catggaggag ctgttcttcg ccaacttggt
1981 ggggcagatg cagatggatg ctcttattcc gttcatactg atgaccagca acaccagtgg
2041 actgtaggcg gaattgagaa gaacagggcg caagcagatt cgctagactg cccaaaagca
2101 agactgaaga tggaccaagt gcgggcaata catgtagcaa ctaggcaaat cccattaatt
2161 atatatttaa tatatacaat atatagttta ggatacaata ttctaacata aaaccatggg
2221 tttattgttg ttcacagata aaatggaatc gatttcccaa taaaagcgaa tatgttttta
2281 aacagaat
27. SEQ ID NO: 27
Accession No. NM_057433 Drosophila melanogaster
ultraspiracle CG4380-PA (usp)
MDNCDQDASFRLSHIKEEVKPDISQLNDSNNSSFSPKAESPVPFMQAMSM
VHVLPGSNSASSNNNSAGDAQMAQAPNSAGGSAAAAVQQQYPPNHPLSGS
KHLCSICGDRASGKHGVYSCEGCKGFFKRTVRKDLTYACRENRNCIIDKR
QRNRCQYCRYQKCLTCGMKREAVQEERQRGARNAAGRLSASGGGSSGPGS
VGQSSSQGGGGGGGVSGGMGSGNGSDDFMTNSVSRDFSIERIIEAEQRAE
TQCGDRALTFLRVGPYSTVQPDYKGAVSALCQVVNKQLFQMVEYARMMPH
FAQVPLDDQVILLKAAWIELLIANVAWCSIVSLDDGGAGGGGGGLGHDGS
FERRSPGLQPQQLFLNQSFSYHRNSAIKAGVSIFDRILSELSVKMKRLNL
DRRELSCLKAIILYNPDIRGIKSRAEIEMCREKVYACLDEHCLEHPGDDG
RFAQLLLRLPALRSISLKCQDHLFLFRITSDRPLEELFLEQLEAPPPPGL
AMKLE
28. SEQ ID NO: 28
Accession No. NM_057433 Drosophila melanogaster
ultraspiracle CG4380-PA (usp)
      1 aaaaatgtcg acgcgaaaaa aggtatttat tcattagtca gaaagtctgg cattctttgt
    61 ttgttggtaa aaagcgcaat tgtttggagg cgagcgaata aagtgcgctg ctccatcggc
  121 tcaagattat gtaaatgcag caacgacccc accaacaacg aaactgcaac ctgctccact
  181 tggcccaacg gaccaatagc ggacggacgg acacggtggc gttggcaaag tgaaacccca
  241 acagagaggc gaaagcgagc caagacacac cacatacaca cgaagagaac gagcaagaag
  301 aaaccggtag gcggaggagg cgctgccccc agttcctcca atatacccag caccacatca
  361 caagcccagg atggacaact gcgaccagga cgccagcttt cggctgagcc acatcaagga
  421 ggaggtcaag ccggacatct cgcagctgaa cgacagcaac aacagcagct tttcgcccaa
  481 ggccgagagt cccgtgccct tcatgcaggc catgtccatg gtccacgtgc tgcccggctc
  541 caactccgcc agctccaaca acaacagcgc tggagatgcc caaatggcgc aggcgcccaa
  601 ttcggctgga ggctctgccg ccgctgcagt ccagcagcag tatccgccta accatccgct
  661 gagcggcagc aagcacctct gctctatttg cggggatcgg gccagtggca agcactacgg
  721 cgtgtacagc tgtgagggct gcaagggctt ctttaaacgc acagtgcgca aggatctcac
  781 atacgcttgc agggagaacc gcaactgcat catagacaag cggcagagga accgctgcca
  841 gtactgccgc taccagaagt gcctaacctg cggcatgaag cgcgaagcgg tccaggagga
  901 gcgtcaacgc ggcgcccgca atgcggcggg taggctcagc gccagcggag gcggcagtag
  961 cggtccaggt tcggtaggcg gatccagctc tcaaggcgga ggaggaggag gcggcgtttc
1021 tggcggaatg ggcagcggca acggttctga tgacttcatg accaatagcg tgtccaggga
1081 tttctcgatc gagcgcatca tagaggccga gcagcgagcg gagacccaat gcggcgatcg
1141 tgcactgacg ttcctgcgcg ttggtcccta ttccacagtc cagccggact acaagggtgc
1201 cgtgtcggcc ctgtgccaag tggtcaacaa acagctcttc cagatggtcg aatacgcgcg
1261 catgatgccg cactttgccc aggtgccgct ggacgaccag gtgattctgc tgaaagccgc
1321 ttggatcgag ctgctcattg cgaacgtggc ctggtgcagc atcgtttcgc tggatgacgg
1381 cggtgccggc ggcgggggcg gtggactagg ccacgatggc tcctttgagc gacgatcacc
1441 gggccttcag ccccagcagc tgttcctcaa ccagagcttc tcgtaccatc gcaacagtgc
1501 gatcaaagcc ggtgtgtcag ccatcttcga ccgcatattg tcggagctga gtgtaaagat
1561 gaagcggctg aatctcgacc gacgcgagct gtcctgcttg aaggccatca tactgtacaa
1621 cccggacata cgcgggatca agagccgggc ggagatcgag atgtgccgcg agaaggtgta
1681 cgcttgcctg gacgagcact gccgcctgga acatccgggc gacgatggac gctttgcgca
1741 actgctgctg cgtctgcccg ctttgcgatc gatcagcctg aagtgccagg atcacctgtt
1801 cctcttccgc attaccagcg accggccgct ggaggagctc tttctcgagc agctggaggc
1861 gccgccgcca cccggcctgg cgatgaaact ggagtagggt cccgactcta aagtctcccc
1921 cgttctccat ccgaaaaatg tttcattgtg attgcgtttg tttgcatttc tcctctctat
1981 cccttatacc ctacaaaagc cccctaatat tacgcaaaat gtgtatgtaa ttgtttattt
2041 tttttttatt acctaatatt attattatta ttgatataga aaatgttttc cttaagatga
2101 agattagcct cctcgacgtt tatgtcccag taaacgaaaa acaaacaaaa tccaaaactt
2161 gaaaagaaca caaaacacga acgagaaaat gcacacaagc aaagtaaaag taaaagttaa
2221 actaaagcta aacgagtaaa gatattaaaa taacggttaa aattaatgca tagttatgat
2281 ctacagacgt atgtaaacat acaaattcag cataaatata tatgtcagca ggcgcatatc
2341 tgcggtgctg gccccgttct aaatcaattg taattacttt ttaacataaa tttacccaaa
2401 acgttatcaa ttagatgcga gatacaaaaa tcaccgacga aaaccaacaa aatatatcta
2461 tgtataaaaa atataaactg cataacaa
29. SEQ ID NO: 29
Accession No. NM_168757 Drosophila melanogaster
Ecdysone-induced protein 75B CG8127-PD
MGEELPILKGILKGNVNYHNAPVRFGRVPKREKARILAAMQQSTQNRGQQ
RALATELDDQPRLLAAVLRHLETCEFTKEKVSAMRQRARDCPSYMPTLLA
CPLNPAPELQSEQEFSQRFAHVIRGVIDFAGMIPGFQLLTQDDKFTLLKA
GLFDALFVRLICMFDSSINSIICLNGQVMRRDAIQNGANARFLVDSTFNF
AERMNSMNLTDAEIGLFCAIVLITPDRPGLRNLELIEKMYSRLKGCLQYI
VAQNRPDQPEFLAKLLETMPDLRTLSTLHTEKLVVFRTEHKELLRQQMWS
MEDGNNSDGQQNKSPSGSWADAMDVEAAKSPLGSVSSTESADLDYGSPSS
SQPQGVSLPSPPQQQPSALASSAPLLAATLSGGCPLRNRANSGSSGDSGA
AEMDIVGSHAHLTQNGLTITPIVRHQQQQQQQQQIGILNNAHSRNLNGGH
AMCQQQQQHPQLHHHLTAGAARYRKLDSPTDSGIESGNEKNECKAVSSGG
SSSCSSPRSSVDDALDGSDAAANHNQVVQHPQLSVVSVSPVRSPQPSTSS
HLKRQIVEDMPVLKRVLQAPPLYDTNSLMDEAYKPHKKFRALRHREFETA
EADASSSTSGSNSLSAGSPRQSPVPNSVATPPPSAASAAAGNPAQSQLHM
HLTRSSPKASMASSHSVLAKSLMAEPRMTPEQMKRSDIIQNYLKRENSTA
ASSTTNGVGNRSPSSSSTPPPSAVQNQQRWGSSSVITTTCQQRQQSVSPH
SNGSSSSSSSSSSSSSSSSSTSSNCSSSSASSCQYFQSPHSTSNGTSAPA
SSSSGSNSATPLLELQVDIADSAQPLNLSKKSPTPPPSKLHALVAAANAV
QRYPTLSADVTVTASNGGPPSAAASPAPSSSPPASVGSPNPGLSAAVHKV
MLEA
30. SEQ ID NO: 30
Accession No. NM_168757 Drosophila melanogaster
Ecdysone-induced protein 75B CG8127-PD
      1 agtcaccgtc gcagtcgcag cagttgaggt tcgctctcct cgatttcggg caaatccgat
    61 accatatagc acagcgtacc gcactctggg tatattcgta acgcgctttg gcttttacag
  121 ttagtcgcgt tcgagacctt gtcgagtttt gtcatgttag ccagcgatcc gcgggatccg
  181 aaataagcca agaatcacaa cgcgagtgcg gcagttgcca gcagtaacta caccaatatt
  241 tataftaatt aaaataaatt aaatgaaaca acatgctgat taatgccaat gaatgttaaa
  301 tgcaattgtt aatgtgaaga aaagtcgacc aagtctcccc aaaacaacac ttattcaaca
  361 tccactacac actcgccttt ctggattacg cgcccaaaaa aaaacaaaaa ttaaaaatta
  421 aaccaaacca acaactaatt tatttgctaa atattccaaa aattcaatca atgtgaaaag
  481 caagcaaaca aagttcctct cacaacaaaa cagcagttaa ttaaaatatc taaccgagat
  541 aaagtgcaaa gaagataaca agtttctcaa gcaaacatcc atatgtacct gagtaccaac
  601 caaaaagctg tgtgtgtgcc aaaaaccgaa gaggaattat ccaaaaatat ttaatgagca
  661 agctcaactg agtggttgat gtgcccccca agggaaaagt gaccaagtca agatattttg
  721 tcaaatcgaa cacagaaaac acaaaaatgg gcgaagaact cccgatattg aagggcatac
  781 ttaaaggcaa cgtcaactat cacaatgcgc ctgtgcgttt tggacgcgtg ccgaagcgcg
  841 aaaaggcgcg tatcctggcg gccatgcaac agagcaccca gaatcgcggc cagcagcgag
  901 ccctcgccac cgagctggat gaccagccac gcctcctcgc cgccgtgctg cgcgcccacc
  961 tcgagacctg tgagttcacc aaggagaagg tctcggcgat gcggcagcgg gcgcgggatt
1021 gcccctccta ctccatgccc acacttctgg cctgtccgct gaaccccgcc cctgaactgc
1081 aatcggagca ggagttctcg cagcgtttcg cccacgtaat tcgcggcgtg atcgactttg
1141 ccggcatgat tcccggcttc cagctgctca cccaggacga taagttcacg ctcctgaagg
1201 cgggactctt cgacgccctg tttgtgcgcc tgatctgcat gtttgactcg tcgataaact
1261 caatcatctg tctaaatggc caggtgatgc gacgggatgc gatccagaac ggagccaatg
1321 cccgcttcct ggtggactcc accttcaatt tcgcggagcg catgaactcg atgaacctga
1381 cagatgccga gataggcctg ttctgcgcca tcgttctgat tacgccggat cgccccggtt
1441 tgcgcaacct ggagctgatc gagaagatgt actcgcgact caagggctgc ctgcagtaca
1501 ttgtcgccca gaataggccc gatcagcccg agttcctggc caagttgctg gagacgatgc
1561 ccgatctgcg caccctgagc accctgcaca ccgagaaact ggtagttttc cgcaccgagc
1621 acaaggagct gctgcgccag cagatgtggt ccatggagga cggcaacaac agcgatggcc
1681 agcagaacaa gtcgccctcg ggcagctggg cggatgccat ggacgtggag gcggccaaga
1741 gtccgcttgg ctcggtatcg agcactgagt ccgccgacct ggactacggc agtccgagca
1801 gttcgcagcc acagggcgtg tctctgccct cgccgcctca gcaacagccc tcggctctgg
1861 ccagctcggc tcctctgctg gcggccaccc tctccggagg atgtcccctg cgcaaccggg
1921 ccaattccgg ctccagcggt gactccggag cagctgagat ggatatcgtt ggctcgcacg
1981 cacatctcac ccagaacggg ctgacaatca cgccgattgt gcgacaccag cagcagcaac
2041 aacagcagca gcagatcgga atactcaata atgcgcattc ccgcaacttg aatgggggac
2101 acgcgatgtg ccagcaacag cagcagcacc cacaactgca ccaccacttg acagccggag
2161 ctgcccgcta cagaaagcta gattcgccca cggattcggg cattgagtcg ggcaacgaga
2221 agaacgagtg caaggcggtg agttcggggg gaagttcctc gtgctccagt ccgcgttcca
2281 gtgtggatga tgcgctggac tgcagcgatg ccgccgccaa tcacaatcag gtggtgcagc
2341 atccgcagct gagtgtggtg tccgtgtcac cagttcgctc gccccagccc tccaccagca
2401 gccatotgaa gcgacagatt gtggaggata tgcccgtgct gaagcgcgtg ctgcaggctc
2461 cccctctgta cgataccaac tcgctgatgg acgaggccta caagccgcac aagaaattcc
2521 gggccctgcg gcatcgcgag ttcgagaccg ccgaggcgga tgccagcagt tccacttccg
2581 gctcgaacag cctgagtgcc ggcagtccgc gacagagtcc agtcccgaac agtgtggcca
2641 cgcccccgcc atcggcggcc agcgccgccg caggtaatcc cgcccagagc cagctgcaca
2701 tgcacctgac ccgcagcagc cccaaggcct cgatggccag ctcgcactcg gtgctggcca
2761 agtctctcat ggccgagccg cgcatgacgc ccgagcagat gaagcgcagc gatattatcc
2821 aaaactactt gaagcgcgag aacagcacag cagccagcag caccaccaat ggcgtgggca
2881 accgcagtcc cagcagcagc tccacaccgc cgccatcggc ggtccagaat cagcagcgtt
2941 ggggcagcag ctcggtgatc accaccacct gccagcagcg ccagcagtcc gtgtcgccgc
3001 acagcaacgg ttccagctcc agttcgagct ctagctccag ctccagttcg tcatcctcct
3061 ccacatcctc caactgcagc tccagctcgg ccagcagctg ccagtatttc cagtcgccgc
3121 actccaccag caacggcacc agtgcaccgg cgagctccag ttcgggatcg aacagcgcca
3181 cgcccctgct ggaactgcag gtggacattg ctgactcggc gcagcctctc aatttgtcca
3241 agaaatcgcc cacgccgccg cccagcaagc tgcacgctct ggtggccgcc gccaatgccg
3301 ttcaaaggta tcccacattg tccgccgacg tcacagtgac agcctccaat ggcggtcctc
3361 cgtcggcggc ggcgagtccg gcgcccagca gcagtccgcc ggcgagtgtg ggctccccca
3421 atccgggcct gagcgccgcc gtgcacaagg taatgctgga ggcgtaagag cgggaggagg
3481 taggtggttt tacgcggaga agtgggagag acagagactg ggagtggcag ttcagcgaag
3541 caggaagcag gatcacttgg agcggcggga gttgaattaa attattttac catttaattg
3601 agacgtgtac aaagtttgaa agcaaaacca acatgcatgc aatttaaaac taatatttaa
3661 agcaacaaca aacaaaacaa ctacaagtta ttaatttaaa aaacaaacaa acaaacaaac
3721 aacaaaaaac ccaagcttga atggtattac
31. SEQ ID NO: 31
Accession No. NM_168892 Drosophila melanogaster
Ecdysone-induced protein 78C CG18023-PBEip78C)
MPSHLQQQQQQHLLQQQQQQQHQPQLQQHHQLQQQPHVSGVRVKTPSTPQ
TPQMCSIASSPSELGGCNSANNNNNNNSSSGNASGGSGVSVGVVVVGGHQ
QLVGGSMVGMAGMGTDAHQVGMCHDGLAGTANELTVYDVIMCVSQAHRLN
CSYTEELTRELMRRPVTVPQNGIASTVAESLEFQKIWLWQQFSARVTPGV
QRIVEFAKRVGFCDFTQDDQLILIKLGFFEVWLTHVARLINEATLTLDDG
AYLTRQQLEILYDSDFVNALLNFANTLNAYGLSDTEIGLFSAMVLLASDR
AGLSEPKVIGRARELVAEALRVQILRSRAGSPQALQLMPALEAKIPELRS
LGAKHFSHLDWLRMNWTKLRLPPLFAEIFDIPKADDEL
32. SEQ ID NO: 32
Accession No. NM_168892 Drosophila melanogaster
Ecdysone-induced protein 78C CG18023-PBEip78C)
      1 aagcattaac gaaagaactg cgcacaaagt agggaggcaa taattacata tgtacatggc
    61 tgggaaaggc cttaactaaa cttagcaaac taataaatag aaaaaaggaa atattggcca
  121 aatattatag tattgggaat attaggttac ttgatatcaa aaattaatgt ctattttata
  181 catttattct tagacttaat gttaacttat cgtacttatt atgattggtt tttcaagatt
  241 accagaactt gatagattgg tctagctttt gaaatcggat agcattttct ttaaaggact
  301 ttgccatatg ctaaagccta acttcttttt tcaattcagc cacagctgac aaaagcgaag
  361 aaaatttgaa agaccgtgaa tccttttgaa acgccctctc cggattcctc attaagtgca
  421 aaagatataa catcgcagag atttcccata aaaatgctga tcaggcgccc tcgcaggttg
  481 ccaacgtcga tttccgccag caggacgatg atgaagatga tggatgccca tctcaccgat
  541 tcgatccgag caacatggat gtataccaaa tagagctgga ggaacaggca caaatccgct
  601 ccaaactgct ggtcgaaacc tgtgtgaagc actcgtcttc ggagcagcag cagctccaag
  661 ttaagcagga ggacctcatc aaggatttca ctcgggacga ggaggaacag ccaagcgaag
  721 aggaggcgga ggaagaggac aacgaagagg acgaggaaga agaaggcgaa gaagaagagg
  781 aggacgagga cgaggaagcc ctgctgccgg tagtcaattt taatgcaaat tcagacttta
  841 atttgcattt ctttgacaca ccggaggact cgtccaccca aggggcctac agtgaggcca
  901 atagcttgga atccgagcag gaagaggaga agcaaacaca gcagcatcag cagcagaagc
  961 agcatcaccg ggatttggag gattgcctaa gtgccattga agctgatcca ttgcagttgt
1021 tgcattgcga cgacttctat agaacatcag ccctagcaga gagtgttgca gccagtctaa
1081 gcccacagca gcagcagcaa cggcagcaca cccaccagca acaacagcaa cagcagcagc
1141 agcagcaaca ccctggacag cagcaacatc agctcaactg cacgctgagc aatggtggag
1201 gtgctttgta caccatcagc agtgtgcatc agttcggtcc ggccagcaac caeaacacca
1261 gcagcagctc cccctcctcc agcgccgccc actcttcgcc ggacagcggc tgctcgtcgg
1321 cctcctcctc cggatcttcg cgatcctgcg gatcctcctc tgcatcctcc tcctcgtcag
1381 cggtcagcag caccatcagc agcggccgca gcagcaacaa cagcgtcgtc aaccccgcag
1441 caacatcttc atctgttgcg catctgaaca aagagcaaca gcagcagcca ctgccgacga
1501 cacagctgca acagcagcag cagcaccagc agcagttgca acacccgcag cagcagcaat
1561 cttttggcct agcagacagc agcagcagca acggcagcag caacaacaac aacggtgtct
1621 cctcgaaatc atttgtgccc tgcaaagtct gtggcgacaa ggcatcggga taccactatg
1681 gtgtaacctc ctgcgagggt tgcaagggat tctttcgtcg cagtatccag aagcaaatcg
1741 aatatcgctg tttgcgggac ggcaagtgcc tggtcatcag actgaaccgc aatcgctgcc
1801 agtactgccg cttcaagaaa tgcctttccg ctggcatgag ccgcgattcc gtacgttatg
1861 gtcgcgttcc caagcgttcc cgtgagctga acggagcggc cgcctcctcc gccgccgctg
1921 gagctcctgc ctccctcaat gtggatgact ctaccagcag cacactgcac ccgagtcacc
1981 tacagcagca gcagcaacag catctactac agcagcaaca gcagcagcaa catcagccac
2041 agctgcagca acaccaccaa ctgcaacagc agccgcatgt aagcggcgta cgtgtgaaga
2101 ccccgagtac tccacaaacg ccacaaatgt gttcgatcgc ctcctcgcca tcggagctgg
2161 gcggttgcaa tagtgccaat aacaataaca ataataacaa caacagtagc agcggtaatg
2221 ccagcggtgg cagcggcgtg agcgtcggcg ttgttgttgt gggcggacac cagcaactgg
2281 tgggaggcag catggtggga atggcgggca tgggcacgga tgcccaccag gtgggcatgt
2341 gtcacgacgg cttggcggga acggcaaacg agctgaccgt ctacgatgtc atcatgtgcg
2401 tgtcgcaggc gcaccgcctc aactgctcct acacggagga actgaccaga gagctcatgc
2461 gtcgtcccgt gacggtgcca caaaatggga ttgccagcac agtggccgag agtctggagt
2521 tccagaagat ctggctgtgg caacagttct cggccagggt gacgcctggc gttcagcgga
2581 ttgtggagtt tgcgaaacgc gtacctggct tctgtgattt cacccaagat gaccagctta
2641 tactaataaa gctgggcttc ttcgaggtct ggttgaccca tgtggcccgg ttgatcaatg
2701 aggcgacatt gacactggac gatggtgcct acctgacgcg ccagcagctt gagatactct
2761 acgattctga ctttgtcaac gccttgctga actttgccaa cacgctgaac gcctacgggc
2821 tgagtgacac cgaaatcgga ctcttctcgg ccatggtgct gcttgcctcg gatcgagctg
2881 gactcagcga gcccaaggtg atcggcaggg ccagggaact ggtggccgag gcgctgcgcg
2941 tacagatcct gcgttcgcgg gcaggatccc cacaggcgct gcagctgatg ccggcgctgg
3001 aagccaagat acccgagctg agatccttgg gggccaagca cttctcacac ctagactggc
3061 tacggatgaa ctggaccaag ctgcgcctgc cgcccctctt cgccgagatc ttcgacatcc
3121 cgaaggctga cgatgagctg taggatgtgg agccaacccc gcgattccag ggccgtgcaa
3181 agcaaaccgc aacaagaaca gaatattcta ccacttgtag gcttaagcaa cgtagctata
3241 gatcgaaatg ggagggccgc agatcagata cacgtctact cagcattacc ggagagatag
3301 tccactaagc ctatatgcat actactatac tagcagtgtt a
33. SEQ ID NO: 33
Accession No. NM_165465 Drosophila melanogaster
Ecdysone receptor CG1765-PB (EcR)
MKRRWSNNGGFMRLPEESSSEVTSSSNGLVLPSGVNMSPSSLDSHDYCDQ
DLWLCGNESGSFGGSNGHGLSQQQQSVITLAMHGCSSTLPAQTTIIPING
NANGNGGSTNGQYVPGATNLGALANGMLNGGFNGMQQQIQNGHGLINSTT
PSTPTTPLHLQQNLGGAGGGGIGGMGILHHANGTPNGLIGVVGGGGGVGL
GVGGGGVGGLGMQHTPRSDSVNSISSGRDDLSPSSSLNGYSANESCDAKK
SKKGPAPRVQEELCLVCGDRASGYHYNALTCEGCKGFFRRSVTKSAVYCC
KFGRACEMDMYCQECRLKKCLAVGMRPECVVPENQCAMKRREKAQKEKDK
MTTSPSSQHGGNGSLASGGGQDFVKKEILDLMTCEPPQHATIPLLPDEIL
AKCQARNIPSLTYNQLAVIYKLIWYQDGYEQPSEEDLRRIMSQPDENESQ
TDVSFRHITEITILTVQLIVEFAKGLPAFTKIPQEDQITLLKACSSEVMM
LRMARRYDHSSDSIFFANNRSYTRDSYKMAGMADNIEDLLHFCRQMFSMK
VDNVEYALLTAIVIFSDRPGLEKAQLVEAIQSYYIDTLRIYILNRHCGDS
MSLVFYAKLLSILTELRTLGNQNAEMCFSLKLKRKLPKFLEEIWDVHAIP
PSVQSHLQITQEENERLERAERMRASVGGAITAGIDCDSASTSAAAAAAQ
HQPQPQPQPQPSSLTQNDSQHQTQPQLQPQLPPQLQGQLQPQLQPQLQTQ
LQPQIQPQPQLLPVSAPVPASVTAPGSLSAVSTSSEYMGGSAAIGPITPA
TTSSITAAVTASSTSAVPMGNGVGVGVGVGGGNVSMYANAQTAMALMGVA
LHSHQEQLIGGVAVKSEHSTTA
34. SEQ ID NO: 34
Accession No. NM_165465 Drosophila melanogaster
Ecdysone receptor CG1765-PB (EcR)
      1 tagtattttt ttggactttg ttgttaacgg ttgttcgctc gcacgtacga agcccgatcg
    61 cgttcgtcaa aaaacaagat acaaaataca gcacacacaa ttgaaaacga caacctaaca
  121 gtacggtttc ccaaagcacc ttacatttca aaaccgaaaa cccccaaaat gttgtaacca
  181 aataatgttt aaatcacata tacacctaca tatatttatg aaaaattgtt agacaaatcc
  241 caaataatac cagttccccc aacaaccgca acaaacacaa gtgcaattca tcggcaaaaa
  301 ttaatataaa gtgcaaatgc attgtagctg aaactcaaac aatagtaaaa atacatacat
  361 aagtggtgaa gaagcaaaag gaaatagttc ttaaaataac gcaaatcgag agcatatatt
  421 catatttgta cagatattat atggcggctg catagtgcaa actgcggctg agggaataca
  481 gcggtatcga aatgtaaata ggaaacaacg aagccagaac tcgaaatcaa acatcagcaa
  541 cgtgacacac agacataaga cgcccgtcta gtcgtggtct gtggaacgct agctccgctt
  601 tgccaggagc cggagacttt ttccgcatcc acaatattac atatgtacat atatcgaaga
  661 tagtgcgcga gtgagtgagg gatttgtgcc gtggatcccg atccccttac atatatataa
  721 aggtagtgaa aagattttac tcaacattcc aaatagtgct ttgtcaactg gaataccttt
  781 tgttcaaata cgcagtgggc ccatggatac ttgtggatta gtagcagaac tggcgcacta
  841 tatcgacgca tatgctctga ttgtttcccg cactaaatga gcagggattc gggcgaaaat
  901 gtattttgaa cgcaaacaag tgcgcaaaaa atactagctc caccacgaaa ctgcacaaaa
  961 caccgccaga agcgagcaga acctcgggcc gcacgaccga gcttcgtaaa gcaacagagg
1021 atottaccag gagatagctc ttctccacat agaccaactg ccagggacaa gctccttgtc
1081 cccagccgac gctaagtgaa cggaaaacgg ccacaaaacg gcgactatcg gctgccagag
1141 gatgaagcgg cgctggtcga acaacggcgg cttcatgcgc ctaccggagg agtcgtcctc
1201 ggaggtcacg tcctcctcga acgggctcgt cctgccctcg ggggtgaaca tgtcgccctc
1261 gtcgctggac tcgcacgact attgcgatca ggacctttgg ctctgcggca acgagtccgg
1321 ttcgtttggc ggctccaacg gccatggcct aagtcagcag cagcagagcg tcatcacgct
1381 ggccatgcac gggtgctcca gcactctgcc cgcgcagaca accatcattc cgatcaacgg
1441 caacgcgaat gggaatggag gctccaccaa tggccaatat gtgccgggtg ccactaatct
1501 gggagcgttg gccaacggga tgctcaatgg gggcttcaat ggaatgcagc aacagattca
1561 gaatggccac ggcctcatca actccacaac gccctcaacg ccgaccaccc cgctccacct
1621 tcagcagaac ctggggggcg cgggcggcgg cggtatcggg ggaatgggta ttcttcacca
1681 cgcgaatggc accccaaatg gccttatcgg agttgtggga ggcggcggcg gagtaggtct
1741 tggagtaggc ggaggcggag tgggaggcct gggaatgcag cacacacccc gaagcgattc
1801 ggtgaattct atatcttcag gtcgcgatga tctctcgcct tcgagcagct tgaacggata
1861 ctcggcgaac gaaagctgcg atgcgaagaa gagcaagaag ggacctgcgc cacgggtgca
1921 agaggagctg tgcctggttt gcggcgacag ggcctccggc taccactaca acgccctCaC
1981 ctgtgagggc tgcaaggggt tctttcgacg cagcgttacg aagagcgccg tctactgctg
2041 caagttcggg cgcgcctgcg aaatggacat gtacatgagg cgaaagtgtc aggagtgccg
2101 cctgaaaaag tgcctggccg tgggtatgcg gccggaatgc gtcgtcccgg agaaccaatg
2161 tgcgatgaag cggcgcgaaa agaaggccca gaaggagaag gacaaaatga ccacttcgcc
2221 gagctctcag catggcggca atggcagctt ggcctctggt ggcggccaag actttgttaa
2281 gaaggagatt cttgacctta tgacatgcga gccgccccag catgccacta ttccgctact
2341 acctgatgaa atattggcca agtgtcaagc gcgcaatata ccttccttaa cgtacaatca
2401 gttggccgtt atatacaagt taatttggta ccaggatggc tatgagcagc catctgaaga
2461 ggatctcagg cgtataatga gtcaacccga tgagaacgag agccaaacgg acgtcagctt
2521 tcggcatata accgagataa ccatactcac ggtccagttg attgttgagt ttgctaaagg
2581 tctaccagcg tttacaaaga taccccagga ggaccagatc acgttactaa aggcctgctc
2641 gtcggaggtg atgatgctgc gtatggcacg acgctatgac cacagctcgg actcaatatt
2701 cttcgcgaat aatagatcat atacgcggga ttcttacaaa atggccggaa tggctgataa
2761 cattgaagac ctgctgcatt tctgccgcca aatgttctcg atgaaggtgg acaacgtcga
2821 atacgcgctt ctcactgcca ttgtgatctt ctcggaccgg ccgggcctgg agaaggccca
2881 actagtcgaa gcgatccaga gctactacat cgacacgcta cgcatttata tactcaaccg
2941 ccactgcggc gactcaatga gcctcgtctt ctacgcaaag ctgctctcga tcctcaccga
3001 gctgcgtacg ctgggcaacc agaacgccga gatg-gtttc tcactaaagc tcaaaaaccg
3061 caaactgccc aagttcctcg aggagatctg ggacgttcat gccatcccgc catcggtcca
3121 gtcgcacctt cagattacec aggaggagaa cgagcgtctc gagcgggctg agcgtatgcg
3181 ggcatcggtt gggggcgcca ttaccgccgg cattgattgc gactctgcct ccacttcggc
3241 ggcggcagcc gcggcccagc atcagcctca gcctcagccc cagccccaac cctcctccct
3301 gacccagaac gattcccagc accagacaca gccgcagcta caacctcagc taccacctca
3361 gctgcaaggt caactgcaac cccagctcca accacagctt cagacgcaac tccagccaca
3421 gattcaacca cagccacagc tccttcccgt ctccgctccc gtgcccgcct ccgtaaccgc
3481 acctggttcc ttgtccgcgg tcagtacgag cagcgaatac atgggcggaa gtgcggccat
3541 aggacccatc acgccggcaa ccaccagcag tatcacggct gccgttaccg ctagctccac
3601 cacatcagcg gtaccgatgg gcaacggagt tggagtcggt gttggggtgg gcggcaacgt
3661 cagcatgtat gcgaacgccc agacggcgat ggccttgatg ggtgtagccc tgcattcgca
3721 ccaagagcag cttatcgggg gagtggcggt taagtcggag cactcgacga ctgcatagca
3781 ggcgcagagt cagctccacc aacatcacca ccacaacatc gacgtcctgc tggagtagaa
3841 agcgcagctg aacccacaca gacatagggg aaatggggaa gttctctcca gagagttcga
3901 gccgaactaa atagtaaaaa gtgaataatt aatggacaag cgtaaaatgc agttatttag
3961 tcttaagcct gcaaatatta cctattattc atacaaatta acatataata cagcctatta
4021 acaattacgc taaagcttaa ttgaaaaagc ttcaacaaca attggacaaa cgcgttgagg
4081 aaccgggaga aaatttaaga aaaaaaaaac cattgaaaat tatgaaattt agtatacatt
4141 ttttttgggt ggatgtatgt cgcatcagac tcacgatcaa ttctcgaatt ttgttaacta
4201 aattgatcct ccaaactgca tgcgaaacag atcagaaaag agaacagaca gtagggcgtg
4261 aacagaggga agagagaaga gaataaagat tgtttatatt taaaaaatat ataaaataat
4321 aattactaac tctaaacgta atgaaagcaa ctgtataata tctaactata actataaatt
4381 cgtactgtag ggaagtgaga aaatctgtta aatgaaacaa aaataatgat aataacatta
4441 tcatccacca taattaaaat catttaaagt aattaaaaac aaaacacttt taaaacacgc
4501 aaaacttgga ctgattttat aaatattttt taatcataaa gaaaggcaac ctgaaaaaaa
4561 tattacaaaa acaaataaca acatatttta ttatgacacc cttatatgtt ttcaaaacga
4621 gaatttaaat tcttagattc ttataatttc atccaaaaat attagccagc aaaaaccttt
4681 attattggca ttgtttttag acatgttttc aaaaaaaact ttgatattga aactaaacaa
4741 aggataatga aatgaaagtg attggagtct tactcaaaaa ccaaaaggca tcaaaaggta
4801 ttaaattaaa aatataatct aatttcgagt tcaagaaaca ctttttggtg gaaaatagtt
4861 ttcaatcact ttgataaaaa ccacacaaat taataaatac atgcatacac caaaagactt
4921 caatatatat ttttaaaatt tacattgata attcgaaatt tgaataagaa tcacatccat
4981 ctaatttggc taaatcaaaa tttttatgaa agccacacaa aaaacgtgca aatttgatta
5041 ctttggcaat ttttatgtta tacaaaattt atgcaattga ttttcaaaat aatttttatt
5101 agattgtatt agtttcattt tgctttggga tgtacatttt aaataaattt tactttaaat
5161 tgttggcctt attttaactt aaatcaaatt tattctaatt ttagtaaaaa aaaatgtgtt
5221 taaaattgaa aataagaaca ctgtaaaata ttaataaaaa attaaagttt aaagtgattc
5281 ttttattatg taaaaagaag acaaaaaata tcttacgtag ctttctactt gaattgtgca
5341 attttttact tttactacta atcctaattt aaatataatt tacacacacg cctacacatc
5401 cagccacata tttttaattt taagtcaacc taatttataa atatgaattt gtataatgac
5461 gaactaaaat tagcatgaca tcatggacat acttggaaat aactctatca aacgagctaa
5521 atgcattgaa gaagaaaatt cttgttaaat atagtctgca cttcgacaaa cgaaaatcag
5581 tgaatt
35. SEQ ID NO: 35
Accession No. NM_165364 Drosophila melanogaster
Hormone receptor-like in 39 CG8676-PDHr39)
MPNMSSIKAEQQSGPLGGSSGYQVPVNMCTTTVANTTTTLGSSAGGATGS
RHNVSVTNIKCELDELPSPNGNMVPVIANYVHGSLRIPLSGHSNGRESDS
EEELASIENLKVRRRTAADKNGPRPMSWEGELSDTEVNGGEELMEMEPTI
KSEVVPAVAPPQPVCALQPIKTELENIAGEMQIQEKCYPQSNTQHHAATK
VAPTQSDPINLKFEPPLGDNSPLLAARSKSSSGGHLPLPTNPSPDSAIHS
VYTHSSPSQSPLTSRHAPYTPSLSRNNSDASHSSCYSYSSEFSPTHSPIQ
ARHAPPAGTLYGNHHGIYRQMKVEASSTVPSSGQEAQNLSMDSASSNLDT
VGLGSSHPASPAGISRQQLINSPCPICGDKISGFHYGIFSCESCKGFFKR
TVQNRKNYVCVRGGPCQVSISTRKKCPACRFEKCLQKGMKLEAIREDRTR
GGRSTYQCSYTLPNSMLSPLLSPDQAAAAAAAAAVASQQQPHQRLHQLNG
FGGVPIPCSTSLPASPSLAGTSVKSEEMAETGKQSLRTGSVPPLLQEIMD
VEHLWQYTDAELARINQPLSAFASGSSSSSSSSGTSSGAHAQLTNPLLAS
AGLSSNGENANPDLIAHLCNVADHRLYKIVKWCKSLPLFKNISIDDQICL
LINSWCELLLFSCCFRSIDTPGEIKMSQGRKITLSQAKSNGLQTCIERML
NLTDHLRRLRVDRYEYVAMKVIVLLQSDTTELQEAVKVRECQEKALQSLQ
AYTLAHYPDTPSKFGELLLRIPDLQRTCQLGKEMLTIKTRDGADFNLLME
LLRGEH
36. SEQ ID NO: 36
Accession No. NM_165364 Drosophila melanogaster
Hormone receptor-like in 39 CG8676-PDHr39)
      1 actaacaaaa caaacatttt gctacttcgt cgcaggcggg actgtgttgc gtcgtgtgat
    61 cgctagagcg gttgtggaat cggattcgag cgcaaaacac cgttcatgct gtgagcgaaa
  121 aagagtggta gcgcctacag tggcatatgt agttaaatcc gtgaataagt gaaaaatccg
  181 atatttgtcg tgcaataatt tcctcgattg gcatcaagtg gcttccagtc gggtacatat
  241 tgcacaagaa atgttatacg cataatgtgc acgcaaatta aacgaattct ctatgaaaat
  301 gtgactagaa tgtgagtcga acaaaacgag taaaacgtga aatcccaact ggcttttggg
  361 taaeaaatct tatcaacaca gcaacggaaa tacattaaaa tcttgataga ctgagaaagg
  421 gacaattgga atacttttag ttatttttaa atgttttaca acacaatgga actgcatcaa
  481 cgacacctct caaactttta caaattgcac aactgagaaa tagtctttga taaataaata
  541 aaatataaga aatcgctact gaaacaagat gccaaacatg tccagcatca aagcggagca
  601 gcaaagcggt cctcttggag gaagtagcgg ctatcaagta ccggtcaaca tgtgcaccac
  661 cacagtcgcg aatacgacga ccactttggg aagctccgcc gggggagcca ctggctcccg
  721 gcacaacgtc tccgtgacaa acatcaagtg cgaactagac gaactaccgt caccgaacgg
  781 caacatggtg ccggttatcg caaactacgt tcacggtagc ttgcgcattc cactcagtgg
  841 acattcaaat catagggagt ccgattcgga ggaggagctg gcaagtattg agaacttgaa
  901 ggttcggcga aggacggcgg cggacaaaaa tggtcctcgt ccaatgtcct gggagggcga
  961 gctgagcgat actgaggtca acgggggcga agagctgatg gaaatggagc caacaattaa
1021 gagtgaggtg gtccctgctg ttgcaccccc acaacccgtc tgcgcactac aaccgataaa
1081 aacagagcta gagaacattg caggcgagat gcagattcaa gagaagtgtt acccccagtc
1141 caacacacaa catcacgctg ccacaaaatt aaaagtggcc ccgacgcaaa gtgatccgat
1201 caatctcaag ttcgaaccgc ctctgggaga caattctccg ctactggctg cacgtagcaa
1261 gtccagcagt ggaggccacc taccactgcc aacgaatccc agtcccgact ccgccataca
1321 ttccgtctac acgcacagct ccccctcgca gtcgcctctg acgtcgcgcc acgcccccta
1381 cactccgtct ctgagccgca acaacagcga cgcctcgcac agtagctgct acagctatag
1441 ctccgaattc agtcccacac actcgcccat tcaagcgcgt catgccccac ccgccggcac
1501 gctctatggc aaccaccatg gtatttaccg ccagatgaag gtggaagcct catccactgt
1561 gccgtccagt gggcaggagg cgcagaacct gagtatggac tctgcctcta gcaatctgga
1621 tacagtgggc ttaggatctt cgcaccccgc atctccggcg ggcatatcac gtcagcagtt
1681 gatcaactcg ccctgcccca tctgcggtga caagatcagc ggatttcatt acgggatttt
1741 ctcctgcgag tcttgcaagg gcttcttcaa gcgcaccgtg caaaatcgca agaactacgt
1801 gtgcgtgcgt ggtggaccat gtcaggtcag catttccacg cgcaagaaat gtccagcctg
1861 ccgcttcgag aagtgtctgc agaagggaat gaaactagaa gcgattcggg aggaccgaac
1921 ccgtggcggc cgctccacat accagtgctc ctacacgctg cccaactcaa tgcttagtcc
1981 gctgcttagt cctgatcaag cggcagcagc tgccgccgca gcagcagtgg caagtcagca
2041 gcagccgcac cagcgactac atcaactaaa tggatttgga ggtgtaccca ttccctgctc
2101 tacttctctt ccagccagcc ctagtttggc aggaacttcg gtcaagtcgg aagagatggc
2161 ggagacgggc aagcaaagcc tccgaacggg aagcgtacca ccactactgc aggaaatcat
2221 ggatgtagag catctgtggc agtacaccga tgcagagctg gcccgcatca accaaccact
2281 gtccgcattc gcctctggca gctcttcgtc gtcgtcatcg tcaggtacat cctcaggcgc
2341 ccatgcacaa ctcaccaatc cactactggc tagtgctggt ctctcgtcca atggcgagaa
2401 tgccaatcct gatcttatcg ctcatctctg caacgtggct gatcaccgtc tttataaaat
2461 cgtcaaatgg tgcaagagct tgccgctttt taagaacatt tcgatcgatg accaaatctg
2521 cttgctcatt aactcgtggt gcgagctgtt gctcttctcc tgctgtttta gatcaattga
2581 tactcctgga gagattaaaa tgtcacaagg caggaagata accctatcgc aggccaaatc
2641 aaatggcttg cagacttgca ttgaacggat gctcaaccta acagatcacc tgaggcgatt
2701 gcgcgttgat cgctacgaat atgttgccat gaaagttatt gtgctgttgc agtcagatac
2761 gacagagtta caggaagcgg taaaggtgcg cgagtgtcag gaaaaagctt tgcagagctt
2821 gcaagcttac accctggcgc attatcctga cacgccatcc aagtttgggg agcttttgct
2881 acgcattcct gatttgcagc gaacgtgcca gcttggcaag gagatgttga cgatcaagac
2941 tcgcgatgga gctgatttca atttgctaat ggagcttttg cgcggagagc attgacaatt
3001 gataactaag acggaaatct tttaccattg gcaaaacaag tttcacatat ttagtattag
3061 atatatatat tctatagata agatccttac tgtaagttct gaaaacatgt gcctaaaaac
3121 caaagccacg atagcagtca catcaggccc actggtcgag attaaatcca agagcaagat
3181 tgccaaattt ttacaccaat atatattttg atatgagcca tgtgcagggc ctcagatcgc
3241 tgttgttgtc ggctaaagtt tcagtaagaa aagtatatat tgattttgct atttatacat
3301 atttgactta tgtatagtgt aaactaaagc acacatggaa aatgaaaaga ctaaacaaat
3361 ttatttaaag attactttta ctattataga aaaaggggaa aaataaaaaa cacaaaggca
3421 gagaagaaaa tttagttaca acaggtagcg acatttttat attttcttat ataaggaaat
3481 attcaatgta ttttaaatat aaagccaaac ccgatttggt ttgggaaaga gctactgaaa
3541 tttttgatat ctatatattc atcactagaa gacgaatgaa tgtatccaat gtttaaatgt
3601 tgtagcgttt agttttagtg caatttcaca catgtctaca tacatgaata ttcagcgaga
3661 tatgtttgca aactattata aagcaaaaga ccactcgaaa tcgccatcac tgggttggct
3721 aagactattc cagttatgct gtttgttgca taaaaaacca caactacgta catcaataaa
3781 atgtataatt ttttattgga gttttagatt tgtattaact tcttccttat aattacgatt
3841 attattatta ttactaattt tatgaatatt gtgtaacact gacttaaata gctgaaaaaa
3901 tcctgcaaca ggatttaaaa cacctgaata cacaaaacat tataacatga atacattttg
3961 cttatggcct agatagtttg atatgtactt tgcatatgta tgcatgtgtc tatatgtgag
4021 tacgtaccat acaaattcct gtcccaccag aaaaatcaca cgcaataaaa aattccaaaa
4081 tactaagctc gtatctacaa agaaagatta aaagacaaat tgatgaatag gaatatgttg
4141 ccggaagtcc aagagatttg gctgaaagta tcgacaaatt ttcaacacat cgttcatgga
4201 tattgtgcta acactctcag tttgaaaatc attttctgtt aaactttcta tataataagt
4261 tctccattcg attttgtatt tacaatttgt ttctttaatt ttcctttatc agttgtatct
4321 atgaaacatg aggatctcag ttcatattga tcgtgttctt ctgccgtaca ccgcttctgt
4381 ccgttaatgt aaaccataag tataaatgaa attagttaaa tgtttattta taaataaagc
4441 gctataataa atttcaatac atttatcata gttaactgat taagaccact gaaatcaaaa
4501 atattttatt tactaagcaa agcacacgca aacaatttat aatgtttatt acgttaacaa
4561 caaactcatt tttaataatt ctttatgaat acacaaagtt acgcaatttt ccctctaggc
4621 gcattgctta aatagttaaa gaaaaataat aaacccatag cgcaatattt aatgtaaaac
4681 agttttcctt gcgtgtgatg tttgctctag ctacgtacaa attcatcatt tattaaattt
4741 aaaactcaat tttgctttta aataaattta ataagtaaaa ttcaacaata attgatatac
4801 aattgtcaat gcaatatttt gtaataaaaa tgcgaaaaat c
37. >SEQ ID NO:37
96_ [symbol] _Ex4_7.55_kb + oligos_Map.seq
GGGCCCCCCCTCGAGGTCGACGGTATCGATAAGCTTGCCGGTGGCGGAGA
AGGTGTATCCGTGATTAAGAAAGAGCCAGCCGATGAGAAGCAGCCACAGC
CACATGACCACGGTGCGTCCGTACACAAGATGGCACTGCTGGTGCCGTTT
CGAGACCGATTTGAGGAACTCCTCCAGTTCGTCCCCCACATGACCGCCTT
TTTGAAGCGGCAGGGCGTGGCGCACCACATCTTTGTGCTGAACCAGGTGG
ACAGGTTCCGCTTCAATCGCGCCTCTCTCATCAACGTGGGTTTCCAGTTT
GCCAGCGATGTGTACGATTACATTGCCATGCACGACGTAGACTTGCTGCC
CTTGAATGACAATCTGCTCTATGATGATCCCAGCAGCTTGGGACCACTGC
ACATCGCCGGACCGAAGCTACATCCCAAATACCACTATGATAACTTCGTT
GGAGGAATATTACTGGTGCGACGCGAGCACTTTAAGCAGATGAACGGCAT
GTCGAACCAGTACTGGGGCTGGGGATTAGAGGACGACGAGTTCTTCGTGC
GCATCCGGGATGCAGGACTGCAGGTGACGCGGCCGCAGAACATTAAGACT
GGCACTAATGATACATTCAGGTGAGACCAGTGCTCCGGATTTCGCAACTA
GACGTGACTACTAATAATTATTGTCATTCAACCTCAGCCATATTCACAAC
CGCTATCATCGTAAGCGGGACACCCAGAAGTGCTTCAACCAGAAGGAGAT
GACCCGCAAGCGGGACCACAAGACGGGCCTGGACAACGTGAAGTACAAAA
TACTTAAGGTGCATGAGATGCTCATTGACCAGGTGCCGGTGACCATCCTC
AACATTTTGCTCGATTGTGATGTTAATAAAACGCCTTGGTGCGACGTCTC
CGGAACGGCAGCGGCTGCATCGGCGGTACAAACCTGATGGGTTGTGTTAA
ACCAAAGATCCTATGTTTATTTCGCTATTATAGTGTGTTGTATTGTATAA
ATGCGCTAATACACGTGCACCATGCCATAGAGGAATGTCCAGAAGAGCAC
GTAGGTGCAAAGGCCGCCCATGAACTGATTGGPCAGCAGATTTCTGCGGT
TAATGAAAAACTTGCGCCACTGGGTGCCCGATTTCACGAGCACCAGAATC
CAGAGCACGAACACGGACAGGAAGTAGAAAAGGAATCCCAGCGTACCACT
CAGGCCCAAAATACCTGCGAATTGGTGGGACATTAACTAAGTTGGTTCAC
CATCAATTGGAGCCAATTACCCGCAGCGCAGCCCGAGATGGCAGCCATCG
ATGTGCGACAGTATTCCACGGCGGATATGTTGTTCCGGATGGCGCCCTCG
CTGTAGGCGATTATTTCGCCAGTCTTGGACTGCGTGGTCTTCACTCGATT
CATTTTATTTAATTAAATTCTACTTTAATTTCTAGCAAAAATATTCCTAG
GCTGTGAACTTCGATTGTGTGCCGATTGTGTTATCGATTGGTGCCGATAA
CTATGCACTGTAAAAATTCACTAGCGGTTTTTGCAGGATAAATAGTTTTT
GTAAATTTTCCGAGATAAACTTGACGAGCTGTTTAATGTTAAATAATGAA
GTTTAATACAATATCAAATATATTTGCTGAAGTGTATATTTATTCTCACC
GCTCTGTGCTTCGATGGCTCACAATTGCGTTTGCCATTCGCCCGGGCACG
TAGATTGTTGTTATTGGGATTGGCCTGGAGCACTCGGACGGACAGTAATT
CATTAAAATATGTGGTGATAACGCGAGCTGCCGAATCTGCGTGCAATTCG
TGCGTTTGACGTGGGTACTAACTGCTATGCTGTCGCGCGGACAGTTGTTC
TGATACGCAGAGTTCCTGCCTCACCACACACGACCACCTCCATTAAAACC
AGCCACCCCCCCCAGCGCCTCCTCCACCGACAGCAGCTGCTCCACCGCAC
CACCAGGAGAGGGGCAATTAAAAAATCAATCAGAGGGCCCATCACTTGCT
TGTAACCGCCGAAGAACTGCGCGGTGTGCGGGGACAAGGCTCTGGGCTAC
AACTTCAATGCGGTCACCTGCGAGAGCTGCAAGGCGTTCTTCCGACGGAA
CGCGCTGGCCAAGAAGCAGTTCACCTGCCCCTTCAACCAAAACTGCGACA
TCACTGTGGTCACTCGACGCTTCTGCCAGAAATGCCGCCTGCGCAAGTGC
CTGGATATCGGGATGAAGAGTGAAAACATTATGTCCGAGGAGGACAAGCT
GATCAAGCGGCGCAAGATCGAGACCAACCGGGCCAAGCGACGCCTCATGG
AGAACGGCACGGATGCGTGCGACGCCGATGGCGGCGAGGAAAGGGATCAC
AAAGCGCCGGCGGATAGCAGCAGCAGCAACCTTGACCACTACTCGGGGTC
ACAGGACTCGCAGAGCTGCGGCTCGGCGGACAGCGGGGCCAATGGGTGCT
CCGGCAGACAGGCCAGTTCGCCGGGCACACAGGTCAATCCGCTTCAGATG
ACGGCCGAGAAGATAGTCGACCAGATCGTATCCGACCCGGATCGAGCCTC
GCAGGCCATCAACCGGTTGATGCGCACGCAGAAAGAGGCTATATCGGTGA
TGGAGAAGGTAATCAGCTCACAAAAGGACGCCTTAAGGCTGGTGTCGCAT
TTGATCGACTATCCAGGTGGGTGCAGACAAGATTTCATCGTTTAGCCTTA
TCCGCTCACCTATGAACGACTTGAATCTTTACAGGCGACGCACTCAAGAT
CATTTCAAAGTTTATGAACTCGCCCTTTAACGCGCTGACAGGTTAGAGTT
TTAAAATTTGTGGTTTTAAACTTAATTTCACATTCCTTGTTAATTTAAAT
ACGCAGTATTCACCAAATTCATGAGCTCACCCACGGACGGCGTTGAAATT
ATCTCAAAGATAGTTGATTCGCCCGCGGACGTGGTGGAGTTCATGCAGAA
CTTGATGCACTCGCCAGAGGACGCCATCGATATAATGAACAAGTTCATGA
ATACCCCAGCGGAGGCGCTGCGCATTCTTAACCGAATCCTAAGCGGCGGA
GGAGCGAACGCAGCCCAGCAGACAGCAGACCGCAAGCCATTGCTGGACAA
GGAGCCGGCGGTGAAGCCTGCAGCGCCAGCGGAGCGAGCTGATACTGTCA
TTCAAAGCATGCTGGGCAACAGTCCGCCAATTTCGCCACATGATGCTGCC
GTGGATCTGCAGTACCACTCGCCCGGTGTCGGGGAGCAGCCCAGTACATC
GAGTAGCCACCCCTTGCCTTACATAGCCAACTCGCCGGACTTCGATCTGA
AGACCTTCATGCAGACCAACTACAACGACGAGCCCAGTCTGGACAGTGAT
TTTAGCATTAACTCAATCGAATCGGTGCTATCCGAGGTGATCCGCATTGA
GTACCAGGCCTTCAATAGCATACAACAAGCGGCATCGCGCGTAAAGGAGG
AGATGTCCTACGGCACTCAGTCTACGTACGGTGGATGCAATTCGGCTGCA
AACAATAGCCAGCCGCACCTGCAGCAACCCATCTGCGCCCCATCCACCCA
GCAGTTGGATCGCGAGCTAAACGAGGCGGAGCAAATGAAGCTGCGGGAGC
TGCGACTGGCCAGCGAGGCTCTTTATGATCCCGTGGACGAGGACCTCAGC
GCCCTGATGATGGGCGATGATCGCATTAAGGTAACCCGCTAGGGATAACA
GGGTAATAACAGTCCACGGTATTAGCCTATAGGTCTTTCTACATTTATAG
CTCCAACACCACGGCTTATCTAATCAGAGTGTGCGAGCTGCGATATATGT
ACACACGGCACCTGGCACTTTTTAGCCATTCGGTGATTCAGTGCGTCTCT
CGATGTTGGCCCACGGGCCGTATCTTCGTCAGCCAGTTTCTGGGTTCCCA
GCAATGCTCGCCTACCAAATGTAAACACACTTTTTAATGGGGTGGCTCAA
AGTTTTTGATTTCCCAAGAGCTTTGGTCGAGTAAAAGAAAATTGATCGAA
CCAGATAAGCTATTTTCCCCCAGAGGGTTAAAGAATTTGAAGTCATGCGA
CTGGGTCTAGTTAAGATATTTGATTACGAAAATTGGCCTTTAATTAAGAC
CCTAAACGTGACAAACTTCCATTCTATATACTTCTTGATGAGTATTTAAA
CAAATATGGCTATTTTCGGAACAAATCGGGCACTCATTTATATCTTTAGC
TTTATCTTTATTTTTTAAGATGTGTCCACACCTTTGATCGACCTCTAGTT
CCCCTGGAGAAATGATTTGGAATTATCCAATAATGATTCATCACTTCCAC
GAATTGTTGTCCCATTAATCGAGCCACCCTAGCTTTCATGCAATCAGAAC
GTCTGGTCTGCCAAGAAGGAGCAGACAGCGGCTTTATCAGCCTCTGGGCG
TGCCAATTGTGACACTATCAACCATTATCAAGAGTACCAGCAGGCGCTCA
TGAGTCTCCAGCCAGCCGTCGATTTGGGCGTTTATTGTCGCTTAGACTGT
TTACCGATTTTGCCTCATCGCAATTAGCACATTTCAGTATTGTTAATTGG
GAAAAACGATACAATTTTGACGAAATATATGGAGCAGCCAGGTGTTGGGC
GCTATGATAAGCAGTGCTCCGCCATTCGATTGAGTCACCTTCCAGGGAGA
AGCCTTTACGATTATGGCGATAATAATGGCCACCAAAGAGAACATGGGCA
ACATACGCACTGACCTGCTCAAGTTTGCCGAAGGCAATATCTACGAGGAG
CACCAAAAGTTCATCACAACGTTTGACGAGAAGTGGCGCATGGACGAGAA
CATAATCCTGATCATGTGTGCCATTGTCCTTTTTACCTCGGCTCGATCGC
GAGTGATACACAAAGACGTGATTAGATTGGAACAGGTGAGTAAGCACTTG
ATACCATACTGCAGTATTACTAACTTTCTTTCATTCGATAGAATTCCTAC
TATTATCTTCTGCGAAGATATCTGGAGAGTGTTTATTCTGGCTGTGAGGC
GAGAAACGCGTTTATCAAGCTAATCCAAAAGATTTCAGATGTGGAGCGTC
TGAACAAGTTCATAATTAATGTCTATTTGAATGTTAACCCATCCCAGGTG
GAGCCCTTGCTGCGTGAAATATTCGATTTGAAAAATCACTAGACAACCGA
TGCGTGTCGGGCATTTAATGCCTATGTTGATGCCCAATGATGAATGGTCA
ACAAGCTGTAGTTGTTGTTGTTGTTGATGTCTGTTTTATCTTGTCGCTTG
TAATGTTAGATTTTAATCGAATGTGATTGTTAGATTTGCATATACTGCAT
AGATTTTATATTTCTACATCAAAGAGAGCATATTTAGGATACCAAGTGCA
AAGCAACACAATCTATATGTAATGTACACCGTTTACCTAGTTTCAAATAA
ACTAGACGATAATGCAATAACTAACTTGGAAGCGTGGGTTCTGTGCAAAA
AGGAAAAAAGACAAAAAAAATAAACTGACTTTGAGAACCAGTGGTAATAA
AATGTCTCGTATTCTTTTCTACTCGAATGAATTTCGAACCCTCCAGGACA
AATTACGCAAACGAGTGATTTTGAACAACAATCCAAAATAATTTAATTCC
GAAAGTCACAAAATAAAAATTCGAAGTAGGAAAAAACAAATAAGATGTTT
GGAAACCAACGAGAGATGTGCTTCGTTAAAGCATCAACCCGGGGAAACAC
CACAGCAACCGCGCATGTGTACCCGCGACCAGTCCTCAGAAATCCACGTC
GTGTACGTATCCGCAGCCAGCGTATGTGTCCGCATCTGCCGACCCCGTCT
TACATAGTCATTTATGTATAATGTAGGTAATATAATAGCTCGAGCTCGCT
CCGCACCACCAATGTGCGTCGTGCAAGTCCATTCCAATTGTTATCCGGTC
CACTCGCCGCGCAAATCGGCTTTCAGGTTGATTCGCGGCAATCCTTGGCC
CATTGCAGAAACTCATCCAACGCGCTGACGGCCAAATTGCGAGAAAGAGC
CTTCACACGCAGATTACGATCGGTTGTAATGAGCACCAATTCCGTTTGAA
TGAAACACTTGCCATCTGCAAAAGAGTTTTAGTTAGAAATGCTATCAGGA
AGGACATTTAACGGAAGCAGCTCACCTGTGCATTGTTCGGTTTTTGCCGT
TTTCGAGACAGCCATTGCCGTTGCCAGAATCTTGTCGTCATTGGACAAAT
ATTCCTCCTCAACTAGGGCAAACACCGATGCATTGACAAACGAGCCCTTT
GTGGTAGCACATCTAGAAAAGAAATCAATAAGGTATTATTGATCAGCAGG
AAAAGCTTTCCTGAACAACTATTACTACTGATTTAAAAGTAAAATTTCAA
TACATTATCAGGAAACTTTTATCTATCTCAATAGCAACCAATGAATTAGA
CAGAATTATAAATAGCTAATCGCTAGTAAACCCTTTATCAGATATCAGTA
ATAAAGGAACTATGAGCTGACGCGCGGAATATAATTAACAATAGCTTACT
TCACATTGCCTTTGGCCGACTTGATGAACTCTAACGACTTTTTGGCCCGC
GACGACACCTCGTCAAAGTGGTGGATGCGCTGCGTCTGCTTCGAACTGCG
GTACGAGTCCAACTTAACGCCCTTGGAGAGGCCATCCAGTTCCTTAACCA
CTGTCAGTGGTATAATAAGTGTGTAGCGTTTAAACTCCGTGGACAGTTTT
TCAAAGTCTTCAAGGCAGTCGATAAAGCAGTTGGTATCCGGTAGAAGATA
GCGCGGTCGCACCTCGATGTATATTTTCGTGTCCACGAACTTGAGAATGT
CCTCCAACTTGCTGGTGCATATCTGCTTAACCCTAGCCTTGGCCTCAAGC
TCTTTCTTCAGCTTGCACAGCTCGGAAACATCGGAATCAGTTGAACGACA
CAACAATTCCTCAACGGCTTTACATTGAATCTTTGAAACGTTCGCCGCAA
GACCACAACTTTCAGCATCATAGTTTTCCAATGCGTTCTTCATCGCTGCG
TTTAGGTGCTCGACAGTGAAGCTTGATTCCAACGGCTGCTGGAGAGCCTC
TTGATGCTGGACGTAGTATTCCTGGAACTGACCAATCCTACGAACACGTT
CGAAAAACTGAAGACGTTCGGATCCTCTTTTGAGATAGTCCATGCTCAGC
GTTTCACGACCCAACGGAGTGAAACCTCTAAGGGCCACATCCTCATCCAA
CAGTATTTCGTTTTTCTCAAGTTTGTGCTTCCCCATTAAGCATTCAATGT
ACTCAAAAAGGATTGTTAGCTCAGCCCAGCAATCGATGAAAGAGTGTCTG
CAATATTGGAGTTAATGAAAAACTAATAAAAGGCATTCAATTTATACATA
CTCTTCCGATCTGACGGGTTCCCACACGTCCAAACTGATGCTCAACCAAC
GTACATAAACATTCACGAACTGTAGGTATGTGTTCACAGTCGCAAAGCTG
AAATAAAAGATTAATTAGCAATAATAAATAAACAAGGCGAATTTTAGCTT
ACTCTTCTTCTGGGAGACACTGGACATTTGTAGAATCCTCTAGATCTACT
AGTCC
38. SEQ ID NO: 38
>GAL4-DHR96_DNA
GAAGCAAGCCTCtaGAAAGATGAAGCTACTGTCTTCTATCGAACAAGCAT
GCGATATTTGCCGACTTAAAAAGCTCAAGTTCGcgatggcggcgaggaaa
gggatcacaaagcgccggcggatagcagcagcagcaaccttgaccactac
tcggcagaaagaggctatatcggtgatggagaaggtaatcagctcacaaa
aggacgccttaacagaggacgccatcgatataatgaacaagttcatgaat
accccagctcgcccggtgtcggggagcagcccagtacattctacgtacgg
tggatgcaatctgaagttcatcacaacgtttgacgagaagtggcgcatgg
acgagaacataatcctgatcatgtgtgccattgtcctttaatgtctattt
gaatgttaacccatcccaggtggagcccttgctgcgtgaaatattcgatc
aaagagagcatatttaggataccaagtgcaaagcaacacaatctataaga
cgataatgcaataactaacttggaagcgtgggttctgtgcaaacc
39. SEQ ID NO:39
>pET24c_Bam + Xho_filled + D171R96
TGGCGAATGGGACGCGCCCTGTAGCGGCGCATTAAGCGCGGCGGGTGTGG
TGGTTACGCGCAGCGTGACCGCTACACTTGTTAGGGTGATGGTTCTTAAT
ACAACCTATTAATTTCCCCTCGTCAAAAATAAGGTTATCAAGTGAGAAAT
CACCATGAGTGACGACTAACCGGCGCAGGAACACTGCCAGCGCATCAACA
ATATTTTCACCTGAATCAGGATATGCTTCCCATACAATCGATAGATTGTC
GCACCTGATTGCCCGACAGATCTTCTTGAGATCCTTTTTTTCTGCGCGTT
GGCGATAAGTCGTGTCTTGGTAGTGAGCGAGGAAGCGGAAGAGCGCCTGA
TGCGGTATTTTCTCCTACGCATCTGTGCGGTATTTCACACCGCAGGGAGC
TGCATGTGTCAGAGGTTTTCACCGTCATCACCGAAACGCGCGAGGCAGCT
GCGGCGATGAAACGAGAGAGGATGCTCACGATACGGGTTACTGATGATGA
AACGGAAACCGAAGACCATTCATGTTGTTGCTCAGAAGATTCCGAATACC
GCAAGCGCTCACTGTCTTCGGTATCGTCGTATCCCACTACCGAGATATCC
GCACCAACGCGCAGCCCGGACTCGGTAATGGCGCGCATTGCCGAGACAGA
ACTTAATGGGCCCGCTAACAGCGCGATTTGCTGGTGACCCAAGCGACCAG
ATCGCTTTACAGGCTTCGACGCCGCTTCGTTCTACCATCGACACCACCAC
GCTTCACCACGCGGGAAACGGTCTGATAAGAGACACCGGAAGGAAGATGG
CGCCCAACAGTCCCTCTAGAAATAAAACCTTGACCACTACTCGGGGTCAC
AGGACTCGCAGAGCTGCGGCTCGGCGGACAGCGGGGCCAATGGGTGCTCC
GGCACCTTAGGCTGGTGTCGCATTTGATCGACTATCCAGGCGACGCACTC
AAGATCATTTCAAAGTTTAGCTGCGCATTCTTAACCGAATCCTAAGCGGC
GGAGGAGCGAACGCAGCCCAGCTACATAGCCAACTCGCCGGACTTCGATC
TGAAGACCTTCAAGCAACCCATCTGCGCCCCATCCACCCAGCATTCCGTG
ACAAACTATATCCGGAT
40. SEQ ID NO: 40
F96Xma
5′-GAGAGATGTGCTTCGTTAAAGCATCAACCC
41. SEQ ID NO: 41
R96SpeBgl
5′-GGACTAGTAGATCTAGAGGATTCTACAAATGTCCAGTGTCTCCC
42. SEQ ID NO: 42
R96Int3
5′-CCATTATTATCGCCATAATCGTAAAGG
43. SEQ ID NO: 43
R96EX3SCE
44. SEQ ID NO: 44
R96endhind
5′-GGAAAGCTTTTCCTGCTGATCAATAATACC
45. SEQ ID NO: 45
FAPA96
5′-TGGGCCCATCACTTGCTTGTAACCGCCGAAGAACTGCGCGG
46. SEQ ID NO: 46
F96INT3SCE
5′CGCTAGGGATAACAGGGTAATAACAGTCCACGGTATTAGCCTATAGG
47. SEQ ID NO: 47
F96EX5Int3
5′CGATTATGGCGATAATAATGGCCAAAGAGAACATGGGCAACATACGC
48. SEQ ID NO: 48
FGALXB
5′-GAAGCAAGCCTCTAGAAAGATGAAGC
49. SEQ ID NO: 49
RGAL96
5′-CGTGCCGTTCTCCATCGATACAGTCAACTGTCTTTGACC
50. SEQ ID NO: 50
R96/936
5′-GCCTGGATAGTCGATCAAATGCG
51. SEQ ID NO: 51
F96BEG
5′-ATGGAGAACGGCACGGATGC
52. SEQ ID NO: 52
F96XBAi
5′-TACATTCTAGAGACCAACTACAACGACGAGCCCAGTCTGG
53. SEQ ID NO: 53
R96BspE1
5′-CATTCATCCGGACATTAATTATGAACTTGTTCAGACGCTCC
54. SEQ ID NO: 54
R96BspE2
5′-GGGCATCAACTCCGGAATTAAATGCCCGACACGCATCGG
55. SEQ ID NO: 55
RPAXCRE-AN
5′-GRCTCACGACGTTTGAACCCAGAAATCGAGCTCGCCCGGGG
56. SEQ ID NO: 56
RPAXCRECO
5′- CACGAATTCCAAACTGTCTCACGACGTTTTGAACCC
57. SEQ ID NO: 57
FPAXFSE-AN
5′-GAGAGCTAGCATGCCGGCTAGATCTCGAGATCGGCCGGCCTAGG
58. SEQ ID NO: 58
FPAXPOLY
5′-GAACTGCAGCTCGAGAGCTAGCATGCCGGC
59. SEQ ID NO: 59
F96ANhe
5′-GGAGATATACATATGGCTAGCATGACTGGTGG
60. SEQ ID NO: 60
R96AHind
5′-TGCTCGAAGCTTCGCAGAAGATAATAGTAGG
```

What is claimed is:

1. A composition comprising an inhibitor of DHR96 activity.

2. A composition comprising an inhibitor of DHR96 activity and a pesticide.

3. The composition of claim 2, wherein the pesticide is selected from the group comprising tebufenozide, DDT, and phenobarbital.

4. An insect comprising a gene, wherein the gene comprises a non-naturally occurring mutation of the DHR96 gene.

5. The insect of claim 4, wherein the mutant has a defect in activation with retention of dimerization ability of DHR96.

6. The insect of claim 4, wherein the mutant has a defect in activation without retention of dimerization ability of DHR96.

7. The insect of claim 4, wherein the insect fails to modulate genes in the xenobiotic pathway.

8. The method of claim 7, wherein the gene is in the cytochrome P450 family.

9. The method of claim 7, wherein the gene is in the carboxylesterases family.

10. The method of claim 7, wherein the gene is in the glutathione S-transferases family.

11. The method of claim 7, wherein the gene is in the UDP-glucoronosyltransferase family.

12. A method of enhancing the effect a pesticide has on an insect comprising administering to the insect an inhibitor of DHR96 activity.

13. The method of claim 12, wherein the pesticide and the inhibitor of DHR96 activity are administered simultaneously.

14. The method of claim 12, wherein the inhibitor of DHR96 activity is administered before the pesticide.

15. The method of claim 12, wherein the pesticide is selected from the group comprising tebufenozide, DDT, or phenobarbital.

16. A method of identifying an inhibitor of DHR96 activity, comprising the steps of:
a. testing compounds for inhibition activity of DR96 and/or inhibition of xenobiotic activity; and
b. comparing the activity of these compounds to known inhibitors of
DHR96.

17. A method of identifying ligands for DHR96, comprising the steps of:
a. creating a fusion product comprising a DNA binding domain, a DHR96 ligand binding domain (LBD), and a reporter gene;
b. expressing the fusion protein of step a, wherein the fusion protein is expressed in the presence of an appropriate ligand; and
c. detecting reporter gene product, wherein said reporter gene product indicates the presence of a ligand that binds DHR96.

18. A method of manufacturing a composition for inhibiting DHR96 activity, comprising admixing the inhibitor with a pesticide.

19. A composition produced by the method of claim 19.
